SEQUENCE LISTING (610 sequences)

SEQ ID NO: 1 - protein, 54 aa, Triticum aestivum
SCCRSTLGRNCYNLCRARGAQKLCAGVCRCKISSGLSCPKGFPKLALESNSDEP

SEQ ID NO: 2 - protein, 56 aa, Triticum aestivum
ESNSDEPDTIEYCNLGCRSSVCDYMVNAAADDEEMKLYVENCGDACVSFCNGDAGL

SEQ ID NO: 3 - protein, 56 aa, Triticum aestivum
SCCRSTLGRNCYNLCRARGAQKLCAGVCRCKISSGLSCPKGFPKLALESNSDEPDT

SEQ ID NO: 4 - protein, 56 aa, Triticum aestivum
SCCRTTLGRNCYNLCRSRGAQKLCSTVCRCKLTSGLSCPKGFPKLALESNSDEPDT

SEQ ID NO: 5 - protein, 56 aa, Triticum aestivum
SFQQPLQQYPLGQGSFRPSQQNPQAQGSVQPQQLPQFEEIRNLALQTLPAMCNVYI

SEQ ID NO: 6 - protein, 56 aa, Triticum aestivum
YPLGQGSFRPSQQNPQAQGSVQPQQLPQFEEIRNLALQTLPAMCNVYIPPYCTIAP

SEQ ID NO: 7 - protein, 56 aa, Triticum aestivum
QQYPSGQGSFQPSQQNPQAQGSVQPQQLPQFEEIRNLALETLPAMCNVYIPPYCTI

SEQ ID NO: 8 - protein, 56 aa, Triticum aestivum
QQQQQQQPSSQVSYQQPQQQYPSGQGSFQPSQQNPQAQGFVQPQQLPQFEEIRNLA

SEQ ID NO: 9 - protein, 56 aa, Triticum aestivum
PQQPISQQQAQLQQQQQQQQQQILQQILQQQLIPCRDVVLQQPNIAHASSQVSQQS

SEQ ID NO: 10 - protein, 56 aa, Triticum aestivum
HAIILHHQQQQQQQQQQQQQQQQQQQQQQQQQQQQPSSQVSYQQPQQQYPSGQGSF

SEQ ID NO: 11 - protein, 56 aa, Triticum aestivum
VQWPQQQPFPQPQQPFCEQPQRTIPQPHQTFHHQPQQTFPQPEQTYPHQPQQQFPQ

SEQ ID NO: 12 - protein, 56 aa, Triticum aestivum
QQEQRQGVQIRRPLFQLVQGQGIIQPQQPAQLEVIRSLALRTLPTMCNVYVSPDCS

SEQ ID NO: 13 - protein, 56 aa, Triticum aestivum
LQQPQQPFPQPQQQLPQPQQPQQSFPQQQRSFIQPSLQQQLNPCKNILLQQCKPAS

SEQ ID NO: 14 - protein, 56 aa, Triticum aestivum
PPFSQQLPPFSRQQQPVLPQQPPFSQQQLPPFSQQQQPVLLQQQIPFVHPSILQQL

SEQ ID NO: 15 - protein, 56 aa, Triticum aestivum
QCVSQPQQQSQQQLGQQPQQQQLAQGTFLQPHQIAQLEVMTSIALRTLPTMCNVNV

SEQ ID NO: 16 - protein, 55 aa, Triticum aestivum
QLAQGTFLQPHQIAQLEVMTSIALRTLPTMCNVNVSLYRTTTRVPFGVGTGVGGY

SEQ ID NO: 17 - protein, 56 aa, Triticum aestivum
QPGQGQQPGQWQQSGQGQHWYYPTSPQLSGQGQRPGQWLQPGQGQQGYYPTSPQQP

SEQ ID NO: 18 - protein, 56 aa, Triticum aestivum
SGQGQRPGQWLQPGQGQQGYYPTSPQQPGQGQQLGQWLQPGQGQQGYYPTSLQQTG

SEQ ID NO: 19 - protein, 56 aa, Triticum aestivum
WQQSGQGQHGYYPTSPQLSGQGQRPGQWLQPGQGQQGYYPTSPQQSGQGQQLGQWL

SEQ ID NO: 20 - protein, 56 aa, Triticum aestivum
QQQQPPFSQHQQPVLPQQQIPSVQPSILQQLNPCKLFLQQQCSPVAMPQSLARSQT

SEQ ID NO: 21 - protein, 56 aa, Triticum aestivum
FSQQQLPPFSQQLPPFSQQQQPVLLQQQIPFVHPSILQQLNPCKVFLQQQCSPVAM

SEQ ID NO: 22 - protein, 56 aa, Triticum aestivum
QLPPFSQQQQPVLPQQPPFSQQQLPPFSQQLPPFSQQQLPPFSQQLPPFSQQQQQV

SEQ ID NO: 23 - protein, 56 aa, Triticum aestivum
IPPATRTNNSPATATTIPPAPQQRFPHTRQKFPRNPNNHSLCSTHHFPAQQPFPQQ

SEQ ID NO: 24 - protein, 56 aa, Triticum aestivum
MSRLLSPSDQQLQSPQQQFPEEQSYPQQPYPQQAFPIPQQYSPHQPQQPFPQPQRP

SEQ ID NO: 25 - protein, 56 aa, Triticum aestivum
QQPFPLQPQQQFPEQSEQIISQQRQQPFSLQPQQPFSQPQQPLSQQPGQIIPQQPQ

SEQ ID NO: 26 - protein, 56 aa, Triticum aestivum
WEPQHPSSPEHQPTPQPQEHPVPHQKLNPCRDALLQQCSPVADMSFLRSQVVQHSS

SEQ ID NO: 27 - protein, 56 aa, Triticum aestivum
SCCKSTLGRNCYNLCRARGAQKLCANVCRCKLTSGLSCPKDFPKLVLESNSDEPDT

SEQ ID NO: 28 - protein, 53 aa, Triticum aestivum
IEYCNLGCRSSVCDYMVNAAADDEEMKLYVENCADACVSFCNGDAGLPSLDAY

SEQ ID NO: 29 - protein, 56 aa, Triticum aestivum
MKSCCRSTLGRNCYNLCRARGAQKLCAGVCRCKISSGLSCPKGFPKLALESNSDEP

SEQ ID NO: 30 - protein, 55 aa, Triticum aestivum
DTIEYCNLGCRSSVCDYMVNAAADDEEMKLYVENCADACVSFCNGDAGLPSLDAY

SEQ ID NO: 31 - protein, 55 aa, Triticum aestivum
PDTMEYCNLGCRSSLCDYIVNAAADDEEMKLYVEQCGDACVNFCNADAGLTSLDA

SEQ ID NO: 32 - protein, 56 aa, Triticum aestivum
PGSKLPEWMTSASIYSPGKPYLAKLYCCQELAEISQQCRCEALRYFIALPVPSQPV

SEQ ID NO: 33 - protein, 56 aa, Triticum aestivum
CVPGVAFRTNLLPHCRDYVLQQTCGTFTPGSKLPEWMTSASIYSPGKPYLAKLYCC

SEQ ID NO: 34 - protein, 56 aa, Triticum aestivum
CVGSQVPEAVLRDCCQQLADVNNEWCRCEDLSSMLRSVYQELGAREGKEVLPGCRK

SEQ ID NO: 35 - protein, 56 aa, Triticum aestivum
IAAEYDAWSCNSGPWMCYPGQAFQVPALPGCRPLLKLQCNGSQVPEAVLRDCCQQL

SEQ ID NO: 36 - protein, 56 aa, Triticum aestivum
EQYPSGQGSFQSSQQNPQAQGSVQPQQLPQFQEIRNLALQTLPAMCNVYIPPYCST

SEQ ID NO: 37 - protein, 56 aa, Triticum aestivum
LSQVCFQQSQQQYPSGQGSFQPSQQNPQAQGSVQPQQLPQFEEIRNLALETLPAMC

SEQ ID NO: 38 - protein, 56 aa, Triticum aestivum
LSQVSFQQPQQQYPSGQGFFQPFQQNPQAQGSFQPQQLPQFEAIRNLALQTLPAMC

SEQ ID NO: 39 - protein, 56 aa, Triticum aestivum
QVSFQPSQLNPQAQGPVQPQQLPQFAEIRNLALQTLPAMCNVYIPPHCSTTIAPFG

SEQ ID NO: 40 - protein, 56 aa, Triticum aestivum
LWQIPEQSRCQAIHNVVHAIILHQQQQQQQQQQQPLSQVSFQQPQQQYPSGQGSFQ

SEQ ID NO: 41 - protein, 56 aa, Triticum aestivum
LVQQLCCQQLWQIPEQSRCQAIHNVVHAIILHQQQQQQQQQQQQPLSQVSFQQPQQ

SEQ ID NO: 42 - protein, 56 aa, Triticum aestivum
QILQQQLIPCRDVVLQQHSIAHGSSQVLQQSTYQLVQQLCCQQLWQIPEQSRCQAI

SEQ ID NO: 43 - protein, 56 aa, Triticum aestivum
ILQQQLIPCRDVVLQQHNIAHASSQVLQQSSYQQLQQLCCQQLFQIPEQSRCQAIH

SEQ ID NO: 44 - protein, 56 aa, Triticum aestivum
QCQAIHNVVHAIILHQQQKQQQQPSSQVSFQQPLQQYPLGQGSFRPSQQNPQAQGS

SEQ ID NO: 45 - protein, 56 aa, Triticum aestivum
SQCQAIHNVVHAIILHQQQKQQQQLSSQVSFQQPQQQYPLGQGSFRPSQQNSQAQG

SEQ ID NO: 46 - protein, 56 aa, Triticum aestivum
QQQQQQPLSQVSFQQPQQQYPSGQGSFQPSQQNPQAQGSVQPQQLPQFEEIRNLAL

SEQ ID NO: 47 - protein, 56 aa, Triticum aestivum
HNVVHAIILHQQQQQQQQQQQQQQQQQPLSQVCFQQSQQQYPSGQGSFQPSQQNPQ

SEQ ID NO: 48 - protein, 56 aa, Triticum aestivum
LQQQLTPCMDVVLQQHNIARGRSQVLQQSTYQLLQELCCQHLWQIPEKLQCQAIHN

SEQ ID NO: 49 - protein, 56 aa, Triticum aestivum
QQFLGQQQPFPPQQPYPQPQPFPSQQPYLQLQPFPQPQLSYSQPQPFRPQQLYPQP

SEQ ID NO: 50 - protein, 56 aa, Triticum aestivum
LQQILQQQLIPCRDVVLQQHNIAHASSQVLQQSTYQLLQQLCCQQLLQIPEQSRCQ

SEQ ID NO: 51 - protein, 56 aa, Triticum aestivum
IHNVVHAIILHQQHHHHQQQQQQQQQQPLSQVSFQQPQQQYPSGQGFFQPFQQNPQ

SEQ ID NO: 52 - protein, 56 aa, Triticum aestivum
VVHAIILHQQQQQQQQQPLSQVCFQQSQQQYPSGQGSFQPSQQNPQAQGSVQPQQL

SEQ ID NO: 53 - protein, 56 aa, Triticum aestivum
QQLIPCMDVVLQQHNIAHGRSQVLQQSTYQLLQELCCQHLWQIPEQSQCQAIHNVV

SEQ ID NO: 54 - protein, 56 aa, Triticum aestivum
NVVHAIILHHHQQQQQQPSSQVSYQQPQEQYPSGQGSFQSSQQNPQAQGSVQPQQL

SEQ ID NO: 55 - protein, 56 aa, Triticum aestivum
QSSYQQLQQLCCQQLFQIPEQSRCQAIHNVVHAIILHHHQQQQQQPSSQVSYQQPQ

SEQ ID NO: 56 - protein, 56 aa, Triticum aestivum
HAIILHQQQKQQQQQPSSQVSFQQPLQQYPLGQGSFRPSQQNPQTQGSVQPQQLPQ

SEQ ID NO: 57 - protein, 56 aa, Triticum aestivum
IILHQQQQQQQQQQQQPLSQVSFQQPQQQYPSGQGSFQPSQQNPQAQGSVQPQQLP

SEQ ID NO: 58 - protein, 56 aa, Triticum aestivum
QQQQQQQQQQQPSSQVSFQQPQQQYPSSQVSFQPSQLNPQAQGSVQPQQLPQFAEI

SEQ ID NO: 59 - protein, 56 aa, Triticum aestivum
HAIILHQQQKQQQQPSSQFSFQQPLQQYPLGQGSFRPSQQNPQAQGSVQPQQLPQF

SEQ ID NO: 60 - protein, 56 aa, Triticum aestivum
QQQQLQQQRQQPSSQVSFQQPQQQYPSSQVSFQPSQLNPQAQGPVQPQQLPQFAEI

SEQ ID NO: 61 - protein, 56 aa, Triticum aestivum
VVHAIILHQQQQKQQQPSSQVSFQQPQQQYPLGQGSFRPSQQNPQAQGSVQPQQLP

SEQ ID NO: 62 - protein, 56 aa, Triticum aestivum
YQLLRELCCQHLWQIPEQSQCQAIHNVVHAIILHQQQKQQQQQPSSQVSFQQPLQQ

SEQ ID NO: 63 - protein, 56 aa, Triticum aestivum
STYQLLQELCCQHLWQIPEKLQCQAIHNVVHAIILHQQQQKQQQPSSQVSFQQPQQ

SEQ ID NO: 64 - protein, 56 aa, Triticum aestivum
QQQQQQQQQQKQQQQQQQQILQQILQQQLIPCRDVVLQQHSIAYGSSQVLQQSTYQ

SEQ ID NO: 65 - protein, 56 aa, Triticum aestivum
QPQYPQPQQPISQQQAQQQQQQQQILQQILQQQLIPCRDVVLQQHNIAHASSQVLQ

SEQ ID NO: 66 - protein, 56 aa, Triticum aestivum
PISQQQQQQQQQQQQQQQEQQILQQILQQQLIPCMDVVLQQHNIAHGRSQVLQQST

SEQ ID NO: 67 - protein, 56 aa, Triticum aestivum
LQPQQPISQQQAQQQQQQQQQQQQQQQILQQILQQQLIPCRDVVLQQHNIAHASSQ

SEQ ID NO: 68 - protein, 56 aa, Triticum aestivum
QPQYSQPQQPISQQQQQQQQQQQQQQQQQQILQQILQQQLIPCMDVVLQQHNIAHG

SEQ ID NO: 69 - protein, 56 aa, Triticum aestivum
QYSQPQQPISQQQQQQQQQQQQQQQILQQILQQQLIPCRDVVLQQHSIAHGSSQVL

SEQ ID NO: 70 - protein, 56 aa, Triticum aestivum
AIHNVAHAIIMHQQQQQQQEQQQQLQQQQQQQLQQQRQQPSSQVSFQQPQQQYPSS

SEQ ID NO: 71 - protein, 56 aa, Triticum aestivum
IHNVVHAIIMHQQEQQQQLQQQQQQQLQQQQQQQQQQQQPSSQVSFQQPQQQYPSS

SEQ ID NO: 72 - protein, 56 aa, Triticum aestivum
QQFLGQQQPFPPQQPYPQPQPFPSQLPYLQLQPFPQPQLPYSQPQPFRPQQPYPQP

SEQ ID NO: 73 - protein, 56 aa, Triticum aestivum
QPQNPSQQLPQEQVPLVQQQQFLGQQQPFPPQQPYPQPQFPSQLPYLQLQPFPQPQ

SEQ ID NO: 74 - protein, 56 aa, Triticum aestivum
QPQYSQPQQPISQQQQQQQQQQQQQQQQQQQQQQQILQQILQQQLIPCMDVVLQQH

SEQ ID NO: 75 - protein, 56 aa, Triticum aestivum
PYPQSQPQYSQPQQPISQQQQQQQQQQQQKQQQQQQQQILQQILQQQLIPCRDVVL

SEQ ID NO: 76 - protein, 56 aa, Triticum aestivum
FPPQQPYPQPQFPSQLPYLQLQPFPQPQLPYSQPQPFRPQQPYPQPQPQYSQPQQP

SEQ ID NO: 77 - protein, 56 aa, Triticum aestivum
QPQNPSQQQPQKQVPLVQQQQFPGQQQPFPPQQPYPQLQPFPSQQPYMQLQPFPQP

SEQ ID NO: 78 - protein, 56 aa, Triticum aestivum
MVRVPVPQLQPQNPSQQHPQEQVPLVQQQQFLGQQQSFPPQQPYPQPQPFPSQQPY

SEQ ID NO: 79 - protein, 56 aa, Triticum aestivum
QQFLGQQQSFPPQQPYPQPQPFPSQQPYLQLQPFPQPQLPYLQPQPFRPQQPYPQP

SEQ ID NO: 80 - protein, 56 aa, Triticum aestivum
LQLQPFPQPQLPYSQPQPFRPQQPYPQPQPQYSQPQQPISQQQQQQQQQQQQQQQQ

SEQ ID NO: 81 - protein, 56 aa, Triticum aestivum
FPPQQPYPQLQPFPSQQPYMQLQPFPQPQLPYPQPRLPYPQPQPFRPQQSYPQPQP

SEQ ID NO: 82 - protein, 56 aa, Triticum aestivum
QPQYSQPQQPISQQQQQQQQQQQQQQQQQQQQQQQQQQILQQILQQQLIPCMDVVL

SEQ ID NO: 83 - protein, 56 aa, Triticum aestivum
QQFPGQQQPFPPQQPYPQPQPFPSQQPYLQLQPFPQPQLPYPQPQLPYPQPQLPYP

SEQ ID NO: 84 - protein, 56 aa, Triticum aestivum
FPPQQPYPQPQPFPSQQPYLQLQPFPQPQLPYPQPQLPYPQPQLPYPQPQPFRPQQ

SEQ ID NO: 85 - protein, 56 aa, Triticum aestivum
LQLQPFPQPQLPYPQPQLPYPQPQLPYPQPQPFRPQQPYPQSQPQYSQPQQPISQQ

SEQ ID NO: 86 - protein, 56 aa, Triticum aestivum
QQFPGQQQPFPPQQPYPQPQPFPSQQPYLQLQPFPRPQLPYPQPQPFRPQQPYPQP

SEQ ID NO: 87 - protein, 56 aa, Triticum aestivum
FPPQQPYPQPQPFPSQQPYLQLQPFPQPQPFPPQLPYPQPQSFPPQQPYPQQQPQY

SEQ ID NO: 88 - protein, 56 aa, Triticum aestivum
QQPYPQPQPFPSQQPYLQLQPFPQPQPFLPQLPYPQPQSFPPQQPYPQQRPKYLQP

SEQ ID NO: 89 - protein, 56 aa, Triticum aestivum
MVRVPVPQLQPQNPSQQQPQEQVPLVQQQQFPGQQQPFPPQQPYPQPQPFPSQQPY

SEQ ID NO: 90 - protein, 56 aa, Triticum aestivum
MVRVPVPQLQPQNPSQQQPQEQVPLMQQQQQFPGQQEQFPPQQPYPHQQPFPSQQP

SEQ ID NO: 91 - protein, 56 aa, Triticum aestivum
MVRVPMPQLQPQDPSQQQPQEQVPLVQQQQFLGQQQPFPPQQPYPQPQPFPSQQPY

SEQ ID NO: 92 - protein, 56 aa, Triticum aestivum
QLPYPQPQLPYPQPQPFRPQQSYPQPQPQYSQPQQPISQQQQQQQQQQQQQILQQI

SEQ ID NO: 93 - protein, 56 aa, Triticum aestivum
QLPYPQPRLPYPQPQPFRPQQSYPQPQPQYSQPQQPISQQQQQQQQQQQQQQQILQ

SEQ ID NO: 94 - protein, 56 aa, Triticum aestivum
YPQPQPFPPQLPYPQTQPFPPQQPYPQPQPQYPQPQQPISQQQAQQQQQQQQILQQ

SEQ ID NO: 95 - protein, 56 aa, Triticum aestivum
QLPYSQPQPFRPQQPYPQPQPQYSQPQQPISQQQQQQQQQQQQQQQQQQQIIQQIL

SEQ ID NO: 96 - protein, 56 aa, Triticum aestivum
QQQFPGQQEQFPPQQPYPHQQPFPSQQPYPQPQPFPPQLPYPQTQPFPPQQPYPQP

SEQ ID NO: 97 - protein, 56 aa, Triticum aestivum
QLPYSQPQPFRPQQPYPQPQPQYSQPQQPISQQQQQQQQQQQQQQQEQQILQQILQ

SEQ ID NO: 98 - protein, 56 aa, Triticum aestivum
QLPYPQPQLPYPQPQLPYPQPQPFRPQQPYPQSQPQYSQPQQPISQQQQQQQQQQQ

SEQ ID NO: 99 - protein, 56 aa, Triticum aestivum
QPQPFRPQQPYPQSQPQYSQPQQPISQQQQQQQQQQQQKQQQQQQQQILQQILQQQ

SEQ ID NO: 100 - protein, 56 aa, Triticum aestivum
QPFPPQLPYPQPQSFPPQQPYPQQQPQYLQPQQPISQQQAQQQQQQQQQQQQQQQI

SEQ ID NO: 101 - protein, 56 aa, Triticum aestivum
VRVPVPQLQPQNPSQQQPQEQVPLVQQQQFLGQQQQHFPGQQQPFPPQQPYPQPQP

SEQ ID NO: 102 - protein, 56 aa, Triticum aestivum
FLPQLPYPQPQPFPPQQSYPQPQPQYPQPQQPISQQQAQLQQQQQQQQQQILQQIL

SEQ ID NO: 103 - protein, 56 aa, Triticum aestivum
QFLGQQQQHFPGQQQPFPPQQPYPQPQPFLPQLPYPQPQPFPPQQSYPQPQPQYPQ

SEQ ID NO: 104 - protein, 56 aa, Triticum aestivum
GVQILVPLSQQQQVGQGTLVQGQGIIQPQQPAQLEVIRSSVLQTLATMCNVYVPPY

SEQ ID NO: 105 - protein, 56 aa, Triticum aestivum
QTFPHQPQQQFPQPQQPQQSFPQQQQPLIQPYLQQQMNPCKNYLLQQCNPVSLVSS

SEQ ID NO: 106 - protein, 56 aa, Triticum aestivum
SQQPQQQFSQPQQQFPQPQQPQQSFPQQQPPFIQPSLQQQVNPCKNFLLQQCKPVS

SEQ ID NO: 107 - protein, 56 aa, Triticum aestivum
QPFPQSQQPQQPFPQPQQPQQSFPQQQQPLIQPYLQQQMNPCKNYLLQQCNPVSLV

SEQ ID NO: 108 - protein, 56 aa, Triticum aestivum
VHSIIMQQEQQQQQQQQQQQQQQQGIQIMRPLFQLVQGQGIIQPQQPAQLEVIRSL

SEQ ID NO: 109 - protein, 56 aa, Triticum aestivum
SQQPQQPFPQPQQQFPQPQQPQQSFPQQQPSLIQQSLQQQLNPCKNFLLQQCKPVS

SEQ ID NO: 110 - protein, 56 aa, Triticum aestivum
SQQPQQPFPQPQQQFLQPQQPQQSFPQQQQPLIQLSLQQQMNPCKNFLLQQCNPVS

SEQ ID NO: 111 - protein, 56 aa, Triticum aestivum
QQCCQQLAQIPQQLQCAAIHSVVHSIIMQQEQRQGVQIRRPLFQLVQGQGIIQPQQ

SEQ ID NO: 112 - protein, 56 aa, Triticum aestivum
QPQQLFPQSQQPQQQFSQPQQQFPQPQQPQQSFPQQQPPFIQPSLQQQVNPCKNFL

SEQ ID NO: 113 - protein, 56 aa, Triticum aestivum
DCQVMRQQCCQQLAQIPQQLQCAAIHSVVHSIVMQQEQQQGIQILRPLFQLVQGQG

SEQ ID NO: 114 - protein, 56 aa, Triticum aestivum
VQWPQQQPVPQPHQPFSQQPQHTFPQPQQTFPHQPQQQFPQPQQPQQQFPQPQRPQ

SEQ ID NO: 115 - protein, 56 aa, Triticum aestivum
QQPQQQFPQSQQPQQPFPQPQQQFLQPPQPQQSFPQQQQPLIQLSLQQQMNPCKNF

SEQ ID NO: 116 - protein, 56 aa, Triticum aestivum
QPFPQPQQPQQPFPQSKQPQQPFPQPQQPQQSFPQQQPSLIQQSLQQQLNPCKNFL

SEQ ID NO: 117 - protein, 56 aa, Triticum aestivum
QQPQQPFPQSQQPQQPFPQPQQQFPQPQQPQQSFPQQQPSLIQQSLQQQLNPCKNL

SEQ ID NO: 118 - protein, 56 aa, Triticum aestivum
IPQQLQCAAIHTVIHSIIMQQEQQQGMHILLPLYQQQQVGQGTLVQGQGIIQPQQP

SEQ ID NO: 119 - protein, 56 aa, Triticum aestivum
VQWPQQQPFRQPQQPFYQQPQHTFPQPQQTCPHQPQQQFPQPQQPQQPFPQPQQPQ

SEQ ID NO: 120 - protein, 56 aa, Triticum aestivum
QTFHHQPQQTFPQPQQTYPHQPQQQFPQPQQPQQSFPQQQQPAIQSFLQQQMNPCK

SEQ ID NO: 121 - protein, 56 aa, Triticum aestivum
VPRPQQQPFPQPHQPFSQQPQQTFPQPQQTFPHQPQQQFSQPQQPQQQFIQPQQPF

SEQ ID NO: 122 - protein, 56 aa, Triticum aestivum
NMQVDPSGQVPWPQQQPFPQPHQPFSQQPQQTFPQPQQTFPHQPQQQFSQPQQPQQ

SEQ ID NO: 123 - protein, 56 aa, Triticum aestivum
VQWPQQQPVLLPQQPFSQQPQQTFPQPQQTFPHQPQQQFSQPQQPQQQFIQPQQPF

SEQ ID NO: 124 - protein, 56 aa, Triticum aestivum
QPQQTFPQPQQTFPHQPQQQFSQPQQPQQQFIQPQQPQQTYPQRPQQPFPQTQQPQ

SEQ ID NO: 125 - protein, 56 aa, Triticum aestivum
NMQVDPSSQVQWPQQQPVPQPHQPFSQQPQQTFPQPQQTFPHQPQQQFPQPQQPQQ

SEQ ID NO: 126 - protein, 56 aa, Triticum aestivum
PQQTFPQPQQTFPHQPQQQFPQPQQPQQQFLQPQQPFPQQPQQPYPQQPQQPFPQT

SEQ ID NO: 127 - protein, 56 aa, Triticum aestivum
VQWPQQQPVPQPHQPFSQQPQQTFPQPQQTFPHQPQQQFPQPQQPQQQFLQPQQPF

SEQ ID NO: 128 - protein, 56 aa, Triticum aestivum
VQWPQQQPVPLPQQPFSQKPQQTFPQPQQTFPFPHQPQQQFPQPQQPQQQFLQPQQ

SEQ ID NO: 129 - protein, 56 aa, Triticum aestivum
VQWPQQQPFRQPQQPFYQQPQHTFPQPQQTFPHQPQQQFPQPQQPQQSFPQQQQPL

SEQ ID NO: 130 - protein, 56 aa, Triticum aestivum
QTFHHQPQQTFPQPQQTYPHQPQQQFPQPQQPQQPFPQPQQAQLPFPQQPQQPFPQ

SEQ ID NO: 131 - protein, 56 aa, Triticum aestivum
QTFHHQPQQTFPQPQQTYPHQPQQQFPQTQQPQQPFPQPQQTFPQQPQLPFPQQRQ

SEQ ID NO: 132 - protein, 56 aa, Triticum aestivum
TQQPQQPFPQPQQTFPQQPQLPFPQQRQQPFPQPQQPQQPFPQSQQPQQPFPQPQQ

SEQ ID NO: 133 - protein, 56 aa, Triticum aestivum
VQWPQQQPVLLPQQPFSQQPQQTFPRPQQTFPHQPQQQVPQPQQPQQPFLQPQQPF

SEQ ID NO: 134 - protein, 56 aa, Triticum aestivum
TQQPQQPFPQPQQTFPQQPQLPFPQQRQQPFPQTQQPQQLFPQSQQPQQQFSQPQQ

SEQ ID NO: 135 - protein, 56 aa, Triticum aestivum
PQQTFPQPQQTFPHQPQQQFSQPQQPQQQFIQPQQPFPQQPQQTYPQRPQQLFPQT

SEQ ID NO: 136 - protein, 56 aa, Triticum aestivum
QTFPHQPQQQFPQPQQPQQQFLQPQQPFPQQPQQPYPQQPQQPFPQTQQPQQLFPQ

SEQ ID NO: 137 - protein, 56 aa, Triticum aestivum
QPFPQPQQPQQPFPQSQQPQQPFPQPQQQFPQPQQPQQSFPQQQQPLIQPYLQQQM

SEQ ID NO: 138 - protein, 56 aa, Triticum aestivum
QTFPHQPQQQFSQPQQPQQQFIQPQQPFPQQPQQTYPQRPQQPFPQTQQPQQPFPQ

SEQ ID NO: 139 - protein, 56 aa, Triticum aestivum
PQQPQQTYPQRPQQPFPQTQQPQQPFPQSQQPQQPFPQPQQQFPQPQQPQQSFPQQ

SEQ ID NO: 140 - protein, 56 aa, Triticum aestivum
VQWPQQQPFRQPQQPFYQQPQQTFPQPQQIFPHQPQQQFPQPQQPQQQFPQPQQPQ

SEQ ID NO: 141 - protein, 56 aa, Triticum aestivum
QIFPHQPQQQFPQPQQPQQQFPQPQQPQQPFPQPQQAQLPFPQQPQQPFPQPQQPQ

SEQ ID NO: 142 - protein, 56 aa, Triticum aestivum
VQWPQQQQPFPQPQQPQQPFPQPQQPQLPFPQQPQQPFPQPQQPQQPFPQLQQPQQ

SEQ ID NO: 143 - protein, 56 aa, Triticum aestivum
VQWPQQQPFRQPQQPFYQQPQQQFPQPQQPQQPFPQPQQAQLPFPQQPQQPFPQPQ

SEQ ID NO: 144 - protein, 56 aa, Triticum aestivum
VQWPQQQPFRQPQQPFCQQPQRTIPQPHQTFHHQPQQTFPQPQQTYPHQPQQQFPQ

SEQ ID NO: 145 - protein, 56 aa, Triticum aestivum
QPFPQPQQPQQPFPQSQQPQQPFPQPQQQFPQPQQPQQSFPQQQQPAIQSFLQQQM

SEQ ID NO: 146 - protein, 56 aa, Triticum aestivum
PQQPQQPYPQQPQQPFPQPQQPQQQFPQSQQPQQPFPQPQQQFLQPQQPQQSFPQQ

SEQ ID NO: 147 - protein, 56 aa, Triticum aestivum
QTFPHQPQQQVPQPQQPQQPFLQPQQPFPQQPQQPFPQTQQPQQPFPQQPQQPFPQ

SEQ ID NO: 148 - protein, 56 aa, Triticum aestivum
PQQPQQPFPQTQQPQQPFPQQPQQPFPQTQQPQQPFPQQPQQPFPQTQQPQQPFPQ

SEQ ID NO: 149 - protein, 56 aa, Triticum aestivum
QTFPHQPQQQFPQPQQPQQQFPQPQQPQQPFPQPQQQFPQPQQPQQSFPQQQQPAI

SEQ ID NO: 150 - protein, 56 aa, Triticum aestivum
QTFPHQPQQQFSQPQQPQQQFIQPQQPFPQQPYPQQPQHPFPQTQQPQQPFPQSQQ

SEQ ID NO: 151 - protein, 56 aa, Triticum aestivum
PQQPYPQQPQHPFPQTQQPQQPFPQSQQPQQPFPQPQQQFPQPQQPQQSFPQQQPS

SEQ ID NO: 152 - protein, 56 aa, Triticum aestivum
QTFPFPHQPQQQFPQPQQPQQQFLQPQQPQQPYPQQPQQPFPQTQQPQQLFPQSQQ

SEQ ID NO: 153 - protein, 56 aa, Triticum aestivum
PQQPQQTYPQRPQQPFPQTQQPQQPFPQSQQPQQPFPQSQQPQQPFPQPQQQFPQP

SEQ ID NO: 154 - protein, 56 aa, Triticum aestivum
QPFPQPQQAQLPFPQQPQQPFPQPQQPQQPFPQSQQPQQPFPQPQQPQQSFPQQQQ

SEQ ID NO: 155 - protein, 56 aa, Triticum aestivum
PFPQQPQQPFPQPQQPQQPFPQLQQPQQPLPQPQQPQQPFPQQQQPLIQPYLQQQM

SEQ ID NO: 156 - protein, 56 aa, Triticum aestivum
VQWPQQQPFRQPQQPYPQQPQQPFPQTQQPQQLFPQSQQPQQQFSQPQQQFPQPQQ

SEQ ID NO: 157 - protein, 56 aa, Triticum aestivum
TQQPQQPFPQQPQQPFPQTQQPQQPFPQLQQPQQPFPQPQQQLPQPQQPQQSFPQQ

SEQ ID NO: 158 - protein, 56 aa, Triticum aestivum
PQQPYPQQPQQPFPQTQQPQQPFPQSKQPQQPFPQPQQPQQSFPQQQPSLIQQSLQ

SEQ ID NO: 159 - protein, 56 aa, Triticum aestivum
QFLQPQQPFPQQPQQPYPQQPQQPFPQTQQPQQQFPQSQQPQQPFPQPQQQFLQPP

SEQ ID NO: 160 - protein, 56 aa, Triticum aestivum
QFIQPQQPFPQQPQQTYPQRPQQLFPQTQQPQQPFPQSQQPQQPFPQPQQQFPQPQ

SEQ ID NO: 161 - protein, 56 aa, Triticum aestivum
PQQPQQPYPQQPQQPFPQTQQPQQLFPQSQQPQQQFSQPQQQFPQPQQPQQSFPQQ

SEQ ID NO: 162 - protein, 55 aa, Triticum aestivum
QLAQGTFLQPHQIAQLEVMTSIALRILPTMCSVNVPLYRTTTSVPFGVGTRVGAY

SEQ ID NO: 163 - protein, 56 aa, Triticum aestivum
FSQQQQPPFSQQQQPVLPQQPSFSQQQLPPFSQQLPPFSRQQQPVLPQQPPFSQQQ

SEQ ID NO: 164 - protein, 56 aa, Triticum aestivum
QQPQQLGQCVSQPQQQSQQQLGQQPQQQQLAQGTFLQPHQIAQLEVMTSIALRILP

SEQ ID NO: 165 - protein, 56 aa, Triticum aestivum
NPCMVFLQQQCSPVAMPQSLARSQMLQQSSCHVMQQQCCRQLPQIPQQSRYEAIRA

SEQ ID NO: 166 - protein, 56 aa, Triticum aestivum
QQQQPPFSQQQQPVLPQQPSFSQQQLPPFSQQQQPPFSQQQQPVLPQQPSFSQQQL

SEQ ID NO: 167 - protein, 56 aa, Triticum aestivum
ILPQQPPFSQQQQLVLPQQPPFSQQQQPALPPQQSPFPQQQQQHQQLVQQQIPVVQ

SEQ ID NO: 168 - protein, 56 aa, Triticum aestivum
PHQFPQQQPCSQQQQQPPLSQQQQPPFSQQQQPPFSQQQQPVLPQQPSFSQQQLPP

SEQ ID NO: 169 - protein, 56 aa, Triticum aestivum
QLFPQQPSFSQQQPPFWQQQPTFSQQQPILPQQPPFSQQQQLVLPQQPPFSQQQQP

SEQ ID NO: 170 - protein, 56 aa, Triticum aestivum
LERPWQQQPLPPQQTFPQQPLFSQQQQQQLFPQQPSFSQQQPPFWQQQPTFSQQQP

SEQ ID NO: 171 - protein, 56 aa, Triticum aestivum
WYYPTSPQLSGQGQRPGQWLQPGQGQQGYYPTSPQQPGQGQQLGQWLQPGQGQQGY

SEQ ID NO: 172 - protein, 56 aa, Triticum aestivum
PGQASPQQPGQGQQPGKWQELGQGQQGYYPTSLHQSGQGQQGYYPSSLQQPGQGQQ

SEQ ID NO: 173 - protein, 56 aa, Triticum aestivum
QGQPGYYPTSPQQPGQEQQLEQWQQSGQGQPGHYPTSPLQPGQGQPGYYPTSPQQI

SEQ ID NO: 174 - protein, 56 aa, Triticum aestivum
GQGQQPGQGQQGQQLGQGQQGYYPTSLQQSGQGQPGYYPTSLQQLGQGQSGYYPTS

SEQ ID NO: 175 - protein, 56 aa, Triticum aestivum
SGQEQQLEQWQQSGQGQPGHYPTSPLQPGQGQPGYYPTSPQQIGQGQQPGQLQQPT

SEQ ID NO: 176 - protein, 56 aa, Triticum aestivum
PISPQQLGQGQQSGQGQLGYYPTSPQQSGQGQSGYYPTSAQQPGQLQQSTQEQQLG

SEQ ID NO: 177 - protein, 56 aa, Triticum aestivum
LQPGQGQQGYYPTSPQQSGQGQQLGQWLQPGQGQQGYYPTSLQQTGQGQQSGQGQQ

SEQ ID NO: 178 - protein, 56 aa, Triticum aestivum
QQSGQGQQPGQGQQPGQGQEGYYPTLGQQPGQWLQIGQGQQGYYPTSPQQLGQGQQ

SEQ ID NO: 179 - protein, 56 aa, Triticum aestivum
GQPGYYPTSPQQSGQGQPGYYPTSPQQSGQLQQPAQGQQPGQEQQGQQPGQGQQPG

SEQ ID NO: 180 - protein, 56 aa, Triticum aestivum
QPGQRQQPGQGKPGYYPTSPQQSGQGQSGYYPTSPQQPGQEQQPGQGQQVQQPGQG

SEQ ID NO: 181 - protein, 56 aa, Triticum aestivum
QPGQGQQGYYPTSLQQTGQGQQSGQGQQGYYSSYHVSVEHQAASLKVAKAQQLAAQ

SEQ ID NO: 182 - protein, 56 aa, Triticum aestivum
GQPGHYPTSPLQPGQGQPGYYPTSPQQIGQGQQPGQLQQPTQGQQGQQPGQGQQGQ

SEQ ID NO: 183 - protein, 56 aa, Triticum aestivum
QQPGQGQQGHCPTSRQQPGQAQQPGQGQQIGQAQKPGQGQQGYYPTSLQQPGQGQQ

SEQ ID NO: 184 - protein, 56 aa, Triticum aestivum
QAYYPTSSQQSGQRQQAGQWQRPGQGQSGYYPTSPQQPGQEQQSGQAQQSGQWQLV

SEQ ID NO: 185 - protein, 56 aa, Triticum aestivum
QQPGQGQPGYYPTSSLQLGQGQQGYYPTSQQQPGQGPQPGQWQQLGQGQQGYYPTS

SEQ ID NO: 186 - protein, 56 aa, Triticum aestivum
PQQSGQGQQPGQWLQSGYYLTSPQQLGQGQQPRQWLQPRQGQQGYYPTSPQQSGQG

SEQ ID NO: 187 - protein, 56 aa, Triticum aestivum
QSGQGQPGYYPTSLQQLGQGQSGYYPTSPQQPGQGQQPGQLQQPAQGQQPGQGQQG

SEQ ID NO: 188 - protein, 56 aa, Triticum aestivum
QGQQGYYPTSPQQSGQGQQPGQWLQPGQGQEGYYPTSPQQPGQGQQPGQWLQIGQG

SEQ ID NO: 189 - protein, 56 aa, Triticum aestivum
PTSPQQSGQGQQPGHEQQPGQWLQPGQGQQGYYPTSSQQSGQGHQSGQGQQGYYPT

SEQ ID NO: 190 - protein, 56 aa, Triticum aestivum
QQPGQGLPGYYPTSSLQPEQGQQGYYPTSQQQPGQGPQPGQWQQSGQGQQGYYPTS

SEQ ID NO: 191 - protein, 56 aa, Triticum aestivum
GQGQPGYYPTSPLQSGQGQSGYYPTSPQQSGQGQQPGQLQQPAQGQQPEQGQQGQQ

SEQ ID NO: 192 - protein, 56 aa, Triticum aestivum
PGQGQPWYYPTSPQESGQGQQPGQWQQPGQGQPGYYLTSPLQLGQGQQGYYPTSLQ

SEQ ID NO: 193 - protein, 56 aa, Triticum aestivum
GQGQPGYYLTSPLQLGQGQQGYYPTSLQQPGQGQQPGQWQQSGQGQHWYYPTSPQL

SEQ ID NO: 194 - protein, 56 aa, Triticum aestivum
QQPEQGQQPGQGQQGYYPTSPQQSGQGKQLGQEQQGYYPTSPQQPGQGQQPGQGQQ

SEQ ID NO: 195 - protein, 56 aa, Triticum aestivum
GHPGQRQQPGQGQQPEQGQQPGQGQQGYYPTSPQQPGQGKQLRQGQQGYYPTSLQQ

SEQ ID NO: 196 - protein, 56 aa, Triticum aestivum
GQGQSGYYPTSAQQPGQLQQSTQEQQLGQEQQDPQSGQGRQGQQSGQRQQDQQSGQ

SEQ ID NO: 197 - protein, 56 aa, Triticum aestivum
YYPTSPQQPGQGQQAGHGQQPGQWLQPGQGQQGYYPTSLQQSGQGQQSGQGQQGYY

SEQ ID NO: 198 - protein, 56 aa, Triticum aestivum
QGQQGQQQRQGEHGQQPGQGQQGQQPGQGQPGYYPTSPQQSGQGQPGYYPTSPQQS

SEQ ID NO: 199 - protein, 56 aa, Triticum aestivum
YYPTSPQQPGQGQQLGQWLQPGQGQQGYYPTSLQQTGQGQQSGQGQQGYYSSYHVS

SEQ ID NO: 200 - protein, 56 aa, Triticum aestivum
TSPQQSGQWQQPGQGQSGYYPTSPQQSGQKQPGYYPTSPWQPEQLQQPTQGQQRQQ

SEQ ID NO: 201 - protein, 56 aa, Triticum aestivum
GQEGYYPTSPQQPGQGQQPGQWLQIGQGQQGYYPTSPQQPGQGQQGYYLTSPQQPG

SEQ ID NO: 202 - protein, 56 aa, Triticum aestivum
QQPGQGQPGYYPTSPQQPGQGQQPGQEQQPGQRQQPGQGKPGYYPTSPQQSGQGQS

SEQ ID NO: 203 - protein, 56 aa, Triticum aestivum
QGYYPTSLQQPGQGQQPGQWQQSGQGQHWYYPTSPQLSGQGQRPGQWLQPGQGQQG

SEQ ID NO: 204 - protein, 56 aa, Triticum aestivum
PQQLGQGQQPRQWLQPRQGQQGYYPTSPQQSGQGQQLGQGQQGYYPTSPQQSGQGQ

SEQ ID NO: 205 - protein, 56 aa, Triticum aestivum
QQPGQGQQSGQGQQGQQPGQGQQAYYPTSSQQSRQRQQAGQWQRPGQGQPGYYPTS

SEQ ID NO: 206 - protein, 56 aa, Triticum aestivum
GQQGQQVGQGQQAQQPGQGQQPGQGQPGYYPTSPQQSGQGQPGYYLTSPQQSGQGQ

SEQ ID NO: 207 - protein, 56 aa, Triticum aestivum
SQQPGQGQQGQQVGQGQQAQQPGHGQQPGQGQPGYYPTSPLQSGQGQSGYYPTSPQ

SEQ ID NO: 208 - protein, 56 aa, Triticum aestivum
GQGQQGHCPTSPQQTGQAQQPGQGQQIGQVQEPGQGQQGYYPISLQQSGQGQQSGQ

SEQ ID NO: 209 - protein, 56 aa, Triticum aestivum
QGQQGQQPGQGQQPGQGQPGYYPTSPQQSGQEQQLEQWQQSGQGQPGHYPTSPLQP

SEQ ID NO: 210 - protein, 56 aa, Triticum aestivum
PQQSGQGQQPGQWLQPGQWLQSGYYLTSPQQLGQGQQPRQWLQPRQGQQGYYPTSP

SEQ ID NO: 211 - protein, 56 aa, Triticum aestivum
YPTSPQQPGQGKQLRQGQQGYYPTSLQQPGQGQQPGQGQQSGQGQQGHCPTSPQQT

SEQ ID NO: 212 - protein, 56 aa, Triticum aestivum
PGQASPQQPGQGQQPGKWQEPGQGQQWYYPTSLQQPGQGQQMGKGKQGYYPTSLQQ

SEQ ID NO: 213 - protein, 56 aa, Triticum aestivum
GQQPGQRQPGYYSTSPQQLGQGQPRYYPTCPQQPGQEQQPRQLQQPEHGQQGQQPE

SEQ ID NO: 214 - protein, 56 aa, Triticum aestivum
SPLQLGQGQQGYYPTSLQQPGQGQQPGQWQQSGQGQHGYYPTSPQLSGQGQRPGQW

SEQ ID NO: 215 - protein, 56 aa, Triticum aestivum
PQESGQGQQPGQWQQPGQGQPGYYLTSPLQLGQGQQGYYPTSLQQPGQGQQPGQWQ

SEQ ID NO: 216 - protein, 56 aa, Triticum aestivum
QVQEPGQGQQGYYPISLQQSGQGQQSGQGQQSGQGHQLGQGQQSGQEQQGYDNPYH

SEQ ID NO: 217 - protein, 56 aa, Triticum aestivum
LQQSGQGQQPGQWQQPGQGQPGYYPTSSLQPEQGQQGYYPTSQQQPGQGPQPGQWQ

SEQ ID NO: 218 - protein, 56 aa, Triticum aestivum
PTSPQESGQGQQPGQWQQPGQWQQPGQGQPGYYLTSPLQLGQGQQGYYPTSLQQPG

SEQ ID NO: 219 - protein, 56 aa, Triticum aestivum
YYPGGPQQPGQGQQLGQWLQPGQGQQGYYPTGLQQTGQGQQSGQGQQGYYSSYHVS

SEQ ID NO: 220 - protein, 56 aa, Triticum aestivum
PGQGQQGYYPTSLQHTGQRQQPVQGQQPEQGQQPGQWQQGYYPTSPQQLGQGQQPR

SEQ ID NO: 221 - protein, 56 aa, Triticum aestivum
QSGQGQQGYYPTSSQQSGQGQQPGQWLQPGQWLQSGYYLTSPQQLGQGQQPRQWSQ

SEQ ID NO: 222 - protein, 56 aa, Triticum aestivum
QGQQPEQGQQGQQPGQGEQGQQPGQGQQGQQPGQGQPGYYPTSPQQSGQGQPGYYP

SEQ ID NO: 223 - protein, 56 aa, Triticum aestivum
GQGQQPGQWQQPGQWQQPGQGQPGYYLTSPLQLGQGQQGYYPTSLQQPGQGQQPGQ

SEQ ID NO: 224 - protein, 56 aa, Triticum aestivum
QQGYYPISPQQSGQGQQPGQGQQGYYPTSPQQSGQGQQPGQWLQPGQGQQGYYPTS

SEQ ID NO: 225 - protein, 56 aa, Triticum aestivum
QPGQWLQIGQGQQGYYPTSPQQLGQGQQGYYLTSPQQPGQGKQPGQGQQSYDSPYH

SEQ ID NO: 226 - protein, 56 aa, Triticum aestivum
GQGQQPGQWQQPGQGQPGYYPTSPLQPGQGQPGYDPTSPQQPGQGQQPGQLQQPAQ

SEQ ID NO: 227 - protein, 56 aa, Triticum aestivum
PRQGQQGYYPTSPQQSGQGQQLGQGQQGYYPTSPQQSGQGQQGYDSPYYVSAEHQA

SEQ ID NO: 228 - protein, 56 aa, Triticum aestivum
QQGQQPGQGQQPGQGQQGQQLGQGQQGYYPTSPQQSGQGQPGYYPTSSQQPTQSQQ

SEQ ID NO: 229 - protein, 56 aa, Triticum aestivum
YPTSLHQSGQGQQGYYPSSLQQPGQGQQTGQGQQGYYPTSLQQPGQGQQIGQGQQG

SEQ ID NO: 230 - protein, 56 aa, Triticum aestivum
GQGQQGYYPTSPQQPGQWQQPEQGQPRYYPTSPQQSGQLQQPAQGQQPGQGQQGQQ

SEQ ID NO: 231 - protein, 56 aa, Triticum aestivum
QQGQQPGQGQQPGQGQQGQQLGQGQQGYYPTSLQQQPGYYPTSLQQLGQGQSGYYP

SEQ ID NO: 232 - protein, 56 aa, Triticum aestivum
QPGQGQQGYYPTSPQQLGQGQQPGQWQQPGQGQPGYYPTSPQQPGQGQPGYYPTSP

SEQ ID NO: 233 - protein, 56 aa, Triticum aestivum
GYYPTSPQQPGQEQQPGQGQQVQQPGQGQQPGQGQQGYYPTSPQQSGQAQQPGQWQ

SEQ ID NO: 234 - protein, 56 aa, Triticum aestivum
GQQPGQGQPGYYPTSLQQSGQGQQPGQWQQPGQGLPGYYPTSSLQPEQGQQGYYPT

SEQ ID NO: 235 - protein, 56 aa, Triticum aestivum
GQQPGQGQPGYYPTSLQQSGQGQQPGQWQQPGQGQPGYYPTSSLQLGQGQQGYYPT

SEQ ID NO: 236 - protein, 56 aa, Triticum aestivum
LQQPTQGQQGYYPTSPQQSGQGQQGYYPTSPQQSGQGQQGYYPTSPQQSGQGQQPG

SEQ ID NO: 237 - protein, 56 aa, Triticum aestivum
QQGYYPTSSQQSGQGHQSGQGQQGYYPTSLWQPGQGQQGYASPYHVSAEYQAARLK

SEQ ID NO: 238 - protein, 56 aa, Triticum aestivum
TGQGQQGYYPTSLQQPGQGQQIGQGQQGYYPTSPQHPGQRQQPGQGQQIGQEQQLG

SEQ ID NO: 239 - protein, 56 aa, Triticum aestivum
PGQGQPGYYPTSPQQPGQGQPGYYPTSPQQPGQLQQPAQGQQGYYPTSPQQPGQEQ

SEQ ID NO: 240 - protein, 56 aa, Triticum aestivum
YYPTSPQQSGQGQPGYYLTSPQQSGQGQQPGQLQQSAQGQKGQQPGQGQQPGQGQQ

SEQ ID NO: 241 - protein, 56 aa, Triticum aestivum
GQGQQGYYPTSLQQPGQGQRQGQGQQGYYPTSPQQPGQGQQGHHPASLQQPGQGQP

SEQ ID NO: 242 - protein, 56 aa, Triticum aestivum
QGQQGYYPTSLQQSGQGQQSGQGQQGYYPTSPQQSGQGQQGYDSPYHVSAEHQAAS

SEQ ID NO: 243 - protein, 56 aa, Triticum aestivum
GQGQQGYYPTSLQQPGQGQQQGQGQQGYYPTSLQQPGQGQQGHYPASLQQPGQGQP

SEQ ID NO: 244 - protein, 56 aa, Triticum aestivum
QQTGQGQQPEQEQQPGQGQQGYYPTSLQQPGQGQQQGQGQQGYYPTSLQQPGQGQQ

SEQ ID NO: 245 - protein, 56 aa, Triticum aestivum
YPTSLQQPGQGQQMGKGKQGYYPTSLQQPGQGQQIGQGQQGYYPTSPQHTGQRQQP

SEQ ID NO: 246 - protein, 56 aa, Triticum aestivum
QQPGQGQQGQQPGQGQQPGQGQPGYYPTSPQQSGQGQPGYYPTSSQQPTQSQQPGQ

SEQ ID NO: 247 - protein, 56 aa, Triticum aestivum
LQPGQGQQGYYPTSSQQSGQGQQSGQGQQGYYPTSLWQPGQGQQPGQRQQGYDSPY

SEQ ID NO: 248 - protein, 56 aa, Triticum aestivum
LQLGQGQQGYYPTSPQQSGQGQQSGQGQQGYYPTSLWQPGQGQQPGQGQQGYDSPY

SEQ ID NO: 249 - protein, 56 aa, Triticum aestivum
HYPGSLRQPGQGQPGQRQQPGQGQQTGQGQQPEQEQQPGQGQQGYYPTSPQQPGQG

SEQ ID NO: 250 - protein, 56 aa, Triticum aestivum
QLGQGQPGYYPTSPQQSGQGQQSGQGQQGYYPTSPQQSGQGQQPGQGQSGYFPTSR

SEQ ID NO: 251 - protein, 56 aa, Triticum aestivum
LQPGQGQQGYYPTSSQQSGQGHQSGQGQQGYYPTSLWQPGQGQQPGQGQQGYASPY

SEQ ID NO: 252 - protein, 56 aa, Triticum aestivum
GQGQPGYYPTSPQQIGQGQQPGQLQQPTQGQQGQQPGQGQQGQQPGQGQQGQQPGQ

SEQ ID NO: 253 - protein, 56 aa, Triticum aestivum
PGQGQQPGRGQPGYYPTSSQQLGQGQQLGQGQQGQQPGQGQPGYYPTSPQQPGQGQ

SEQ ID NO: 254 - protein, 56 aa, Triticum aestivum
QAYYPTSSQQSRQRQQAGQWQRPGQGQPGYYPTPPQQPGQEQQSGQAQQSGQWQLV

SEQ ID NO: 255 - protein, 56 aa, Triticum aestivum
GQRQQPGQGQHPEQGQQPGQGQQGYYPTSPQQPGQGQQLGQGQQGYYPTSPQQPGQ

SEQ ID NO: 256 - protein, 56 aa, Triticum aestivum
GQGQQGYYPTSPQQPGQWQQPEQGQQGYYPTSPQQPGQLQQPAQGQQPGQGQQGQQ

SEQ ID NO: 257 - protein, 56 aa, Triticum aestivum
VASPQQVSYYPGQASSRRPGQGQQEYYLTSPQQSGQWQQPGQGQSGYYPTSPQQSG

SEQ ID NO: 258 - protein, 56 aa, Triticum aestivum
QQPGQGQQGYYPTSLQQPGQGQQPHYPASQQQPGQGQQGHYPTSLLQPGQGQQGHY

SEQ ID NO: 259 - protein, 56 aa, Triticum aestivum
WYYPTSPQESGQGQQPGQWQQPGQWQQPGQGQQGQQPGQGQPGYYPTSPQQSGQGQ

SEQ ID NO: 260 - protein, 56 aa, Triticum aestivum
SLQQPGQGQQPGQGQPGYYPTSQQSEQGQQPGQGKQPGQGQQGYYPTSSQQSGQGQ

SEQ ID NO: 261 - protein, 56 aa, Triticum aestivum
GQQPGQGKQPGQGQQGYYPTSPQHPGQGQQPGQGQQPGQGKPGYYPTSPQLPGQGQ

SEQ ID NO: 262 - protein, 56 aa, Triticum aestivum
GQGQQGYYPTSLQQPGQGQQGHYPASLQQPGQGQPGQRQQPGQGQHPEQGQQPGQG

SEQ ID NO: 263 - protein, 56 aa, Triticum aestivum
QGRQIGQGQQSGQGQQGYYPTSPQQLGQGQQPGQWQQSGQGQQGYYPTSQQQPGQG

SEQ ID NO: 264 - protein, 56 aa, Triticum aestivum
SPQQPGEGQQPGQGRQPGQGQQPGQGQQGQQPGQGKQPGQGQQGYYPTSPQHPGQG

SEQ ID NO: 265 - protein, 56 aa, Triticum aestivum
TPQQPGQWQQPGQVQPGYYLTPPQQTGQAQQPGQGQQPGQGQPGYYPTSPRQPGQG

SEQ ID NO: 266 - protein, 56 aa, Triticum aestivum
GYYPTSLQQPGQGQQPGQGQPGYYPTSPQQPGQGKQPGQGQQRYYPTSSQQSGQGQ

SEQ ID NO: 267 - protein, 56 aa, Triticum aestivum
SGQGQQGQQPGQGQRPGQGQQGYYPTSLQQPRQGQQSGQGQPGYYPTSSRQPGQWQ

SEQ ID NO: 268 - protein, 56 aa, Triticum aestivum
YPTSLQQPGQGQQGHYPASLQQPGQGQPGQRQQPGQGQQPGQGQQGYYPTSPQQPG

SEQ ID NO: 269 - protein, 56 aa, Triticum aestivum
GQLQQPAQGQQPGQEQQGQQPGQGQQPGQGQPGYYPTSPQQPGQEQQLEQWQQSGQ

SEQ ID NO: 270 - protein, 56 aa, Triticum aestivum
YYPTSPQHPGQRQQPGQGQQIGQEQQLGQGRQIGQGQQSGQGQQGYYPTSPQQLGQ

SEQ ID NO: 271 - protein, 56 aa, Triticum aestivum
QGYYSTSLQQPGQGQQGHYPTSLQQPGQGHPGQRQQPGQGQQPEQGQQPGQGQQGY

SEQ ID NO: 272 - protein, 56 aa, Triticum aestivum
QGQQPGQGQQPEQEQQPGQGQQGYYITSLQQPGQGKQLGQWQQPGQGQEGYYPTSP

SEQ ID NO: 273 - protein, 56 aa, Triticum aestivum
QQSGQGQQRYYPTSPQQSGQGQQPGQGQPGYYPISPQQSEQWQQPGQGQQPGQGQQ

SEQ ID NO: 274 - protein, 56 aa, Triticum aestivum
QGRQIGQGQQSGQEQQGYYATSPQQLGQGQQPGQWQQSGQGQQRYYPTSQQQPGQG

SEQ ID NO: 275 - protein, 56 aa, Triticum aestivum
AQEQQPGQAQQSGQWQLVYYPTSPQQSGQGQQGYYPTSPQQSGQGQQPGQGQQPRQ

SEQ ID NO: 276 - protein, 56 aa, Triticum aestivum
GQGQPGYYPTSQQPGQKQQAGQGQQSGQGQQGYYPTSPQQSGQGQQPGQGQAGYYP

SEQ ID NO: 277 - protein, 56 aa, Triticum aestivum
GQIPASQQQPGQGQQGHYPASLQQPGQQGHYPTSLQQLGQGQQIGQPGQKQQPGQG

SEQ ID NO: 278 - protein, 56 aa, Triticum aestivum
QQGYYPTSPQQPGQGQQLGQGQQGYYPTSPQQPGQGQQPGQGQQGHCPMSPQQTGQ

SEQ ID NO: 279 - protein, 56 aa, Triticum aestivum
QEQQPGQAQQSGQWQLVYYPTSPQQPGQLQQPTQGQQGYYPTSPQQSGQGQQGYYP

SEQ ID NO: 280 - protein, 56 aa, Triticum aestivum
QEQQDPQSGQGRQGQQSGQRQQDQQSGQGQQPGQRQPGYYSTSPQQLGQGQPRYYP

SEQ ID NO: 281 - protein, 56 aa, Triticum aestivum
GQGQQGHYPASQQEPGQGQQGQIPASQQQPGQGQQGHYPASLQQPGQQGHYPTSLQ

SEQ ID NO: 282 - protein, 56 aa, Triticum aestivum
GQQGYYPTSPQQPGQGQQPGQGQQGHCPMSPQQTGQAQQLGQGQQIGQVQQPGQGQ

SEQ ID NO: 283 - protein, 56 aa, Triticum aestivum
GQQGYYPISPQQSGQGQQPGQGQQGYYPTSPQQSGQGQQPGHEQQPGQWLQPGQGQ

SEQ ID NO: 284 - protein, 56 aa, Triticum aestivum
QKQPGYYPTSPWQPEQLQQPTQGQQRQQPGQGQQLRQGQQGQQSGQGQPRYYPTSS

SEQ ID NO: 285 - protein, 56 aa, Triticum aestivum
QQSGQGQQLGQGQQGYYPTSPQQSGQGQQGYYPTSPQQSGQGQQLGQGQQGYYPTS

SEQ ID NO: 286 - protein, 56 aa, Triticum aestivum
QQGYYPTSPQQSGQGQQPGQSQQPGQGQQGYYSTSLQQPGQGQQGHYPTSLQQPGQ

SEQ ID NO: 287 - protein, 56 aa, Triticum aestivum
GQQPGQGQQPGQGQPWYYPTSPQESGQGQQPGQWQQPGQGQPGYYLTSPLQLGQGQ

SEQ ID NO: 288 - protein, 56 aa, Triticum aestivum
PGQGQPGYYPTSSQLQPGQLQQPAQGQQGQQPGQGQQGQQPGQGQQPGQGQQGQQP

SEQ ID NO: 289 - protein, 56 aa, Triticum aestivum
SPLQPGQGQPGYDPTSPQQPGQGQQPGQLQQPAQGQQGQQLAQGQQGQQPAQVQQE

SEQ ID NO: 290 - protein, 56 aa, Triticum aestivum
GQQGYYPTSPQQSGQGQQPGQGQAGYYPTSPQQSGQWQQPGQGQQPGQGQQSGQGQ

SEQ ID NO: 291 - protein, 56 aa, Triticum aestivum
QGQQPAQGQQGQQPGQGQQGQQPGQGQQGQQPGQGQQPGQGQPWYYPTSPQESGQG

SEQ ID NO: 292 - protein, 56 aa, Triticum aestivum
PEQEQQPGQGQQGYYPTSLQQPGQGQQQGQGQQGYYPTSLQQPGQGQQGHYPASLQ

SEQ ID NO: 293 - protein, 56 aa, Triticum aestivum
SQQQPGQGPQPGQWQQLGQGQQGYYPTSPQQSGQGQQPGQWLQSGYYLTSPQQLGQ

SEQ ID NO: 294 - protein, 56 aa, Triticum aestivum
QQSGQGQQLGQGQQGYYPTSPQQSGQGQQGYDSPYHVSAEHQAASLKVAKAQQLAA

SEQ ID NO: 295 - protein, 56 aa, Triticum aestivum
QGQQLGQGQQGQQPGQKQQSGQGQQGYYPISPQQLGQGQQSGQGQLGYYPTSPQQS

SEQ ID NO: 296 - protein, 56 aa, Triticum aestivum
QGQQPGQGQQGYYPTSPQQPGQGQQLGQGQQGYYPTSPQQPGQGQQPGQGQQGHCP

SEQ ID NO: 297 - protein, 56 aa, Triticum aestivum
SQQQPGQGPQPGQWQQSGQGQQGYYPTSPQQSGQGQQPGQWLQPGQWLQSGYYLTS

SEQ ID NO: 298 - protein, 56 aa, Triticum aestivum
SPQQSGQGQQPGQSQQPGQGQQGYYSTSLQQPGQGQQGHYPASLQQPGQGHPGQRQ

SEQ ID NO: 299 - protein, 56 aa, Triticum aestivum
GQRQQPGQGQQPGQGQQGYYPTSPQQPGQGQQLGQGQQGYYPTSPQQPGQGQQPGQ

SEQ ID NO: 300 - protein, 56 aa, Triticum aestivum
GQQPGQGQQGQQPGQGQPGYYPTSPQQSGQGQQPGQWQQPGQGQPGYYPTSPLQPG

SEQ ID NO: 301 - protein, 56 aa, Triticum aestivum
QPGQGQQPKQGQQPGQGQQGYYPTSSQQPGQGKQLGQGQQGYYPTSPQQPGQGQQP

SEQ ID NO: 302 - protein, 56 aa, Triticum aestivum
QQPGQGKQPGQGQQGYYPTSSQQSGQGQQLGQGQPGYYPTSPQQSGQGQQSGQGQQ

SEQ ID NO: 303 - protein, 56 aa, Triticum aestivum
QGQQGYYPTSPQQSGQGQQPGQGQQPRQGQQGYYPISPQQSGQGQQPGQGQQGYYP

SEQ ID NO: 304 - protein, 56 aa, Triticum aestivum
PGQGQQPGQGQQGYYPTSPQQPGQGQQPGQGQLEYYPTSPQQPGQGQPGYYPTFPQ

SEQ ID NO: 305 - protein, 56 aa, Triticum aestivum
VTSSQQGSYYPGQAFPQQSGQGQQPGQGQQPGQRQQDQQPGQGQQGYYPTSPQQPG

SEQ ID NO: 306 - protein, 56 aa, Triticum aestivum
GQGQPRYYPTSQQPGQKQQAGQGQQSGQGQQGYYPTSPQQSGQGQQPGQGQSRYYP

SEQ ID NO: 307 - protein, 56 aa, Triticum aestivum
GQQGYYPTSPQQSGQGQQPGHEQQPGQWLQLGQGQQGYYPTSPQQSGQGQQSGQGQ

SEQ ID NO: 308 - protein, 56 aa, Triticum aestivum
QGYYPTSPQQPGQGQQPGQWQQPGQGQQGYYPTSPQQPGQGQQPGQWLQPGQEQQG

SEQ ID NO: 309 - protein, 56 aa, Triticum aestivum
QGQQPRQGQQGYYPISPQQSGQGQQTGQGQQGYYPTSPQQSGQGQQPRHEQQPGQW

SEQ ID NO: 310 - protein, 56 aa, Triticum aestivum
EQQDQQPGQRQQGYYPTSPQQPGQGQQLGQGQPGYYPTSQQPGQKQQAGQGQQSGQ

SEQ ID NO: 311 - protein, 56 aa, Triticum aestivum
QQPGQGQQDQQPEQGQQPGKGQQGYYPTTPQQPGQWQQPGQVQPGYYLTPPQQTGQ

SEQ ID NO: 312 - protein, 56 aa, Triticum aestivum
GQLQQPAQGQQGYYPTTPQQPGQGQQPGQGQQGYYPTSPQQSGQGQQPGQWLQPGQ

SEQ ID NO: 313 - protein, 56 aa, Triticum aestivum
QQPGQLQQPAQGQQGYYPTSPQQPGQEQQGYYPTSPQQPGQGQQPGQWQQPGQGQQ

SEQ ID NO: 314 - protein, 56 aa, Triticum aestivum
ASQQQPGQGQQGHYPASQQQPGQRQQGHYPASQQQPGQGQQGHYPASQQEPGQGQQ

SEQ ID NO: 315 - protein, 56 aa, Triticum aestivum
LPGQLQQPAQGQQGYYPTSPQQPGQGQQKYYPTSPQQPGQWQQPGQGQQGYYITSP

SEQ ID NO: 316 - protein, 56 aa, Triticum aestivum
GQGQLEYYPTSPQQPGQGQPGYYPTFPQLPGQLQQPAQGQQGYYPTSPQQPGQGQQ

SEQ ID NO: 317 - protein, 56 aa, Triticum aestivum
YPTSLQQPGQGQQIGKGQQGYYPTSLQQPGQGQQGYYPTSLQHTGQRQQPVQGQQP

SEQ ID NO: 318 - protein, 56 aa, Triticum aestivum
QGQQPGQGQRPGQGQQGYYPTSPQQPGQGQQSGQGQPGYYPTSLRQPGQWQQPGQG

SEQ ID NO: 319 - protein, 56 aa, Triticum aestivum
QGYYPTSPQQPGQGQQPGQGQQPGEGQPGYYPTSPQQPGQGQQPGQGQPGYYPTSS

SEQ ID NO: 320 - protein, 56 aa, Triticum aestivum
GQLGYYPTSPQQPGQGQPGYYPTSPQLPGQLQQPAQGQQGYYPTTPQQPGQGQQPG

SEQ ID NO: 321 - protein, 56 aa, Triticum aestivum
QQPGQGQQGYYPTSPQQSGQAQQPGQWQQPGQGQSGYYPTSQQQPGQGQQPGQGQQ

SEQ ID NO: 322 - protein, 56 aa, Triticum aestivum
GYYPTSPQQPEQGQQPGQGQQPGQGQPGYYPTSPQQPGQGQQPGQEQQPGQGQQPG

SEQ ID NO: 323 - protein, 56 aa, Triticum aestivum
PGQGQQPGQGQQGYYPTSPQQPGQGQQGHYPGSLQQPGQGQPGQRQQPGHGQQTGQ

SEQ ID NO: 324 - protein, 56 aa, Triticum aestivum
QGQQPGQGQRPGQGQQGYYPISPQQPGQGQQSGQGQPGYYPTSFAQPRTMAATQDK

SEQ ID NO: 325 - protein, 56 aa, Triticum aestivum
QQPGQGKQPGQGQQRYYPTSSQQSGQGQQPGQGQPGYYPTSPQQSGQGQQSGQAQQ

SEQ ID NO: 326 - protein, 56 aa, Triticum aestivum
QPGQGQPGQRQQPGQGQHPEQGQQPGQGQQGYYPTSPQQPGQGQQLGQGQQGYYPT

SEQ ID NO: 327 - protein, 56 aa, Triticum aestivum
QGRQPRQGQQGYYPISPQQSGQGQQPGQGQQGYYPTSPQQSGQGQQPGHEQQPGQW

SEQ ID NO: 328 - protein, 56 aa, Triticum aestivum
GQGQQTRQGQQLEQGQQPGQGQQGYYPTSPQQSGQGQQPGQSQQPGQGQQGYYSTS

SEQ ID NO: 329 - protein, 56 aa, Triticum aestivum
PGQGQQPGQGQQGQQPGQGQQPGQGQQGYYPTSPQHLGQGQQPGQGKPGSYPTSPQ

SEQ ID NO: 330 - protein, 56 aa, Triticum aestivum
GQQPEQEQQPGQGQQGYYPTSPQQPGRGQQPGQGQQGYYPTSLQQPGQGQQPHYPA

SEQ ID NO: 331 - protein, 56 aa, Triticum aestivum
QQPGQGQQGQQPGQRQQSGQGQQGYYPTSLQQPGQGQQLGQGQPGYYPTSQQSEQG

SEQ ID NO: 332 - protein, 56 aa, Triticum aestivum
QQPGQGQQGQQPGQGQQPGQGQQGYYPTSLQQPGQGQQPGQGQPGYYPTSQQSEQG

SEQ ID NO: 333 - protein, 56 aa, Triticum aestivum
QPGQGQQQGQGQQGYYPTSLQQPGQGQQGHYPASLQQPGQGQPGQRQQPGQGQHPE

SEQ ID NO: 334 - protein, 56 aa, Triticum aestivum
GQQSGQGQPGYYPTSFAQPRTMAATQDKGSNQDKGNKVSSQDKDNNQDKDNKDTTQ

SEQ ID NO: 335 - protein, 56 aa, Triticum aestivum
GQQPGQWQQSGQGQQGYYPTSQQQPGQGQQGQYPASQQQPGQGQQGQYPASQQQPG

SEQ ID NO: 336 - protein, 56 aa, Triticum aestivum
YPSVTCPQQVSYYPGQASPQRPGQGQQPGQGQQGYYPTSPQQPGQWQQPEQGQQGY

SEQ ID NO: 337 - protein, 56 aa, Triticum aestivum
GYYPTSPQQPGQGQQPGQGQPGYYPTSSQQPGQGQQPGQGQQPGQGQQGQQPGQGQ

SEQ ID NO: 338 - protein, 56 aa, Triticum aestivum
YYPTSPQQLGQGQQPGQGQQPGQGQPGYYPTSPQQPGQGQQTGQGQQPGQGQQGQQ

SEQ ID NO: 339 - protein, 56 aa, Triticum aestivum
SLQQPGQGQQLGQGQPGYYPTSQQSEQGQQPGQGQQGYYPTSPQQSGQGQQLGQGQ

SEQ ID NO: 340 - protein, 56 aa, Triticum aestivum
LQQPGQGQQGHYPASLQQPGQGHPGQRQQPGQGQQPKQGQQPGQGQQGYYPTSSQQ

SEQ ID NO: 341 - protein, 56 aa, Triticum aestivum
PGQGQQIGQGQQGYYPTSPQHTGQRQQPVQGQQIGQGQQPEQGQQPGQWQQGYYPT

SEQ ID NO: 342 - protein, 56 aa, Triticum aestivum
YPSVTSPQQVSYYPGQASPQRPGQGQQPGQGQQGYYPTSPQQPGQWQQPEQGQPRY

SEQ ID NO: 343 - protein, 56 aa, Triticum aestivum
YPSVTSPQQVSYYPGQASPQRPGQGQQPGQGQQSGQGQQGYYPTSPQQPGQWQQPE

SEQ ID NO: 344 - protein, 56 aa, Triticum aestivum
GQQGHCPMSPQQTGQAQQQGQGQQIGQVQQPGQGQQGYYPTSLQQPGQGQQSGQGQ

SEQ ID NO: 345 - protein, 56 aa, Triticum aestivum
SPQQSGQGQPGYYPTSSQQPTQSQQPGQGQQGQQVGQGQQAQQPGQGQQPGQGQPG

SEQ ID NO: 346 - protein, 56 aa, Triticum aestivum
QQPGQGQQPGQGKPGYYPTSPQLPGQGQQPGQGQSGYYPTSPQQLGQGQQPGQGWQ

SEQ ID NO: 347 - protein, 56 aa, Triticum aestivum
EQGQQPGQWQQGYYPTSPQQLGQGQQPRQWQQSGQGQQGHYPTSLQQPGQGQQGHY

SEQ ID NO: 348 - protein, 56 aa, Triticum aestivum
AGQGQQVQQPGQGQQSGQGQQGYYPTSPQLSGQAQQPGQWQQPGQGQPGYYPTSQQ

SEQ ID NO: 349 - protein, 56 aa, Triticum aestivum
GQQPGEGQQGYYPTFPQQPGQVQQPGQGQQPGQGQPGYYPTSPQQPGQGQQPGQEQ

SEQ ID NO: 350 - protein, 56 aa, Triticum aestivum
PGQRQQPGQGQQTEQGQQLEQGQQPGQGQQGYYPTSPQQSGQGQQPGQSQQPGQGQ

SEQ ID NO: 351 - protein, 56 aa, Triticum aestivum
QIGQGQQIRQGQQPGQGQQGYYQTHPQQPGQGQQPGQGQQGYYPTSPQQPGQGQQG

SEQ ID NO: 352 - protein, 56 aa, Triticum aestivum
IGQPGQKQQPGQGQQTGQGQQPEQEQQPGQGQQGYYPTSLQQPGQGQQQGQGQQGY

SEQ ID NO: 353 - protein, 56 aa, Triticum aestivum
QWQQSGQGQQGHYPTSLQQPGQGQQGHYLASQQQPGQGQQGHYPASQQQPGQGQQG

SEQ ID NO: 354 - protein, 56 aa, Triticum aestivum
HCPTSPQQSGQAQQPGQGQQIGQVQQPGQGQQGYYPTSVQQPGQGQQSGQGQQSGQ

SEQ ID NO: 355 - protein, 56 aa, Triticum aestivum
AQQPGQGQQPGQGQPGYYPTSPRQPGQGQQSGQGQQGQLPGQWQQPGQEQPGYNPT

SEQ ID NO: 356 - protein, 56 aa, Triticum aestivum
GYYPTSPQQSGQGQQPGQGQSGYFPTSRQQSGQGQQPGQGQQSGQGQQGQQPGQGQ

SEQ ID NO: 357 - protein, 56 aa, Triticum aestivum
IGQPGQRQQPGQGQQTGQGQQPEHEQQPGQGQQGYYPTSLQQPGQGQRQGQGQQGY

SEQ ID NO: 358 - protein, 56 aa, Triticum aestivum
TSPQQSGQGQQGYYPTSPQQSGQGQQPGQGRQPRQGQQGYYPISPQQSGQGQQPGQ

SEQ ID NO: 359 - protein, 56 aa, Triticum aestivum
QQPGQGQQPGQGQQSQQPGQGQHPGQGQQGYYPTSPQQPGQGQQPGQGQQPGEGQP

SEQ ID NO: 360 - protein, 56 aa, Triticum aestivum
QGQQLGQGQQGYYPTSPQQPGQGQQPGQGQQGHCPMSPQQTGQAQQQGQGQQIGQV

SEQ ID NO: 361 - protein, 56 aa, Triticum aestivum
QQPGQGQQGYYPTSLQQPGQGQQSGQGQQSGQGHQPGQGQQSGQEKQGYDSPYHVS

SEQ ID NO: 362 - protein, 56 aa, Triticum aestivum
MSPQQTGQAQQLGQGQQIGQVQQPGQGQQGYYPTSLQQPGQGQQSGQGQQSGQGHQ

SEQ ID NO: 363 - protein, 56 aa, Triticum aestivum
QPGQGQQPVQGQSGYYPTSPQQPGQGQQAGQGQQVQQPGQGQQSGQGQQGYYPTSP

SEQ ID NO: 364 - protein, 56 aa, Triticum aestivum
KYYPTSPQQPGQWQQPGQGQQGYYITSPQQSGQGQQPGQGQQPGQGQEGYYPTLGQ

SEQ ID NO: 365 - protein, 56 aa, Triticum aestivum
GQQGQQPGQGQQPGQGQPWYYPTSPQESGQGQQPGQWQQPGQWQQPGQGQPGYYLT

SEQ ID NO: 366 - protein, 56 aa, Triticum aestivum
VQGQQIGQGQQPEQGQQPGQWQQGYYPTSPQQLGQGQQPGQWQQSGQGQQGHYPTS

SEQ ID NO: 367 - protein, 56 aa, Triticum aestivum
QPGQLQQSAQGQKGQQPGQGQQPGQGQQGQQPGQGQQGQQPGQGQPGYYPTSPQQS

SEQ ID NO: 368 - protein, 56 aa, Triticum aestivum
GQQPGQWQQSGQGQQRYYPTSQQQPGQGQQGQYPASQQQPAQGQQGQYPASQQQPA

SEQ ID NO: 369 - protein, 56 aa, Triticum aestivum
LQQPGQGQQGHYLASQQQPAQGQQGHYPASQQQPGQGQQGHYPASQQQPGQGQQGH

SEQ ID NO: 370 - protein, 56 aa, Triticum aestivum
YPTSPQQPGQGQQGHHPASLQQPGQGQPGQRQQPGQRQHPEQGQQPGQGQQGYYPT

SEQ ID NO: 371 - protein, 56 aa, Triticum aestivum
YPTSLQQPGQGQQGHYPASLQQPGQGQPGQRQQPGQGQHPEQGQQPGQGQQGYYPT

SEQ ID NO: 372 - protein, 56 aa, Triticum aestivum
QLGQGQQIGQPGQKQQPGQGQQTGQGQQPEQEQQPGQGQQGYYPTSLQQPGQGQQQ

SEQ ID NO: 373 - protein, 56 aa, Triticum aestivum
QGQQPGQGQQGQQPGQGQQPGQGQPWYYPTSPQESGQGQQPGQWQQPGQWQQPGQG

SEQ ID NO: 374 - protein, 56 aa, Triticum aestivum
AQQLGQGQQIGQVQQPGQGQQGYYPTSLQQPGQGQQSGQGQQSGQGHQPGQGQQSG

SEQ ID NO: 375 - protein, 56 aa, Triticum aestivum
QPGEGQQGQQPGQGQQPGQGQPGYYPTSLQQSGQGQQPGQWQQPGQGQPGYYPTSS

SEQ ID NO: 376 - protein, 56 aa, Triticum aestivum
QGSYYPGQASPQQLGQGQQPGQGQQPRQEQQDQQPGQRQQGYYPTSPQQPGQGQQL

SEQ ID NO: 377 - protein, 56 aa, Triticum aestivum
PGQGQQPGQGQQGQQPGQGQQAGQGQQGYYPTSPQQLGQGQQPGQGQQPGQGQPGY

SEQ ID NO: 378 - protein, 56 aa, Triticum aestivum
QPGQGQQPGQEQQDQQPGQGQQQGQGQQGYYPTSPQQPEQGQQPGQGQQPGQGQPG

SEQ ID NO: 379 - protein, 56 aa, Triticum aestivum
QQPGQGQQGHYTASLQQPGQGQQGHYPASLQQVGQGQQIGQLGQRQQPGQGQQTRQ

SEQ ID NO: 380 - protein, 55 aa, Triticum aestivum
QQSGQAQQPGQWQQPGQGQSGYYPTSQQQPGQGQQPGQGQQPGQGQQPGQGQQGQ

SEQ ID NO: 381 - protein, 56 aa, Triticum aestivum
QGQQGQYPASQQQPGQGQQGHYLASQQQPGQGQQRHYPASLQQPGQGQQGHYTASL

SEQ ID NO: 382 - protein, 56 aa, Triticum aestivum
QGQQPGQGQQGQQPGQGQQGQQPGQGQQPGQGQPWYYPTSPQESGQGQQPGQWQQP

SEQ ID NO: 383 - protein, 56 aa, Triticum aestivum
PGYYPISPQQSEQWQQPGQGQQPGQGQQSGQGQQGQQPGQGQRPGQGQQGYYPTSL

SEQ ID NO: 384 - protein, 56 aa, Triticum aestivum
QQPGQGQQPGQGQQPGQGQQGQQPGQGQQPGQGQQGYYPTSPQQLGQGQQPGQWQQ

SEQ ID NO: 385 - protein, 56 aa, Triticum aestivum
QGSYYPGQASPQQSGQGQQPGQEQQPGQGQQDQQPGQRQQGYYPTSPQQPGQGQQL

SEQ ID NO: 386 - protein, 56 aa, Triticum aestivum
QQSGQGQQPGQGQQSGQGQQGQQPGQGQQAYYPTSSQQSGQRQQAGQWQRPGQGQS

SEQ ID NO: 387 - protein, 56 aa, Triticum aestivum
QGQQGQQPGQGQQGQQPGQGQQGQQPGQGQQPGQGQPGYYPTSLQQSGQGQQPGQW

SEQ ID NO: 388 - protein, 56 aa, Triticum aestivum
SLQQPGQGQQGHYPASLQQVGQGQQIGQPGQRQQPGQGQQTEQGQQLEQGQQPGQG

SEQ ID NO: 389 - protein, 56 aa, Triticum aestivum
YPTTPQQPGQGQQPEQGQPGYYLTSSQQPGQGQQPGRGQPGYYPTSSQQLGQGQQL

SEQ ID NO: 390 - protein, 56 aa, Triticum aestivum
QQGQYPASQQQPGQGQQGQYPASQQQPGQGQQGQYPASQQQPAQGQQGQYPASQQQ

SEQ ID NO: 391 - protein, 56 aa, Triticum aestivum
GQGQQIGQVQQPGQGQQGYYPISLQQSGQGQQSGQGQQSGQGHQLGQGQQSGQEQQ

SEQ ID NO: 392 - protein, 56 aa, Triticum aestivum
QGQQGQYPASQQQPAQGQQGQYPASQQQPGQGQQGHYLASQQQPGQGQQRHYPASL

SEQ ID NO: 393 - protein, 56 aa, Triticum aestivum
PGQGQQPGQGQQGHCPTSPQQTGQAQQPGQGQQIGQVQQPGQGQQGYYPISLQQSG

SEQ ID NO: 394 - protein, 56 aa, Triticum aestivum
GQQSGQGQPGYYPTSLRQPGQWQQPGQGQQPGQGQQGQQPGQGQQPGQGQQGYYPT

SEQ ID NO: 395 - protein, 56 aa, Triticum aestivum
QLGQGQQGQQPGQGQQPAQGQQGQQPGQGQQGQQPGQGQQPGQGQPWYYPTSPQES

SEQ ID NO: 396 - protein, 56 aa, Triticum aestivum
TSPQQSGQLQQPAQGQQPGQEQQGQQPGQGQQGQQPGQGQQPGQGQPGYYPTSPQQ

SEQ ID NO: 397 - protein, 56 aa, Triticum aestivum
QQGQYPASQQQPAQGQQGQYPASQQQPAQGQQGQYPASQQQPGQGQQGQYPASQQQ

SEQ ID NO: 398 - protein, 56 aa, Triticum aestivum
GHYPTSLQQLGQGQQIGQPGQKQQPGQGQQTGQGQQPEQEQQPGQGQQGYYPTSLQ

SEQ ID NO: 399 - protein, 56 aa, Triticum aestivum
QGQQGQQPGQGQQGQQPGQGQQPGQGQPWYYPTSPQQPGQWQQPGQWQQPGQGQPG

SEQ ID NO: 400 - protein, 56 aa, Triticum aestivum
YPTSPQQSGQLQQPAQGQQPGQGQQGQQPGQGQPGYYPTSSQLQPGQLQQPAQGQQ

SEQ ID NO: 401 - protein, 56 aa, Triticum aestivum
QPGQGQQPGQGQQGQQPGQGQQGQQPGQGQQPGEGQQGYYPTFPQQPGQVQQPGQG

SEQ ID NO: 402 - protein, 56 aa, Triticum aestivum
GQGQQPGQLQQPTQGQQGQQPGQGQQGQQPGEGQQGQQPGQGQQPGQGQPGYYPTS

SEQ ID NO: 403 - protein, 56 aa, Triticum aestivum
QGQPGYDPTSPQQPGQGQQPGQLQQPAQGQQGQQLAQGQQGQQPAQVQQGQRPAQG

SEQ ID NO: 404 - protein, 56 aa, Triticum aestivum
GQQLEQGQQPGQGQQTRQGQQLEQGQQPGQGQQTRQGQQLEQGQQPGQGQQGYYPT

SEQ ID NO: 405 - protein, 56 aa, Triticum aestivum
GQQPGQGQQGQQPGQGQQPGQGQQGQQPGQGQQPGQGQQGQQLGQGQQGYYPTSLQ

SEQ ID NO: 406 - protein, 56 aa, Triticum aestivum
PQQPGQGQQPGQLQQPAQGQQPGQGQQGQQPGQGQQGQQPGQGQQPGQGQPGYYPT

SEQ ID NO: 407 - protein, 56 aa, Triticum aestivum
VTSPHQGSYYPGQTSPQQPGQAQQPGQEQQPGQGQQDQQPEQGQQPGKGQQGYYPT

SEQ ID NO: 408 - protein, 56 aa, Triticum aestivum
QQLGQGQQGQQPGQGQQGQQPAQGQQGQQPGQGQQGQQPGQGQQPGQGQPWYYPTS

SEQ ID NO: 409 - protein, 55 aa, Triticum aestivum
QGQQPGQGQQGYDSPYHVSAEYQAARLKVAKAQQLAASLPAMCRLEGSDALSTRQ

SEQ ID NO: 410 - protein, 56 aa, Triticum aestivum
QQGQQPGQGQQGQQPGQGQQGQQPAQGQQGQQPGQGQQGQQPGQGQQGQQPGQGQQ

SEQ ID NO: 411 - protein, 56 aa, Triticum aestivum
SLQQVGQGQQIGQLGQRQQPGQGQQTRQGQQLEQGQQPGQGQQTRQGQQLEQGQQP

SEQ ID NO: 412 - protein, 56 aa, Triticum aestivum
QQQQQPILLQQPPFSQHQQPVLPQQQIPSVQPSILQQLNPCKVFLQQQCSPVAMPQ

SEQ ID NO: 413 - protein, 56 aa, Triticum aestivum
QQQQPVLPQQPPFSQQQQQQPILPQQPPFSQHQQPVLPQQQIPYVQPSILQQLNPC

SEQ ID NO: 414 - protein, 56 aa, Triticum aestivum
QPPFSQQQQPVLPQQPSFSQQQLPPFSQQLPPFSQQQQPVLLQQQIPFVHPSILQQ

SEQ ID NO: 415 - protein, 56 aa, Triticum aestivum
QQPLLPQQPPFSQQRPPFSQQQQQPVLPQQPPFSQHQQPVLPQQQIPYVQPSILQQ

SEQ ID NO: 416 - protein, 56 aa, Triticum aestivum
QQPLLLQQPPLSQQQPPFSRQQQQPPFSQQQQQPILLQQPPFSQHQQPVLPQQQIP

SEQ ID NO: 417 - protein, 56 aa, Triticum aestivum
QQPIQQQPQPFPQQPPCSQQQQPPLSQQQQPPFSQQQPPFSQQELPILPQQPPFSQ

SEQ ID NO: 418 - protein, 56 aa, Triticum aestivum
QQRPPFSQQQQQPVLPQQPPFSQQQQQQPILPQQPPFSQHQQPVLPQQQIPYVQPS

SEQ ID NO: 419 - protein, 56 aa, Triticum aestivum
QQPLLPQQPPFSQQQPPFSQQQQQPILPQQPPFSQHQQPVLPQQQIPSVQPSILQQ

SEQ ID NO: 420 - protein, 56 aa, Triticum aestivum
FSQQQQPVLPQQPPFSQQQQQPVLPQQQIPFVHPSILQQLNPCKVFLQQQCSPVAM

SEQ ID NO: 421 - protein, 56 aa, Triticum aestivum
QQPLLPQQPPFSQQQPPFSQQQQQPPFSQHQQPVLPQQQIPSVQPSILQQLNPCKV

SEQ ID NO: 422 - protein, 56 aa, Triticum aestivum
QQQLAQGTFLQPHQIAQLEVMTSIALRTLPMMCRVNVPLYRTTTSVPFGVGTGVGA

SEQ ID NO: 423 - protein, 56 aa, Triticum aestivum
PPFLQQQQPSLPQQPPFSQQQQQLVLPQQQIPFVHPSILQQLNPCKVFLQQQCSPV

SEQ ID NO: 424 - protein, 56 aa, Triticum aestivum
QQLPPFSQQQQVLPQQPPFSQQQQPVLLQQQIPFVHPSILQQLNPCKVFLQQQCSP

SEQ ID NO: 425 - protein, 56 aa, Triticum aestivum
QQPLLPQQPPFSQQQPPFSQQQQQPPFSQQQQQPILLQQPPFSQHQQPVLPQQQIP

SEQ ID NO: 426 - protein, 56 aa, Triticum aestivum
QQQPILPQQPPFSQQQQPVLLQQQIPFVHPSILQQLNPCKVFLQQQCSPVAMPQSL

SEQ ID NO: 427 - protein, 55 aa, Triticum aestivum
GQQPQQQQLAQGTFLQPHQIAQLEVMTSIALRTLPTMCNVNVSLYRTTTRVPFGV

SEQ ID NO: 428 - protein, 56 aa, Triticum aestivum
GQCSFQQPQQQLGQQPQQQQVQKGTFLQPHQIARLEVMTSIALRTLPTMCSVNVPL

SEQ ID NO: 429 - protein, 55 aa, Triticum aestivum
QLAQGTFLQPHQIAQLEVMTSIALRILPTMCRVNVPLYRTTTSVPFDVGTGVGAY

SEQ ID NO: 430 - protein, 55 aa, Triticum aestivum
QLAHGTFLQPHQIAQLEVMTSIALHNLPMMCSVNVPLYETTTSVPLGIGIGVGVY

SEQ ID NO: 431 - protein, 55 aa, Triticum aestivum
QLAHGTFLQPHQIAQLEVMTSIALRTLPKMCSVNVPLYETPPSVPLGVGIGVGVY

SEQ ID NO: 432 - protein, 55 aa, Triticum aestivum
QLTHGAFLQPHQIAQLEVMTSIALRNLPRMCSVNVPLYETTTSVPLGVGIGVGVY

SEQ ID NO: 433 - protein, 55 aa, Triticum aestivum
QLAHGTFLQPHQIAQLEVMTSIALRTLPTMCSVNVPLYETTTSVPLGVGIGVGVY

SEQ ID NO: 434 - protein, 55 aa, Triticum aestivum
QLAQGTFLQPHQIAQLEVMTSIALRTLPMMCRVNVPLYRTTTSVPFGVGTGVGAY

SEQ ID NO: 435 - protein, 56 aa, Triticum aestivum
QQLAQGTFLQPHQIAQLEVMTSIALRTLPTMCNVNVPLYRTTTRVPFGVGTGVGGY

SEQ ID NO: 436 - protein, 55 aa, Triticum aestivum
QLAQGTFLQPHQIAQLELMTSIALRTLPTMCNVNVPLYRTTTRVPFGVGTGVGAY

SEQ ID NO: 437 - protein, 55 aa, Triticum aestivum
QLAQGTFLQPHQIAQLEVMTSIALRTLPTMCRVNVPLYRTTTSVPFGVSAGVGAY

SEQ ID NO: 438 - protein, 55 aa, Triticum aestivum
QQLAQGTFLQPHQIAQLEVMTSIALRTLPTMCNVNVPLYRTTTRVPFGVGTGVGG

SEQ ID NO: 439 - protein, 56 aa, Triticum aestivum
QPQQLGQCVSQPQQQLQQQLGQQPQQQQLAHGTFLQPHQIAQLEVMTSIALHNLPM

SEQ ID NO: 440 - protein, 56 aa, Triticum aestivum
QLGQCVSQPQQQLQQQLGQQPQQQQLAHGTFLQPHQIAQLEVMTSIAPRTLPTMCS

SEQ ID NO: 441 - protein, 55 aa, Triticum aestivum
QIPQGIFLQPHQISQLEVMTSIALRTLPTMCGVNVPLYSSTTIMPFSIGTGVGGY

SEQ ID NO: 442 - protein, 55 aa, Triticum aestivum
QVPQGTLLQPHQIAQLELMTSIALRTLPMMCSVNVPVYGTTTSVPFGVGTQVGAY

SEQ ID NO: 443 - protein, 55 aa, Triticum aestivum
QVPQGTLLQPHQIAQLEVMTSIALRTLPTMCSVNVPVYGTTTIVPFGVGTRVGAY

SEQ ID NO: 444 - protein, 56 aa, Triticum aestivum
LGQWPQQQQVPQGTLLQPHQIAQLEVMTSIALRTLPTMCSVNVPVYGTTTIVPFGV

SEQ ID NO: 445 - protein, 56 aa, Triticum aestivum
CSFQQPQQLQQLGQQPQQQQIPQGIFLQPHQISQLEVMTSIALRTLPTMCGVNVPL

SEQ ID NO: 446 - protein, 56 aa, Triticum aestivum
QQQQPVLPQQPPFSQQQQQPILPQQPPFSQQQQPVLLQQQIPFVHPSILQQLNPCK

SEQ ID NO: 447 - protein, 56 aa, Triticum aestivum
QQQPFPQQQQPLRPQQPPFSQQQPPFSQQQQQPVLPQQPPFSQHQQPVLPQQQIPS

SEQ ID NO: 448 - protein, 56 aa, Triticum aestivum
PPFSQQQQTPFSQQQQIPVIHPSVLQQLNPCKVFLQQQCIPVAMQRCLARSQMLQQ

SEQ ID NO: 449 - protein, 56 aa, Triticum aestivum
LPPFSQQLPPFSQQQQPVLPQQPPFSQQQQQPILPQQPPFSQQQQPVLLQQQIPFV

SEQ ID NO: 450 - protein, 56 aa, Triticum aestivum
QQPPFSQQQQPSFSQQQQPPFSQQQPPFSQQQQQPVLPQQQIPFVHPSILQQLNPC

SEQ ID NO: 451 - protein, 56 aa, Triticum aestivum
FSQQPPISQQQQPPFSQQQQPPFSQQQQIPVIHPSVLQQLNPCKVFLQQQCIPVAM

SEQ ID NO: 452 - protein, 56 aa, Triticum aestivum
SQPQQQQKQLGQCSFQRPQQQQLGQWPQQQQVPQGTLLQPHQIAQLELMTSIALRT

SEQ ID NO: 453 - protein, 56 aa, Triticum aestivum
FSQQQQPVLPQQPPFSQQQQPILPQQPPFSQQQQQPVLPQQQIPFVHPSILQQLNP

SEQ ID NO: 454 - protein, 56 aa, Triticum aestivum
PFTQQQQPPFSQQPPISQQQQPPFSQQQQPPFSQQQQIPVIHPSVLQQLNPCKVFL

SEQ ID NO: 455 - protein, 56 aa, Triticum aestivum
SQQQQPVLPQQPSFSQQQLPPFSQQQPPFSQQQQQPVLPQQQILFVHPSILQQLNP

SEQ ID NO: 456 - protein, 56 aa, Triticum aestivum
QQPVLPQQPSFSQQQLPPFSQQLPPFSQQQQPVLPQQPPFLQQQLPPFSQQLPPFS

SEQ ID NO: 457 - protein, 56 aa, Triticum aestivum
FSQQQQQPVLPQQTPLSQQQQPVLQQQPPFSQQQQQPVLPQQQIPFVHPSILQQLN

SEQ ID NO: 458 - protein, 56 aa, Triticum aestivum
GSIQTPQQQPQQLGQCVSQPQQQSQQQLGQQPQQQQLAQGTFLQPHQIAQLEVMTS

SEQ ID NO: 459 - protein, 56 aa, Triticum aestivum
VQGVSQPQQQQKQLGQCPFQQPQQQQLGQWPQQQQVPQGTLLQPHQIAQLEVMTSI

SEQ ID NO: 460 - protein, 56 aa, Triticum aestivum
QPPFSQQQPPFSQQQQQPPISQQQQQQIIPQQPPFSQHQQPVLPQQQIPSVQPSIL

SEQ ID NO: 461 - protein, 56 aa, Triticum aestivum
QQQGQSVIQYQQQQPQQLGQCVSQPQQQLQQQLGQQPQQQQLAHGTFLQPHQIAQL

SEQ ID NO: 462 - protein, 56 aa, Triticum aestivum
QQQLPPFSQQQQPPFSQQQQPVLPQQPSFSQQQLPPFSQQLPPFSQQQQPVLLQQQ

SEQ ID NO: 463 - protein, 56 aa, Triticum aestivum
PVLPQQPSFSQQQLPPFSQQQQPPFSQQQQPVLPQQPSFSQQQLPPFSQQLPPFSQ

SEQ ID NO: 464 - protein, 56 aa, Triticum aestivum
QQQQQPFTQQQQPPFSQQPPISQQQQPPFLQQQRPPFSRQQQIPVIHPSVLQQLNP

SEQ ID NO: 465 - protein, 56 aa, Triticum aestivum
NQPLLPQQPPFSQQQPPFSQQQQQPPFSQQQQPPFSQHQQPVLPQQQIPSVQPSIL

SEQ ID NO: 466 - protein, 56 aa, Triticum aestivum
QQLPPFSQQQQQVLPQQPPFSQQQQQQPIPPQQPPFSQQQQPVLLQQQIPFVHPSI

SEQ ID NO: 467 - protein, 56 aa, Triticum aestivum
PQQFPQQQPCSQQQQQQQQQQQQQQQPLSQQQQPPFSQQQPPFSQQQQPVLPQQPS

SEQ ID NO: 468 - protein, 56 aa, Triticum aestivum
FSQQQLPPFSQQQPPFSQQQQPVLPQQPPFSQQQPVLPPQQSPFPQQQQHQQLVQQ

SEQ ID NO: 469 - protein, 56 aa, Triticum aestivum
FSQQQLPPFSQQQPPFSQQQQPVLPQQPPFSQQQQQQPILPQQPSFSQQQQQLVLP

SEQ ID NO: 470 - protein, 56 aa, Triticum aestivum
LERPWQQQPLPPQQTFPQQPPFSQQQQQPFPQQPPFSQQQPPFSQQQQPVLPQQPS

SEQ ID NO: 471 - protein, 56 aa, Triticum aestivum
PFPQQPPFSQQQPPFSQQQQPVLPQQPSFSQQQLPPFSQQQPPFSQQQQPVLPQQP

SEQ ID NO: 472 - protein, 56 aa, Triticum aestivum
QQPPFSQQQQPSFSQQQQPPFSQQQPPFSQQQQPVIPQQPSFSQQQLPPFSQQQPP

SEQ ID NO: 473 - protein, 56 aa, Triticum aestivum
FSQQQQPPFSQQQPPFSQQQQPVLPQQPSFSQQQLPPFSQQQSPFSQQQQIVLQQQ

SEQ ID NO: 474 - protein, 56 aa, Triticum aestivum
PPFSQQLPPFLQQQQPVLPQQPPFSQQQLPPFSQQLPPFSQQQQPVLPQQPPFSQQ

SEQ ID NO: 475 - protein, 56 aa, Triticum aestivum
QPFPQQPPCSQQQQPPLSQQQQPPFSQQQPPFSQQQQPVLPQQPPFSQQQQQFPQQ

SEQ ID NO: 476 - protein, 56 aa, Triticum aestivum
SQQQQPVIPQQPSFSQQQLPPFSQQQPPFSQQQQPVLPQQPPFSQQQQPILPQQPP

SEQ ID NO: 477 - protein, 56 aa, Triticum aestivum
QQPPFSQQQQPVLPQQPPFSQQQQQQPILPQQPSFSQQQQQLVLPQQQIPFVHPSI

SEQ ID NO: 478 - protein, 56 aa, Triticum aestivum
QPFPQQPPCSQQQQPPLLQQQQPPFSQQQPPFSQQQQPVLPQQPPFSQQQQPLLPQ

SEQ ID NO: 479 - protein, 56 aa, Triticum aestivum
QQQPVLPQQPPFLQQQLPPFSQQLPPFSQQQQPVLPQQPPFSQQQQQPILPQQPPF

SEQ ID NO: 480 - protein, 56 aa, Triticum aestivum
QPFPQQPACSQQQQPPLLQQQQPPFSQQQPPFSQQQQPVLPQQPPFSQQQQPQFAQ

SEQ ID NO: 481 - protein, 56 aa, Triticum aestivum
QQQQQQQPLSQQQQPPFSQQQQPPFSQQQPPFSQQQQPVLPQQPSFSQQQLPPFSQ

SEQ ID NO: 482 - protein, 56 aa, Triticum aestivum
QQQPPFSQQQQQPLSQQQQPPFSQQQPPFSQQQQPPFSQQQPPFSQQQQPVLPQQP

SEQ ID NO: 483 - protein, 56 aa, Triticum aestivum
QPPFSQQELPVLPQQPPFSQQQQPFPQQQQPLLPQQPPFSQQRPPFSQQQQQPVLP

SEQ ID NO: 484 - protein, 56 aa, Triticum aestivum
FSQQQQPVLPQQPPFSQQQQPILPQQPPFSQQQQQPVLPQQTPLSQQQQPVLQQQP

SEQ ID NO: 485 - protein, 56 aa, Triticum aestivum
QPFPQQPPCSQQQQPPLSQQQQPPFSQQQPPFSQQELPILPQQPPFSQQQQPQFSQ

SEQ ID NO: 486 - protein, 56 aa, Triticum aestivum
PQPFPQQQPCSQQQQPPLSQQQQPPFSQQQPPFSQQQQPSFSQQQQPPFSQQQPPF

SEQ ID NO: 487 - protein, 56 aa, Triticum aestivum
PQQPPFSQQQQLVLPQQPPFSQQQQPVLPPQQSPFPQQQRHQQLVQQQIPVVQPSI

SEQ ID NO: 488 - protein, 56 aa, Triticum aestivum
PQPFSQQQPCSQQQQQQPLSQQQQPPFSQQQPPFSQQQQQPLSQQQQPPFSQQQPP

SEQ ID NO: 489 - protein, 56 aa, Triticum aestivum
SFSQQQLPPFSQQQSPFSQQQQIVLQQQPPFLQQQQPSLPQQPPFSQQQQQLVLPQ

SEQ ID NO: 490 - protein, 56 aa, Triticum aestivum
QQPPFSQQQPPFSQQELPILPQQPPFSQQQQPQFSQQQQPFPQQQQPLLLQQPPFS

SEQ ID NO: 491 - protein, 56 aa, Triticum aestivum
LERPWQQQPLPPQQTLAQQPLFSQQQQLFAKQPSFSQQQPPFWQQQPPFSHQQPIL

SEQ ID NO: 492 - protein, 56 aa, Triticum aestivum
QPPFSQQQQPILPQQPPFSQQQQQFPQQQQPLLLQQPPLSQQQPPFSRQQQQPPFS

SEQ ID NO: 493 - protein, 56 aa, Triticum aestivum
QQQQPPLSQQQQPPFSQQQQPPFSQQQQPVLPQQPSFSQQQLPPFSQQQQPPFSQQ

SEQ ID NO: 494 - protein, 56 aa, Triticum aestivum
PFSQQQQPVLPQQPPFSQRQLPPFSQQQQPPFSQQQQPVLPQQPPFSQQQQPVLLQ

SEQ ID NO: 495 - protein, 56 aa, Triticum aestivum
QPPFSQQELPILPQQPPFSQQQQPQFSQQQQPFPQQQQPLLLQQPPFSQQRPPFSQ

SEQ ID NO: 496 - protein, 56 aa, Triticum aestivum
LERPWQQQPLPPQQTFPQQPLFSQQQQLFPQQPSFSQQQPPFWQQQPPFSQQQPIL

SEQ ID NO: 497 - protein, 56 aa, Triticum aestivum
QPPFSQQQQPVLPQQPPFSQQQQPLLPQQPPFSQQQPPFSQQQQQPPISQQQQQQI

SEQ ID NO: 498 - protein, 56 aa, Triticum aestivum
QPPFSQQQQPVLPQQPPFSQQQQPQFAQQQQPFPQQQQPLRPQQPPFSQQQPPFSQ

SEQ ID NO: 499 - protein, 56 aa, Triticum aestivum
LERPWQQQPLQQKETFPQQPPSSQQQQPFPQQPPFLQQQPSFSQQPLFSQKQQPVL

SEQ ID NO: 500 - protein, 56 aa, Triticum aestivum
LERPWQQQPLQQKETFPQQPPSSQQQQPLPQQPPFLQQQPSFSQQPLFSQKQQPVL

SEQ ID NO: 501 - protein, 56 aa, Triticum aestivum
QPPFSQQQQPVLPQQPPFSQQQQQFPQQQQPLLPQQPPFSQQQPPFSQQQQQPPFS

SEQ ID NO: 502 - protein, 56 aa, Triticum aestivum
PFSQQQQPPFSQQQQPPFSQQQQSPFSQQQEQQQQPPFLQQQQPPFSQQPPISQQQ

SEQ ID NO: 503 - protein, 56 aa, Triticum aestivum
FAKQPSFSQQQPPFWQQQPPFSHQQPILPQQPPFSQQQQLVLPQQPPFSQQQQPVL

SEQ ID NO: 504 - protein, 56 aa, Triticum aestivum
QQQPQFSQQQQPFPQQQQPLLLQQPPFSQQRPPFSQQQQQPVLPQQPPFSQQQQQQ

SEQ ID NO: 505 - protein, 56 aa, Triticum aestivum
QQQPFPQQQQPLLLQQPPFSQQRPPFSQQQQQPVLPQQPPFSQQQQQQPILPQQPP

SEQ ID NO: 506 - protein, 56 aa, Triticum aestivum
SQQQQPPFSQQQQPPFSQQQQPPFSQQQQQQQQQQQQPFTQQQPPFSQQPPISQQQ

SEQ ID NO: 507 - protein, 56 aa, Triticum aestivum
LERPWHHHPLPPQHTFPQQPLFSQQQQLFPQQPSFSQQQPPFWQQQPPFSQQQPIL

SEQ ID NO: 508 - protein, 56 aa, Triticum aestivum
QQPIIILQQSPFSQQQQIVLQQQPPFLQQQQPSLPQQPPFSQQQQQLVLPQQQIPF

SEQ ID NO: 509 - protein, 56 aa, Triticum aestivum
LFSQQQQPPFSQQQQPPFSQQQQSPFSQQQQQPPFLQQQQPPFSQQPPISQQQQPP

SEQ ID NO: 510 - protein, 56 aa, Triticum aestivum
PFSQQQQPPFSQQQQSPFSQQQQPPFLQQQQPPFSQQPPISQQQQPPFSQQQQPQF

SEQ ID NO: 511 - protein, 56 aa, Triticum aestivum
QPPFSQQQQPPFSQQQQPPFSQQQQQQQPPFTQQQQPPFSQQPPISQQQQPPFSQQ

SEQ ID NO: 512 - protein, 56 aa, Triticum aestivum
PFSQQQQPPFSQQQQPPFSQQQQSPFSQQQQQPPFSQQQQPPFSQQPLISQQQQLP

SEQ ID NO: 513 - protein, 56 aa, Triticum aestivum
QQPPFSQQQQPPFSQQQQQPPFTQQQQQQQQQQPFTQQQQPPFSQQPPISQQQQPP

SEQ ID NO: 514 - protein, 56 aa, Triticum aestivum
QPPFSQEQQPPFSQQQQPPFSQQQQPPYSQQQQPPFSQQQQPPFSQQQQPPFSQQQ

SEQ ID NO: 515 - protein, 56 aa, Triticum aestivum
FSQQQQPQFSQQQQPPYSQQQQPPYSQQQQPPFSQQQQPPFSQQQQPPFSQQQQQP

SEQ ID NO: 516 - protein, 56 aa, Triticum aestivum
QQPPFSQQQQPPFSQQQQPPFSQQQQQPPFTQQQQPSFSQQPPISQQQQQQQQQQQ

SEQ ID NO: 517 - protein, 56 aa, Triticum aestivum
QQQPPFSQQPPISQQQQPPFSQQQQPQFSQQQQPPYSQQQQPPYSQQQQPPFSQQQ

SEQ ID NO: 518 - protein, 56 aa, Triticum aestivum
SQQQQPPYSQQQQPPYSQQQQPPFSQQQQPPFSQQQQPPFLQQQQQPPFTQQQQPS

SEQ ID NO: 519 - protein, 56 aa, Triticum aestivum
QPPFSQQQQPPFLQQQQQPPFTQQQQPSFSQRPPISQQQQQQQQQQQPFTQQQQPP

SEQ ID NO: 520 - protein, 56 aa, Triticum aestivum
FSQRPPISQQQQQQQQQQQPFTQQQQPPFSQQPPISQQQQPPFSQQQQPPFSQQQQ

SEQ ID NO: 521 - protein, 56 aa, Triticum aestivum
PQQFPQQQPCSQQQQQQQQQQQQQQQQQQQQQQQQPLSQQQQPPFSQQQQPPFSQQ

SEQ ID NO: 522 - protein, 56 aa, Triticum aestivum
QQTLSHHHQQQPIQQQPQQFPQQQPCSQQQQQPPLSQQQQPPFSQQQQPPFSQQQQ

SEQ ID NO: 523 - protein, 56 aa, Triticum aestivum
PFSQQQQPQFSQQPPFSQQQQPPFSQQQQQPPFAQQQQPPFSQQPPISQQQQPPFS

SEQ ID NO: 524 - protein, 56 aa, Triticum aestivum
QQQPPFSQQQQPPFSQQPPISQQQQPQFLQQQQPPFSQQQQPPFSQQQQPPYSQQQ

SEQ ID NO: 525 - protein, 56 aa, Triticum aestivum
QQQQPPFSQQQQPPFSQQPLISQQQQLPFSQQQQPQFSQQQQPPYSQQQQPPYSQQ

SEQ ID NO: 526 - protein, 56 aa, Triticum aestivum
QQPPFSQQQQPPFSQQQQPSFSQQQQQPPFTQQQQPPFSQQSPISQQQQQQQQQQQ

SEQ ID NO: 527 - protein, 56 aa, Triticum aestivum
SQQQQPPFSQQQQPPFSQQQQPPFSQQQQQTNKQQQQQPFTQQQPPFSQQPPISQQ

SEQ ID NO: 528 - protein, 56 aa, Triticum aestivum
QQTNKQQQQQPFTQQQPPFSQQPPISQQQQQQQQQQQPFTQQQPPFSQQPPISQQQ

SEQ ID NO: 529 - protein, 56 aa, Triticum aestivum
LEKPSQQQPLPLQQTLSHHQQQQPVQQQPQPFPQQQPCSQQQQPPLSQQQQPPFSQ

SEQ ID NO: 530 - protein, 56 aa, Triticum aestivum
QQQQPPFLQQQQPPFSQQPPISQQQQPPFSQQQQPQFSQQQQPPYSQQQQPPYSQQ

SEQ ID NO: 531 - protein, 56 aa, Triticum aestivum
PFTQQQQPSFSQQPPISQQQQQQQQQQQPFTQQQQPPFSQQPPISQQQQPPFSQQQ

SEQ ID NO: 532 - protein, 56 aa, Triticum aestivum
LEKPLQQQPLPLQQILWYQQQQPIQQQPQPFPQQPPCSQQQQPPLSQQQQPPFSQQ

SEQ ID NO: 533 - protein, 56 aa, Triticum aestivum
QQQQQQQQQPFTQQQPPFSQQPPISQQQQQQQQQPQPFTQQQPPFSQQPPISQQQQ

SEQ ID NO: 534 - protein, 56 aa, Triticum aestivum
LERPSQQQPLPPQQTLSHHHHQQPIQQQPQQFPQQQPCSQQQQQPPLSQQQQPPFS

SEQ ID NO: 535 - protein, 56 aa, Triticum aestivum
PQQFPQQQPCSQQQQQQQQQQQQQQQQQQQQQQQQQQQQPLSQQQQPPFSQQQQPP

SEQ ID NO: 536 - protein, 56 aa, Triticum aestivum
LERPSQQQPLPPQQTLSHHQQQQPIQQQPQPFSQQQPCSQQQQQQPLSQQQQPPFS

SEQ ID NO: 537 - protein, 56 aa, Triticum aestivum
LEKPSQQQPLPLQQILWYHQQQPIQQQPQPFPQQPACSQQQQPPLLQQQQPPFSQQ

SEQ ID NO: 538 - protein, 56 aa, Triticum aestivum
LEKPWQQQPLPPQQQPPCSQQQQPFPQQQQPIIILQQSPFSQQQQIVLQQQPPFLQ

SEQ ID NO: 539 - protein, 56 aa, Triticum aestivum
MGRLLSPRGKELHTPQEQFPQQQQFPQPQQFPQQQILQQHQIPQQPQQFPQQQQFL

SEQ ID NO: 540 - protein, 56 aa, Triticum aestivum
QQQFPQQQFPQQQFPQQEFPQQQQFPQQQIAQQPQQLPQQQFPIPYPPQQSQEPSP

SEQ ID NO: 541 - protein, 56 aa, Triticum aestivum
QQQQIPQQQIPQQHQIPQQPQQFPQQQFPQQQQFPQQHQSPQQQFPQQQFPQQQLP

SEQ ID NO: 542 - protein, 56 aa, Triticum aestivum
PQQQQFPQQHQSPQQQFPQQQFPQQQLPQQEFSQQQISQQPQQLPQQQQIPQQPQQ

SEQ ID NO: 543 - protein, 56 aa, Triticum aestivum
IPQQQQIPQQPQQIPQQQQIPQQPKQFPQQQFPQQQFPQQQFPQQEFPQQQQFPQQ

SEQ ID NO: 544 - protein, 56 aa, Triticum aestivum
AACQASQLAVCASAILSGAKPSGECCGNLRAQQGCFCQYAKDPTYGQYIRSPHARD

SEQ ID NO: 545 - protein, 56 aa, Triticum aestivum
QFPQQQFPQQKLPQQEFPQQQISQQPQQLPQQQQIPQQPQQFLQQQQFPQQQPPQQ

SEQ ID NO: 546 - protein, 56 aa, Triticum aestivum
LQSQQPFHQQPEQIISQQPQQPFSLQPQQPFSQPQQPFPQQPGQIIPQQPQQPFPL

SEQ ID NO: 547 - protein, 56 aa, Triticum aestivum
PQQPFPQPQLPFPQQSEQIIPQQLQQPFPLQPQQPFPQQPQQPFPQPQQPIPVQPQ

SEQ ID NO: 548 - protein, 56 aa, Triticum aestivum
QQQQIPQQPQQFPQQQFPQQQFPQQQFPQQEFPQQQQFPQQQIARQPQQLPQQQQI

SEQ ID NO: 549 - protein, 56 aa, Triticum aestivum
PQQPFPQQPQRPQQSFPQQPEQIIPQQPQQPFPLQPQQQFPEQSEQIISQQRQQPF

SEQ ID NO: 550 - protein, 56 aa, Triticum aestivum
QPSPLQPQQPFPQQPEQIIPQQPQQPFLLQSQQPFHQQPEQIISQQPQQPFSLQPQ

SEQ ID NO: 551 - protein, 55 aa, Triticum aestivum
PAQQPFPQQPGQIIPQQPQQPLPLQPQQPFPWQPEQRSSQQPQQPFSLQPQQPFS

SEQ ID NO: 552 - protein, 56 aa, Triticum aestivum
PFPWQPQQPFPQTQQSFPLQPQQPFPQQPQQPFPQPQLPFPQQSEQIIPQQLQQPF

SEQ ID NO: 553 - protein, 55 aa, Triticum aestivum
QQPQQPFPLQPQQPFPQQPQQPFPQQPQQSFPQQPQQPYPQQQPYGSSLTSIGGQ

SEQ ID NO: 554 - protein, 56 aa, Triticum aestivum
PQQQQLTQQQFPRPQQSPEQQQFPQQQFPQQPPQQFPQQQFPIPYPPQQSEEPSPY

SEQ ID NO: 555 - protein, 56 aa, Triticum aestivum
QPQQPFPQQPQQPFPLQPQQSFPQQPQQPFPQPQQAFPEQGEQIIPQQPQQPFPLQ

SEQ ID NO: 556 - protein, 55 aa, Triticum aestivum
PFPLQPQQSFPQQPQQPFPQPQQAFPEQGEQIIPQQPQQPFPLQPHQPQQPYPQQ

SEQ ID NO: 557 - protein, 56 aa, Triticum aestivum
ELQSPQQSFSYQQQPFPQQPYPQQPYPSQQPYPSQQPFPTPQQQFPEQSQQPFTQP

SEQ ID NO: 558 - protein, 56 aa, Triticum aestivum
QQPTPIQPQQPFPQQPQQPQQPFPQPQQPFPWQPQQPFPQTQQSFPLQPQQPFPQQ

SEQ ID NO: 559 - protein, 56 aa, Triticum aestivum
PLQPQQPFPQQPQQPFPQPQQPIPVQPQQSFPQQSQQSQQPFAQPQQLFPELQQPI

SEQ ID NO: 560 - protein, 56 aa, Triticum aestivum
PQQPQQPFPLQPQQPFPQQPQQPFPQQPQQSFPQQPQQPYPQQQPYGSSLTSIGGQ

SEQ ID NO: 561 - protein, 55 aa, Triticum aestivum
QQFPQQQFPQQPPQQFPQQQFPIPYPPQQSEEPSPYQQYPQQQPSGSDVISISGL

SEQ ID NO: 562 - protein, 56 aa, Triticum aestivum
PYPQQAFPIPQQYSPHQPQQPFPQPQRPTPLQSQQPFPQQPKQPQQSFSQPQQQFP

SEQ ID NO: 563 - protein, 56 aa, Triticum aestivum
TPLQSQQPFPQQPKQPQQSFSQPQQQFPLQPQQPFPQPQQPIPQQPRQPFPEQPQR

SEQ ID NO: 564 - protein, 56 aa, Triticum aestivum
PQQPQQSFPQPQQQFPLQPQQPFPQPQQPQQPFPQQPQRPQQSFPQQPEQIIPQQP

SEQ ID NO: 565 - protein, 56 aa, Triticum aestivum
QPFSQPQQPFPQQPGQIIPQQPQQPFPLQPQQPFPQQPQQPFPLQPQQSFPQQPQQ

SEQ ID NO: 566 - protein, 56 aa, Triticum aestivum
APTIISPATRTNNSLATPTTIPPATATTIPPATRTNNSPATATTIPPAPQQRFPHT

SEQ ID NO: 567 - protein, 56 aa, Triticum aestivum
RQKFPRNPNNHSLCSTHHFPAQQPFPQQPGQIIPQQPQQPLPLQPQQPFPWQPEQR

SEQ ID NO: 568 - protein, 56 aa, Triticum aestivum
QSFPQQSQQSQQPFAQPQQLFPELQQPIPQQPQQPFPLQPQQPFPQQPQQPFPQQP

SEQ ID NO: 569 - protein, 56 aa, Triticum aestivum
QQEFPQQQQFPQQQIARQPQQLPQQQQIPQQPQQFPQQQQFPQQQSPQQQQFPQQQ

SEQ ID NO: 570 - protein, 56 aa, Triticum aestivum
PQQPQQFPQQQQFPQQQSPQQQQFPQQQFPQQQQLPQKQFPQPQQIPQQQQIPQQP

SEQ ID NO: 571 - protein, 56 aa, Triticum aestivum
FPQQQQLPQKQFPQPQQIPQQQQIPQQPQQFPQQQFPQQQQFPQQQEFPQQQFPQQ

SEQ ID NO: 572 - protein, 56 aa, Triticum aestivum
QQFPQQQFPQQQQFPQQQEFPQQQFPQQQFHQQQLPQQQFPQQQFPQQQFPQQQQF

SEQ ID NO: 573 - protein, 56 aa, Triticum aestivum
LQPQQPFPQPQQPIPQQPRQPFPEQPQRPQQPQQSFPQPQQQFPLQPQQPFPQPQQ

SEQ ID NO: 574 - protein, 56 aa, Triticum aestivum
QQPYPSQQPFPTPQQQFPEQSQQPFTQPQQPTPIQPQQPFPQQPQQPQQPFPQPQQ

SEQ ID NO: 575 - protein, 56 aa, Triticum aestivum
QFHQQQLPQQQFPQQQFPQQQFPQQQQFPQQQQLTQQQFPRPQQSPEQQQFPQQQF

SEQ ID NO: 576 - protein, 56 aa, Triticum aestivum
HQFPQQQLPQQQQIPQQQQIPQQPQQIPQQQQIPQQPQQFPQQQFPQQQFPQQQFP

SEQ ID NO: 577 - protein, 56 aa, Triticum aestivum
SLQPQQPFSQPQQPLSQQPGQIIPQQPQQPSPLQPQQPFPQQPEQIIPQQPQQPFL

SEQ ID NO: 578 - protein, 56 aa, Triticum aestivum
SGPWMCYPGQAFQVPALPGCRPLLKLQCNGSQVPEAVLRDCCQQLADISEWCRCGA

SEQ ID NO: 579 - protein, 56 aa, Triticum aestivum
ISPECHPVVVSPVAGQYEQQIVVPPKGGSFYPGETTPPQQLQQRIFWGIPALLKRY

SEQ ID NO: 580 - protein, 56 aa, Triticum aestivum
MGSKGFKGVIVCLLILGLVLEQLQVEGKSCCRSTLGRNCYNLCRARGAQKLCAGVC

SEQ ID NO: 581 - protein, 56 aa, Triticum aestivum
RCKISSGLSCPKGFPKLALESNSDEPDTIEYCNLGCRSSVCDYMVNAAADDEEMKL

SEQ ID NO: 582 - protein, 56 aa, Triticum aestivum
MGSKGLKGVMVCLLILGLVLEQVQVEGKSCCRTTLGRNCYNLCRSRGAQKLCSTVC

SEQ ID NO: 583 - protein, 55 aa, Triticum aestivum
PDTIEYCNLGCRSSVCDYMVNAAADDEEMKLYVENCGDACVNFCNGDAGLTSLDA

SEQ ID NO: 584 - protein, 56 aa, Triticum aestivum
AQKLCAGVCRCKIASGLSCPKGFPKLALESNSDEPDTIEYCNLGCRSSVCDYMVNA

SEQ ID NO: 585 - protein, 56 aa, Triticum aestivum
MKPHHDGYKYTCSIIVTFHYPNFKHQDQKHQFQESIKHKSKMKTFIIFVLLSMPMS

SEQ ID NO: 586 - protein, 56 aa, Triticum aestivum
KHQFQESIKHKSKMKTFIIFVLLSMPMSIVIAARHLNPSDQELQSPQQQFLEKTII

SEQ ID NO: 587 - protein, 56 aa, Triticum aestivum
IVIAARHLNPSDQELQSPQQQFLEKTIISAATISTSTIFTTTTISHTPTIFPPSTT

SEQ ID NO: 588 - protein, 56 aa, Triticum aestivum
SAATISTSTIFTTTTISHTPTIFPPSTTTTISPTPTTNPPTTTMTIPLATPTTTTT

SEQ ID NO: 589 - protein, 56 aa, Triticum aestivum
TTISPTPTTNPPTTTMTIPLATPTTTTTFSPAPTTISLATTTTISLAPTTNSPITT

SEQ ID NO: 590 - protein, 56 aa, Triticum aestivum
QQQLIPCRDVVLQQPNIAHASSQVSQQSYQLLQQLCCQQLWQTPEQSRCQAIHNVI

SEQ ID NO: 591 - protein, 56 aa, Triticum aestivum
YQLLQQLCCQQLWQTPEQSRCQAIHNVIHAIILHHQQQQQQQQQQQQQQQQQQQQQ

SEQ ID NO: 592 - protein, 55 aa, Triticum aestivum
SQQNPQAQGFVQPQQLPQFEEIRNLALQTLPAMCNVYIPPYCSTTIAPFGIFSTN

SEQ ID NO: 593 - protein, 56 aa, Triticum aestivum
MKKFLVLALIVVAATTTAAEPFTAFRSAWEPQHPSSPEHQPTPQPQEHPVPHQKLN

SEQ ID NO: 594 - protein, 56 aa, Triticum aestivum
CLVMWEQCCQQLKAIPKQSRCEAIHNVVHAIILQQQQQLVQATSTQPQQQQQQGQQ

SEQ ID NO: 595 - protein, 56 aa, Triticum aestivum
HAIILQQQQQLVQATSTQPQQQQQQGQQQQQGLGSSQPQQQTQLDQGWIAVIGTWV

SEQ ID NO: 596 - protein, 56 aa, Triticum aestivum
QQQGLGSSQPQQQTQLDQGWIAVIGTWVIQTIPAMCDVHVPPYCYTTISPSSDVTT

SEQ ID NO: 597 - protein, 33 aa, Triticum aestivum
IQTIPAMCDVHVPPYCYTTISPSSDVTTDMGGY

SEQ ID NO: 598 - protein, 56 aa, Triticum aestivum
MKTFLIFALLAVAATSAIAQMENSHIPGLERPSQQQPLPPQQTLTHHQQQQPIQQQ

SEQ ID NO: 599 - protein, 56 aa, Triticum aestivum
SSCHVMQQQCCRQLPQIPQQSRYEAIRAIVYSIILQEQQQVQGSIQTQQQQPQQLG

SEQ ID NO: 600 - protein, 56 aa, Triticum aestivum
IVYSIILQEQQQVQGSIQTQQQQPQQLGQCVSQPQQQSQQQLGQQPQQQQLAQGTF

SEQ ID NO: 601 - protein, 56 aa, Triticum aestivum
MKTLLILTILVMAVTIGTANMQVDPSGQVQWPQQQPVLLPQQPFSQQPQQTFPRPQ

SEQ ID NO: 602 - protein, 56 aa, Triticum aestivum
QRSFIQPSLQQQLNPCKNILLQQCKPASLVSSLWSIIWPQSDCQVMRQQCCQQLAQ

SEQ ID NO: 603 - protein, 56 aa, Triticum aestivum
LVSSLWSIIWPQSDCQVMRQQCCQQLAQIPQQLQCAAIHSVVHSIIMQQQQQQQQQ

SEQ ID NO: 604 - protein, 56 aa, Triticum aestivum
QPQQSFPQQQQPLIQLSLQQQMNPCKNFLLQQCNPVSLVSSLISMILPRSDCQVMQ

SEQ ID NO: 605 - protein, 56 aa, Triticum aestivum
LLQQCNPVSLVSSLISMILPRSDCQVMQQQCCQQLAQIPQQLQCAAIHSVVHSIIM

SEQ ID NO: 606 - protein, 55 aa, Triticum aestivum
VQGQGIIQPQQPAQLEVIRSLALRTLPTMCNVYVSPDCSTINAPFASIVVGIGGQ

SEQ ID NO: 607 - protein, 56 aa, Triticum aestivum
IQTQQQQPQQLGQCVSQPQQQSQQQLGQCSFQQPQQLQQLGQQPQQQQIPQGIFLQ

SEQ ID NO: 608 - protein, 56 aa, Triticum aestivum
MAKRLVLFVAVVVALVALTVAEGEASEQLQCERELQELQERELKACQQVMDQQLRD

SEQ ID NO: 609 - protein, 56 aa, Triticum aestivum
LQCERELQELQERELKACQQVMDQQLRDISPECHPVVVSPVAGQYEQQIVVPPKGG

SEQ ID NO: 610 - protein, 54 aa, Triticum aestivum
SCCKSTLGRNCYNLCRARGAQKLCANVCRCKLTSGLSCPKDFPKLVLESNSDEP

(19) United States
(12) Patent Application Publication
(10) Pub. No.: US 2023/0251269 A1
(43) Pub. Date: Aug. 10, 2023

(54) COMPREHENSIVE ANALYSIS OF ANTI-ALLERGEN ANTIBODIES USING PHAGE DISPLAY

(71) Applicant: THE JOHNS HOPKINS UNIVERSITY, Baltimore, MD (US)

(72) Inventors: Harry B. Larman, Baltimore, MD (US); Daniel Monaco, Baltimore, MD (US)

(21) Appl. No.: 18/015,684

(22) PCT Filed: Jul. 15, 2021
(86) PCT No.: PCT/US21/41754

§ 371 Date: Jan. 11, 2023

Related U.S. Application Data
(60) Provisional application No. 63/140,051, filed on Jan. 21, 2021.
(60) Provisional application No. 63/052,109, filed on Jul. 15, 2020.

(51) Int. Cl. G01N 33/68; G01N 33/543; C12N 15/10; C12Q 1/6844

(52) U.S. Cl. CPC G01N 33/6854; G01N 33/5436; C12N 15/1037; C12Q 1/6844; G01N 2800/24

(57) ABSTRACT

The present invention relates to the field of allergies. More specifically, the present invention provides compositions and methods useful for identifying anti-allergen antibodies in a patient sample using phage display. In one embodiment, a method for detecting the presence of an antibody against an allergen in subject includes the steps of (a) contacting a reaction sample comprising a display library with a biological sample comprising antibodies, wherein the display library includes a plurality of peptides derived from a plurality of allergens; and (b) detecting at least one antibody bound to at least one peptide expressed by the display library, thereby detecting an antibody against the at least one peptide in the biological sample.

### CROSS REFERENCE TO RELATED APPLICATIONS

This application claims the benefit of U.S. Provisional Application 63/052,109 filed on Jul. 15, 2020 and U.S. Provisional Application 63/140,051 filed on Jan. 21, 2021. The entire contents of these applications are incorporated herein by reference in its entirety.

### GOVERNMENT SUPPORT CLAUSE

This invention was made with government support under grant no. AI118633, awarded by the National Institutes of Health. The government has certain rights in the invention.

### FIELD

The present disclosure relates to the field of allergies. More specifically, the present invention provides compositions and methods useful for identifying anti-allergen antibodies in a patient sample using phage display.

### INCORPORATION-BY-REFERENCE OF MATERIAL SUBMITTED ELECTRONICALLY

This application contains two tables that were originally filed with U.S. Provisional Application 63/140,051 filed on Jan. 21, 2021 and that have been submitted electronically via EFS-Web as an ASCII text file. These tables are hereby incorporated by reference in their entireties.

### BACKGROUND

In recent decades, food allergy has emerged as a major public health issue, affecting up to 10% of the population in Westernized countries. In patients with IgE-mediated food allergy, exposure to the allergenic food results in cross-linking of pre-existing food-specific IgE (fs-IgE) bound to the high affinity IgE receptor FcεRI on the surface of mast cells and basophils, causing potentially life-threatening allergic reactions. The high prevalence of food allergy has led to an ever-increasing need for food allergy testing in clinical practice. While the oral food challenge is the gold standard for diagnosing food allergy, this procedure is time-consuming, requires highly trained personnel, and can cause an acute allergic reaction. Therefore, the diagnosis of food allergy is often based on a combination of the clinical history and the results of fs-IgE and skin prick testing (SPTs). These tests, however, often detect sensitization to foods that are not associated with symptoms upon ingestion, which can lead to unnecessary food avoidance. The shortcomings of fs-IgE testing are exemplified in the diagnosis of IgE-mediated wheat allergy, where a recent meta-analysis found that wheat specific IgE levels have a specificity of only 43% in predicting wheat allergy¹. Strong cross-reactivity between grass pollen and wheat is likely a contributing factor to the high rate of false positive testing.

While component testing is emerging as a useful adjunct for diagnosing food allergy, this approach is limited to testing single allergens at a time and requires relatively large sample volumes and significant expense. Other strategies to refine the diagnosis of food allergy have relied on epitope mapping, which evaluates IgE binding to a library of contiguous short peptides that compose allergenic proteins. Several methodologies have been developed, including SPOT membranes, microarray based immunoassays (MIA), and most recently Bead-Based Epitope Assays (BBEA)^{3,4}. These approaches revealed that certain immunodominant peptides, as well as overall greater diversity of IgE epitopes recognized, were associated with more severe reactions and a greater likelihood of having persistent allergy in patients with milk and egg allergy³. However, these approaches are largely only capable of assessing antibody reactivities to a select number of components (typically one to a few dozen) and are frequently difficult to interpret. Importantly, the identification of novel allergenic epitopes is both costly and time-intensive; inexpensive high-throughput approaches that efficiently identify novel epitopes would therefore have great utility to inform clinical component test development.

### SUMMARY

As described herein, the present disclosure provides a programmable phage display-based method to comprehensively analyze anti-allergen IgE and IgG antibodies to 1,847 allergenic proteins, tiled with overlapping 56 amino acid peptides, in a single multiplex reaction. In certain embodiments, oligonucleotide library synthesis was used to encode a database of allergenic peptide sequences for display on T7 bacteriophages (the “AllerScan” library), which can be analyzed using high-throughput DNA sequencing. Such embodiments enable the analysis of longer, higher quality peptides than is otherwise possible with synthetic peptide microarrays, and at a dramatically reduced per-sample cost.

One aspect of the present technology is that, unlike existing phage display techniques which rely on cDNA, programmable microarrays enable construction of a starting library that is uniformly distributed. The synthetic programmable microarray approach eliminates skewed initial distributions in cDNA libraries resulting from incorrect reading frame or differential gene expression obstacles, which ultimately hamper accurate detection of peptide enrichment. Further, when coupled with high throughput sequencing for analysis, the programmable microarray approach compares favorably to traditional Sanger sequencing or microarray hybridization techniques, as high throughput phage immunoprecipitation sequencing (PhIP-Seq) allows sensitive quantification of a larger number of library members and with a wider dynamic range.

Incidence of allergy and allergic asthma is increasing, and there are still many gaps in our understanding of allergic disease. The combination of low-specificity testing and food avoidance recommendations, results in many patients needlessly avoiding foods. The IgE antibody profiling technology described herein may provide a more specific system for diagnosing allergies than skin-pricks and RAST. Unlike conventional assays, PhIP-Seq with the allergome library detects IgE binding at a peptide (not protein) level, enabling high-resolution identification of specific allergenic motifs on a per-patient basis. In particular embodiments, such information may be used to design personalized avoidance patterns or tolerizing immunotherapies. Furthermore, the present disclosure enables robust investigations into epitope level allergenic IgE cross-reactivity.

In one aspect, the present disclosure provides compositions and methods for detecting the presence of an antibody against an allergen in a subject. In certain embodiments, a method comprises (a) contacting a reaction sample comprising a display library with a biological sample comprising antibodies, wherein the display library comprises a plurality of peptides derived from a plurality of allergens; and (b) detecting at least one antibody bound to at least one peptide expressed by the display library, thereby detecting an antibody against the at least one peptide in the biological sample. The display library can be a viral display library, a bacteriophage display library, a yeast display library, a bacterial display library, a retroviral display library, a ribosome display library or an mRNA display library. In particular embodiments, the display library is a phage display library.

In a specific embodiment, the antibodies are immobilized to a solid support adapted for binding immunoglobulin E (IgE) subclass. In a more specific embodiment, the antibodies are immobilized by contacting the display library and antibodies from the biological sample with anti-IgE antibodies. In certain embodiments, the anti-IgE antibodies are immobilized to a solid support. In other embodiments, the antibodies are immobilized by contacting the display library and antibodies from the biological sample with anti-G or anti-A antibodies. In an alternative embodiment, the anti-IgG or anti-IgA antibodies are immobilized to a solid support. In an alternative embodiment, the antibodies are immobilized by contacting the display library and antibodies from the biological sample with Protein A and/or Protein G. In certain embodiments, the Protein A and/or Protein G are immobilized to a solid support.

In particular embodiments, the detection of the antibody comprises a step of lysing the phage and amplifying the DNA. In certain embodiments, amplifying the DNA by polymerase chain reaction (PCR) includes a denaturation step that also lyses the phage. In specific embodiments, each peptide of the plurality of peptides comprises a common adapter region appended to the end of the nucleic acid sequence encoding the peptide. In other embodiments, the method further comprises removing unbound antibody and peptides of the display library.

In particular embodiments, the plurality of peptides are each less than 100, 200, 300, 500, 500, 600, 700, 800, or 900 amino acids long. Other lengths are also within the scope of the invention. In one embodiment, the plurality of peptides are each less than 100, 200, or 300 amino acids long. In a more specific embodiment, the plurality of peptides are each less than 75 amino acids long.

In certain embodiments of the present disclosure, at least two antibodies are detected. In more specific embodiments, the at least two antibodies are detected simultaneously. In particular embodiments, deoxyribonucleic acid (DNA) within a vector of the display library encoding each peptide of the plurality of peptides comprises common adapter regions flanking the ends of the nucleic acid sequences encoding the peptides.

In specific embodiments, the detection step comprises amplifying DNA within the display library vector that encodes the displayed peptide. In a more specific embodiment, the method further comprises the step of sequencing the amplified DNA. In an even more specific embodiment, the sequencing step comprises next generation sequencing. In an alternative embodiment, the method further comprises the step of performing microarray hybridization to detect the amplified sequences. In a more specific embodiment, the amplification step comprises real-time polymerase chain reaction (PCR).

In other embodiments, the detection step comprises amplifying a DNA proxy within the library display vector that encodes the displayed peptide. In particular embodiments, the DNA proxy is a peptide-specific barcode sequence. In one embodiment, the method further comprises the step of sequencing the amplified DNA proxy. In a specific embodiment, the sequencing step comprises next generation sequencing. In an alternative embodiment, the method further comprises the step of performing microarray hybridization to detect the amplified DNA proxy. In another embodiment, the amplification step comprises real-time PCR.

The compositions and methods of the present disclosure can also be used to diagnose allergies of an individual from which the biological sample is obtained. In particular embodiments, the present disclosure is used to identify an individual from which the biological sample is obtained as being sensitized or allergic. In other embodiments, the present disclosure is used to identify cross reactivity of an individual from which the biological sample is obtained to tailor treatment or design allergen avoidance strategies.

In particular embodiments, the display library comprises a predetermined number of peptides from the peptides listed in Table 1 of U.S. Provisional Application 63/052,109 filed on Jul. 15, 2020 which is incorporated herein by reference in its entirety.

In another aspect, the present disclosure provides a phage display library. In one embodiment, a phage library displays a plurality of allergen peptides, wherein the plurality of allergen peptides represents a set of peptides from allergens known to affect humans, or suspected of affecting humans. In a specific embodiment, the phage library includes a plurality of allergen peptides from a predetermined number of allergens known to affect humans, or suspected of affecting humans.

In one embodiment, the phage library comprises at least 20 peptide sequences. The plurality of peptides can each be less than 100, 200 or 300 amino acids long, or can be longer. In particular embodiments, the plurality of peptides are each less than 75 amino acids long.

In specific embodiments, each peptide of the plurality of peptides comprises a common adapter region appended to the end of the nucleic acid sequence encoding the peptide.

In another aspect, the present disclosure provides for a method of producing a library of proteins, comprising: synthesizing synthetic oligonucleotides encoding for one or a plurality of peptides, wherein the oligonucleotides comprise from about one hundred to about three hundred nucleotides; amplifying the oligonucleotides; cloning the amplified oligonucleotides into a display library; thereby, producing a library of proteins. In certain embodiments, the one or plurality of peptides comprise one or more IgE binding sites. In certain embodiments, the plurality of peptides comprise from about 20 to about 40 amino acid overlap between successive peptides. In certain embodiments, the plurality of peptides comprise about 28 amino acid overlap between successive peptides. In certain embodiments, the one or plurality of peptides are allergens. In certain embodiments, the one or plurality of peptides are each less than 100, 200, or 300 amino acids long. In certain embodiments, the one or plurality of peptides are each less than 75 amino acids long. In certain embodiments, the nucleic acid sequence(s) within a vector of the display library encoding each peptide of the plurality of peptides comprises common adapter regions flanking the ends of the nucleic acid sequences encoding the peptides. In certain embodiments, the display library is a viral display library, a bacteriophage display library, a yeast display library, a bacterial display library, a ribosome display library or an mRNA display library. In certain embodiments, the display library is a bacteriophage library.

In certain embodiments, the amplification step comprises polymerase chain reaction (PCR). In certain embodiments, the amplification step comprises real-time polymerase chain reaction (PCR) real-time PCR (quantitative PCR or qPCR), reverse-transcriptase (RT-PCR), multiplex PCR, nested PCR, hot start PCR, long-range PCR, assembly PCR, asymmetric PCR or in situ PCR.

In particular embodiments, the phage library includes a predetermined number of peptides from the peptides listed in Table 1 of U.S. Provisional Application 63/052,109 filed on Jul. 15, 2020.

In another aspect, the present disclosure provides compositions and methods useful for anti-wheat vaccines. In particular embodiments, a vaccine comprises a monoclonal antibody against the wheat allergy epitopes described in Table 1 and/or Table 2. In certain embodiments, the present disclosure provides antibodies that specifically bind to one or more of the epitopes listed in Table 2.

### BRIEF DESCRIPTION OF THE FIGURES

FIG. 1A-1F. IgE profiling with an exemplary T7 phage displayed allergome library. FIG. 1A. (i) The exemplary allergome library is composed of 19,331 56 amino acids (aa) peptide tiles that overlap by 28 aa. (ii) The peptide-encoding DNA sequences were synthesized as 200-mer oligonucleotides and cloned into a T7 phage display vector. (iii) The allergome phage library is incubated with serum. (iv) IgE and bound phage are immunocaptured onto omalizumab-coated magnetic beads and sequenced. FIG. 1B. Representation of the allergome phage library: 95.84% of all library members were detected; 68.2% of the library members were within one log of abundance. FIG. 1C. Dilution series showing top 10 wheat peptides (red) and peanut peptides (blue) using serum from a wheat allergic, peanut tolerant individual. FIG. 1D. Dilution series showing top 10 wheat peptides (red) and peanut peptides (blue) using serum from a peanut allergic, wheat tolerant individual. FIG. 1E. Reproducibility of IgE AllerScan assay. FIG. 1F. Discordance of IgE and IgG reactivity against the allergome library.

FIG. 2A-2C. AllerScan Profiles. IgE (FIG. 2A) and IgG (FIG. 2B) reactivity profiles for all test samples (n=86). Columns correspond to all peptides recognized by at least three samples; columns are arranged taxonomically via phylogenetic clustering. Each row corresponds to a unique samples. IgE rows were hierarchically clustered via a binary distance metric; the IgE row order was maintained for the IgG reactivity profiles. FIG. 2C. Pie charts corresponding to library's peanut and wheat representation in the starting library (top) and in the immunoprecipitated fractions (bottom).

FIG. 3A-3D. IgE and IgG reactivity of wheat peptides. Serum donors (columns in FIG. 3A-3B) had been clinically defined as wheat allergic (n=32), wheat non-allergic (n=27) and wheat sensitized (n=27). Rows correspond to wheat peptides tiled from each protein's N- to C-terminus, arranged by increasing size (top to bottom). Data in FIG. 3A are from IgE profiling; data in B are from IgG profiling. FIG. 3C. Comparison of the IgE anti-wheat antibody breadths between patient groups. FIG. 3D. Comparison of the IgG anti-wheat antibody breadths between patient groups.

FIG. 4A-4D. Identification of a discriminatory IgG-reactive wheat epitope. FIG. 4A. Multiple sequence alignment of three purothionin peptides, which are (FIG. 4B) preferentially IgG-reactive in the wheat non-allergic and sensitized populations, versus wheat allergic individuals. FIG. 4C. IgE reactivities to the purothionin epitope among the same three patient populations. FIG. 4D. Comparison of the IgE versus the IgG reactivities to the purothionin epitope.

FIG. 5A-5D. Identification of wheat allergic antibody subgroups. FIG. 5A. Hierarchically clustered heatmap of IgE reactivity to wheat peptides among allergic individuals. Rows are wheat peptides recognized by at least two wheat allergic sera. FIG. 5B. Overall wheat IgE antibody breadth. All comparisons between populations were performed via Wilcoxon signed-rank test. FIG. 5C. Scatterplot comparing IgE vs IgG reactivity to all peptides from FIG. 5A. Each point compares a given antibody reactivity for a given allergic individual. FIG. 5D. Network graph of all peptides in FIG. 5A. Nodes are peptides; nodes are linked if they share sequence similarity. A multiple sequence alignment logo generated from the HMW glutenin cluster is shown in the top left.

FIG. 6A-6E. Wheat Immunotolerance Trial Results. FIG. 6A. Pairwise plots of IgE (left) and IgG (right) Allergome read count data from one patient comparing serum at start of trial plotted against serum after one year of placebo treatment. Wheat peptides are red, all other peptides in the Allergome are black. FIG. 6B. Pairwise plots of IgE (left) and IgG (right) Allergome read count data from same patient as in FIG. 6A, comparing serum prior to WOIT treatment against serum after one year of WOIT treatment. FIG. 6C. Scatterplot comparing IgE vs IgG reactivity to all peptides from trial before and after WOIT treatment. FIG. 6D. Violin plots evaluating overall wheat antibody breadth and purothionin reactivity from samples before and after receiving placebo treatment. No significant changes were observed for any metric after placebo (Wilcox test). FIG. 6E. Violin plots evaluating overall wheat antibody breadth and purothionin reactivity from samples before and after receiving WOIT treatment. There was a significant decrease in IgE wheat breadth and a significant increase in IgG purothionin reactivity after treatment (p<0.01 for both comparisons, Wilcoxon signed-rank test).

FIG. 7. Peptide sequence similarity-based network graph of wheat epitope peptides. Nodes are peptides and nodes are linked if they possess sequence similarity.

FIG. 8. Connectivity of 24 wheat epitope candidates for vaccine induced anti-allergic immune responses.

FIG. 9A-9D. Identification of a discriminatory IgG-reactive wheat epitope. FIG. 9A: Multiple sequence alignment of three alpha purothionin peptides, which are (FIG. 9B) preferentially IgG-reactive in the wheat non-allergic and sensitized populations, versus wheat allergic individuals. FIG. 9C: IgE reactivities to the alpha purothionin epitope among the same three patient populations. FIG. 9D: Comparison of the IgE versus the IgG reactivities to the alpha purothionin epitope. For FIG. 9B, 9C, 9D—wheat allergic (n=32), wheat non-allergic (n=27) and wheat sensitized (n=27).

### DETAILED DESCRIPTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Allergic reactions to environmental agents (i.e., allergens) can affect almost all organs of the body. While allergic rhinitis and contact dermatitis are the most common reactions, conjunctivitis, edema, asthma and, most dangerously, anaphylaxis are all possible outcomes of an allergic reaction Immunoglobulin E (IgE), an antibody isotype, together with mast cells, eosinophils and basophils are the key drivers in the manifestation of allergic diseases. An allergic response is initiated by IgE binding to an allergen, such as peanut, and a receptor on mast cells or basophils; this binding triggers these cells to release inflammatory cytokines such as histamine.

One of the current inventors previously utilized programmable microarrays to synthesize oligonucleotides for the complete human peptidome, coupled with high throughput sequencing to analyze the results after selection. Larman et al., 29 NAT. BIOTECH. 535-41 (2011), incorporated herein by reference. Described herein is a specific implementation of a new approach wherein synthetic representations of a complete set of allergen peptides can be generated, such as a set of allergen peptides derived from allergens known to affect humans, or suspected of affecting humans.

The precise molecular determinants associated with common allergens and the mechanistic basis for shared reactivity among different food allergens are still areas of intense research. As describe herein, the present inventors demonstrate an efficient bacteriophage (phage) display based allergic antibody profiling platform. This system enables sensitive and unbiased characterization of allergen-associated antibody reactivity against thousands of allergenic proteins from hundreds of organisms at the peptide level.

The three most commonly used tests for the diagnosis of food allergies are oral food challenge (OFC), skin prick tests and in vitro blood assays (e.g. enzyme-linked immunosorbent assay, ELISA, and radioallergosorbent test, RAST). OFC requires highly-trained personnel, can be dangerous, and is not typically feasible for testing large numbers of allergens. Skin-prick assays are cheaper and safer than OFC, but they suffer from low specificity. Finally, in vitro blood assays, despite being safe, are typically either single-plex or low-multiplex, such that many different assays must be carried out to comprehensively characterize IgE binding patterns.

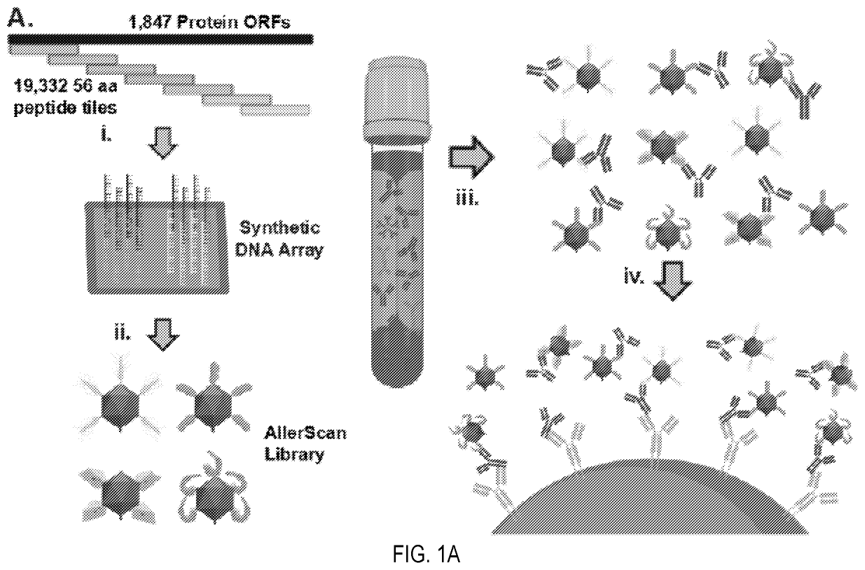

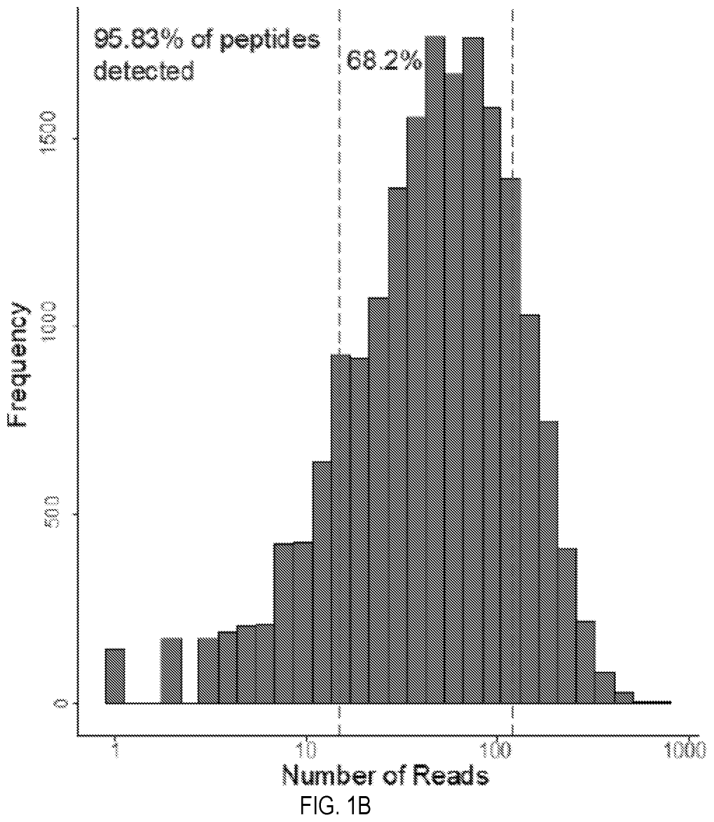

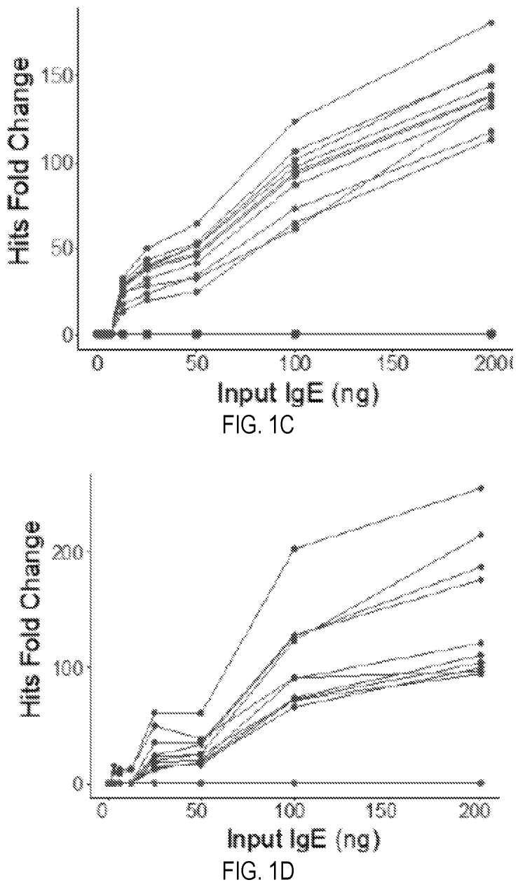

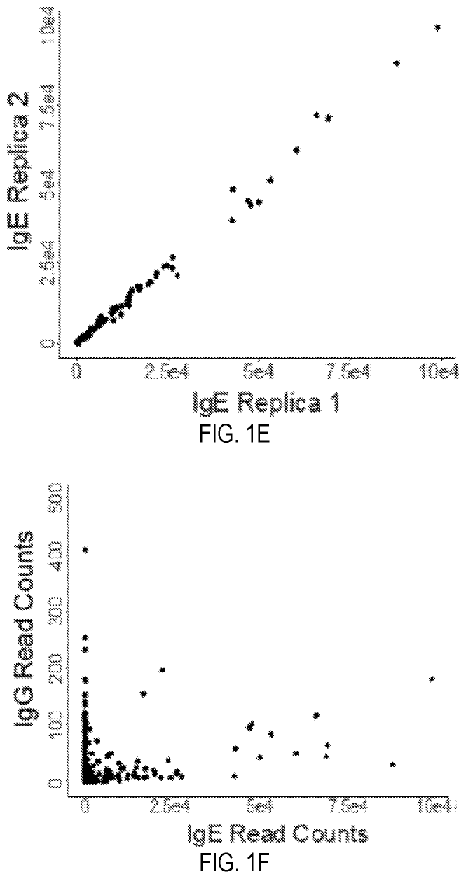

As described herein, an embodiment of the present disclosure uses high throughput DNA synthesis to produce a DNA library of >19,000 200-mer oligonucleotides, which encodes 56 amino acid peptides that ‘tile’, with 28 amino acid overlaps, the human allergome (FIG. 1A). This DNA library may be cloned into the T7 bacteriophage system to produce a phage library. This phage library may be then used in the Phage ImmunoPrecipitation Sequencing (PhIP-Seq) assay to profile the IgE antibodies from patients known to have, or suspected of having, food allergies.

In order to determine antibody reactivities specific to IgE, the present disclosure uses magnetic beads coated with omalizumab (a monoclonal anti-IgE antibody used as an asthma therapeutic, also known as Xolair) to specifically capture IgE-bound phage particles (FIG. 1A). After removing unbound phage particles by washing the beads, PCR is performed to amplify the peptide-coding DNA sequences and uses Illumina sequencing is used to determine which peptides had been IgE precipitated.

### Definitions

As used herein, the term “display library” refers to a library comprising a plurality of peptides derived from a plurality of allergens that are displayed on the surface of a virus or cell e.g., bacteriophage, yeast, or bacteria.

As used herein, the term “antibody-peptide complex” refers to a complex formed when an antibody recognizes an epitope on a peptide and binds to the epitope under low or normal stringent conditions. It will be appreciated that an antibody-peptide complex can dissociate under high stringent conditions, such as low or high pH, or high temperatures.

As used herein, the term “to the allergen from which it is derived” refers to a step of correlating or mapping at least one peptide in an antibody-peptide complex to a sequence in the known sequences of the allergens, thereby identifying the allergen that comprises the peptide sequence.

As used herein, the term “allergen” refers to an antigen that is capable of stimulating a type-I hypersensitivity reaction in atopic and/or allergic individuals through immunoglobulin E (IgE) responses. “Allergenic peptides” refers to peptides derived from antigens that stimulate a type-I hypersensitivity reaction in atopic and/or allergic individuals through immunoglobulin E (IgE) responses. Examples include peptides derived from milk proteins, tree nut proteins, shellfish proteins, grain proteins and the like.

As used herein, the term “enriched” indicates that a peptide from a given allergen is represented at a higher proportion in the display library after immunoprecipitation with a subject's antibodies compared to its representation in the starting library or the library after “mock” immunoprecipitation in which no IgE was input into the reaction. In some embodiments, the peptides from a given allergen are enriched by some measurable predetermined degree as compared to the general population. In other embodiments, the peptides for a given allergen are enriched by at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 25-fold, at least 50-fold, at least 100-fold, at least 1000-fold, or more, compared to the general population.

As used herein the term “oligonucleotide primers” refers to nucleic acid sequences that are 5 to 100 nucleotides in length, preferably from. 17 to 45 nucleotides, although primers of different length are of use. Primers for synthesizing cDNAs are preferably 10-45 nucleotides, while primers for amplification are preferably about 17-25 nucleotides. Primers useful in the methods described herein are also designed to have a particular melting temperature (Tm) by the method of melting temperature estimation. Commercial programs, including OLIGO™, Primer Design and programs available on the internet, including PRIMERS and OLIGO CALCULATOR can be used to calculate a Tm of a polynucleotide sequence useful according to the methods and assays described herein. Preferably, the Tm of an amplification primer useful according to the disclosure, as calculated for example by OLIGO CALCULATOR, is preferably between about 45 and 65° C. In other embodiments, the Tm of the amplification primer is between about 50 and 60° C.

As used herein, the term “sample” refers to a biological material which is isolated from its natural environment and contains at least one antibody. A sample according to the methods described herein, may consist of purified or isolated antibody, or it may comprise a biological sample such as a tissue sample, a biological fluid sample, or a cell sample comprising an antibody. A biological fluid includes, but is not limited to, blood, plasma, sputum, urine, cerebrospinal fluid, lavages, and leukapheresis samples, for example.

As used herein the term “adapter sequence” refers to a nucleic acid sequence appended to a nucleic acid sequence encoding a phage-displayed peptide. In one embodiment, the identical adaptor sequence is appended to the end of each phage-displayed peptide encoding DNA in the phage display library; that is, the adaptor sequence is a common sequence on each nucleic acid of the plurality of nucleic acids encoding a peptide in the phage display library. In one embodiment, the adaptor sequence is of sufficient length to permit annealing of a common PCR primer. For example, adaptor sequences useful with the methods described herein are preferably heterologous or artificial nucleotide sequences of at least 15, and preferably 20 to 30 nucleotides in length. An adapter sequence may comprise a barcode sequence.

As used herein, the term “amplified product” refers to polynucleotides which are copies of a portion of a particular polynucleotide sequence and/or its complementary sequence, which correspond in nucleotide sequence to the template polynucleotide sequence and its complementary sequence. An “amplified product,” can be DNA or RNA, and it may be double-stranded or single-stranded.

The term “specifically binds” refers to an agent, compound or, in certain embodiments, an antibody that recognizes and binds a peptide of the disclosure, but which does not substantially recognize and bind other molecules in a sample, for example, a biological sample, which may comprise a peptide of the disclosure. The term specifically refers to the binding of an affinity tag to a corresponding capture agent to which it specifically binds (e.g., biotin-streptavidin).

A recited range is meant to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Allergens

Allergens are well known to persons skilled in the art. Common environmental allergens which induce allergic conditions are found in pollen (e.g., tree, herb, weed and grass pollen allergens), food, dust mites, animal hair, dander and/or saliva, molds, fungal spores and venoms (e.g., from insects). A non-exhaustive list of environmental allergens may be found at the online allergenic molecules (allergens) database, the Allergome or the International Union of Immunological Societies (IUIS) official database of allergens.

As used herein, the term “allergome” refers to all proteins which may give rise to allergies. This includes proteins recorded in allergen databases such as that represented in the Allergome, IUIS, as well as allergens included in UniProt, Swiss-Prot, etc.

The term “allergen” refers to an antigenic substance capable of producing immediate hypersensitivity and includes both synthetic as well as natural immunostimulant peptides and proteins. In particular embodiments, an “allergen” refers to a molecule capable of inducing an IgE response and/or a Type I allergic reaction.

In more particular embodiments, the term “allergen” refers to a type of antigen that in the native form produces an abnormally vigorous immune response in which the immune system fights off all perceived threats that would otherwise be harmless to the subject. Typically, these kinds of reactions result in the phenotype known as allergy. Various types of allergens are described in the prior art, including foods, drugs, animal products or natural or synthetic material.

Such allergens may include food allergens (e.g., peanut, wheat, etc.), air-borne allergens (e.g., pollen from grass, tree, herb and weeds, dust mites, fungi and molds), insect allergens (e.g., cockroach, fleas, bee and wasp venom) and epithelial allergens (animal hair, animal dander, e.g., cat and dog dander). Pollen allergens from trees, grasses and weeds derive from the taxonomic order group of Fagales (e.g., Alnus and Betula), Lamiales (e.g., Olea and Plantago), Poales (e.g., Phleum pratense), Asterales (e.g., Ambrosia and Artemisia), Cayophyllales (e.g., Chenopodium and Salsola), Rosales (e.g., Parietaria), Proteales (e.g., Platanus) etc. Dust mites belong to the order group of Astigmata (e.g., Dermatophagoides and Euroglyphus). Airborne allergens derived from moulds and fungi belong to the order Pleosporales (e.g., Alternaria), Capnodiales (e.g., Cladosporium) etc.

Air borne allergens may be selected from/or selected from the groups of: Tree pollen (Alnus glutinosa, Betula alba, Corylus avellana, Cupressus arizonica, Olea europea, Platanus sp), grass pollen (Cynodon dactylon, Dactylis glomerata, Festuca elatior, Holcus lanatus, Lolium perenne, Phleum pratense, Phragmites communis, Poa pratensis), weed pollen (Ambrosia elatior, Artemisia vulgaris, Chenopodium album, Parietaria judaica, Plantago lanceolata, Salsola kali) and cereal pollen (Avena sativa, Hordeum vulgare, Secale cereal, Triticum aestivum, Zea mays), dust mites (Acarus siro, Blomia tropicalis, Dermatophagoides farinae, Dermatophagoides microceras, Dermatophagoides pteronyssinus, Euroglyphus maynei, lepidoglyphus destructor, Tyrophagus putrescentiae), fungi and moulds (Alternaria alternate, Cladosporium herbarum, Aspergillus fumigatus). Other airborne allergens are also within the scope of the invention.

Epithelial allergens may be selected from any animal including, but not limited to, cat hair and dander, dog hair and dander, horse hair and dander, human hair and dander, rabbit hair and dander, and feathers. Other epithelial allergens are also within the scope of the invention.

Insect Allergens may be selected from ant, flea, mites (Acarus siro, Blomia tropicalis, Dermatophagoides farinae, Dermatophagoides microceras, Dermatophagoides pteronyssinus, Euroglyphus maynei, lepidoglyphus destructor, Tyrophagus putrescentiae), cockroach, wasp venom and bee venom. Other insect allergens are also within the scope of the invention.

Production of a Phage Display Library

General methods for producing a phage display library are known to those of skill in the art and/or are described in, for example, Larman et al., 29(6) NAT. BIOTECH. 535-41 (2011), which is incorporated herein by reference in its entirety.

Unlike the conventional art, contemplated herein are phage display libraries that comprise a plurality of peptides derived from a plurality of allergens. In one embodiment, it is contemplated herein that the plurality of peptides will represent a substantially complete set of peptides from a group of allergens. In one embodiment, the phage display library comprises a substantially complete set of peptides from allergens known to affect humans, or suspected of affecting humans, (or a subgroup thereof). Similarly, phage display libraries comprising a substantially complete set of peptides from allergenic pollens (or a subgroup thereof) or allergenic fungi (or a subgroup thereof) are also contemplated herein. As used herein, the term “subgroup” refers to a related grouping of allergens that would benefit from simultaneous testing. For example, one of skill in the art can generate a phage display library comprising a substantially complete set of peptides from a genus of allergens (e.g., a subgroup of pollen allergens, such as the Amrosia genus (ragweed species). Such a library would permit one of skill in the art to distinguish between highly related allergens in an antibody sample.

In some embodiments, the phage display library includes a predetermined number of peptide sequences (e.g., less than 10,000). In other embodiments, the phage display library comprises at least 100, at least 200, at least 500, at least 1000, at least 5000, at least 10,000 peptide sequences or more. It will be appreciated by one of ordinary skill in the art that as the length of the individual peptide sequences increases, the total number of peptide sequences in the library can decrease without loss of any allergen sequences (and vice versa).

In some embodiments, the phage display library includes peptides derived from a predetermined set (e.g., at least 10) of peptide sequences e.g. allergenic peptides. In some embodiments, the phage display library comprises peptides derived from at least 10 protein sequences, at least 20 protein sequences, at least 30 protein sequences, at least 40 protein sequences, at least 50 protein sequences, at least 60 protein sequences, at least 70 protein sequences, at least 80 protein sequences, at least 90 protein sequences, at least 100 protein sequences, at least 200 protein sequences, at least 300 protein sequences, at least 400 protein sequences, at least 500 protein sequences, at least 600 protein sequences, at least 700 protein sequences, at least 800 protein sequences, at least 900 protein sequences, at least 1000 protein sequences, at least 2000 protein sequences, at least 3000 protein sequences, at least 4000 protein sequences, at least 5000 protein sequences, at least 6000 protein sequences, at least 6500 protein sequences, at least 7000 protein sequences, at least 7500 protein sequences, at least 8000 protein sequences, at least 8500 protein sequences, at least 9000 protein sequences, at least 10,000 protein sequences or more.

In some embodiments, the phage display library comprises a plurality of proteins sequence that have less than 90% shared identity; in other embodiments the plurality of protein sequences have less than 85% shared identity, less than 80% shared identity, less than 75% shared identity, less than 70% shared identity, less than 65% shared identity, less than 60% shared identity, less than 55% shared identity, less than 50% shared identity or even less.

In some embodiments, the phage display library comprises protein sequences from at least 3 unique allergens or at least 5 unique allergens; in other embodiments the library comprises protein sequences from at least 10, at least 20, at least 50, at least 75, at least 100, at least 200, at least 300, at least 400, at least 500, at least 600, at least 700, at least 800, at least 900, at least 1000 unique allergens up to and including protein sequences from all allergens known to cause allergies, or suspected of causing allergies, in a human or other mammal.

In some embodiments, the protein sequences of the phage display library are at least 1 amino acids long; in other embodiments the protein sequences are at least 20, at least 30, at least 40, at least 50, at least 60, at least 70, at least 80, at least 90, at least 100, at least 150, at least 200, at least 250, at least 300, at least 350, at least 400, at least 450 amino acids or more in length.

In some embodiments, each peptide of the phage library will overlap at least one other peptide by at least 5 ammo acids. In other embodiments, each peptide of the phage library will overlap at least one other peptide by at least 10, at least 15, at least 20, at least 21, at least 22, at least 23, at least 24, at least 25, at least 26, at least 27, at least 28, at least 29, at least 30, at least 32, at least 35, at least 40, at least 45, at least 50, at least 55, at least 60, at least 65, at least 70, at least 75, at least 80, at least 85, at least 90, at least 95, at least 100 amino acids or more.

In some embodiments, the display library includes at least 2 peptides from Table 1 of U.S. Provisional Application 63/052,109 filed on Jul. 15, 2020. In some embodiments, the display library comprises at least 2 allergenic peptides. In other embodiments, the display library comprises at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10000, at least 11000, at least 12000, at least 13000, at least 14000, at least 15000, at least 16000, at least 17000, at least 18000, at least 19000 allergenic peptides or more. In certain embodiments, the peptides are selected in any combination from Table 1 of U.S. Provisional Application 63/052,109 filed on Jul. 15, 2020.

In certain embodiments, the display library can include peptides from at least 1 family (e.g., Fabaceae) or sub-family (e.g., Faboideae) of related peanuts. In other embodiments, the display library—can include peptides from at least 2 families, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 peptides from at least 1 family (e.g., Fabaceae) or sub-family (e.g., Faboideae) of peanuts, in any desired combination. In one embodiment, the display library includes peptides from each of the known peanut families or subfamilies.

While the disclosure specifically recites phage display libraries, it is specifically contemplated herein that other display libraries can be used with the methods and assays described herein including, but not limited to, a yeast display library, a bacterial display library, a retroviral display library, a ribosome display library or an mRNA display library. It is within the skills of one of ordinary skill in the art to apply the methods and assays exemplified herein using a phage display library to the use of a different type of display library.

Reaction Samples

As used herein, the term “reaction sample” refers to a sample that, at a minimum, comprises a phage display library, for example, the phage display library described herein. The reaction sample can also comprise additional buffers, salts, osmotic agents, etc., to facilitate the formation of complexes between the peptides in the phage display library when the reaction sample is contacted with a biological sample comprising an antibody. A “biological sample” as that term is used herein refers to a fluid or tissue sample derived from a subject that comprises or is suspected of comprising at least one antibody.

A biological sample can be obtained from any organ or tissue in the individual to be tested, provided that the biological sample comprises, or is suspected of comprising, an antibody. Typically, the biological sample will comprise a blood sample, however other biological samples are contemplated herein, for example, mucosal secretions.

In some embodiments, a biological sample is treated to remove cells or other biological particulates. Methods for removing cells from a blood or other biological sample are well known in the art and can include, e.g., centrifugation, ultrafiltration, immune selection, sedimentation, etc. Antibodies can be detected from a biological sample or a sample that has been treated as described above or as known to those of skill in the art. Some non-limiting examples of biological samples include a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, or a combination of such samples. For the methods described herein, it is preferred that a biological sample is from whole blood, plasma, saliva, serum, and/or urine. In one embodiment, the biological sample is serum.

In some embodiments, samples can be obtained from an individual with an allergy. In certain embodiments, samples from a normal demographically matched individual and/or from a patient not having the allergy are used in the analysis to provide controls. The samples can comprise a plurality of sera or plasma from individuals sharing a trait. For example, the trait shared can be gender, age, allergy, exposure to the same environmental condition (e.g., such as an allergen), and the like.

Removal of Unbound Phage

In some embodiments, the methods and assays described herein comprise a step of contacting modified bacteriophage or the phage display library as described herein with a biological sample that comprises, or is suspected of comprising, at least one antibody. Any anti-allergen antibodies present in the biological sample will bind to bacteriophage(s) that display the cognate antigen.

In certain embodiments, it is desirable to separate the bacteriophage(s) bound to an antibody in the biological sample from any free bacteriophage(s) that are not bound to an antibody in the sample. In one embodiment, antibodies from the reaction sample are immobilized on a solid support to permit one to separate out the unbound phage. Antibody immobilization can be achieved using a variety of methods that permits one to specifically immobilize IgE and/or IgG or subclasses can be used to immobilize antibodies from the sample, including antibodies that are complexed to one or more bacteriophage. In particular embodiments, an anti-IgE antibody is used to immobilize the antibody to permit removal of unbound phage. In a specific embodiment, the anti-IgE antibody is a monoclonal antibody. In a more specific embodiment, the anti-IgE antibody comprises omalizumab (XOLAIR®). In other embodiments, Protein A, Protein G or a combination thereof is/are used to immobilize IgG antibody to permit removal of unbound phage.

In some embodiments, the peptide or protein used to immobilize antibodies from the reaction mixture can be attached to a solid support, such as, for example, magnetic beads (e.g., micron-sized magnetic beads), Sepharose beads, agarose beads, a nitrocellulose membrane, a nylon membrane, a column chromatography matrix, a high performance liquid chromatography (HPLC) matrix or a fast performance liquid chromatography (FPLC) matrix for purification. For example, the reaction mixture comprising bacteriophage and antibodies can be contacted with magnetic beads coated with anti-IgE, anti-IgG or anti-IgA antibodies. In other embodiments, the magnetic beads can be coated with specific IgG subisotype capture antibodies, such as IgG1, IgG2, IgG3 and/or IgG4. In other embodiments, the magnetic beads can be coated with Protein A and/or Protein G. In such embodiments, the anti-IgE antibodies, Protein A and/or Protein G, as the case may be, will bind to antibodies in the mixture and immobilize them on the beads. This process also immobilizes any phage particles bound by the antibodies. In one embodiment, a magnet can be used to separate the immobilized phage from unbound phage. In one approach, one may a) first include phage in a container (such as a test tube); b) add serum to the container; c) prepare beads (as described herein) with capture molecules; and d) add the prepared beads to the phage and serum in the container. In another approach, one may a) first include phage in a container (such as a test tube); b) add serum to the container; c) add capture molecules to the phage and serum in the container; and d) add beads (as described herein) into the container containing the phage, serum and capture molecules. Other methods to immobilize antibodies from the reaction mixture may be used and are within the scope of the present disclosure.

As used herein, the term “magnetic bead” means any solid support that is attracted by a magnetic field; such solid supports include, without limitation, DYNABEADS®, BIOMAG® Streptavidin, MPG7 Streptavidin, Streptavidin MAGNESPHERE™ Streptavidin Magnetic Particles, AFFINITIP™, any of the MAGA™ line of magnetizable particles, BIOMAG™ Superparamagnetic Particles, or any other magnetic bead to which a molecule (e.g., an oligonucleotide primer) may be attached or immobilized.

The above steps may be performed by one or more devices so configured.

Methods for Peptide Detection

Following a step of removing any unbound phage, the peptides in the bound phage/antibody complexes can be identified using, e.g., one or more devices configured for PCR and/or DNA sequencing. In some embodiments, the bound phage/antibody complexes can first be released from the solid support using appropriate conditions e.g., temperature, pH, etc. In some embodiments, the sample is subjected to conditions that will permit lysis of the phage (e.g., heat denaturation). In one embodiment, the nucleic acids from the lysed phage is subjected to an amplification reaction, such as a PCR reaction. In other embodiments, the PCR reaction includes a denaturation step that lyses the phage. In one embodiment, the nucleic acids encoding a phage-displayed peptide include a common adapter sequence for PCR amplification. In such embodiments, a PCR primer is designed to bind to the common adapter sequence for amplification of the DNA corresponding to a phage-displayed peptide.

In particular embodiments, the amplified DNA encoding the peptide can be detected by sequencing. In certain embodiments, a microarray hybridization approach can be used. In another embodiment, real time PCR amplification of specific DNA sequences can be used.

In certain embodiments, one of the PCR primers contains a common adaptor sequence which can be amplified in a second PCR reaction by another set of primers to prepare the DNA for high throughput sequencing. Unique barcoded oligonucleotides in the second PCR reaction can be used to amplify different samples and pool them together in one sequencing run to, for example, reduce cost and/or permit simultaneous detection of multiple phage-displayed peptides.

In some embodiments, the detection of a phage-displayed peptide includes PCR with barcoded oligonucleotides. As used herein, the term “barcode” refers to a unique oligonucleotide sequence that allows a corresponding nucleic acid base and/or nucleic acid sequence to be identified. In certain aspects, the nucleic acid base and/or nucleic acid sequence is located at a specific position on a larger polynucleotide sequence (e.g., a polynucleotide covalently attached to a bead). In certain embodiments, barcodes can each have a length within a range of from about 4 to about 36 nucleotides, or from about 6 to about 30 nucleotides, or from about 8 to about 20 nucleotides. In certain aspects, the melting temperatures of barcodes within a set are within about 10° C. of one another, within about 5° C. of one another, or within about 2° C. of one another. In other aspects, barcodes are members of a minimally cross-hybridizing set. That is, the nucleotide sequence of each member of such a set is sufficiently different from that of every other member of the set that no member can form a stable duplex with the complement of any other member under stringent hybridization conditions. In one aspect, the nucleotide sequence of each member of a minimally cross-hybridizing set differs from those of every other member by at least two nucleotides. Barcode technologies are known in the art and are described in e.g., Winzeler et al., 285 SCIENCE 901 (1999); Brenner, C., 1(1) GENOME BIOL. 103.1-103.4 (2000); Kumar et al., 2 NATURE REV 302 (2001); Giaever et al., 101 PROC. NATL. ACAD SCI. USA 793 (2004); Eason et al., 101 PROC. NATL. ACAD. SCI. USA 1046 (2004); and Brenner, C., 5 GENOME BIOL. 240 (2004).

In some embodiments, a detectable label is used in the amplification reaction to permit detection of different amplification products. As used herein, “label” or “detectable label” refers to any atom or molecule which can be used to provide a detectable (in some embodiments, quantifiable) signal, and which can be operatively linked to a polynucleotide, such as a PCR primer or proxy DNA sequence (often referred to as a DNA barcode). Labels may provide signals detectable by fluorescence, radioactivity, colorimetry, gravimetry, X-ray diffraction or absorption, magnetism, enzymatic activity, mass spectrometry, binding affinity, hybridization radiofrequency, nanocrystals and the like. A primer of the present disclosure may be labeled so that the amplification reaction product may be “detected” by “detecting” the detectable label. “Qualitative or quantitative” detection refers to visual or automated assessments based upon the magnitude (strength) or number of signals generated by the label. A labeled polynucleotide (e.g., an oligonucleotide primer) according to the methods of the disclosure can be labeled at the 5′ end, the 3′ end, or both ends, or internally. The label can be “direct”, e.g., a dye, or “indirect”, e.g., biotin, digoxin, alkaline phosphatase (AP), horse radish peroxidase (HRP). For detection of “indirect labels” it is necessary to add additional components such as labeled antibodies, or enzyme substrates to visualize the captured, released, labeled polynucleotide fragment.

In specific embodiments, an oligonucleotide primer is labeled with a fluorescent label. Labels include, but are not limited to, light-emitting, light-scattering, and light-absorbing compounds which generate or quench a detectable fluorescent, chemiluminescent, or bioluminescent signal. See, e.g., Garman A., Non-Radioactive Labeling, Academic Press (1997) and Kricka, L., Nonisotopic DNA Probe Techniques, Academic Press, San Diego (1992). Fluorescent reporter dyes useful as labels include, but are not limited to, fluoresceins (see, e.g., U.S. Pat. Nos. 6,020,481; 6,008,379; and 5,188,934), rhodamines (see, e.g., U.S. Pat. Nos. 6,191,278; 6,051,719; 5,936,087; 5,847,162; and 5,366,860), benzophenoxazines (see, e.g., U.S. Pat. No. 6,140,500), energy-transfer fluorescent dyes, comprising pairs of donors and acceptors (see, e.g., U.S. Pat. Nos. 5,945,526; 5,863,727; and 5,800,996; and), and cyanines (see, e.g., WO 9745539), lissamine, phycoerythrin, Cy2, Cy3, Cy3.5, Cy5, Cy5.5, Cy7, FluorX (Amersham Biosciences, Inc. (Piscataway, N.J.)), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′,1,4,-tetrachlorofluorescein, and 2′,4′,5′,7′,1,4-hexachlorofluorescein. In certain aspects, the fluorescent label is selected from SYBR-Green, 6-carboxyfluorescein (“FAM”), TET, ROX, VICTM, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In further embodiments, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In certain embodiments, the detection of a phage-displayed peptide comprises high throughput detection of a plurality of peptides simultaneously, or near simultaneously. In some embodiments, the high-throughput systems use methods similar to DNA sequencing techniques. Any conventional DNA sequencing technique may be used.

A number of DNA sequencing techniques are known in the art, including fluorescence-based sequencing methodologies (See, e.g., Birren et al., Genome Analysis: Analyzing DNA, 1, Cold Spring Harbor, N.Y.). In some embodiments, automated sequencing techniques understood in the art are utilized. In some embodiments, the high-throughput systems described herein use methods that provide parallel sequencing of partitioned amplicons (e.g., WO2006084132). In some embodiments, DNA sequencing is achieved by parallel oligonucleotide extension (See, e.g., U.S. Pat. Nos. 5,750,341, and 6,306,597). Additional examples of sequencing techniques include the Church polony technology (Mitra et al., 320 ANAL. BIOCHEM. 55-65 (2003); Shendure et al., 309 SCIENCE 1728-32 (2005); U.S. Pat. Nos. 6,432,360; 6,485,944; 6,511,803), the 454 picotiter pyrosequencmg technology (Margulies et al., 437 NATURE 376-80 (2005); US20050130173), the Solexa single base addition technology (Bennett et al., 6 PHARMACOGENOMICS 373-82 (2005); U.S. Pat. Nos. 6,787,308; 6,833,246), the Lynx massively parallel signature sequencing technology (Brenner et al., 18 NAT. BIOTECHNOL. 630-34 (2000).; U.S. Pat. Nos. 5,695,934; 5,714,330), and the Adessi PCR colony technology (Adessi et al., 28 NUCLEIC ACID RES. E87 (2000).; WO00018957).

Next-generation sequencing (NGS) methods share the common feature of massively parallel, high-throughput strategies, with the goal of lower costs in comparison to older sequencing methods (see, e.g., Voelkerding et al., 55 CLINICAL CHEM. 641-58 (2009); MacLean et al., 7(4) NAT. REV. MICROBIOL. 287-96 (2009)). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by ILLUMINA™, and the Supported Oligonucleotide Ligation and Detection™ (SOLiD) platform commercialized by APPLIED BIOSYSTEMS™. Non-amplification approaches, also known as single-molecule sequencing, are exemplified by the HELISCOPE™ platform commercialized by HELICOS BIOSYSTEMS™, and emerging platforms commercialized by VISIGEN™, OXFORD NANOPORE TECHNOLOGIES LTD., and PACIFIC BIOSCIENCES™, respectively.

In pyrosequencing (Voelkerding et al. (2009)); MacLean et al, Nature Rev. Microbial., 7:287-296; U.S. Pat. Nos. 6,210,891; 6,258,568), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a charge-coupled device (CCD) camera. It is possible to achieve read lengths greater than or equal to 400 bases, resulting in up to 500 million base pairs (Mb) of sequence.

In certain embodiments, nanopore sequencing is employed (see, e.g., Astier et al., 128(5) J. AM. CHEM. SOC. 1705-10 (2006)). The theory behind nanopore sequencing has to do with what occurs when a nanopore is immersed in a conducting fluid and a potential (voltage) is applied across it. Under these conditions, a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, HELISCOPE™ by HELICOS BIOSCIENCES™ is employed (Voelkerding et al. (2009); MacLean et al. (2009); U.S. Pat. Nos. 7,169,560; 7,282,337; 7,482,120; 7,501,245: 6,818,395; 6,911,345: 7,501,245). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in a fluor signal corresponding to the dNTP, and the fluor signal is captured by a CCD camera before each round of dNTP addition. Sequence read length ranges from about 25-50 nucleotides with overall output exceeding 1 billion nucleotide pairs per analytical run.

The Ion Torrent technology is a method of DNA sequencing based on the detection of hydrogen ions that are released during the polymerization of DNA (see, e.g., 327(5970) SCIENCE 1190 (2010); U.S. Patent Appl. Pub. Nos. 20090026082, 20090127589, 20100301398, 20100197507, 20100188073, and 20100137143). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is about 99.6% for 50 base reads, with −100 Mb generated per run. The read-length is 100 base pairs. The accuracy for homopolymer repeats of 5 repeats in length is about 98%.

Another example of a nucleic acid sequencing approach that can be adapted for use with the methods described herein was developed by STRATOS GENOMICS, Inc. and involves the use of XPANDOMERS™. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits include a tether, at least one probe or nucleobase residue, and at least one selectively cleavable bond. The selectively cleavable bond(s) is/are cleaved to yield an XPANDOMER™ of a length longer than the plurality of the subunits of the daughter strand. The XPANDOMER™ typically includes the tethers and reporter elements for parsing genetic information in a sequence corresponding to the contiguous nucleotide sequence of all or a portion of the target nucleic acid. Reporter elements of the XPANDOMER™ are then detected. Additional details relating to XPANDOMER™-based approaches are described in, for example, U.S. Pat. Pub No. 20090035777, entitled “HIGH THROUGHPUT NUCLEIC ACID SEQUENCING BY EXPANSION,” filed Jun. 19, 2008, which is incorporated herein in its entirety.

Other single molecule sequencing methods include real-time sequencing by synthesis using a VISIGEN™ platform (Voelkerding et al. (2009); U.S. Pat. Nos. 7,329,492: 7,668,697; WO2009014614) in which immobilized, primed DNA template is subjected to strand extension using a fluorescently-modified polymerase and florescent acceptor molecules, resulting in detectible fluorescence resonance energy transfer (FRET) upon nucleotide addition.

Another real-time single molecule sequencing system developed by PACIFIC BIOSCIENCES™ (Voelkerding et al. (2009); MacLean et al. (2009); U.S. Pat. Nos. 7,170,050; 7,302,146; 7,313,308; 7,476,503) utilizes reaction wells 50-100 nm in diameter and encompassing a reaction volume of approximately 20 zeptoliters (10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10-21 L). At this volume, the activity of a single molecule can he detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

Processes and systems for such real time sequencing that can be adapted for use with the methods described herein include, for example, but are not limited to U.S. Pat. Nos. 7,405,281, 7,315,019, 7,313,308, 7,302,146, 7,170,050, U.S. Pat. Pub. Nos. 20080212960, 20080206764, 20080199932, 20080176769, 20080176316, 20080176241, 20080165346, 20080160531, 20080157005, 20080153100, 20080153095, 20080152281, 20080152280, 20080145278, 20080128627, 20080108082, 20080095488, 20080080059, 20080050747, 20080032301, 20080030628, 20080009007, 20070238679, 20070231804, 20070206187, 20070196846, 20070188750, 20070161017, 20070141598, 20070134128, 20070128133, 20070077564, 20070072196, 20070036511, and Koriach et al., 105(4) PROC. NATL. ACAD. SCI. USA 1176-81 (2008), all of which are herein incorporated by reference in their entireties.

Sequence Analysis

Subsequently, in some embodiments, the data produced from the AllerScan assay includes sequence data from multiple barcoded DNAs. Using the known association between the barcode and the source of the DNA, the data can be deconvoluted to assign sequences to the source subjects, samples, organisms, etc.

Some embodiments include a processor, data storage, data transfer, and software comprising instructions to assign genotypes. Some embodiments of the technology provided herein further include functionalities for collecting, storing, and/or analyzing data. For example, some embodiments include the use of a processor, a memory, and/or a database for, e.g., storing and executing instructions, analyzing data, performing calculations using the data, transforming the data, and storing the data. In some embodiments, the processor is configured to calculate a function of data derived from the sequences and/or genotypes determined. In some embodiments, the processor performs instructions in software configured for medical or clinical results reporting and in some embodiments the processor performs instructions in software to support non-clinical results reporting. In some embodiments, there is a non-tangible computer-readable product that contains instructions to cause a computing device to perform any of the methods described herein.

Without further elaboration, it is believed that one skilled in the art, using the preceding description, can utilize the present disclosure to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Profiling Serum Antibodies with a Novel Pan Allergen Phage Library to Identify Key Wheat Epitopes

As described herein, the present invention provides a programmable phage display-based method to comprehensively analyze anti-allergen IgE and IgG antibodies to 1,847 allergenic proteins, tiled with overlapping 56 amino acid peptides, in a single multiplex reaction. In certain embodiments, oligonucleotide library synthesis was used to encode a database of allergenic peptide sequences for display on T7 bacteriophages (the “AllerScan” library), which can be analyzed using high-throughput DNA sequencing. Such embodiments enable the analysis of longer, higher quality peptides than is otherwise possible with synthetic peptide microarrays, and at a dramatically reduced per-sample cost.

Materials and Methods

Human donor samples. While the present invention is not dependent on any particular sera, the following describes characteristics of sera used in two proof-of-concept experiments by the present inventors. In both of the proof-of-concept experiments, the invention described herein accurately detected allergy reactivities.

In one proof-of-concept experiment by the inventors, sera was obtained from 58 patients known to have an IgE-mediated food allergy along with 25 age-matched healthy controls who were enrolled on a Natural History of Food Allergy protocol at the National Institutes of Health (NIH). In this NIH protocol, subjects were defined as having a wheat allergy based on having a convincing history of a type I hypersensitivity reaction to wheat within the last 2 years immediately after ingesting wheat along with positive wheat-specific IgE testing, with the exception of 3 subjects who were classified as wheat allergic even though their most recent reaction was 5 and 6 years ago, and one who had been avoiding wheat due to positive testing. Wheat sensitized subjects were tolerating wheat in their diet with no overt symptoms, and all healthy controls were following an unrestricted diet. Peanut allergy was similarly defined by a positive immediate reaction upon peanut ingestion along with positive peanut-specific IgE testing, although 13 subjects were avoiding peanut due to positive testing alone. Peanut and wheat specific IgE levels and total IgE were determined by ImmunoCAP (Phadia).

In another proof-of-concept experiment by the inventors, sera was obtained from 23 wheat allergic subjects who had participated in a randomized, double-blind, placebo-controlled wheat oral immunotherapy trial. Here, subjects with wheat allergy confirmed by a positive double-blind, placebo-controlled oral food challenge (DBPCFC) to wheat at baseline were randomized 1:1 to oral immune therapy (OIT) with vital wheat gluten or placebo. Subjects underwent dose escalation every two weeks until they reached a daily maintenance dose of 1445 mg of wheat or placebo protein. After approximately 1 year of treatment (minimum of 8 weeks of maintenance dosing), subjects underwent a DBPCFC to wheat (cumulative dose of 7443 mg wheat protein) to evaluate for desensitization and were subsequently unblinded. Subjects in the active arm continued on active wheat OIT, while the placebo arm crossed over to active treatment (maximum maintenance dose of 2748 mg wheat protein) for approximately one year.

Design and cloning of allergy peptide library sequences. AllerScan screening was performed following the standard PhIP-Seq library design protocol⁶. Briefly, For the Allergome peptide library, all protein sequences were downloaded from UniProt database included in the Allergome and collapsed on 90% sequence similarity [query=uniprot:(allergome)+identity: 0.9]. The UniProt clustering algorithm returned representative sequences for each protein cluster. The present inventors designed peptide sequences 56 amino acids in length (that overlap by 28 amino acids) that tiled through all representative allergen proteins. Next, these sequences were reverse-translated into DNA nucleotide sequences optimized for E. coli expression and added the adapter sequences to both the 5′ and 3′ ends these sequences. These 200 nucleotide sequences were commercially synthesized on a cleavable DNA microarray. These sequences were then cloned into the T7FNS2 and the resulting library was then packaged into the T7 bacteriophage via the T7 Select Packaging Kit (EMD Millipore).

Phage immunoprecipitation and sequencing. IgG screening of the Allergome library was performed as described previously^{6,9,7}. The above-described mid-copy T7 bacteriophage display library spanning the Allergome was used. An IgG-specific ELISA was used to quantify serum concentrations (Southern Biotech). Two Kg of IgG was added to 1 mL of Allergome library at an average of 1×10⁵ pfu for each peptide in the reaction. Serum and phage library were rotated overnight at 4° C., after which 20 μL of protein A and 20 μL of protein G magnetic beads (Invitrogen, 10002D and 10004D) were added to each reaction which were rotated an additional 4 hr at 4° C. Beads were subsequently washed three times in 0.1% NP-40 and then re-suspended in a Herculase II Polymerase (Agilent cat #600679) PCR master mix. These PCR mixes underwent 20 cycles of PCR. Two μL of these reactions were added to new PCR master mixes which used sample specific barcoding primers that underwent an additional 20 cycles of PCR. The final amplified product was pooled and sequenced using an Illumina NovaSeq6000 or an Illumina NextSeq 500 instrument.

For IgE Phip-Seq, biotinylated Xolair was conjugated to Streptavidin-bound M280 magnetic beads (ThermoFisher-11205D) at 4×binding capacity. Excess unbound Xolair was washed in 0.01% PBST. 100 ng of IgE was added to 1 mL of Allergome library at 1×10¹⁰ pfu for each reaction and 10 μL of Xolair-coated M280s were used for each immunoprecipitation. All other steps for IgE PhIP-Seq were identical to the above-described IgG PhIP-Seq methodology.

Informatics and statistical analysis. In order to quantify the levels of antibody reactivity the samples had to Allergome peptides, sequencing reads were mapped to the Allergome library requiring perfect matches and the number of times a clone was detected for each sample was counted, creating a counts matrix. Next, the R “edgeR” software package was utilized which compares the signal detected in each sample against a set of control “mock” immunoprecipitations that were performed without serum using a negative binomial model, returning both a test statistic and fold change value for each peptide in every sample, creating enrichment and fold change matrices. Significantly enriched peptides, called “hits”, required counts, p-values and fold changes of at least 100, 0.001, and 5 respectively. Hits fold-change matrices report the fold change value for “hits” and “1” for peptides that are not hits.

All subsequent analysis was developed and implemented in R 3.6.1. All heatmaps were constructed using the pheatmap package and all network graph analyses utilized the igraph software package; all other plots were built using the tidyverse package suite. All performed statistical tests were Wilcoxon signed-rank tests unless otherwise specified.

Results

In order to construct a comprehensive library of allergenic proteins, the curated Allergome database⁸ was downloaded from UniProt (accessed Aug. 6, 2017) and used as input to the PhIP-Seq pepsyn library design pipeline⁶. The 1,847 proteins of the Allergome database were represented as a set of 19,332 56 amino acid peptide tiles with 28 amino acid overlaps, which were encoded by a library of synthetic 200-mer oligonucleotides (FIG. 1A). This library was amplified and cloned into the T7 phage display system⁹ for automated serological profiling⁶. This library is referred to as the T7 AllerScan library.

One of the present inventors has previously utilized protein A and protein G coated magnetic beads to immunoprecipitate predominantly IgG-bound phage. In contrast to the highly abundant and relatively consistent levels of IgG in blood, IgE tends to be logs lower in abundance and highly variable between individuals¹⁰. To enable specific IgE immunoprecipitation of the AllerScan library, in certain embodiments, biotin was covalently conjugated to the therapeutic monoclonal anti-IgE antibody omalizumab¹¹; streptavidin coupled magnetic beads could then be irreversibly coated with this IgE capture antibody (FIG. 1A).

To assess AllerScan library quality, the phage library was sequenced at 66-fold coverage; 95.8% of the expected clones were detected, which were drawn from a relatively uniform distribution (FIG. 1B). Next, the present inventors serially diluted serum from two patients, one with a known wheat allergy and one with a known peanut allergy. In both cases, concentration-dependent enrichments of the expected allergenic peptides were observed in the immunoprecipitated fraction (FIG. 1C-D). Based on these data, 100 ng of total, volume normalized IgE input was chosen for subsequent analyses. Next, to establish the reproducibility and specificity of the IgE immunocapture, the present inventors performed two IgE and two protein A/G (hereafter referred to as IgG) immunocaptures using serum from a wheat allergic individual. The present inventors noted a high concordance when comparing replicas from the same immunocapture technique (IgE: R²=0.996, FIG. 1E; IgG: R²=0.966, not shown), and a high level of discordance when comparing IgE to IgG (FIG. 1F). This discordance, particularly for the strongest reactivities, highlights the isotype specificity of omalizumab-based IgE immunocapture.

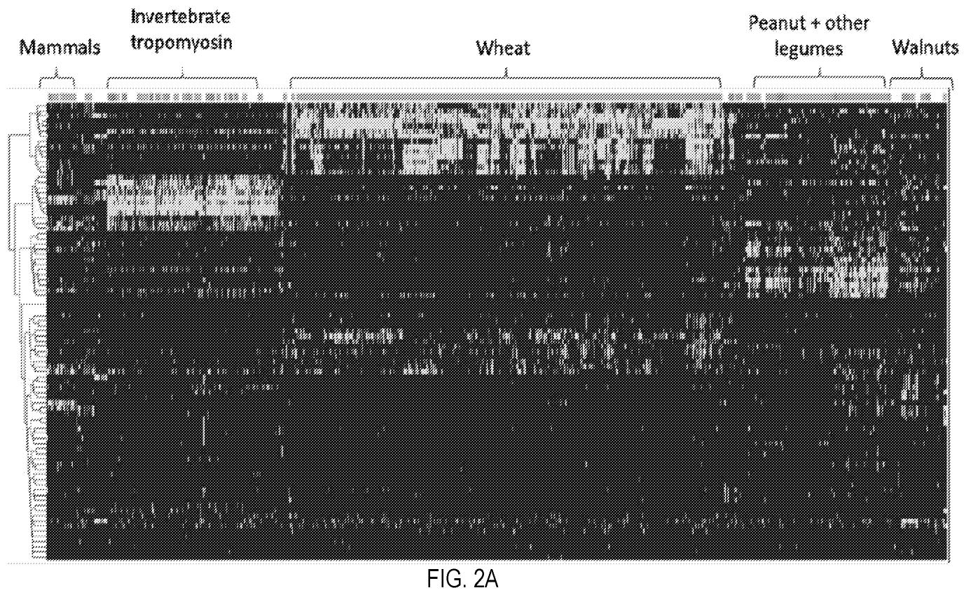

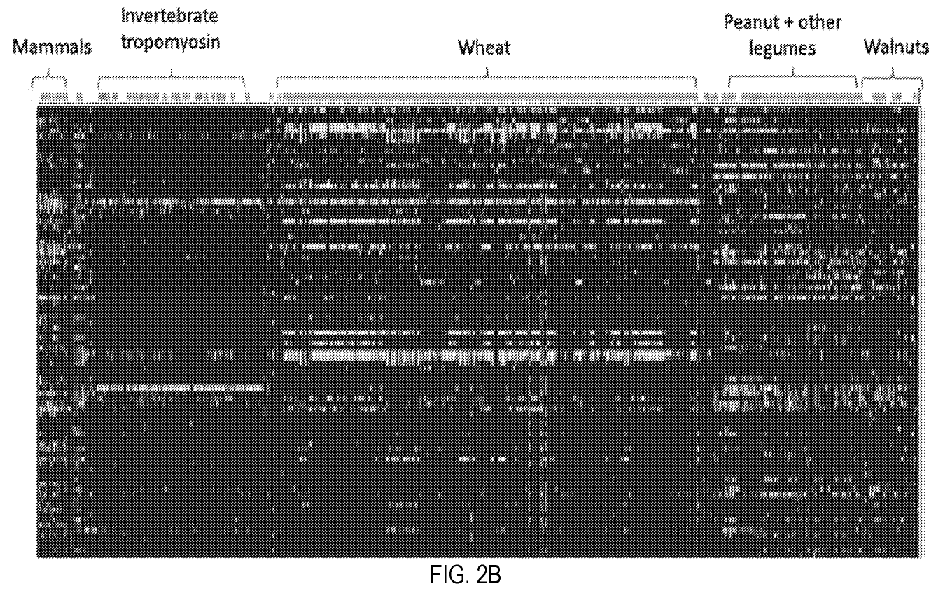

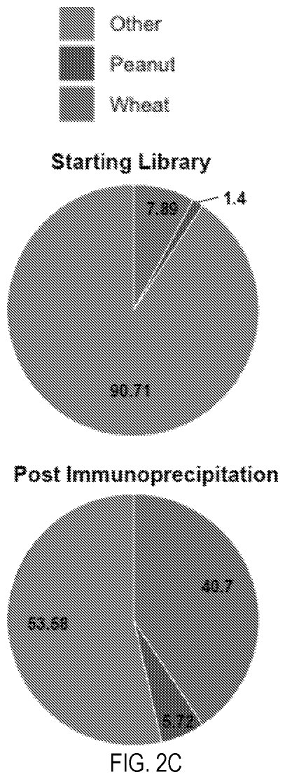

The present inventors next assembled a cross-sectional cohort of individuals with clinically characterized allergies to peanut and/or wheat, as well as healthy controls. FIG. 2A-B displays the results of both IgE and IgG immunocaptures. In these heatmaps, each column corresponds to a peptide that reacted with at least 2 sera. The order of the columns corresponds to the taxonomic identity of the organism from which each peptide is derived. Groups of related organisms, rather than individual organisms, are indicated where convenient. Rows correspond to individual samples and are organized by hierarchical clustering of IgE profiles. The order of the rows and columns is maintained for the IgG heatmap (FIG. 2B). Reactivity to wheat and peanut peptides were observed to correlate with the expected allergic group, while many additional allergic reactivities were also observed. FIG. 2C highlights the change in the library's peanut and wheat representation in the immunoprecipitated fractions, compared to the AllerScan library. As expected for this cohort, both wheat and peanut showed large increases in representation post-immunoprecipitation.

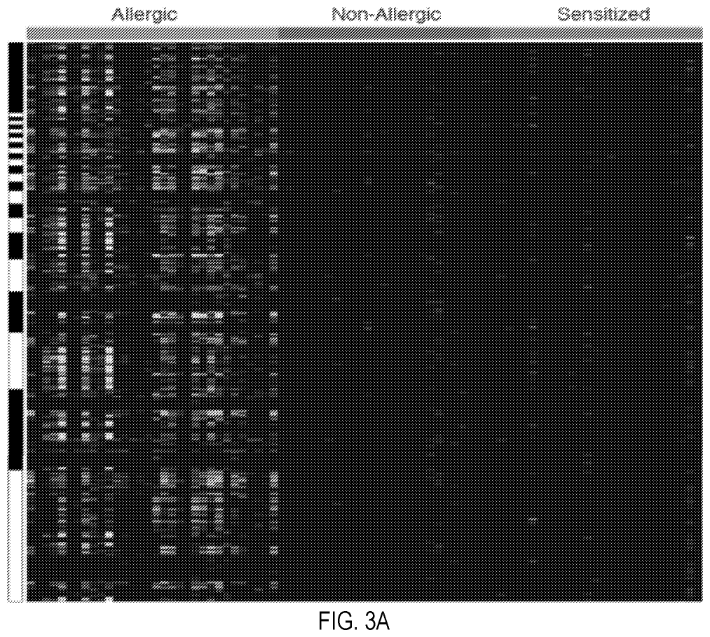

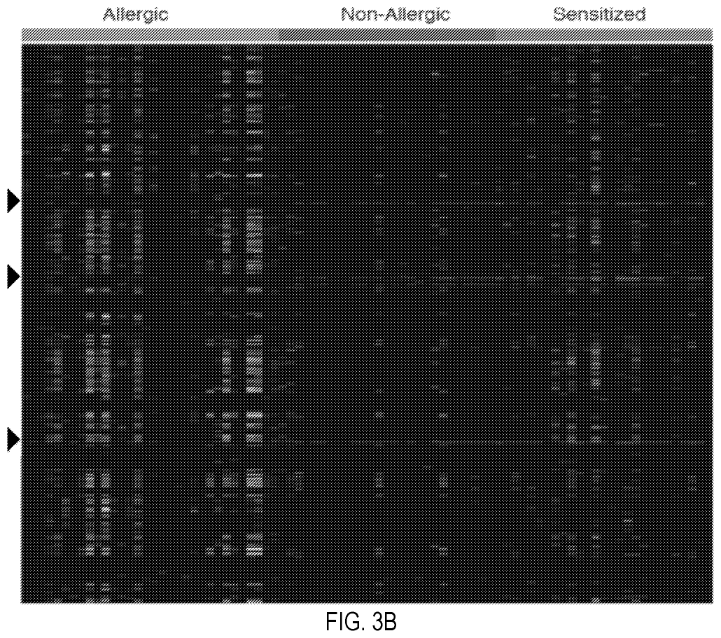

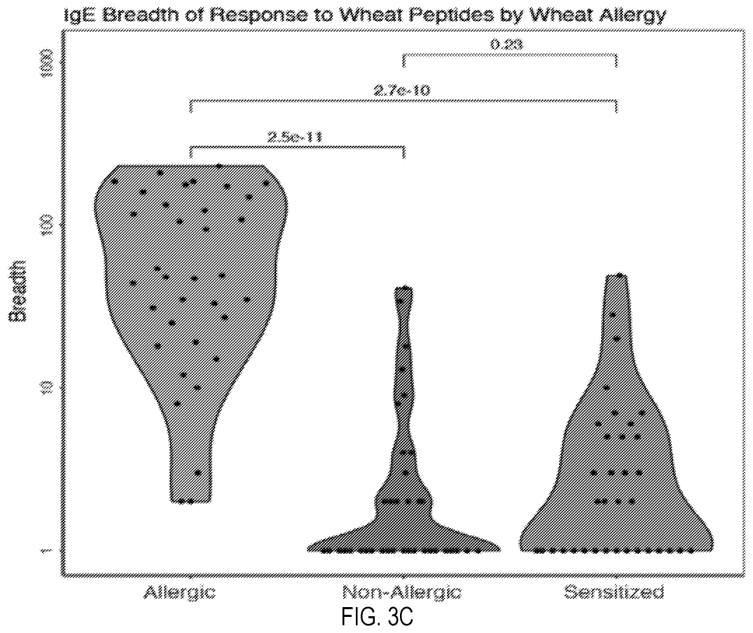

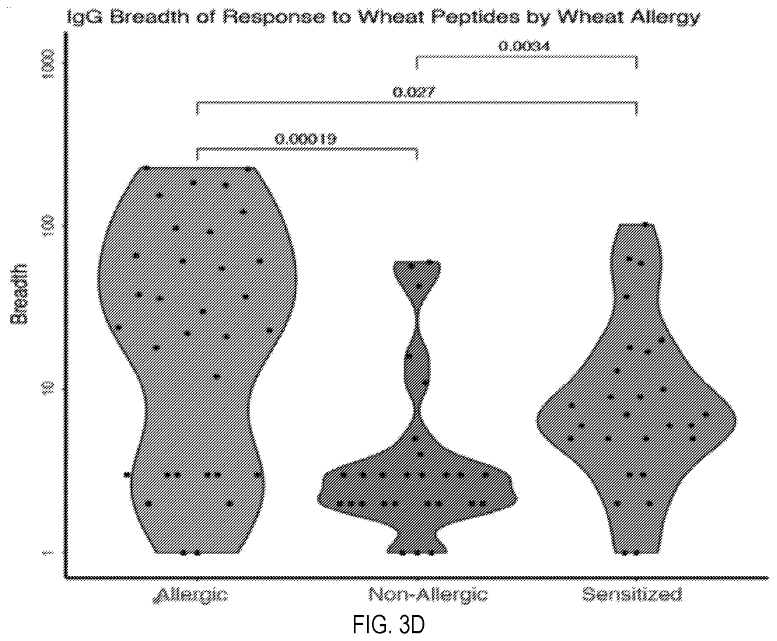

Since few studies have investigated anti-wheat reactivity at the epitope level, the present inventors sought to broadly characterize the fine specificities of anti-wheat antibodies using the AllerScan system. To this end, the present inventors performed both IgE and IgG immunocaptures with serum samples from three groups: individuals with proven wheat allergy, individuals with wheat “sensitivity” (positive clinical test but symptomless in food challenge), and non-allergics. An expansive set of wheat peptides were found to be IgE-reactive in the wheat allergic group, whereas minimal reactivity was detected to the phage-displayed wheat peptides in sera from either sensitized or non-allergic individuals (FIG. 3A). Among wheat allergic IgE responses, the present inventors noted extensive immunodominance and seemingly distinct patterns of reactivity. Upon examination of IgG reactivity to wheat peptides among the same three groups, the present inventors noted dramatic elevation of reactivity among allergic individuals—largely to the same IgE immunodominant epitopes, along with detectable but relatively less reactivity among the sensitized individuals, and minimal reactivity among the non-allergic individuals (FIG. 3B). Several previously identified wheat epitopes are composed of repetitive and redundant peptide sequences^{12,13,14}. The present inventors therefore utilized a network graph-based approach to determine, for each individual immunocapture, the “maximal independent vertex set” of reactive peptides that do not share any sequence homology. The present inventors have previously utilized this metric as a surrogate for the “breadth” of a polyclonal response^{15,16}. Wheat allergic individuals exhibited significantly greater breadths of both IgE and IgG reactivity (FIG. 3C-D), versus non-allergic individuals (p=2.5×10⁻¹⁰ for IgE and p=1.9×10⁻⁴ for IgG). Only in the IgG immunocaptures were the breadths of responses to wheat peptides significantly higher in the sensitized versus the non-allergic individuals (p=0.0034). Taken together, these findings suggest that the breadth of the anti-wheat peptide IgE repertoire may have utility in distinguishing between wheat allergy and sensitization.

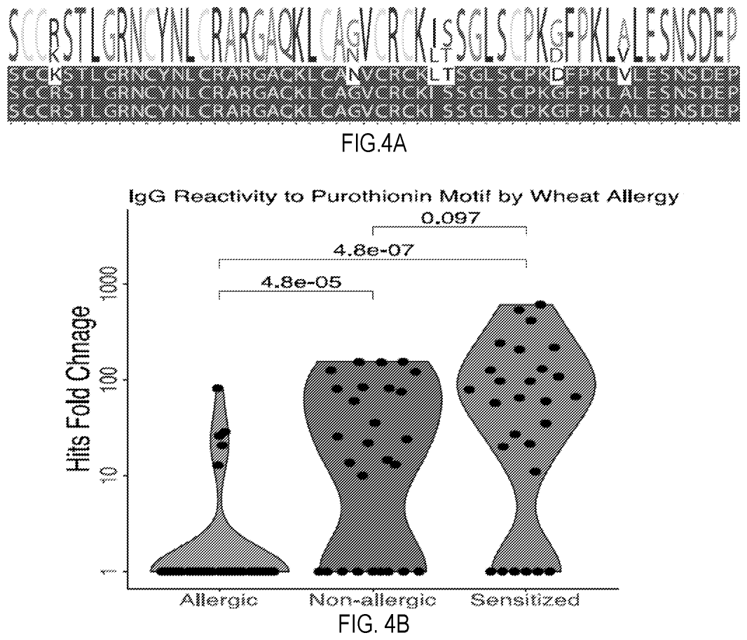

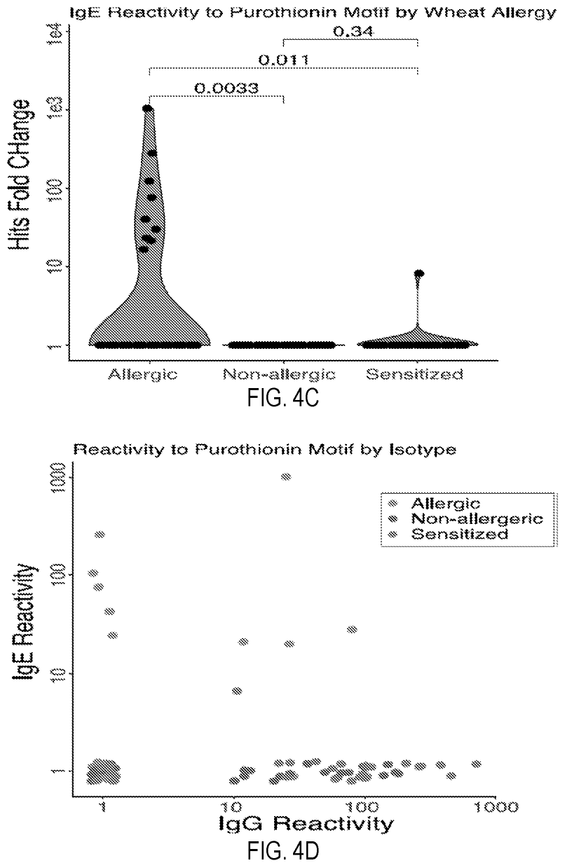

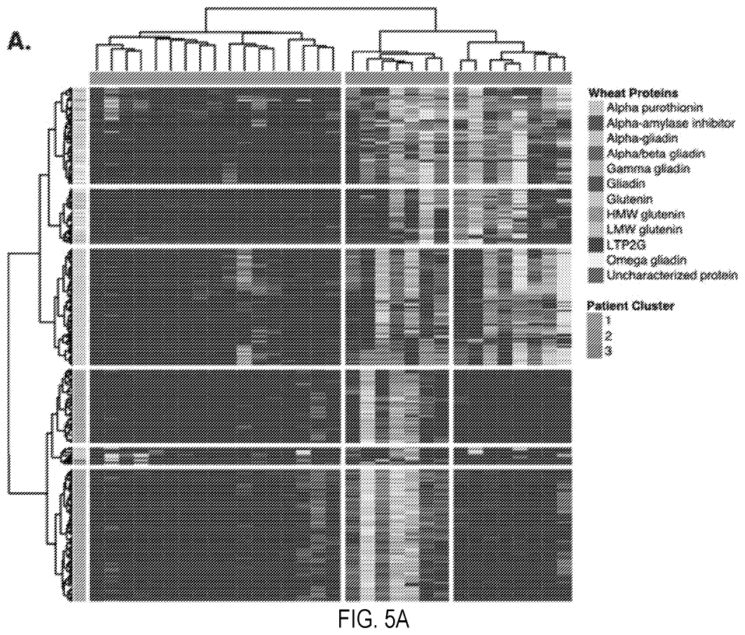

Upon examination of the wheat peptide reactivity matrix, the present inventors noted that whereas allergic individuals displayed higher IgG-reactivity for nearly every dominant epitope, a seemingly distinct set of three peptides (FIG. 3B, arrows) were frequently recognized by both non-allergic and sensitized individuals' IgG, but infrequently by allergic individuals' IgG. Interestingly, all three peptides were derived from an overlapping region of the Tri a 37 protein, which is also known as alpha purothionin (FIG. 4A-B). IgE reactivity to this Tri a 37 motif was frequent among allergic individuals, but almost never detected among the non-allergic or sensitized individuals (FIG. 4C). These data suggest that the relative amount of IgE versus IgG anti-Tri a 37 reactivity may also have utility in distinguishing allergic from sensitized individuals (FIG. 4D).

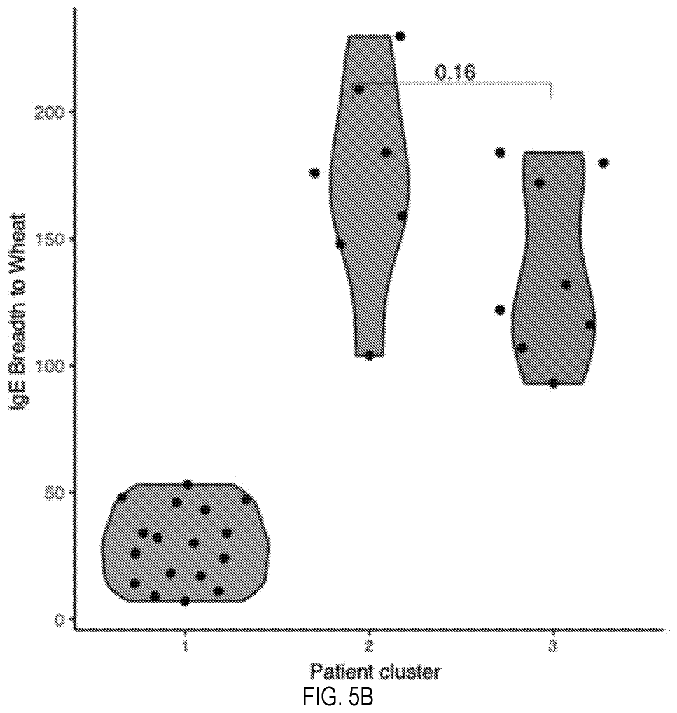

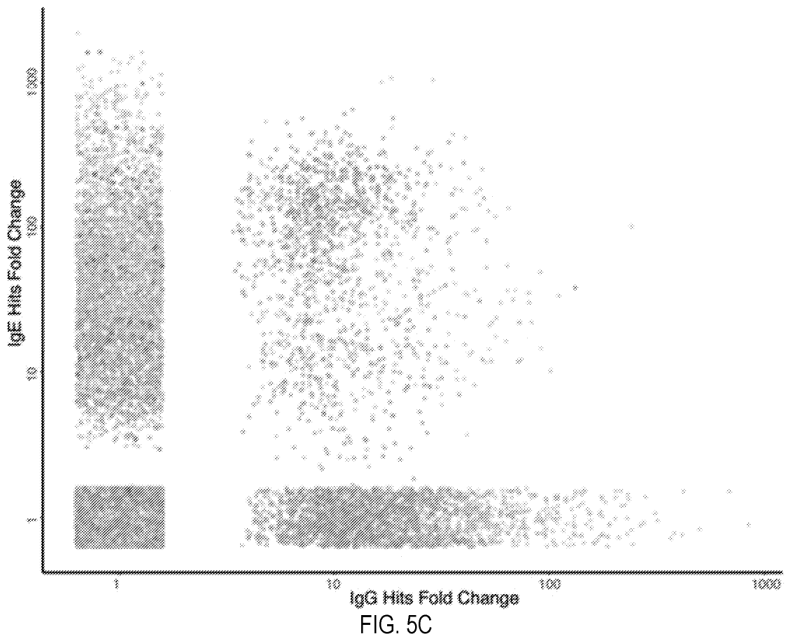

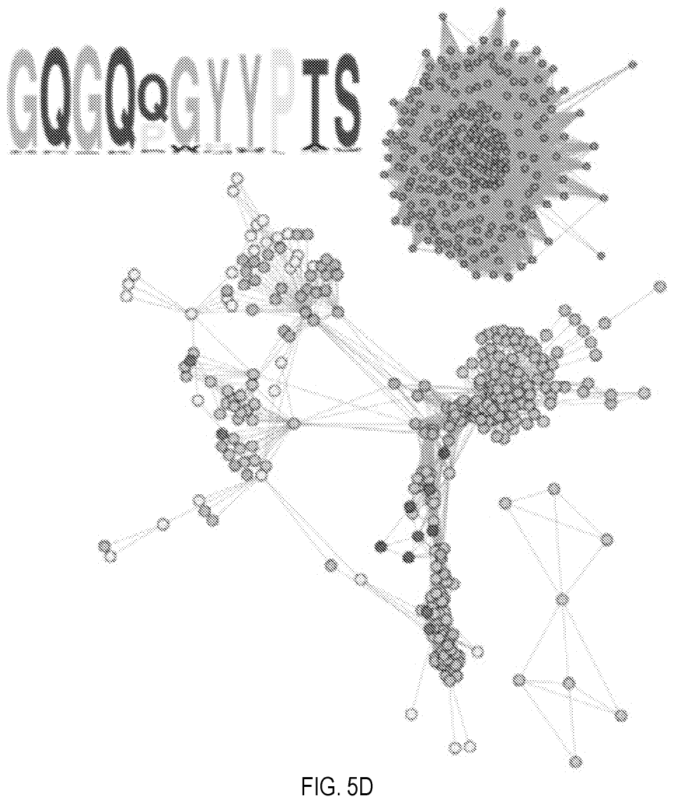

The present inventors next sought to determine whether there were distinguishable patient and peptide subgroups within the wheat allergic cohort based upon their anti-wheat IgE reactivity profiles. To this end, the IgE wheat reactivity data were subset to include only peptides recognized by at least 4 individuals and subjected to hierarchical clustering, which revealed six distinct peptide clusters and three main patient populations (FIG. 5A). Two of these peptide clusters were composed entirely of HWM glutenin peptides, one group was composed almost entirely of LWM glutenin peptides, and two other groups were composed primarily of alpha, gamma and omega gliadin peptides. At the population level, patient cluster two was the only cluster to recognize high molecular weight (HMW) glutenin, whereas patients in both cluster two and cluster three both demonstrated reactivities to low molecular weight (LMW) glutenin. Groups two and three had significantly higher anti-wheat IgE breadth than group one (FIG. 5B). The present inventors then sought to identify any clinical correlates of the patient clusters, assessing the severity of clinical symptoms, overall wheat IgE breadth between groups, and reactivity to Tri a 37. The present inventors found that there was no significant difference in the severity of symptoms or in Tri a 37 reactivity between the groups. The present inventors additionally investigated potential differences in IgE and IgG reactivity to specific peptides (FIG. 5C) and found overall discordance in peptide reactivity at the isotype level, with more than 80% of all peptides displaying only IgE or IgG reactivity.

The present inventors sought to determine whether the observed clustering of peptide reactivities might be explained at least in part by peptide sequence homology. A peptide sequence alignment-based network graph was therefore constructed, and the peptide nodes colored according to the protein from which they were derived (FIG. 5D—top). This graph separated into three disconnected subgraphs: one cluster of HWM glutenin peptides, one cluster composed of gliadins and LMW glutenins, and one cluster composed exclusively of Tri a 37. There were also 22 singleton peptides lacking any alignment to other enriched peptides (not shown). The present inventors noted dense families of homologous peptides represented among LMW and HMW glutenin peptides, which reflects the repetitive sequences found among the glutenins¹³. Indeed, all peptides in the HMW glutenin subgraph shared one tightly conserved epitope (FIG. 5D—bottom).

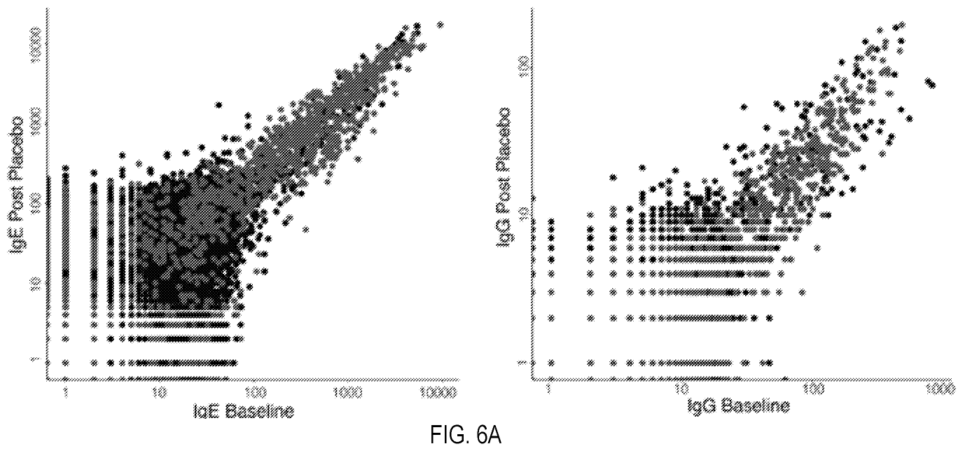

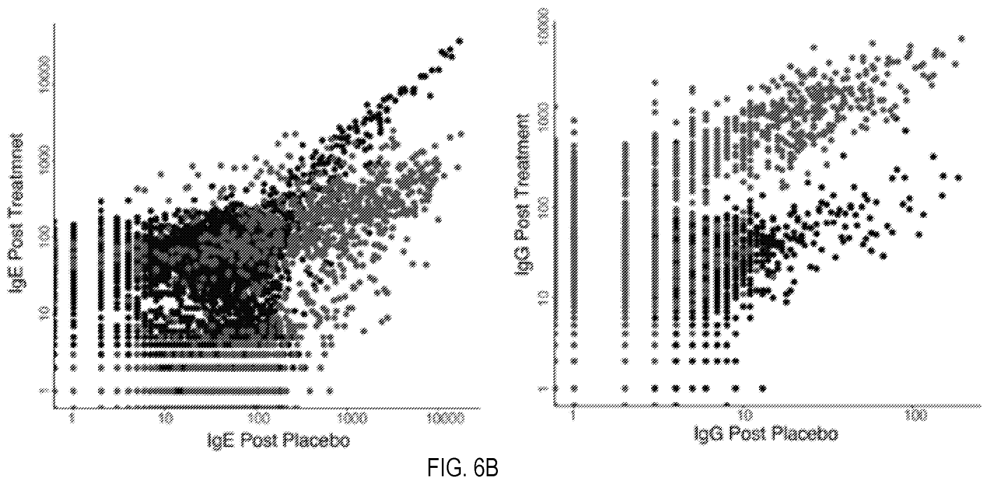

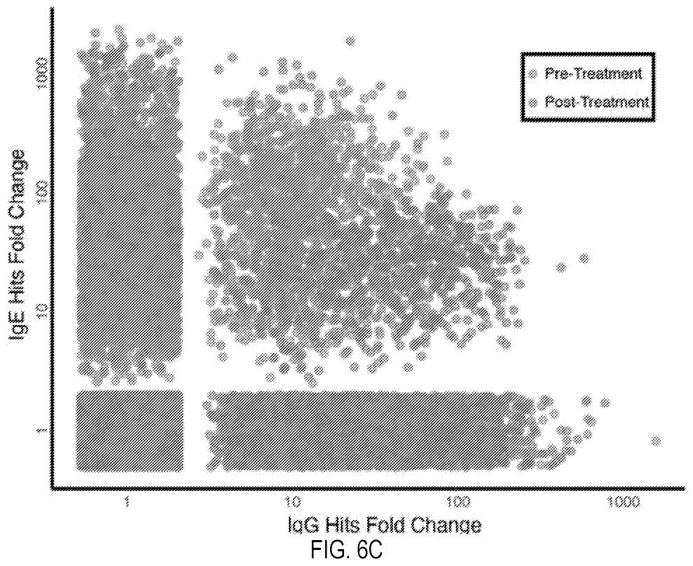

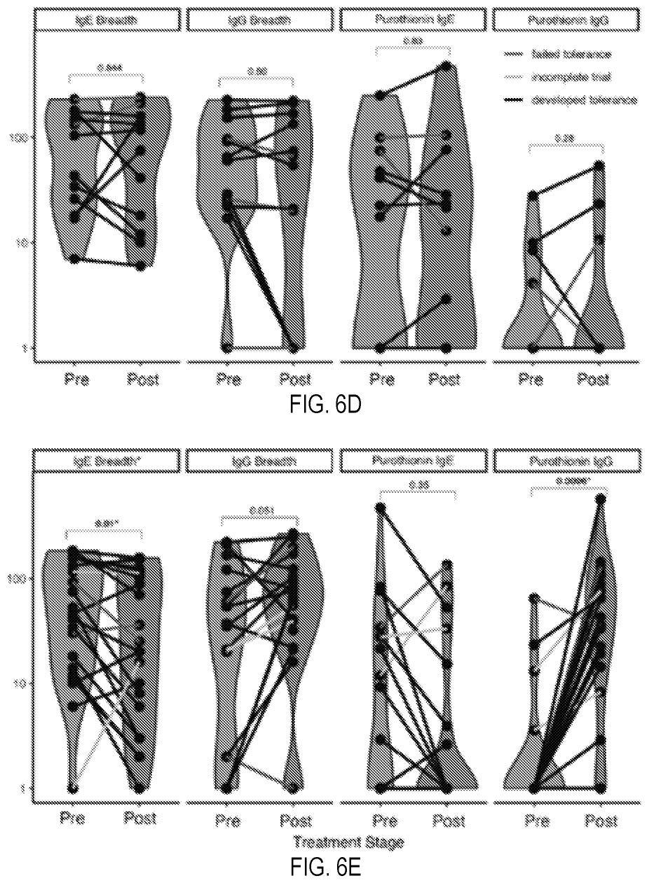

The present inventors next used the AllerScan library to characterize changes in wheat IgE and IgG reactivity in response to wheat oral immunotherapy (WOIT). Serum was analyzed from 23 participants in a randomized, double-blind, placebo-controlled clinical trial of WOIT. Participants in this trial received on a daily basis, either vital wheat gluten OIT or placebo, with biweekly escalations. After one year, participants receiving placebo crossed over to the treatment arm. In these AllerScan analyses, data are plotted in a pairwise fashion; unchanged reactivities fall along the y=x diagonal, whereas reactivities that increase or decrease over time appear in the upper left or lower right quadrant, respectively. Most individuals in the placebo arm of the trial exhibited minimal changes to their anti-allergen IgE or IgG profiles (see FIG. 6A for representative example). In stark contrast, however, the present inventors observed dramatic wheat-specific alterations in IgE and IgG reactivity during WOIT therapy in many individuals (see FIG. 6B for representative example of a tolerized individual); as expected, reactivity to non-wheat allergens remained stable over this time period. This trend of decreasing IgE and increasing IgG reactivity after wheat treatment was observed across this WOIT cohort (see FIG. 6C for aggregated data). During the placebo phase of the trial, neither anti-wheat breadth nor specific reactivity to Tri a 37 changed significantly, either in IgE or IgG (FIG. 6D). In contrast, the breadth of the anti-wheat IgE repertoire decreased significantly in response to WOIT (p=0.01, FIG. 6E), while the specific anti-Tri a 37 IgG response increased dramatically (p=0.0006, FIG. 6E), there was no association between anti-Tri a 37 IgE and WIT treatment. Taken together, our data point to Tri a 37 reactivity as an important biomarker of exposure and/or tolerance to wheat.

Discussion

The present inventors have created the AllerScan phage display library, which is composed of all protein sequences present in the Allergome database, and used it to characterize both IgE and IgG antibody reactivities in a cohort of peanut and wheat allergic individuals. Similar to previous PhIP-Seq libraries, AllerScan employs high throughput oligonucleotide synthesis to efficiently encode high quality, 56 amino acid peptide sequences. Currently, polypeptides of this length cannot be reliably synthesized chemically. IgE-specific immunoprecipitation of phage displayed peptides, followed by high throughput DNA sequencing is used to identify reactive peptides. Incorporation of liquid-handling automation, according to an embodiment, increases sample throughput and enhances assay reproducibility. High levels of sample multiplexing can be achieved by incorporating DNA barcodes into the PCR amplicon, prior to pooling and sequencing; at a sequencing depth of ˜10-fold, ˜500 AllerScan assays can be analyzed on a single run of an Illumina NextSeq 500. Batched analyses can therefore bring down the assay cost to just a few dollars per sample.

PhIP-Seq with the AllerScan library provides quantitative antibody reactivity data at the epitope level for about two-thousand proteins derived from hundreds of organisms. This unbiased approach evaluates antibody reactivities to major and minor allergens, including both well-defined and poorly studied epitopes. The library can be readily updated by simply adding new allergens as they are identified. Future studies using additional libraries, such as the human or VirScan phage libraries^{17,7} will enable a broader analysis of IgE (auto)reactivity.

Limitations of all programmable phage-based assays include the lack of post-translational modifications and discontinuous epitopes. While these two types of epitopes are likely critical for certain allergens¹⁸, protein denaturation during digestion is thought to reduce their functional significance for food allergies¹⁹. It is therefore possible that the AllerScan library may exhibit reduced sensitivity for non-food allergic antibodies. This possibility will be addressed in future work.

Here the present inventors analyzed over one million antibody-allergen peptide interactions in a comprehensive study of pan-allergen serology from a cross sectional cohort of patients with peanut and/or wheat allergies, as well as healthy controls. Peanut and wheat allergens were the most widely recognized in this cohort, but the present inventors also detected reactivities against many other organisms included tree nuts, invertebrate tropomyosin and milk proteins. This observation is not unexpected since patients with one allergy frequently have multiple additional unrelated allergies¹⁹. The present inventors sought to characterize anti-wheat antibody responses due to the current gap in knowledge about the relevant fine specificities. Our results suggest that peptide-level IgE reactivity may enhance discrimination between wheat allergy and sensitization. The present inventors also found that sensitized individuals harbored elevated anti-wheat IgG responses compared to their non-allergic counterparts. These data are consistent with the possibility that sensitized individuals also harbor IgE responses to wheat, but that their IgG can effectively block pathogenic IgE binding.

The present inventors observed consistent patterns of wheat reactivity amongst the allergic cohort; some peptides were reactive in over 80% of wheat allergics, whereas most peptides were recognized by no one. Dominant wheat reactivities tended to occur in highly repetitive regions of allergenic proteins, which has also been described for other allergens²¹. Additionally, the present inventors compared isotype differences in wheat peptide reactivity and found notable discordance in IgE and IgG reactivity for most peptides; only 16% of peptides were bound by both IgE and IgG isotypes. It is possible that this discordance may be partially explained by competition between the two antibody classes; future experiments in which purified IgE and IgG are used for AllerScan analysis will address this question.

In the wheat allergic individuals, the most dominant wheat epitopes were derived from alpha, beta, gamma and omega gliadin and both high and low molecular weight glutenin (HMW and LMW respectively). These proteins all harbor highly repetitive domains¹⁸. The present inventors then used sequence alignment to characterize homology among all dominant epitopes, which revealed modest homology between all gliadin peptides. Reactive omega and gamma gliadin epitopes were particularly homologous, which has been previously reported²². IgE reactive LMW glutenin peptides clustered with gliadins but did not share homology with high molecular weight glutenin peptides. The reactive peptides derived from HMW glutenin were characterized by a small number of highly repetitive motifs. All reactive HMW glutenin peptides possessed at least one IgE binding epitope with strong consensus to previously reported HMW epitopes¹⁸.

Tri a 37 is a plant defense protein abundantly expressed in wheat seeds, IgE to which has been associated with a four-fold increased risk of experiencing severe allergic reactions to wheat. Interestingly, while the present inventors did not detect an association between allergy severity and Tri a 37 reactivity in our study, the present inventors did identify anti-Tri a 37 IgG reactivity as the most prevalent (72%) reactivity in non-allergic and sensitized individuals, whereas IgG reactivity was low among the allergics. Conversely, IgE reactivity to Tri a 37 was found in 28% of allergics and only 2% of non-allergic/sensitized individuals. IgG and IgE reactivity to Tri a 37 may therefore be quite useful in distinguishing between allergy and sensitization.

The quantitative nature of AllerScan data enabled a longitudinal analysis of response to WOIT in a placebo-controlled, double blinded cross-over study involving 25 wheat allergic individuals. In response to WOIT, the present inventors noted a dramatic shift from IgE to IgG reactivity towards wheat peptides, whereas reactivity to other allergens were not affected. As expected, the placebo arm experienced no change in anti-wheat reactivity. Nearly every participant receiving treatment exhibited an overall reduction in anti-wheat IgE repertoire breadth and a concurrent increase in IgG repertoire breadth, an observation which has been reported in other food tolerance trials. Interestingly, almost all allergic patients receiving WOIT experienced an increase in IgG reactivity to Tri a 37.

In conclusion, AllerScan is a novel approach for epitope-level characterization of allergen-associated IgE and IgG responses in a large number of individuals. The present inventors have demonstrated its effectiveness in broad pan-allergen analysis as well as its utility in characterizing the fine specificities of anti-wheat antibodies. Our initial research has confirmed numerous known properties of wheat allergy as well as revealed several unreported properties, including reactivity towards a novel epitope, which discriminates between wheat allergy and sensitization. AllerScan is therefore a valuable new research tool for allergy research.

### REFERENCES

1. Soares-Weiser, K. et al. The diagnosis of food allergy: a systematic review and meta-analysis. Allergy 69, 76-86 (2014).
2. Constantin, C. et al. Micro-arrayed wheat seed and grass pollen allergens for component-resolved diagnosis. Allergy 64, 1030-1037 (2009).
3. Lin, J. & Sampson, H. A. The role of immunoglobulin E-binding epitopes in the characterization of food allergy. Curr. Opin. Allergy Clin. Immunol. 9, 357-363 (2009).
4. Suprun, M. et al. Novel Bead-Based Epitope Assay is a sensitive and reliable tool for profiling epitope-specific antibody repertoire in food allergy. Sci. Rep. 9, 1-14 (2019).
5. Larman, H. B. et al. Application of a synthetic human proteome to autoantigen discovery through PhIP-Seq. Nat. Biotechnol. 29, 535-541 (2011).
6. Mohan, D. et al. PhIP-Seq characterization of serum antibodies using oligonucleotide-encoded peptidomes. Nat. Protoc. 13, 1958-1978 (2018).
7. Xu, G. J. et al. Comprehensive serological profiling of human populations using a synthetic human virome. Science 348, aaa0698 (2015).
8. Mari, A. et al. Bioinformatics applied to allergy: allergen databases, from collecting sequence information to data integration. The Allergome platform as a model. Cell. Immunol. 244, 97-100 (2006).
9. Larman, H. B. et al. Application of a synthetic human proteome to autoantigen discovery through PhIP-Seq. Nat. Biotechnol. 29, 535-541 (2011).
10. Charles A Janeway, J., Travers, P., Walport, M. & Shlomchik, M. J. The distribution and functions of immunoglobulin isotypes. Immunobiol. Immune Syst. Health Dis. 5th Ed. (2001).
11. Schulman, E. S. Development of a monoclonal anti-immunoglobulin E antibody (omalizumab) for the treatment of allergic respiratory disorders. Am. J. Respir. Crit. Care Med. 164, S6-11 (2001).
12. Battais, F. et al. Identification of IgE-binding epitopes on gliadins for patients with food allergy to wheat. Allergy 60, 815-821 (2005).
13. Balakireva, A. V. & Zamyatnin, A. A. Properties of Gluten Intolerance: Gluten Structure, Evolution, Pathogenicity and Detoxification Capabilities. Nutrients 8, (2016).
14. Shewry, P. R., Halford, N. G. & Lafiandra, D. Genetics of Wheat Gluten Proteins. in Advances in Genetics (eds. Hall, J. C., Dunlap, J. C. & Friedmann, T.) vol. 49 111-184 (Academic Press, 2003).
15. Eshleman, S. H. et al. Comprehensive Profiling of HIV Antibody Evolution. Cell Rep. 27, 1422-1433.e4 (2019).
16. Monaco, D. R. et al. Deconvoluting Virome-Wide Antiviral Antibody Profiling Data. bioRxiv 333625 (2018) doi:10.1101/333625.
17. Larman, H. B. et al. Autoantigen discovery with a synthetic human peptidome. Nat. Biotechnol. 29, 535-541 (2011).
18. Matsuo, H., Yokooji, T. & Taogoshi, T. Common food allergens and their IgE-binding epitopes. Allergol. Int. 64, 332-343 (2015).
19. Verhoeckx, K. C. M. et al. Food processing and allergenicity. Food Chem.

Toxicol. 80, 223-240 (2015).
20. Dreskin, S. C. Genetics of food allergy. Curr. Allergy Asthma Rep. 6, 58-64 (2006).
21. Gupta, R. S. et al. Food Allergy Knowledge, Attitudes, and Beliefs of Primary Care Physicians. Pediatrics 125, 126-132 (2010).
22. Sackesen, C. et al. A new Luminex-based peptide assay to identify reactivity to baked, fermented, and whole milk. Allergy 74, 327-336 (2019).
23. Denery-Papini, S. et al. Allergy to deamidated gluten in patients tolerant to wheat: specific epitopes linked to deamidation. Allergy 67, 1023-1032 (2012).

### Example 2: Wheat Peptide Epitopes for Use in Passive or Active Immunization

Many wheat epitope reactivities are very “public”, meaning that allergic responses to wheat proteins are stereotyped and recognize a core set of epitopes. The present inventors used to AllerScan define the most public epitopes, and then used sequence alignment to determine their relationships. The most commonly targeted epitopes are candidate targets of passive immunotherapy—in certain embodiments, cocktails of monoclonal antibodies that block interactions between the allergens and patient IgE molecules. In other embodiments, these epitopes could serve as vaccine components under certain conditions.

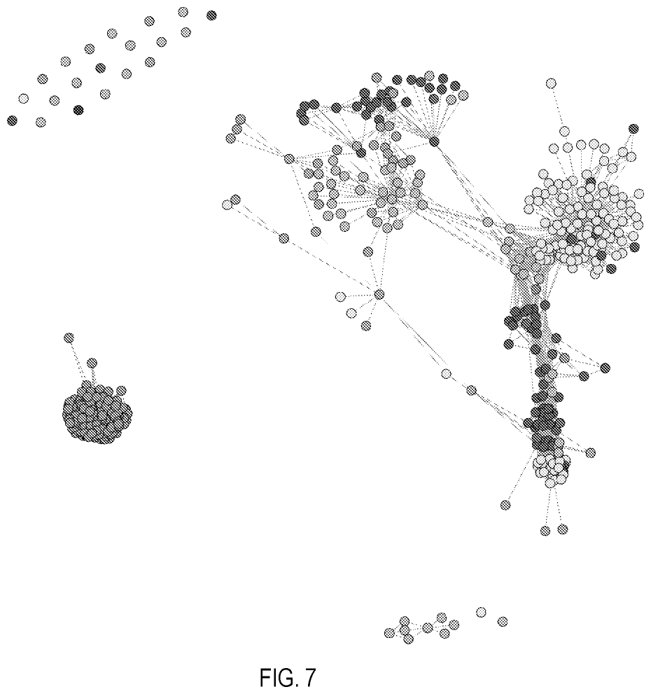

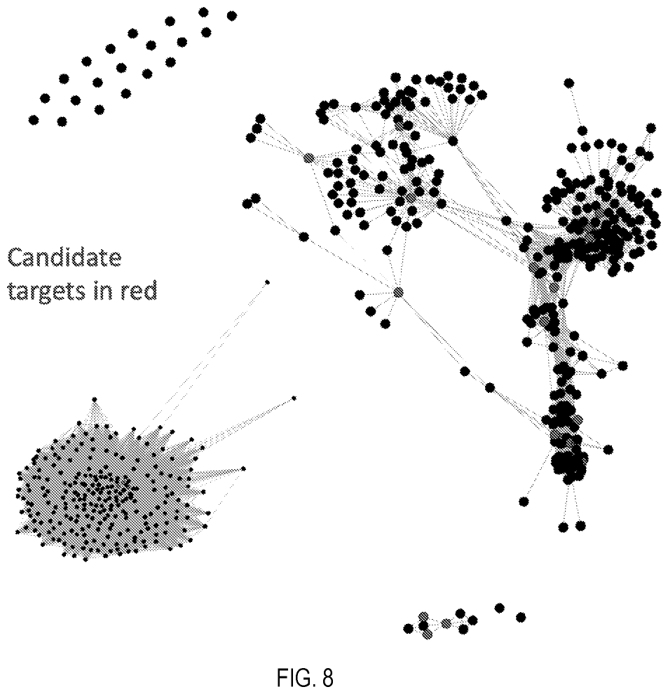

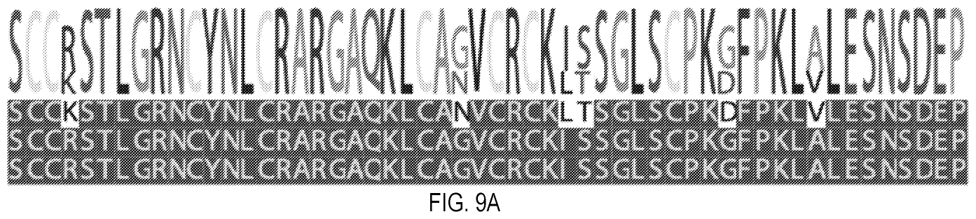

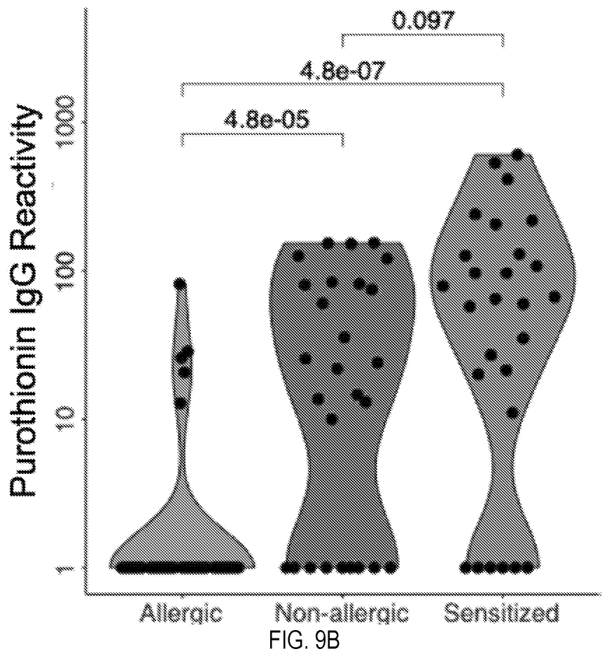

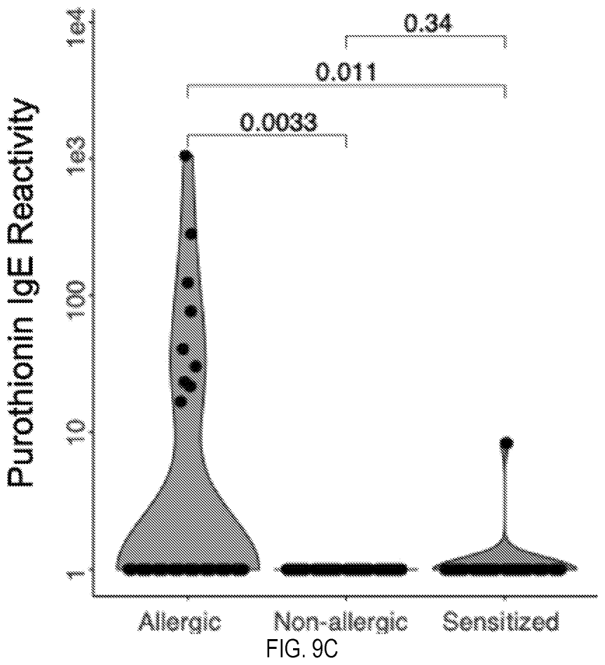

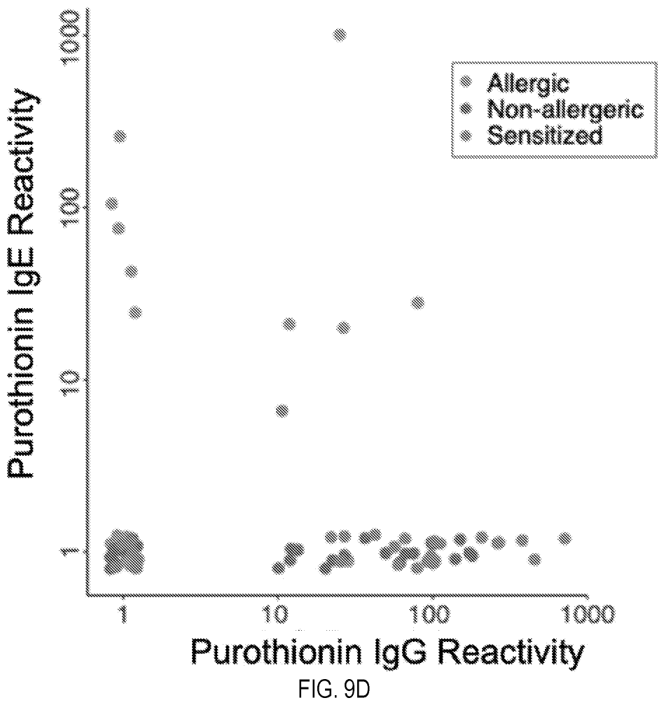

Table 1 is a list of all wheat designed peptides in the AllerScan library that have been recognized by IgE antibodies in 4 or more wheat allergic individuals (577 peptides). The amino acid sequence, protein product, peptide position and Uniprot ID have been provided for all peptides. From these 577 peptides, a peptide sequence similarity-based network graph was constructed; nodes are peptides and nodes are linked if they possess sequence similarity (FIG. 7). From this network graph, the top 3 most interconnected peptides from each of 8 different protein products were selected as the 3 most useful targets of passive antibody therapies (total of 24 peptide targets). The connectivity of these 24 candidate targets, relative to all 577 peptides, is shown in FIG. 8.

Active Immunization Rationale: One may consider vaccinating an allergic individual to elicit IgG reactivities that recognize epitopes that are contiguous, but do not overlap with the targets of public, wheat specific IgE molecules (IgE targets are provided in Table 1). Vaccine induced (actively immunized) responses may interfere with the ability of IgE-targeted epitopes to cause allergic symptoms, including anaphylaxis. The present inventors therefore mined the IgE reactivity database to identify wheat peptides from the same proteins corresponding to the 24 best targets (lines 2-25 in Table 1), which were found to lack any IgE reactivity in any of our study participants. The protein identifiers of the 24 top peptides in Table 1 (18 unique identifiers) were used to capture all peptides corresponding to the 18 unique identifiers, which also exhibited no IgE reactivity in any of our study participants. In one embodiment, the present inventors propose that these peptides (listed in Table 2) are candidates for vaccine induced anti-allergic immune responses.

In addition to the peptides that were selected for (a) targeting by “passive” antibody treatment, and (b) targeting by vaccine induced immune responses, the present inventors identified a peptide (SCCRSTLGRNCYNLCRARGAQKLCAGVCRCKISSGLSCPKGFPKLALESNSDEP) that offers utility in distinguishing between wheat-allergy and wheat-sensitization (individuals who can eat wheat but test positive in clinical assays).

The present inventors found that this peptide is frequently recognized by both non-allergic and sensitized individuals' IgG, but infrequently by allergic individuals' IgG. It was additionally found that IgE reactivity to this alpha purothionin motif was frequent among allergic individuals, but almost never detected among the non-allergic or sensitized individuals. Failure to distinguish between wheat-allergy and wheat-sensitization is a key shortcoming in many clinical assays which can result in which can lead to unnecessary food avoidance.

TABLE 1

| | | | | | | | IgE | |
|---|---|---|---|---|---|---|---|---|
| | Degree | | | | | | reac- | Opti- |
| | of con- | | | | | | tive | mal_ |
| | nect- | | pos_ | pos_ | | | sam- | block- |
| Amino Acid Sequence | ivity | product | start | end | pep_id | Median | ples | ing |
| ESNSDEPDTIEYCNLGCRSSVCDYMVNAAA | 6 | Alpha | 28 | 84 | Q9S6Y2\|28-84 | 63.36 | 5 | yes |
| DDEEMKLYVENCGDACVSFCNGDAGL | | purothionin | | | | | | |
| SCCRSTLGRNCYNLCRARGAQKLCAGVCRC | 4 | Alpha | 0 | NA | Q9T0P1_2 | 58.605 | 8 | yes |
| KISSGLSCPKGFPKLALESNSDEPDT | | purothionin | | | | | | |
| SCCRTTLGRNCYNLCRSRGAQKLCSTVCRC | 4 | Alpha | 28 | 84 | P32032\|28-84 | 69.465 | 10 | yes |
| KLTSGLSCPKGFPKLALESNSDEPDT | | purothionin | | | | | | |
| SFQQPLQQYPLGQGSFRPSQQNPQAQGSVQP | 54 | Alpha- | 196 | 252 | Q9M4L7\|196-252 | 72.2775 | 8 | yes |
| QQLPQFEEIRNLALQTLPAMCNVYI | | gliadin | | | | | | |
| YPLGQGSFRPSQQNPQAQGSVQPQQLPQFEE | 51 | Alpha- | 224 | 280 | Q41509\|224-280 | 71.415 | 10 | yes |
| IRNLALQTLPAMCNVYIPPYCTIAP | | gliadin | | | | | | |
| QQYPSGQGSFQPSQQNPQAQGSVQPQQLPQF | 51 | Alpha- | 224 | 280 | Q306F9\|224-280 | 50.29 | 14 | yes |
| EEIRNLALETLPAMCNVYIPPYCTI | | gliadin | | | | | | |
| QQQQQQQPSSQVSYQQPQQQYPSGQGSFQP | 45 | Alpha/beta | 196 | 252 | D2T2K3\|196-252 | 50.15 | 19 | yes |
| SQQNPQAQGFVQPQQLPQFEEIRNLA | | gliadin | | | | | | |
| PQQPISQQQAQLQQQQQQQQQQILQQILQQQ | 19 | Alpha/beta | 84 | 140 | D2T2K3\|84-140 | 10.3 | 11 | yes |
| LIPCRDVVLQQPNIAHASSQVSQQS | | gliadin | | | | | | |
| HAIILHHQQQQQQQQQQQQQQQQQQQQQQ | 17 | Alpha/beta | 168 | 224 | D2T2K3\|168-224 | 39.74 | 17 | yes |
| QQQQQQPSSQVSYQQPQQQYPSGQGSF | | gliadin | | | | | | |
| VQWPQQQPFPQPQQPFCEQPQRTIPQPHQTF | 49 | Gamma | 28 | 84 | D0EMA4\|28-84 | 59.635 | 12 | yes |
| HHQPQQTFPQPEQTYPHQPQQQFPQ | | gliadin | | | | | | |
| QQEQRQGVQIRRPLFQLVQGQGIIQPQQPAQ | 41 | Gamma | 196 | 252 | B6DQB1\|196-252 | 27.325 | 8 | yes |
| LEVIRSLALRTLPTMCNVYVSPDCS | | gliadin | | | | | | |
| LQQPQQPFPQPQQQLPQPQQPQQSFPQQQRS | 40 | Gamma | 140 | 196 | B6UKN8\|140-196 | 50 | 9 | yes |
| FIQPSLQQQLNPCKNILLQQCKPAS | | gliadin | | | | | | |
| PPFSQQLPPFSRQQQPVLPQQPPFSQQQLPPF | 50 | Glutenin | 140 | 196 | A2IBV5\|140-196 | 39.94 | 14 | yes |
| SQQQQPVLLQQQIPFVHPSILQQL | | | | | | | | |
| QCVSQPQQQSQQQLGQQPQQQQLAQGTFLQ | 38 | Glutenin | 280 | 336 | A2IBV5\|280-336 | 59.725 | 11 | yes |
| PHQIAQLEVMTSIALRTLPTMCNVNV | | | | | | | | |
| QLAQGTFLQPHQIAQLEVMTSIALRTLPTMC | 38 | Glutenin | CTERM | NA | A2IBV5\|CTERM | 47.0275 | 12 | yes |
| NVNVSLYRTTTRVPFGVGTGVGGY | | | | | | | | |
| QPGQGQQPGQWQQSGQGQHWYYPTSPQLS | 242 | HMW | 700 | 756 | A9YSK4\|700-756 | 11.75 | 12 | yes |
| GQGQRPGQWLQPGQGQQGYYPTSPQQP | | glutenin | | | | | | |
| SGQGQRPGQWLQPGQGQQGYYPTSPQQPGQ | 242 | HMW | 728 | 784 | A9YSK4\|728-784 | 36.84 | 9 | yes |
| GQQLGQWLQPGQGQQGYYPTSLQQTG | | glutenin | | | | | | |
| WQQSGQGQHGYYPTSPQLSGQGQRPGQWL | 242 | HMW | 896 | 952 | B1B520\|896-952 | 19.74 | 11 | yes |
| QPGQGQQGYYPTSPQQSGQGQQLGQWL | | glutenin | | | | | | |
| QQQQPPFSQHQQPVLPQQQIPSVQPSILQQLN | 108 | LMW | 140 | 196 | Q8W3X5\|140-196 | 64.97 | 4 | yes |
| PCKLFLQQQCSPVAMPQSLARSQT | | glutenin | | | | | | |
| FSQQQLPPFSQQLPPFSQQQQPVLLQQQIPFV | 103 | LMW | 112 | 168 | Q571Q5\|112-168 | 36.225 | 10 | yes |
| HPSILQQLNPCKVFLQQQCSPVAM | | glutenin | | | | | | |
| QLPPFSQQQQPVLPQQPPFSQQQLPPFSQQLP | 97 | LMW | 140 | 196 | Q8W3X3\|140-196 | 19.37 | 17 | yes |
| PFSQQQLPPFSQQLPPFSQQQQQV | | glutenin | | | | | | |
| IPPATRTNNSPATATTIPPAPQQRFPHTRQKFP | 10 | Omega | 252 | 308 | Q6PNA3\|252-308 | 21.63 | 8 | yes |
| RNPNNHSLCSTHHFPAQQPFPQQ | | gliadin | | | | | | |
| MSRLLSPSDQQLQSPQQQFPEEQSYPQQPYP | 9 | Omega | 0 | 56 | Q571R2\|0-56 | 21.85 | 15 | yes |
| QQAFPIPQQYSPHQPQQPFPQPQRP | | gliadin | | | | | | |
| QQPFPLQPQQQFPEQSEQIISQQRQQPFSLQP | 9 | Omega | 168 | 224 | Q571R2\|168-224 | 78.9525 | 8 | yes |
| QQPFSQPQQPLSQQPGQIIPQQPQ | | gliadin | | | | | | |
| WEPQHPSSPEHQPTPQPQEHPVPHQKLNPCR | 31 | Gliadin | 0 | NA | D2KFG9_2 | 53.05 | 14 | no |
| DALLQQCSPVADMSFLRSQVVQHSS | | | | | | | | |
| SCCKSTLGRNCYNLCRARGAQKLCANVCRC | 4 | Alpha | 28 | 84 | P01543\|28-84 | 62.91 | 13 | no |
| KLTSGLSCPKDFPKLVLESNSDEPDT | | purothionin | | | | | | |
| IEYCNLGCRSSVCDYMVNAAADDEEMKLY | 3 | Alpha | 0 | NA | Q9T0P1_4 | 79.68 | 5 | no |
| VENCADACVSFCNGDAGLPSLDAY | | purothionin | | | | | | |
| MKSCCRSTLGRNCYNLCRARGAQKLCAGV | 3 | Alpha | 0 | 56 | J7K291\|0-56 | 38.355 | 12 | no |
| CRCKISSGLSCPKGFPKLALESNSDEP | | purothionin | | | | | | |
| DTIEYCNLGCRSSVCDYMVNAAADDEEMKL | 3 | Alpha | CTERM | NA | J7K291\|CTERM | 68.68 | 5 | no |
| YVENCADACVSFCNGDAGLPSLDAY | | purothionin | | | | | | |
| PDTMEYCNLGCRSSLCDYIVNAAADDEEMK | 3 | Alpha | CTERM | NA | P01543\|CTERM | 58.86 | 5 | no |
| LYVEQCGDACVNFCNADAGLTSLDA | | purothionin | | | | | | |
| PGSKLPEWMTSASIYSPGKPYLAKLYCCQEL | 14 | Alpha- | 56 | 112 | P17314\|56-112 | 57.81 | 19 | no |
| AEISQQCRCEALRYFIALPVPSQPV | | amylase | | | | | | |
| | | inhibitor | | | | | | |
| CVPGVAFRTNLLPHCRDYVLQQTCGTFTPGS | 11 | Alpha- | 28 | 84 | P17314\|28-84 | 32.8 | 22 | no |
| KLPEWMTSASIYSPGKPYLAKLYCC | | amylase | | | | | | |
| | | inhibitor | | | | | | |
| CVGSQVPEAVLRDCCQQLADVNNEWCRCE | 6 | Alpha- | 28 | 84 | D2TGC3\|28-84 | 204.005 | 4 | no |
| DLSSMLRSVYQELGAREGKEVLPGCRK | | amylase | | | | | | |
| | | inhibitor | | | | | | |
| IAAEYDAWSCNSGPWMCYPGQAFQVPALPG | 3 | Alpha- | 0 | 56 | I6Q083\|0-56 | 91.22 | 7 | no |
| CRPLLKLQCNGSQVPEAVLRDCCQQL | | amylase | | | | | | |
| | | inhibitor | | | | | | |
| EQYPSGQGSFQSSQQNPQAQGSVQPQQLPQF | 50 | Alpha- | 196 | 252 | Q9M4M3\|196-252 | 23.545 | 7 | no |
| QEIRNLALQTLPAMCNVYIPPYCST | | gliadin | | | | | | |
| LSQVCFQQSQQQYPSGQGSFQPSQQNPQAQ | 50 | Alpha- | 224 | 280 | Q306F8\|224-280 | 43.315 | 15 | no |
| GSVQPQQLPQFEEIRNLALETLPAMC | | gliadin | | | | | | |
| LSQVSFQQPQQQYPSGQGFFQPFQQNPQAQG | 49 | Alpha- | 196 | 252 | Q9M4M4\|196-252 | 53.725 | 7 | no |
| SFQPQQLPQFEAIRNLALQTLPAMC | | gliadin | | | | | | |
| QVSFQPSQLNPQAQGPVQPQQLPQFAEIRNL | 43 | Alpha- | 252 | 308 | Q1WA40\|252-308 | 41.635 | 7 | no |
| ALQTLPAMCNVYIPPHCSTTIAPFG | | gliadin | | | | | | |
| LWQIPEQSRCQAIHNVVHAIILHQQQQQQQQ | 35 | Alpha- | 196 | 252 | Q1WA39\|196-252 | 66.675 | 13 | no |
| QQQPLSQVSFQQPQQQYPSGQGSFQ | | gliadin | | | | | | |
| LVQQLCCQQLWQIPEQSRCQAIHNVVHAIIL | 33 | Alpha- | 168 | 224 | Q9M4L6\|168-224 | 50.79 | 11 | no |
| HQQQQQQQQQQQQPLSQVSFQQPQQ | | gliadin | | | | | | |
| QILQQQLIPCRDVVLQQHSIAHGSSQVLQQST | 33 | Alpha- | 140 | 196 | Q306F8\|140-196 | 21.63 | 11 | no |
| YQLVQQLCCQQLWQIPEQSRCQAI | | gliadin | | | | | | |
| ILQQQLIPCRDVVLQQHNIAHASSQVLQQSS | 32 | Alpha- | 112 | 168 | Q9M4M3\|112-168 | 17.2625 | 6 | no |
| YQQLQQLCCQQLFQIPEQSRCQAIH | | gliadin | | | | | | |
| QCQAIHNVVHAIILHQQQKQQQQPSSQVSFQ | 32 | Alpha- | 168 | 224 | Q9M4L7\|168-224 | 37.94 | 21 | no |
| QPLQQYPLGQGSFRPSQQNPQAQGS | | gliadin | | | | | | |
| SQCQAIHNVVHAIILHQQQKQQQQLSSQVSF | 31 | Alpha- | 168 | 224 | Q9M4L9\|168-224 | 44.7125 | 20 | no |
| QQPQQQYPLGQGSFRPSQQNSQAQG | | gliadin | | | | | | |
| QQQQQQPLSQVSFQQPQQQYPSGQGSFQPSQ | 30 | Alpha- | 224 | 280 | Q1WA39\|224-280 | 44.95 | 21 | no |
| QNPQAQGSVQPQQLPQFEEIRNLAL | | gliadin | | | | | | |
| HNVVHAIILHQQQQQQQQQQQQQQQQQPLS | 30 | Alpha- | 196 | 252 | Q306F8\|196-252 | 52.65 | 12 | no |
| QVCFQQSQQQYPSGQGSFQPSQQNPQ | | gliadin | | | | | | |
| LQQQLTPCMDVVLQQHNIARGRSQVLQQST | 30 | Alpha- | 140 | 196 | Q41530\|140-196 | 45.38 | 6 | no |
| YQLLQELCCQHLWQIPEKLQCQAIHN | | gliadin | | | | | | |
| QQFLGQQQPFPPQQPYPQPQPFPSQQPYLQL | 29 | Alpha- | 28 | 84 | Q9M4M1\|28-84 | 61.01 | 13 | no |
| QPFPQPQLSYSQPQPFRPQQLYPQP | | gliadin | | | | | | |
| LQQILQQQLIPCRDVVLQQHNIAHASSQVLQ | 29 | Alpha- | 140 | 196 | Q1WA40\|140-196 | 43.81 | 20 | no |
| QSTYQLLQQLCCQQLLQIPEQSRCQ | | gliadin | | | | | | |
| IHNVVHAIILHQQHHHHQQQQQQQQQQPLS | 28 | Alpha- | 168 | 224 | Q9M4M4\|168-224 | 65.65 | 14 | no |
| QVSFQQPQQQYPSGQGFFQPFQQNPQ | | gliadin | | | | | | |
| VVHAIILHQQQQQQQQQPLSQVCFQQSQQQ | 28 | Alpha- | 196 | 252 | Q306F9\|196-252 | 55.615 | 14 | no |
| YPSGQGSFQPSQQNPQAQGSVQPQQL | | gliadin | | | | | | |
| QQLIPCMDVVLQQHNIAHGRSQVLQQSTYQ | 27 | Alpha- | 140 | 196 | Q306G0\|140-196 | 26.56 | 6 | no |
| LLQELCCQHLWQIPEQSQCQAIHNVV | | gliadin | | | | | | |
| NVVHAIILHHHQQQQQQPSSQVSYQQPQEQ | 25 | Alpha- | 168 | 224 | Q9M4M3\|168-224 | 41.705 | 16 | no |
| YPSGQGSFQSSQQNPQAQGSVQPQQL | | gliadin | | | | | | |
| QSSYQQLQQLCCQQLFQIPEQSRCQAIHNVV | 24 | Alpha- | 140 | 196 | Q9M4M3\|140-196 | 46.4475 | 8 | no |
| HAIILHHHQQQQQQPSSQVSYQQPQ | | gliadin | | | | | | |
| HAIILHQQQKQQQQQPSSQVSFQQPLQQYPL | 24 | Alpha- | 196 | 252 | Q41528\|196-252 | 38.635 | 22 | no |
| GQGSFRPSQQNPQTQGSVQPQQLPQ | | gliadin | | | | | | |
| IILHQQQQQQQQQQQQPLSQVSFQQPQQQYP | 23 | Alpha- | 196 | 252 | Q9M4L6\|196-252 | 46.75 | 21 | no |
| SGQGSFQPSQQNPQAQGSVQPQQLP | | gliadin | | | | | | |
| QQQQQQQQQQQPSSQVSFQQPQQQYPSSQV | 23 | Alpha- | 224 | 280 | Q41529\|224-280 | 34.61 | 21 | no |
| SFQPSQLNPQAQGSVQPQQLPQFAEI | | gliadin | | | | | | |
| HAIILHQQQKQQQQPSSQFSFQQPLQQYPLG | 22 | Alpha- | 196 | 252 | Q306G0\|196-252 | 34.14 | 21 | no |
| QGSFRPSQQNPQAQGSVQPQQLPQF | | gliadin | | | | | | |
| QQQQLQQQRQQPSSQVSFQQPQQQYPSSQV | 22 | Alpha- | 224 | 280 | Q1WA40\|224-280 | 38.95 | 21 | no |
| SFQPSQLNPQAQGPVQPQQLPQFAEI | | gliadin | | | | | | |
| VVHAIILHQQQQKQQQPSSQVSFQQPQQQYP | 22 | Alpha- | 196 | 252 | Q41530\|196-252 | 42.165 | 20 | no |
| LGQGSFRPSQQNPQAQGSVQPQQLP | | gliadin | | | | | | |
| YQLLRELCCQHLWQIPEQSQCQAIHNVVHAI | 21 | Alpha- | 168 | 224 | Q41528\|168-224 | 66.5425 | 8 | no |
| ILHQQQKQQQQQPSSQVSFQQPLQQ | | gliadin | | | | | | |
| STYQLLQELCCQHLWQIPEKLQCQAIHNVVH | 21 | Alpha- | 168 | 224 | Q41530\|168-224 | 48.03 | 8 | no |
| AIILHQQQQKQQQPSSQVSFQQPQQ | | gliadin | | | | | | |
| QQQQQQQQQQKQQQQQQQQILQQILQQQLI | 19 | Alpha- | 112 | 168 | Q9M4L6\|112-168 | 9.45 | 7 | no |
| PCRDVVLQQHSIAYGSSQVLQQSTYQ | | gliadin | | | | | | |
| QPQYPQPQQPISQQQAQQQQQQQQILQQILQ | 17 | Alpha- | 84 | 140 | Q9M4M3\|84-140 | 10.55 | 13 | no |
| QQLIPCRDVVLQQHNIAHASSQVLQ | | gliadin | | | | | | |
| PISQQQQQQQQQQQQQQQEQQILQQILQQQL | 17 | Alpha- | 112 | 168 | Q306G0\|112-168 | 15.05 | 7 | no |
| IPCMDVVLQQHNIAHGRSQVLQQST | | gliadin | | | | | | |
| LQPQQPISQQQAQQQQQQQQQQQQQQQILQ | 17 | Alpha- | 112 | 168 | Q1WA40\|112-168 | 21.595 | 7 | no |
| QILQQQLIPCRDVVLQQHNIAHASSQ | | gliadin | | | | | | |
| QPQYSQPQQPISQQQQQQQQQQQQQQQQQQ | 15 | Alpha- | 84 | 140 | Q9M4L7\|84-140 | 15.78 | 11 | no |
| ILQQILQQQLIPCMDVVLQQHNIAHG | | gliadin | | | | | | |
| QYSQPQQPISQQQQQQQQQQQQQQQILQQIL | 15 | Alpha- | 112 | 168 | Q306F8\|112-168 | 42.34 | 8 | no |
| QQQLIPCRDVVLQQHSIAHGSSQVL | | gliadin | | | | | | |
| AIHNVAHAIIMHQQQQQQQEQQQQLQQQQQ | 15 | Alpha- | 196 | 252 | Q1WA40\|196-252 | 48.48 | 16 | no |
| QQLQQQRQQPSSQVSFQQPQQQYPSS | | gliadin | | | | | | |
| IHNVVHAIIMHQQEQQQQLQQQQQQQLQQQ | 13 | Alpha- | 196 | 252 | Q41529\|196-252 | 41.18 | 17 | no |
| QQQQQQQQQPSSQVSFQQPQQQYPSS | | gliadin | | | | | | |
| QQFLGQQQPFPPQQPYPQPQPFPSQLPYLQL | 12 | Alpha- | 28 | 84 | Q9M4L7\|28-84 | 23.0825 | 22 | no |
| QPFPQPQLPYSQPQPFRPQQPYPQP | | gliadin | | | | | | |
| QPQNPSQQLPQEQVPLVQQQQFLGQQQPFPP | 12 | Alpha- | 28 | 84 | Q41528\|28-84 | 32.18 | 22 | no |
| QQPYPQPQFPSQLPYLQLQPFPQPQ | | gliadin | | | | | | |
| QPQYSQPQQPISQQQQQQQQQQQQQQQQQQ | 12 | Alpha- | 84 | 140 | Q9M4M5\|84-140 | 18.13 | 11 | no |
| QQQQQILQQILQQQLIPCMDVVLQQH | | gliadin | | | | | | |
| PYPQSQPQYSQPQQPISQQQQQQQQQQQQK | 10 | Alpha- | 112 | 168 | Q1WA39\|112-168 | 54.8425 | 6 | no |
| QQQQQQQQILQQILQQQLIPCRDVVL | | gliadin | | | | | | |
| FPPQQPYPQPQFPSQLPYLQLQPFPQPQLPYS | 9 | Alpha- | 56 | 112 | Q41528\|56-112 | 39.275 | 17 | no |
| QPQPFRPQQPYPQPQPQYSQPQQP | | gliadin | | | | | | |
| QPQNPSQQQPQKQVPLVQQQQFPGQQQPFPP | 9 | Alpha- | 28 | 84 | Q306F8\|28-84 | 37.665 | 19 | no |
| QQPYPQLQPFPSQQPYMQLQPFPQP | | gliadin | | | | | | |
| MVRVPVPQLQPQNPSQQHPQEQVPLVQQQQ | 8 | Alpha- | 0 | 56 | Q9M4L8\|0-56 | 24.2475 | 22 | no |
| FLGQQQSFPPQQPYPQPQPFPSQQPY | | gliadin | | | | | | |
| QQFLGQQQSFPPQQPYPQPQPFPSQQPYLQL | 8 | Alpha- | 28 | 84 | Q9M4L8\|28-84 | 17.995 | 22 | no |
| QPFPQPQLPYLQPQPFRPQQPYPQP | | gliadin | | | | | | |
| LQLQPFPQPQLPYSQPQPFRPQQPYPQPQPQY | 8 | Alpha- | 56 | 112 | Q9M4L7\|56-112 | 103.33 | 9 | no |
| SQPQQPISQQQQQQQQQQQQQQQQ | | gliadin | | | | | | |
| FPPQQPYPQLQPFPSQQPYMQLQPFPQPQLP | 7 | Alpha- | 56 | 112 | Q306F8\|56-112 | 23.78 | 21 | no |
| YPQPRLPYPQPQPFRPQQSYPQPQP | | gliadin | | | | | | |
| QPQYSQPQQPISQQQQQQQQQQQQQQQQQQ | 6 | Alpha- | 84 | 140 | Q9M4L8\|84-140 | 67.94 | 5 | no |
| QQQQQQQQILQQILQQQLIPCMDVVL | | gliadin | | | | | | |
| QQFPGQQQPFPPQQPYPQPQPFPSQQPYLQL | 6 | Alpha- | 28 | 84 | Q9M4L6\|28-84 | 19.28 | 23 | no |
| QPFPQPQLPYPQPQLPYPQPQLPYP | | gliadin | | | | | | |
| FPPQQPYPQPQPFPSQQPYLQLQPFPQPQLPY | 6 | Alpha- | 56 | 112 | Q1WA39\|56-112 | 36.6575 | 18 | no |
| PQPQLPYPQPQLPYPQPQPFRPQQ | | gliadin | | | | | | |
| LQLQPFPQPQLPYPQPQLPYPQPQLPYPQPQP | 4 | Alpha- | 56 | 112 | Q9M4L6\|56-112 | 62.835 | 14 | no |
| FRPQQPYPQSQPQYSQPQQPISQQ | | gliadin | | | | | | |
| QQFPGQQQPFPPQQPYPQPQPFPSQQPYLQL | 3 | Alpha- | 28 | 84 | Q9M4M4\|28-84 | 19.115 | 23 | no |
| QPFPRPQLPYPQPQPFRPQQPYPQP | | gliadin | | | | | | |
| FPPQQPYPQPQPFPSQQPYLQLQPFPQPQPFP | 3 | Alpha- | 56 | 112 | Q1WA40\|56-112 | 74.7 | 15 | no |
| PQLPYPQPQSFPPQQPYPQQQPQY | | gliadin | | | | | | |
| QQPYPQPQPFPSQQPYLQLQPFPQPQPFLPQL | 3 | Alpha- | 56 | 112 | Q41529\|56-112 | 21.645 | 22 | no |
| PYPQPQSFPPQQPYPQQRPKYLQP | | gliadin | | | | | | |
| MVRVPVPQLQPQNPSQQQPQEQVPLVQQQQ | 2 | Alpha- | 0 | 56 | Q9M4L6\|0-56 | 24.8075 | 22 | no |
| FPGQQQPFPPQQPYPQPQPFPSQQPY | | gliadin | | | | | | |
| MVRVPVPQLQPQNPSQQQPQEQVPLMQQQQ | 2 | Alpha- | 0 | 56 | Q9M4M3\|0-56 | 23.0525 | 24 | no |
| QFPGQQEQFPPQQPYPHQQPFPSQQP | | gliadin | | | | | | |
| MVRVPMPQLQPQDPSQQQPQEQVPLVQQQQ | 2 | Alpha- | 0 | 56 | Q9M4L9\|0-56 | 28.78 | 21 | no |
| FLGQQQPFPPQQPYPQPQPFPSQQPY | | gliadin | | | | | | |
| QLPYPQPQLPYPQPQPFRPQQSYPQPQPQYS | 2 | Alpha- | 84 | 140 | Q306F9\|84-140 | 33.51 | 14 | no |
| QPQQPISQQQQQQQQQQQQQILQQI | | gliadin | | | | | | |
| QLPYPQPRLPYPQPQPFRPQQSYPQPQPQYS | 2 | Alpha- | 84 | 140 | Q306F8\|84-140 | 70.555 | 6 | no |
| QPQQPISQQQQQQQQQQQQQQQILQ | | gliadin | | | | | | |
| YPQPQPFPPQLPYPQTQPFPPQQPYPQPQPQY | 1 | Alpha- | 56 | 112 | Q9M4M3\|56-112 | 67.175 | 10 | no |
| PQPQQPISQQQAQQQQQQQQILQQ | | gliadin | | | | | | |
| QLPYSQPQPFRPQQPYPQPQPQYSQPQQPISQ | 1 | Alpha- | 84 | 140 | Q41509\|84-140 | 28.64 | 13 | no |
| QQQQQQQQQQQQQQQQQQIIQQIL | | gliadin | | | | | | |
| QQQFPGQQEQFPPQQPYPHQQPFPSQQPYPQ | 1 | Alpha- | 28 | 84 | Q9M4M3\|28-84 | 58.1675 | 12 | no |
| PQPFPPQLPYPQTQPFPPQQPYPQP | | gliadin | | | | | | |
| QLPYSQPQPFRPQQPYPQPQPQYSQPQQPISQ | 1 | Alpha- | 84 | 140 | Q306G0\|84-140 | 47.12 | 10 | no |
| QQQQQQQQQQQQQQEQQILQQILQ | | gliadin | | | | | | |
| QLPYPQPQLPYPQPQLPYPQPQPFRPQQPYPQ | 1 | Alpha- | 84 | 140 | Q1WA39\|84-140 | 75.905 | 10 | no |
| SQPQYSQPQQPISQQQQQQQQQQQ | | gliadin | | | | | | |
| QPQPFRPQQPYPQSQPQYSQPQQPISQQQQQ | 0 | Alpha- | 84 | 140 | Q9M4L6\|84-140 | 42.98 | 8 | no |
| QQQQQQQKQQQQQQQQILQQILQQQ | | gliadin | | | | | | |
| QPFPPQLPYPQPQSFPPQQPYPQQQPQYLQPQ | 0 | Alpha- | 84 | 140 | Q1WA40\|84-140 | 76.6075 | 8 | no |
| QPISQQQAQQQQQQQQQQQQQQQI | | gliadin | | | | | | |
| VRVPVPQLQPQNPSQQQPQEQVPLVQQQQF | 2 | Alpha/beta | 0 | 56 | D2T2K3\|0-56 | 54.17 | 19 | no |
| LGQQQQHFPGQQQPFPPQQPYPQPQP | | gliadin | | | | | | |
| FLPQLPYPQPQPFPPQQSYPQPQPQYPQPQQP | 1 | Alpha/beta | 56 | 112 | D2T2K3\|56-112 | 31.385 | 7 | no |
| ISQQQAQLQQQQQQQQQQILQQIL | | gliadin | | | | | | |
| QFLGQQQQHFPGQQQPFPPQQPYPQPQPFLP | 0 | Alpha/beta | 28 | 84 | D2T2K3\|28-84 | 44.295 | 11 | no |
| QLPYPQPQPFPPQQSYPQPQPQYPQ | | gliadin | | | | | | |
| GVQILVPLSQQQQVGQGTLVQGQGIIQPQQP | 37 | Gamma | 224 | 280 | D0ES84\|224-280 | 27.865 | 4 | no |
| AQLEVIRSSVLQTLATMCNVYVPPY | | gliadin | | | | | | |
| QTFPHQPQQQFPQPQQPQQSFPQQQQPLIQP | 35 | Gamma | 56 | 112 | B6UKP3\|56-112 | 54.2425 | 6 | no |
| YLQQQMNPCKNYLLQQCNPVSLVSS | | gliadin | | | | | | |
| SQQPQQQFSQPQQQFPQPQQPQQSFPQQQPP | 34 | Gamma | 112 | 168 | A1EHE7\|112-168 | 46.1625 | 8 | no |
| FIQPSLQQQVNPCKNFLLQQCKPVS | | gliadin | | | | | | |
| QPFPQSQQPQQPFPQPQQPQQSFPQQQQPLIQ | 32 | Gamma | 112 | 168 | A7XDG6\|112-168 | 27.085 | 10 | no |
| PYLQQQMNPCKNYLLQQCNPVSLV | | gliadin | | | | | | |
| VHSIIMQQEQQQQQQQQQQQQQQQGIQIMR | 29 | Gamma | 224 | 280 | D0ES81\|224-280 | 100.5 | 5 | no |
| PLFQLVQGQGIIQPQQPAQLEVIRSL | | gliadin | | | | | | |
| SQQPQQPFPQPQQQFPQPQQPQQSFPQQQPS | 26 | Gamma | 112 | 168 | D0ES84\|112-168 | 46.5125 | 10 | no |
| LIQQSLQQQLNPCKNFLLQQCKPVS | | gliadin | | | | | | |
| SQQPQQPFPQPQQQFLQPQQPQQSFPQQQQP | 26 | Gamma | 112 | 168 | B6UKM8\|112-168 | 35.44 | 11 | no |
| LIQLSLQQQMNPCKNFLLQQCNPVS | | gliadin | | | | | | |
| QQCCQQLAQIPQQLQCAAIHSVVHSIIMQQE | 24 | Gamma | 168 | 224 | B6DQB1\|168-224 | 70.37 | 5 | no |
| QRQGVQIRRPLFQLVQGQGIIQPQQ | | gliadin | | | | | | |
| QPQQLFPQSQQPQQQFSQPQQQFPQPQQPQQ | 22 | Gamma | 56 | 112 | B6UKM6\|56-112 | 88.295 | 7 | no |
| SFPQQQPPFIQPSLQQQVNPCKNFL | | gliadin | | | | | | |
| DCQVMRQQCCQQLAQIPQQLQCAAIHSVVH | 22 | Gamma | 196 | 252 | B6UKN1\|196-252 | 57.54 | 5 | no |
| SIVMQQEQQQGIQILRPLFQLVQGQG | | gliadin | | | | | | |
| VQWPQQQPVPQPHQPFSQQPQHTFPQPQQTF | 20 | Gamma | 28 | 84 | B6UKM4\|28-84 | 76.6675 | 8 | no |
| PHQPQQQFPQPQQPQQQFPQPQRPQ | | gliadin | | | | | | |
| QQPQQQFPQSQQPQQPFPQPQQQFLQPPQPQ | 20 | Gamma | 84 | 140 | B6DQB1\|84-140 | 60.98 | 9 | no |
| QSFPQQQQPLIQLSLQQQMNPCKNF | | gliadin | | | | | | |
| QPFPQPQQPQQPFPQSKQPQQPFPQPQQPQQ | 19 | Gamma | 112 | 168 | B6UKQ2\|112-168 | 86.015 | 9 | no |
| SFPQQQPSLIQQSLQQQLNPCKNFL | | gliadin | | | | | | |
| QQPQQPFPQSQQPQQPFPQPQQQFPQPQQPQ | 16 | Gamma | 84 | 140 | B6DQB2\|84-140 | 82.115 | 7 | no |
| QSFPQQQPSLIQQSLQQQLNPCKNL | | gliadin | | | | | | |
| IPQQLQCAAIHTVIHSIIMQQEQQQGMHILLP | 16 | Gamma | 196 | 252 | A1EHE7\|196-252 | 30.425 | 6 | no |
| LYQQQQVGQGTLVQGQGIIQPQQP | | gliadin | | | | | | |
| VQWPQQQPFRQPQQPFYQQPQHTFPQPQQT | 12 | Gamma | 28 | 84 | Q94G94\|28-84 | 37.07 | 13 | no |
| CPHQPQQQFPQPQQPQQPFPQPQQPQ | | gliadin | | | | | | |
| QTFHHQPQQTFPQPQQTYPHQPQQQFPQPQQ | 10 | Gamma | 56 | 112 | B6UKP7\|56-112 | 51.71 | 9 | no |
| PQQSFPQQQQPAIQSFLQQQMNPCK | | gliadin | | | | | | |
| VPRPQQQPFPQPHQPFSQQPQQTFPQPQQTFP | 4 | Gamma | 28 | 84 | D0ES82\|28-84 | 35.975 | 11 | no |
| HQPQQQFSQPQQPQQQFIQPQQPF | | gliadin | | | | | | |
| NMQVDPSGQVPWPQQQPFPQPHQPFSQQPQ | 4 | Gamma | 0 | 56 | B6DQB2\|0-56 | 51.04 | 13 | no |
| QTFPQPQQTFPHQPQQQFSQPQQPQQ | | gliadin | | | | | | |
| VQWPQQQPVLLPQQPFSQQPQQTFPQPQQTF | 3 | Gamma | 28 | 84 | D0ES84\|28-84 | 33.9 | 12 | no |
| PHQPQQQFSQPQQPQQQFIQPQQPF | | gliadin | | | | | | |
| QPQQTFPQPQQTFPHQPQQQFSQPQQPQQQF | 3 | Gamma | 28 | 84 | Q9M4L5\|28-84 | 56.585 | 8 | no |
| IQPQQPQQTYPQRPQQPFPQTQQPQ | | gliadin | | | | | | |
| NMQVDPSSQVQWPQQQPVPQPHQPFSQQPQ | 3 | Gamma | 0 | 56 | B6DQB1\|0-56 | 39.2775 | 12 | no |
| QTFPQPQQTFPHQPQQQFPQPQQPQQ | | gliadin | | | | | | |
| PQQTFPQPQQTFPHQPQQQFPQPQQPQQQFL | 3 | Gamma | 28 | 84 | B6DQB1\|28-84 | 82.1875 | 10 | no |
| QPQQPFPQQPQQPYPQQPQQPFPQT | | gliadin | | | | | | |
| VQWPQQQPVPQPHQPFSQQPQQTFPQPQQTF | 3 | Gamma | 28 | 84 | A1EHE7\|28-84 | 77.335 | 7 | no |
| PHQPQQQFPQPQQPQQQFLQPQQPF | | gliadin | | | | | | |
| VQWPQQQPVPLPQQPFSQKPQQTFPQPQQTF | 2 | Gamma | 28 | 84 | F6KV47\|28-84 | 69.1075 | 10 | no |
| PFPHQPQQQFPQPQQPQQQFLQPQQ | | gliadin | | | | | | |
| VQWPQQQPFRQPQQPFYQQPQHTFPQPQQT | 2 | Gamma | 28 | 84 | B6UKP3\|28-84 | 52.38 | 13 | no |
| FPHQPQQQFPQPQQPQQSFPQQQQPL | | gliadin | | | | | | |
| QTFHHQPQQTFPQPQQTYPHQPQQQFPQPQQ | 2 | Gamma | 56 | 112 | B6UKQ6\|56-112 | 79.09 | 11 | no |
| PQQPFPQPQQAQLPFPQQPQQPFPQ | | gliadin | | | | | | |
| QTFHHQPQQTFPQPQQTYPHQPQQQFPQTQQ | 2 | Gamma | 56 | 112 | B6UKM5\|56-112 | 55.98 | 14 | no |
| PQQPFPQPQQTFPQQPQLPFPQQRQ | | gliadin | | | | | | |
| TQQPQQPFPQPQQTFPQQPQLPFPQQRQQPFP | 2 | Gamma | 84 | 140 | B6UKM5\|84-140 | 46.54 | 13 | no |
| QPQQPQQPFPQSQQPQQPFPQPQQ | | gliadin | | | | | | |
| VQWPQQQPVLLPQQPFSQQPQQTFPRPQQTF | 2 | Gamma | 28 | 84 | B6UKN8\|28-84 | 61.72 | 8 | no |
| PHQPQQQVPQPQQPQQPFLQPQQPF | | gliadin | | | | | | |
| TQQPQQPFPQPQQTFPQQPQLPFPQQRQQPFP | 2 | Gamma | 84 | 140 | B6UKN9\|84-140 | 59.44 | 9 | no |
| QTQQPQQLFPQSQQPQQQFSQPQQ | | gliadin | | | | | | |
| PQQTFPQPQQTFPHQPQQQFSQPQQPQQQFI | 2 | Gamma | 28 | 84 | B6DQB2\|28-84 | 71.67 | 13 | no |
| QPQQPFPQQPQQTYPQRPQQLFPQT | | gliadin | | | | | | |
| QTFPHQPQQQFPQPQQPQQQFLQPQQPFPQQ | 2 | Gamma | 56 | 112 | A1EHE7\|56-112 | 81.51 | 11 | no |
| PQQPYPQQPQQPFPQTQQPQQLFPQ | | gliadin | | | | | | |
| QPFPQPQQPQQPFPQSQQPQQPFPQPQQQFP | 1 | Gamma | 112 | 168 | D0ES81\|112-168 | 33.88 | 12 | no |
| QPQQPQQSFPQQQQPLIQPYLQQQM | | gliadin | | | | | | |
| QTFPHQPQQQFSQPQQPQQQFIQPQQPFPQQ | 1 | Gamma | 56 | 112 | D0ES82\|56-112 | 40.53 | 14 | no |
| PQQTYPQRPQQPFPQTQQPQQPFPQ | | gliadin | | | | | | |
| PQQPQQTYPQRPQQPFPQTQQPQQPFPQSQQ | 1 | Gamma | 84 | 140 | D0ES84\|84-140 | 78.8925 | 4 | no |
| PQQPFPQPQQQFPQPQQPQQSFPQQ | | gliadin | | | | | | |
| VQWPQQQPFRQPQQPFYQQPQQTFPQPQQIF | 1 | Gamma | 28 | 84 | A7XDG6\|28-84 | 69.435 | 12 | no |
| PHQPQQQFPQPQQPQQQFPQPQQPQ | | gliadin | | | | | | |
| QIFPHQPQQQFPQPQQPQQQFPQPQQPQQPFP | 1 | Gamma | 56 | 112 | A7XDG6\|56-112 | 44.6 | 13 | no |
| QPQQAQLPFPQQPQQPFPQPQQPQ | | gliadin | | | | | | |
| VQWPQQQQPFPQPQQPQQPFPQPQQPQLPFP | 1 | Gamma | 28 | 84 | Q94G97\|28-84 | 48.37 | 13 | no |
| QQPQQPFPQPQQPQQPFPQLQQPQQ | | gliadin | | | | | | |
| VQWPQQQPFRQPQQPFYQQPQQQFPQPQQP | 1 | Gamma | 28 | 84 | B6UKN2\|28-84 | 59.425 | 10 | no |
| QQPFPQPQQAQLPFPQQPQQPFPQPQ | | gliadin | | | | | | |
| VQWPQQQPFRQPQQPFCQQPQRTIPQPHQTF | 1 | Gamma | 28 | 84 | B6UKM5\|28-84 | 48.91 | 10 | no |
| HHQPQQTFPQPQQTYPHQPQQQFPQ | | gliadin | | | | | | |
| QPFPQPQQPQQPFPQSQQPQQPFPQPQQQFP | 1 | Gamma | 112 | 168 | B6UKM5\|112-168 | 65.745 | 7 | no |
| QPQQPQQSFPQQQQPAIQSFLQQQM | | gliadin | | | | | | |
| PQQPQQPYPQQPQQPFPQPQQPQQQFPQSQQ | 1 | Gamma | 84 | 140 | B6UKM8\|84-140 | 70.33 | 10 | no |
| PQQPFPQPQQQFLQPQQPQQSFPQQ | | gliadin | | | | | | |
| QTFPHQPQQQVPQPQQPQQPFLQPQQPFPQQ | 1 | Gamma | 56 | 112 | B6UKN8\|56-112 | 55.56 | 14 | no |
| PQQPFPQTQQPQQPFPQQPQQPFPQ | | gliadin | | | | | | |
| PQQPQQPFPQTQQPQQPFPQQPQQPFPQTQQ | 1 | Gamma | 84 | 140 | B6UKN8\|84-140 | 59.05 | 15 | no |
| PQQPFPQQPQQPFPQTQQPQQPFPQ | | gliadin | | | | | | |
| QTFPHQPQQQFPQPQQPQQQFPQPQQPQQPF | 1 | Gamma | 56 | 112 | B6UKP8\|56-112 | 64.565 | 5 | no |
| PQPQQQFPQPQQPQQSFPQQQQPAI | | gliadin | | | | | | |
| QTFPHQPQQQFSQPQQPQQQFIQPQQPFPQQ | 0 | Gamma | 56 | 112 | F2X0K7\|56-112 | 73.24 | 6 | no |
| PYPQQPQHPFPQTQQPQQPFPQSQQ | | gliadin | | | | | | |
| PQQPYPQQPQHPFPQTQQPQQPFPQSQQPQQ | 0 | Gamma | 84 | 140 | F2X0K7\|84-140 | 86.235 | 6 | no |
| PFPQPQQQFPQPQQPQQSFPQQQPS | | gliadin | | | | | | |
| QTFPFPHQPQQQFPQPQQPQQQFLQPQQPQQ | 0 | Gamma | 56 | 112 | F6KV47\|56-112 | 72.0625 | 8 | no |
| PYPQQPQQPFPQTQQPQQLFPQSQQ | | gliadin | | | | | | |
| PQQPQQTYPQRPQQPFPQTQQPQQPFPQSQQ | 0 | Gamma | 84 | 140 | D0ES82\|84-140 | 52.845 | 14 | no |
| PQQPFPQSQQPQQPFPQPQQQFPQP | | gliadin | | | | | | |
| QPFPQPQQAQLPFPQQPQQPFPQPQQPQQPFP | 0 | Gamma | 84 | 140 | A7XDG6\|84-140 | 38.2 | 15 | no |
| QSQQPQQPFPQPQQPQQSFPQQQQ | | gliadin | | | | | | |
| PFPQQPQQPFPQPQQPQQPFPQLQQPQQPLPQ | 0 | Gamma | 56 | 112 | Q94G97\|56-112 | 65.18 | 9 | no |
| PQQPQQPFPQQQQPLIQPYLQQQM | | gliadin | | | | | | |
| VQWPQQQPFRQPQQPYPQQPQQPFPQTQQP | 0 | Gamma | 28 | 84 | B6UKM6\|28-84 | 74.625 | 8 | no |
| QQLFPQSQQPQQQFSQPQQQFPQPQQ | | gliadin | | | | | | |
| TQQPQQPFPQQPQQPFPQTQQPQQPFPQLQQ | 0 | Gamma | 112 | 168 | B6UKN8\|112-168 | 45.83 | 15 | no |
| PQQPFPQPQQQLPQPQQPQQSFPQQ | | gliadin | | | | | | |
| PQQPYPQQPQQPFPQTQQPQQPFPQSKQPQQ | 0 | Gamma | 56 | 112 | B6UKP2\|56-112 | 76.92 | 9 | no |
| PFPQPQQPQQSFPQQQPSLIQQSLQ | | gliadin | | | | | | |
| QFLQPQQPFPQQPQQPYPQQPQQPFPQTQQP | 0 | Gamma | 56 | 112 | B6DQB1\|56-112 | 49.86 | 5 | no |
| QQQFPQSQQPQQPFPQPQQQFLQPP | | gliadin | | | | | | |
| QFIQPQQPFPQQPQQTYPQRPQQLFPQTQQP | 0 | Gamma | 56 | 112 | B6DQB2\|56-112 | 55.39 | 9 | no |
| QQPFPQSQQPQQPFPQPQQQFPQPQ | | gliadin | | | | | | |
| PQQPQQPYPQQPQQPFPQTQQPQQLFPQSQQ | 0 | Gamma | 84 | 140 | A1EHE7\|84-140 | 120 | 9 | no |
| PQQQFSQPQQQFPQPQQPQQSFPQQ | | gliadin | | | | | | |
| QLAQGTFLQPHQIAQLEVMTSIALRILPTMCS | 38 | Glutenin | CTERM | NA | A2IBV7\|CTERM | 44.3025 | 4 | no |
| VNVPLYRTTTSVPFGVGTRVGAY | | | | | | | | |
| FSQQQQPPFSQQQQPVLPQQPSFSQQQLPPFS | 37 | Glutenin | 112 | 168 | A2IBV5\|112-168 | 28.125 | 16 | no |
| QQLPPFSRQQQPVLPQQPPFSQQQ | | | | | | | | |
| QQPQQLGQCVSQPQQQSQQQLGQQPQQQQL | 33 | Glutenin | 224 | 280 | A2IBV7\|224-280 | 131.48 | 6 | no |
| AQGTFLQPHQIAQLEVMTSIALRILP | | | | | | | | |
| NPCMVFLQQQCSPVAMPQSLARSQMLQQSS | 28 | Glutenin | 196 | 252 | A2IBV5\|196-252 | 17.18 | 9 | no |
| CHVMQQQCCRQLPQIPQQSRYEAIRA | | | | | | | | |
| QQQQPPFSQQQQPVLPQQPSFSQQQLPPFSQ | 10 | Glutenin | 84 | 140 | A2IBV5\|84-140 | 48.63 | 9 | no |
| QQQPPFSQQQQPVLPQQPSFSQQQL | | | | | | | | |
| ILPQQPPFSQQQQLVLPQQPPFSQQQQPALPP | 5 | Glutenin | 84 | 140 | A2IBV7\|84-140 | 39.18 | 14 | no |
| QQSPFPQQQQQHQQLVQQQIPVVQ | | | | | | | | |
| PHQFPQQQPCSQQQQQPPLSQQQQPPFSQQQ | 4 | Glutenin | 56 | 112 | A2IBV5\|56-112 | 42.51 | 13 | no |
| QPPFSQQQQPVLPQQPSFSQQQLPP | | | | | | | | |
| QLFPQQPSFSQQQPPFWQQQPTFSQQQPILPQ | 4 | Glutenin | 56 | 112 | A2IBV7\|56-112 | 84.6025 | 10 | no |
| QPPFSQQQQLVLPQQPPFSQQQQP | | | | | | | | |
| LERPWQQQPLPPQQTFPQQPLFSQQQQQQLF | 3 | Glutenin | 28 | 84 | A2IBV7\|28-84 | 30.425 | 7 | no |
| PQQPSFSQQQPPFWQQQPTFSQQQP | | | | | | | | |
| WYYPTSPQLSGQGQRPGQWLQPGQGQQGY | 242 | HMW | 728 | 784 | C0SUC3\|728-784 | 22.33 | 7 | no |
| YPTSPQQPGQGQQLGQWLQPGQGQQGY | | glutenin | | | | | | |
| PGQASPQQPGQGQQPGKWQELGQGQQGYY | 242 | HMW | 140 | 196 | A5HMG2\|140-196 | 55.64 | 6 | no |
| PTSLHQSGQGQQGYYPSSLQQPGQGQQ | | glutenin | | | | | | |
| QGQPGYYPTSPQQPGQEQQLEQWQQSGQGQ | 240 | HMW | 504 | 560 | A0MZ38\|504-560 | 39.02 | 9 | no |
| PGHYPTSPLQPGQGQPGYYPTSPQQI | | glutenin | | | | | | |
| GQGQQPGQGQQGQQLGQGQQGYYPTSLQQ | 240 | HMW | 252 | 308 | A9YSK4\|252-308 | 32.6875 | 8 | no |
| SGQGQPGYYPTSLQQLGQGQSGYYPTS | | glutenin | | | | | | |
| SGQEQQLEQWQQSGQGQPGHYPTSPLQPGQ | 240 | HMW | 532 | 588 | G3K725\|532-588 | 38.69 | 6 | no |
| GQPGYYPTSPQQIGQGQQPGQLQQPT | | glutenin | | | | | | |
| PISPQQLGQGQQSGQGQLGYYPTSPQQSGQG | 239 | HMW | 280 | 336 | A0MZ38\|280-336 | 27.8 | 11 | no |
| QSGYYPTSAQQPGQLQQSTQEQQLG | | glutenin | | | | | | |
| LQPGQGQQGYYPTSPQQSGQGQQLGQWLQP | 239 | HMW | 924 | 980 | B1B520\|924-980 | 25.795 | 8 | no |
| GQGQQGYYPTSLQQTGQGQQSGQGQQ | | glutenin | | | | | | |
| QQSGQGQQPGQGQQPGQGQEGYYPTLGQQ | 238 | HMW | 644 | 700 | Q9SDM3\|644-700 | 23.03 | 10 | no |
| PGQWLQIGQGQQGYYPTSPQQLGQGQQ | | glutenin | | | | | | |
| GQPGYYPTSPQQSGQGQPGYYPTSPQQSGQL | 238 | HMW | 448 | 504 | A0MZ38\|448-504 | 33.67 | 7 | no |
| QQPAQGQQPGQEQQGQQPGQGQQPG | | glutenin | | | | | | |
| QPGQRQQPGQGKPGYYPTSPQQSGQGQSGY | 238 | HMW | 336 | 392 | Q94IJ9\|336-392 | 44.635 | 6 | no |
| YPTSPQQPGQEQQPGQGQQVQQPGQG | | glutenin | | | | | | |
| QPGQGQQGYYPTSLQQTGQGQQSGQGQQG | 238 | HMW | 952 | 1008 | B1B520\|952-1008 | 33.34 | 8 | no |
| YYSSYHVSVEHQAASLKVAKAQQLAAQ | | glutenin | | | | | | |
| GQPGHYPTSPLQPGQGQPGYYPTSPQQIGQG | 237 | HMW | 532 | 588 | A0MZ38\|532-588 | 17.02 | 8 | no |
| QQPGQLQQPTQGQQGQQPGQGQQGQ | | glutenin | | | | | | |
| QQPGQGQQGHCPTSRQQPGQAQQPGQGQQI | 237 | HMW | 588 | 644 | Q94IJ8\|588-644 | 77.82 | 5 | no |
| GQAQKPGQGQQGYYPTSLQQPGQGQQ | | glutenin | | | | | | |
| QAYYPTSSQQSGQRQQAGQWQRPGQGQSG | 237 | HMW | 476 | 532 | A5HMG1\|476-532 | 17.87 | 9 | no |
| YYPTSPQQPGQEQQSGQAQQSGQWQLV | | glutenin | | | | | | |
| QQPGQGQPGYYPTSSLQLGQGQQGYYPTSQ | 236 | HMW | 644 | 700 | Q19AE4\|644-700 | 50.65 | 9 | no |
| QQPGQGPQPGQWQQLGQGQQGYYPTS | | glutenin | | | | | | |
| PQQSGQGQQPGQWLQSGYYLTSPQQLGQGQ | 235 | HMW | 700 | 756 | Q19AE4\|700-756 | 159.8 | 4 | no |
| QPRQWLQPRQGQQGYYPTSPQQSGQG | | glutenin | | | | | | |
| QSGQGQPGYYPTSLQQLGQGQSGYYPTSPQ | 235 | HMW | 280 | 336 | A9YSK4\|280-336 | 16.91 | 11 | no |
| QPGQGQQPGQLQQPAQGQQPGQGQQG | | glutenin | | | | | | |
| QGQQGYYPTSPQQSGQGQQPGQWLQPGQG | 235 | HMW | 672 | 728 | Q94IJ7\|672-728 | 19.29 | 9 | no |
| QEGYYPTSPQQPGQGQQPGQWLQIGQG | | glutenin | | | | | | |
| PTSPQQSGQGQQPGHEQQPGQWLQPGQGQQ | 234 | HMW | 672 | 728 | A9YSK5\|672-728 | 34.0925 | 8 | no |
| GYYPTSSQQSGQGHQSGQGQQGYYPT | | glutenin | | | | | | |
| QQPGQGLPGYYPTSSLQPEQGQQGYYPTSQ | 234 | HMW | 644 | 700 | G3K725\|644-700 | 41.395 | 7 | no |
| QQPGQGPQPGQWQQSGQGQQGYYPTS | | glutenin | | | | | | |
| GQGQPGYYPTSPLQSGQGQSGYYPTSPQQSG | 234 | HMW | 420 | 476 | G3FLC7\|420-476 | 20.25 | 11 | no |
| QGQQPGQLQQPAQGQQPEQGQQGQQ | | glutenin | | | | | | |
| PGQGQPWYYPTSPQESGQGQQPGQWQQPG | 233 | HMW | 644 | 700 | A9YSK4\|644-700 | 15.6 | 10 | no |
| QGQPGYYLTSPLQLGQGQQGYYPTSLQ | | glutenin | | | | | | |
| GQGQPGYYLTSPLQLGQGQQGYYPTSLQQP | 232 | HMW | 672 | 728 | A9YSK4\|672-728 | 25.48 | 7 | no |
| GQGQQPGQWQQSGQGQHWYYPTSPQL | | glutenin | | | | | | |
| QQPEQGQQPGQGQQGYYPTSPQQSGQGKQL | 232 | HMW | 532 | 588 | Q52JL2\|532-588 | 32.41 | 5 | no |
| GQEQQGYYPTSPQQPGQGQQPGQGQQ | | glutenin | | | | | | |
| GHPGQRQQPGQGQQPEQGQQPGQGQQGYY | 231 | HMW | 532 | 588 | B8PSA6\|532-588 | 28.095 | 10 | no |
| PTSPQQPGQGKQLRQGQQGYYPTSLQQ | | glutenin | | | | | | |
| GQGQSGYYPTSAQQPGQLQQSTQEQQLGQE | 230 | HMW | 308 | 364 | A0MZ38\|308-364 | 77.02 | 4 | no |
| QQDPQSGQGRQGQQSGQRQQDQQSGQ | | glutenin | | | | | | |
| YYPTSPQQPGQGQQAGHGQQPGQWLQPGQ | 229 | HMW | 392 | 448 | L0G7U5\|392-448 | 17.56 | 1 | no |
| GQQGYYPTSLQQSGQGQQSGQGQQGYY | | glutenin | | | | | | |
| QGQQGQQQRQGEHGQQPGQGQQGQQPGQG | 227 | HMW | 420 | 476 | A0MZ38\|420-476 | 20.86 | 13 | no |
| QPGYYPTSPQQSGQGQPGYYPTSPQQS | | glutenin | | | | | | |
| YYPTSPQQPGQGQQLGQWLQPGQGQQGYY | 227 | HMW | 756 | 812 | C0SUC3\|756-812 | 24.57 | 7 | no |
| PTSLQQTGQGQQSGQGQQGYYSSYHVS | | glutenin | | | | | | |
| TSPQQSGQWQQPGQGQSGYYPTSPQQSGQK | 226 | HMW | 140 | 196 | A0MZ38\|140-196 | 58.955 | 4 | no |
| QPGYYPTSPWQPEQLQQPTQGQQRQQ | | glutenin | | | | | | |
| GQEGYYPTSPQQPGQGQQPGQWLQIGQGQQ | 225 | HMW | 700 | 756 | Q94IJ7\|700-756 | 34.3825 | 8 | no |
| GYYPTSPQQPGQGQQGYYLTSPQQPG | | glutenin | | | | | | |
| QQPGQGQPGYYPTSPQQPGQGQQPGQEQQP | 225 | HMW | 308 | 364 | Q94IJ9\|308-364 | 21.18 | 8 | no |
| GQRQQPGQGKPGYYPTSPQQSGQGQS | | glutenin | | | | | | |
| QGYYPTSLQQPGQGQQPGQWQQSGQGQHW | 225 | HMW | 700 | 756 | C0SUC3\|700-756 | 53.98 | 4 | no |
| YYPTSPQLSGQGQRPGQWLQPGQGQQG | | glutenin | | | | | | |
| PQQLGQGQQPRQWLQPRQGQQGYYPTSPQQ | 224 | HMW | 728 | 784 | G3K725\|728-784 | 39.54 | 9 | no |
| SGQGQQLGQGQQGYYPTSPQQSGQGQ | | glutenin | | | | | | |
| QQPGQGQQSGQGQQGQQPGQGQQAYYPTS | 223 | HMW | 448 | 504 | A9YSK5\|448-504 | 42.7425 | 8 | no |
| SQQSRQRQQAGQWQRPGQGQPGYYPTS | | glutenin | | | | | | |
| GQQGQQVGQGQQAQQPGQGQQPGQGQPGY | 223 | HMW | 392 | 448 | A9YSK4\|392-448 | 22.4325 | 8 | no |
| YPTSPQQSGQGQPGYYLTSPQQSGQGQ | | glutenin | | | | | | |
| SQQPGQGQQGQQVGQGQQAQQPGHGQQPG | 223 | HMW | 392 | 448 | G3FLC7\|392-448 | 35.24 | 7 | no |
| QGQPGYYPTSPLQSGQGQSGYYPTSPQ | | glutenin | | | | | | |
| GQGQQGHCPTSPQQTGQAQQPGQGQQIGQV | 223 | HMW | 616 | 672 | A5HMG2\|616-672 | 42.755 | 6 | no |
| QEPGQGQQGYYPISLQQSGQGQQSGQ | | glutenin | | | | | | |
| QGQQGQQPGQGQQPGQGQPGYYPTSPQQSG | 221 | HMW | 504 | 560 | G3K725\|504-560 | 15.79 | 13 | no |
| QEQQLEQWQQSGQGQPGHYPTSPLQP | | glutenin | | | | | | |
| PQQSGQGQQPGQWLQPGQWLQSGYYLTSP | 221 | HMW | 700 | 756 | G3K725\|700-756 | 18.53 | 8 | no |
| QQLGQGQQPRQWLQPRQGQQGYYPTSP | | glutenin | | | | | | |
| YPTSPQQPGQGKQLRQGQQGYYPTSLQQPG | 221 | HMW | 560 | 616 | Q4JHY1\|560-616 | 24.625 | 9 | no |
| QGQQPGQGQQSGQGQQGHCPTSPQQT | | glutenin | | | | | | |
| PGQASPQQPGQGQQPGKWQEPGQGQQWYY | 220 | HMW | 140 | 196 | A4URY8\|140-196 | 14.855 | 8 | no |
| PTSLQQPGQGQQMGKGKQGYYPTSLQQ | | glutenin | | | | | | |
| GQQPGQRQPGYYSTSPQQLGQGQPRYYPTC | 220 | HMW | 364 | 420 | A0MZ38\|364-420 | 32.355 | 4 | no |
| PQQPGQEQQPRQLQQPEHGQQGQQPE | | glutenin | | | | | | |
| SPLQLGQGQQGYYPTSLQQPGQGQQPGQWQ | 220 | HMW | 868 | 924 | B1B520\|868-924 | 50.77 | 8 | no |
| QSGQGQHGYYPTSPQLSGQGQRPGQW | | glutenin | | | | | | |
| PQESGQGQQPGQWQQPGQGQPGYYLTSPLQ | 219 | HMW | 644 | 700 | E2CT66\|644-700 | 78.98 | 5 | no |
| LGQGQQGYYPTSLQQPGQGQQPGQWQ | | glutenin | | | | | | |
| QVQEPGQGQQGYYPISLQQSGQGQQSGQGQ | 219 | HMW | 644 | 700 | A5HMG2\|644-700 | 14.12 | 8 | no |
| QSGQGHQLGQGQQSGQEQQGYDNPYH | | glutenin | | | | | | |
| LQQSGQGQQPGQWQQPGQGQPGYYPTSSLQ | 217 | HMW | 616 | 672 | A0MZ38\|616-672 | 82.49 | 5 | no |
| PEQGQQGYYPTSQQQPGQGPQPGQWQ | | glutenin | | | | | | |
| PTSPQESGQGQQPGQWQQPGQWQQPGQGQ | 216 | HMW | 560 | 616 | C6L669\|560-616 | 17.415 | 10 | no |
| PGYYLTSPLQLGQGQQGYYPTSLQQPG | | glutenin | | | | | | |
| YYPGGPQQPGQGQQLGQWLQPGQGQQGYY | 216 | HMW | 756 | 812 | D0IQ05\|756-812 | 26.6 | 7 | no |
| PTGLQQTGQGQQSGQGQQGYYSSYHVS | | glutenin | | | | | | |
| PGQGQQGYYPTSLQHTGQRQQPVQGQQPEQ | 215 | HMW | 196 | 252 | A9YSK3\|196-252 | 52.5675 | 6 | no |
| GQQPGQWQQGYYPTSPQQLGQGQQPR | | glutenin | | | | | | |
| QSGQGQQGYYPTSSQQSGQGQQPGQWLQP | 214 | HMW | 672 | 728 | A0MZ38\|672-728 | 21.045 | 8 | no |
| GQWLQSGYYLTSPQQLGQGQQPRQWSQ | | glutenin | | | | | | |
| QGQQPEQGQQGQQPGQGEQGQQPGQGQQG | 214 | HMW | 420 | 476 | G3K725\|420-476 | 19.395 | 14 | no |
| QQPGQGQPGYYPTSPQQSGQGQPGYYP | | glutenin | | | | | | |
| GQGQQPGQWQQPGQWQQPGQGQPGYYLTS | 213 | HMW | 840 | 896 | B1B520\|840-896 | 10.22 | 8 | no |
| PLQLGQGQQGYYPTSLQQPGQGQQPGQ | | glutenin | | | | | | |
| QQGYYPISPQQSGQGQQPGQGQQGYYPTSP | 211 | HMW | 616 | 672 | Q18MZ6\|616-672 | 32.5575 | 8 | no |
| QQSGQGQQPGQWLQPGQGQQGYYPTS | | glutenin | | | | | | |
| QPGQWLQIGQGQQGYYPTSPQQLGQGQQG | 210 | HMW | 672 | 728 | Q9SDM3\|672-728 | 54.285 | 6 | no |
| YYLTSPQQPGQGKQPGQGQQSYDSPYH | | glutenin | | | | | | |
| GQGQQPGQWQQPGQGQPGYYPTSPLQPGQ | 210 | HMW | 504 | 560 | A9YSK4\|504-560 | 11.49 | 11 | no |
| GQPGYDPTSPQQPGQGQQPGQLQQPAQ | | glutenin | | | | | | |
| PRQGQQGYYPTSPQQSGQGQQLGQGQQGY | 209 | HMW | 728 | 784 | A0MZ38\|728-784 | 56.8 | 9 | no |
| YPTSPQQSGQGQQGYDSPYYVSAEHQA | | glutenin | | | | | | |
| QQGQQPGQGQQPGQGQQGQQLGQGQQGY | 209 | HMW | 252 | 308 | C6L669\|252-308 | 20.41 | 13 | no |
| YPTSPQQSGQGQPGYYPTSSQQPTQSQQ | | glutenin | | | | | | |
| YPTSLHQSGQGQQGYYPSSLQQPGQGQQTG | 208 | HMW | 168 | 224 | A5HMG2\|168-224 | 77.8 | 6 | no |
| QGQQGYYPTSLQQPGQGQQIGQGQQG | | glutenin | | | | | | |
| GQGQQGYYPTSPQQPGQWQQPEQGQPRYYP | 207 | HMW | 140 | 196 | A9YSK4\|140-196 | 20.645 | 7 | no |
| TSPQQSGQLQQPAQGQQPGQGQQGQQ | | glutenin | | | | | | |
| QQGQQPGQGQQPGQGQQGQQLGQGQQGY | 207 | HMW | 252 | 308 | D0IQ07\|252-308 | 16.84 | 14 | no |
| YPTSLQQQPGYYPTSLQQLGQGQSGYYP | | glutenin | | | | | | |
| QPGQGQQGYYPTSPQQLGQGQQPGQWQQP | 207 | HMW | 252 | 308 | L0G7U5\|252-308 | 20.3 | 14 | no |
| GQGQPGYYPTSPQQPGQGQPGYYPTSP | | glutenin | | | | | | |
| GYYPTSPQQPGQEQQPGQGQQVQQPGQGQQ | 206 | HMW | 364 | 420 | Q94IJ9\|364-420 | 67.24 | 5 | no |
| PGQGQQGYYPTSPQQSGQAQQPGQWQ | | glutenin | | | | | | |
| GQQPGQGQPGYYPTSLQQSGQGQQPGQWQ | 205 | HMW | 616 | 672 | G3K725\|616-672 | 20.1 | 10 | no |
| QPGQGLPGYYPTSSLQPEQGQQGYYPT | | glutenin | | | | | | |
| GQQPGQGQPGYYPTSLQQSGQGQQPGQWQ | 205 | HMW | 616 | 672 | Q19AE4\|616-672 | 56.41 | 9 | no |
| QPGQGQPGYYPTSSLQLGQGQQGYYPT | | glutenin | | | | | | |
| LQQPTQGQQGYYPTSPQQSGQGQQGYYPTS | 205 | HMW | 588 | 644 | A5HMG1\|588-644 | 23.42 | 12 | no |
| PQQSGQGQQGYYPTSPQQSGQGQQPG | | glutenin | | | | | | |
| QQGYYPTSSQQSGQGHQSGQGQQGYYPTSL | 201 | HMW | 700 | 756 | A9YSK5\|700-756 | 19.76 | 9 | no |
| WQPGQGQQGYASPYHVSAEYQAARLK | | glutenin | | | | | | |
| TGQGQQGYYPTSLQQPGQGQQIGQGQQGYY | 201 | HMW | 196 | 252 | A5HMG2\|196-252 | 97.73 | 6 | no |
| PTSPQHPGQRQQPGQGQQIGQEQQLG | | glutenin | | | | | | |
| PGQGQPGYYPTSPQQPGQGQPGYYPTSPQQP | 200 | HMW | 280 | 336 | L0G7U5\|280-336 | 36.82 | 11 | no |
| GQLQQPAQGQQGYYPTSPQQPGQEQ | | glutenin | | | | | | |
| YYPTSPQQSGQGQPGYYLTSPQQSGQGQQP | 199 | HMW | 420 | 476 | A9YSK4\|420-476 | 20.97 | 6 | no |
| GQLQQSAQGQKGQQPGQGQQPGQGQQ | | glutenin | | | | | | |
| GQGQQGYYPTSLQQPGQGQRQGQGQQGYY | 199 | HMW | 420 | 476 | Q52JL3\|420-476 | 12.35 | 11 | no |
| PTSPQQPGQGQQGHHPASLQQPGQGQP | | glutenin | | | | | | |
| QGQQGYYPTSLQQSGQGQQSGQGQQGYYP | 199 | HMW | 420 | 476 | L0G7U5\|420-476 | 37.465 | 9 | no |
| TSPQQSGQGQQGYDSPYHVSAEHQAAS | | glutenin | | | | | | |
| GQGQQGYYPTSLQQPGQGQQQGQGQQGYY | 196 | HMW | 420 | 476 | A4URY8\|420-476 | 23.7475 | 10 | no |
| PTSLQQPGQGQQGHYPASLQQPGQGQP | | glutenin | | | | | | |
| QQTGQGQQPEQEQQPGQGQQGYYPTSLQQP | 195 | HMW | 420 | 476 | A9QUS3\|420-476 | 98.91 | 6 | no |
| GQGQQQGQGQQGYYPTSLQQPGQGQQ | | glutenin | | | | | | |
| YPTSLQQPGQGQQMGKGKQGYYPTSLQQPG | 193 | HMW | 168 | 224 | A4URY8\|168-224 | 16.88 | 12 | no |
| QGQQIGQGQQGYYPTSPQHTGQRQQP | | glutenin | | | | | | |
| QQPGQGQQGQQPGQGQQPGQGQPGYYPTSP | 193 | HMW | 336 | 392 | A9YSK4\|336-392 | 14.08 | 11 | no |
| QQSGQGQPGYYPTSSQQPTQSQQPGQ | | glutenin | | | | | | |
| LQPGQGQQGYYPTSSQQSGQGQQSGQGQQG | 191 | HMW | 700 | 756 | Q6UKZ5\|700-756 | 35.1 | 7 | no |
| YYPTSLWQPGQGQQPGQRQQGYDSPY | | glutenin | | | | | | |
| LQLGQGQQGYYPTSPQQSGQGQQSGQGQQ | 191 | HMW | 700 | 756 | A5HMG1\|700-756 | 20.4 | 13 | no |
| GYYPTSLWQPGQGQQPGQGQQGYDSPY | | glutenin | | | | | | |
| HYPGSLRQPGQGQPGQRQQPGQGQQTGQG | 190 | HMW | 308 | 364 | Q9SDM2\|308-364 | 55.86 | 5 | no |
| QQPEQEQQPGQGQQGYYPTSPQQPGQG | | glutenin | | | | | | |
| QLGQGQPGYYPTSPQQSGQGQQSGQGQQGY | 190 | HMW | 392 | 448 | A5HMG1\|392-448 | 29.19 | 16 | no |
| YPTSPQQSGQGQQPGQGQSGYFPTSR | | glutenin | | | | | | |
| LQPGQGQQGYYPTSSQQSGQGHQSGQGQQG | 186 | HMW | 700 | 756 | G3FLC5\|700-756 | 29.0975 | 10 | no |
| YYPTSLWQPGQGQQPGQGQQGYASPY | | glutenin | | | | | | |
| GQGQPGYYPTSPQQIGQGQQPGQLQQPTQG | 184 | HMW | 560 | 616 | G3K725\|560-616 | 30.155 | 6 | no |
| QQGQQPGQGQQGQQPGQGQQGQQPGQ | | glutenin | | | | | | |
| PGQGQQPGRGQPGYYPTSSQQLGQGQQLGQ | 183 | HMW | 196 | 252 | Q94IJ9\|196-252 | 36.13 | 11 | no |
| GQQGQQPGQGQPGYYPTSPQQPGQGQ | | glutenin | | | | | | |
| QAYYPTSSQQSRQRQQAGQWQRPGQGQPG | 181 | HMW | 476 | 532 | G4Y3Y1\|476-532 | 43.61 | 5 | no |
| YYPTPPQQPGQEQQSGQAQQSGQWQLV | | glutenin | | | | | | |
| GQRQQPGQGQHPEQGQQPGQGQQGYYPTSP | 177 | HMW | 476 | 532 | E4W506\|476-532 | 22.305 | 12 | no |
| QQPGQGQQLGQGQQGYYPTSPQQPGQ | | glutenin | | | | | | |
| GQGQQGYYPTSPQQPGQWQQPEQGQQGYY | 175 | HMW | 140 | 196 | Q38LF5\|140-196 | 39.32 | 8 | no |
| PTSPQQPGQLQQPAQGQQPGQGQQGQQ | | glutenin | | | | | | |
| VASPQQVSYYPGQASSRRPGQGQQEYYLTSP | 173 | HMW | 112 | 168 | A0MZ38\|112-168 | 14.1475 | 10 | no |
| QQSGQWQQPGQGQSGYYPTSPQQSG | | glutenin | | | | | | |
| QQPGQGQQGYYPTSLQQPGQGQQPHYPASQ | 171 | HMW | 364 | 420 | Q94IJ8\|364-420 | 54.17 | 5 | no |
| QQPGQGQQGHYPTSLLQPGQGQQGHY | | glutenin | | | | | | |
| WYYPTSPQESGQGQQPGQWQQPGQWQQPG | 171 | HMW | 644 | 700 | B1B520\|644-700 | 41.515 | 6 | no |
| QGQQGQQPGQGQPGYYPTSPQQSGQGQ | | glutenin | | | | | | |
| SLQQPGQGQQPGQGQPGYYPTSQQSEQGQQ | 169 | HMW | 336 | 392 | A5HMG1\|336-392 | 29.74 | 11 | no |
| PGQGKQPGQGQQGYYPTSSQQSGQGQ | | glutenin | | | | | | |
| GQQPGQGKQPGQGQQGYYPTSPQHPGQGQ | 165 | HMW | 280 | 336 | Q94IJ7\|280-336 | 81.94 | 4 | no |
| QPGQGQQPGQGKPGYYPTSPQLPGQGQ | | glutenin | | | | | | |
| GQGQQGYYPTSLQQPGQGQQGHYPASLQQP | 165 | HMW | 420 | 476 | D2CPI7\|420-476 | 27.11 | 9 | no |
| GQGQPGQRQQPGQGQHPEQGQQPGQG | | glutenin | | | | | | |
| QGRQIGQGQQSGQGQQGYYPTSPQQLGQGQ | 163 | HMW | 252 | 308 | A5HMG2\|252-308 | 16.52 | 11 | no |
| QPGQWQQSGQGQQGYYPTSQQQPGQG | | glutenin | | | | | | |
| SPQQPGEGQQPGQGRQPGQGQQPGQGQQG | 162 | HMW | 252 | 308 | Q94IJ7\|252-308 | 75.965 | 4 | no |
| QQPGQGKQPGQGQQGYYPTSPQHPGQG | | glutenin | | | | | | |
| TPQQPGQWQQPGQVQPGYYLTPPQQTGQAQ | 159 | HMW | 168 | 224 | Q94IJ7\|168-224 | 37.77 | 7 | no |
| QPGQGQQPGQGQPGYYPTSPRQPGQG | | glutenin | | | | | | |
| GYYPTSLQQPGQGQQPGQGQPGYYPTSPQQ | 159 | HMW | 336 | 392 | Q6UKZ5\|336-392 | 35.93 | 7 | no |
| PGQGKQPGQGQQRYYPTSSQQSGQGQ | | glutenin | | | | | | |
| SGQGQQGQQPGQGQRPGQGQQGYYPTSLQ | 158 | HMW | 252 | 308 | Q6UKZ5\|252-308 | 15.96 | 15 | no |
| QPRQGQQSGQGQPGYYPTSSRQPGQWQ | | glutenin | | | | | | |
| YPTSLQQPGQGQQGHYPASLQQPGQGQPGQ | 157 | HMW | 448 | 504 | A4URY8\|448-504 | 54.77 | 7 | no |
| RQQPGQGQQPGQGQQGYYPTSPQQPG | | glutenin | | | | | | |
| GQLQQPAQGQQPGQEQQGQQPGQGQQPGQ | 157 | HMW | 476 | 532 | A0MZ38\|476-532 | 29.94 | 8 | no |
| GQPGYYPTSPQQPGQEQQLEQWQQSGQ | | glutenin | | | | | | |
| YYPTSPQHPGQRQQPGQGQQIGQEQQLGQG | 154 | HMW | 224 | 280 | A5HMG2\|224-280 | 30.95 | 7 | no |
| RQIGQGQQSGQGQQGYYPTSPQQLGQ | | glutenin | | | | | | |
| QGYYSTSLQQPGQGQQGHYPTSLQQPGQGH | 152 | HMW | 504 | 560 | B8PSA6\|504-560 | 33.19 | 5 | no |
| PGQRQQPGQGQQPEQGQQPGQGQQGY | | glutenin | | | | | | |
| QGQQPGQGQQPEQEQQPGQGQQGYYITSLQ | 148 | HMW | 532 | 588 | Q94IJ8\|532-588 | 94.805 | 6 | no |
| QPGQGKQLGQWQQPGQGQEGYYPTSP | | glutenin | | | | | | |
| QQSGQGQQRYYPTSPQQSGQGQQPGQGQPG | 147 | HMW | 196 | 252 | Q6UKZ5\|196-252 | 52.05 | 6 | no |
| YYPISPQQSEQWQQPGQGQQPGQGQQ | | glutenin | | | | | | |
| QGRQIGQGQQSGQEQQGYYATSPQQLGQGQ | 144 | HMW | 252 | 308 | B8PSA6\|252-308 | 29.01 | 4 | no |
| QPGQWQQSGQGQQRYYPTSQQQPGQG | | glutenin | | | | | | |
| AQEQQPGQAQQSGQWQLVYYPTSPQQSGQ | 144 | HMW | 560 | 616 | M4M8L5\|560-616 | 36.48 | 6 | no |
| GQQGYYPTSPQQSGQGQQPGQGQQPRQ | | glutenin | | | | | | |
| GQGQPGYYPTSQQPGQKQQAGQGQQSGQG | 143 | HMW | 168 | 224 | A5HMG1\|168-224 | 18.37 | 15 | no |
| QQGYYPTSPQQSGQGQQPGQGQAGYYP | | glutenin | | | | | | |
| GQIPASQQQPGQGQQGHYPASLQQPGQQGH | 142 | HMW | 364 | 420 | A9QUS3\|364-420 | 32.845 | 4 | no |
| YPTSLQQLGQGQQIGQPGQKQQPGQG | | glutenin | | | | | | |
| QQGYYPTSPQQPGQGQQLGQGQQGYYPTSP | 142 | HMW | 476 | 532 | D2CPI7\|476-532 | 17.315 | 9 | no |
| QQPGQGQQPGQGQQGHCPMSPQQTGQ | | glutenin | | | | | | |
| QEQQPGQAQQSGQWQLVYYPTSPQQPGQLQ | 142 | HMW | 560 | 616 | A5HMG1\|560-616 | 29.64 | 9 | no |
| QPTQGQQGYYPTSPQQSGQGQQGYYP | | glutenin | | | | | | |
| QEQQDPQSGQGRQGQQSGQRQQDQQSGQG | 139 | HMW | 336 | 392 | A0MZ38\|336-392 | 15.63 | 5 | no |
| QQPGQRQPGYYSTSPQQLGQGQPRYYP | | glutenin | | | | | | |
| GQGQQGHYPASQQEPGQGQQGQIPASQQQP | 139 | HMW | 308 | 364 | D2CPI7\|308-364 | 33.32 | 5 | no |
| GQGQQGHYPASLQQPGQQGHYPTSLQ | | glutenin | | | | | | |
| GQQGYYPTSPQQPGQGQQPGQGQQGHCPM | 138 | HMW | 532 | 588 | A9QUS3\|532-588 | 17.675 | 5 | no |
| SPQQTGQAQQLGQGQQIGQVQQPGQGQ | | glutenin | | | | | | |
| GQQGYYPISPQQSGQGQQPGQGQQGYYPTS | 138 | HMW | 616 | 672 | M4M8L5\|616-672 | 25.135 | 10 | no |
| PQQSGQGQQPGHEQQPGQWLQPGQGQ | | glutenin | | | | | | |
| QKQPGYYPTSPWQPEQLQQPTQGQQRQQPG | 135 | HMW | 168 | 224 | A0MZ38\|168-224 | 33.82 | 5 | no |
| QGQQLRQGQQGQQSGQGQPRYYPTSS | | glutenin | | | | | | |
| QQSGQGQQLGQGQQGYYPTSPQQSGQGQQ | 135 | HMW | 756 | 812 | G3K725\|756-812 | 25.28 | 12 | no |
| GYYPTSPQQSGQGQQLGQGQQGYYPTS | | glutenin | | | | | | |
| QQGYYPTSPQQSGQGQQPGQSQQPGQGQQG | 134 | HMW | 476 | 532 | B8PSA6\|476-532 | 16.4175 | 10 | no |
| YYSTSLQQPGQGQQGHYPTSLQQPGQ | | glutenin | | | | | | |
| GQQPGQGQQPGQGQPWYYPTSPQESGQGQ | 134 | HMW | 644 | 700 | C0SUC3\|644-700 | 93.65 | 4 | no |
| QPGQWQQPGQGQPGYYLTSPLQLGQGQ | | glutenin | | | | | | |
| PGQGQPGYYPTSSQLQPGQLQQPAQGQQGQ | 132 | HMW | 196 | 252 | A9YSK4\|196-252 | 35.94 | 5 | no |
| QPGQGQQGQQPGQGQQPGQGQQGQQP | | glutenin | | | | | | |
| SPLQPGQGQPGYDPTSPQQPGQGQQPGQLQ | 132 | HMW | 532 | 588 | Q6R2V1\|532-588 | 17.58 | 5 | no |
| QPAQGQQGQQLAQGQQGQQPAQVQQE | | glutenin | | | | | | |
| GQQGYYPTSPQQSGQGQQPGQGQAGYYPTS | 132 | HMW | 196 | 252 | A5HMG1\|196-252 | 22.86 | 14 | no |
| PQQSGQWQQPGQGQQPGQGQQSGQGQ | | glutenin | | | | | | |
| QGQQPAQGQQGQQPGQGQQGQQPGQGQQG | 131 | HMW | 616 | 672 | C0SUC3\|616-672 | 16.21 | 7 | no |
| QQPGQGQQPGQGQPWYYPTSPQESGQG | | glutenin | | | | | | |
| PEQEQQPGQGQQGYYPTSLQQPGQGQQQGQ | 131 | HMW | 392 | 448 | D2CPI7\|392-448 | 37.455 | 8 | no |
| GQQGYYPTSLQQPGQGQQGHYPASLQ | | glutenin | | | | | | |
| SQQQPGQGPQPGQWQQLGQGQQGYYPTSP | 130 | HMW | 672 | 728 | Q19AE4\|672-728 | 114.43 | 5 | no |
| QQSGQGQQPGQWLQSGYYLTSPQQLGQ | | glutenin | | | | | | |
| QQSGQGQQLGQGQQGYYPTSPQQSGQGQQ | 130 | HMW | 756 | 812 | Q03872\|756-812 | 48.94 | 5 | no |
| GYDSPYHVSAEHQAASLKVAKAQQLAA | | glutenin | | | | | | |
| QGQQLGQGQQGQQPGQKQQSGQGQQGYYP | 129 | HMW | 252 | 308 | A0MZ38\|252-308 | 22.22 | 9 | no |
| ISPQQLGQGQQSGQGQLGYYPTSPQQS | | glutenin | | | | | | |
| QGQQPGQGQQGYYPTSPQQPGQGQQLGQG | 127 | HMW | 504 | 560 | A9QUS3\|504-560 | 34.8825 | 10 | no |
| QQGYYPTSPQQPGQGQQPGQGQQGHCP | | glutenin | | | | | | |
| SQQQPGQGPQPGQWQQSGQGQQGYYPTSPQ | 127 | HMW | 672 | 728 | G3K725\|672-728 | 43.02 | 5 | no |
| QSGQGQQPGQWLQPGQWLQSGYYLTS | | glutenin | | | | | | |
| SPQQSGQGQQPGQSQQPGQGQQGYYSTSLQ | 127 | HMW | 504 | 560 | A5HMG2\|504-560 | 39.85 | 6 | no |
| QPGQGQQGHYPASLQQPGQGHPGQRQ | | glutenin | | | | | | |
| GQRQQPGQGQQPGQGQQGYYPTSPQQPGQ | 126 | HMW | 476 | 532 | A4URY8\|476-532 | 39.62 | 9 | no |
| GQQLGQGQQGYYPTSPQQPGQGQQPGQ | | glutenin | | | | | | |
| GQQPGQGQQGQQPGQGQPGYYPTSPQQSGQ | 126 | HMW | 476 | 532 | A9YSK4\|476-532 | 11.03 | 16 | no |
| GQQPGQWQQPGQGQPGYYPTSPLQPG | | glutenin | | | | | | |
| QPGQGQQPKQGQQPGQGQQGYYPTSSQQPG | 126 | HMW | 560 | 616 | A5HMG2\|560-616 | 25.745 | 8 | no |
| QGKQLGQGQQGYYPTSPQQPGQGQQP | | glutenin | | | | | | |
| QQPGQGKQPGQGQQGYYPTSSQQSGQGQQL | 125 | HMW | 364 | 420 | A5HMG1\|364-420 | 29.58 | 10 | no |
| GQGQPGYYPTSPQQSGQGQQSGQGQQ | | glutenin | | | | | | |
| QGQQGYYPTSPQQSGQGQQPGQGQQPRQG | 124 | HMW | 588 | 644 | M4M8L5\|588-644 | 22.5 | 13 | no |
| QQGYYPISPQQSGQGQQPGQGQQGYYP | | glutenin | | | | | | |
| PGQGQQPGQGQQGYYPTSPQQPGQGQQPGQ | 122 | HMW | 532 | 588 | Q9SDM3\|532-588 | 18.09 | 13 | no |
| GQLEYYPTSPQQPGQGQPGYYPTFPQ | | glutenin | | | | | | |
| VTSSQQGSYYPGQAFPQQSGQGQQPGQGQQ | 122 | HMW | 112 | 168 | Q6UKZ5\|112-168 | 36.64 | 5 | no |
| PGQRQQDQQPGQGQQGYYPTSPQQPG | | glutenin | | | | | | |
| GQGQPRYYPTSQQPGQKQQAGQGQQSGQG | 121 | HMW | 168 | 224 | Q6Q7J1\|168-224 | 24.6975 | 10 | no |
| QQGYYPTSPQQSGQGQQPGQGQSRYYP | | glutenin | | | | | | |
| GQQGYYPTSPQQSGQGQQPGHEQQPGQWL | 121 | HMW | 672 | 728 | A5HMG1\|672-728 | 14.96 | 15 | no |
| QLGQGQQGYYPTSPQQSGQGQQSGQGQ | | glutenin | | | | | | |
| QGYYPTSPQQPGQGQQPGQWQQPGQGQQG | 121 | HMW | 336 | 392 | L0G7U5\|336-392 | 13.105 | 11 | no |
| YYPTSPQQPGQGQQPGQWLQPGQEQQG | | glutenin | | | | | | |
| QGQQPRQGQQGYYPISPQQSGQGQQTGQGQ | 120 | HMW | 644 | 700 | Q6UKZ5\|644-700 | 74.2525 | 6 | no |
| QGYYPTSPQQSGQGQQPRHEQQPGQW | | glutenin | | | | | | |
| EQQDQQPGQRQQGYYPTSPQQPGQGQQLGQ | 120 | HMW | 140 | 196 | A5HMG1\|140-196 | 17.22 | 11 | no |
| GQPGYYPTSQQPGQKQQAGQGQQSGQ | | glutenin | | | | | | |
| QQPGQGQQDQQPEQGQQPGKGQQGYYPTT | 119 | HMW | 140 | 196 | Q94IJ7\|140-196 | 96.22 | 4 | no |
| PQQPGQWQQPGQVQPGYYLTPPQQTGQ | | glutenin | | | | | | |
| GQLQQPAQGQQGYYPTTPQQPGQGQQPGQ | 119 | HMW | 644 | 700 | Q94IJ7\|644-700 | 34.2875 | 8 | no |
| GQQGYYPTSPQQSGQGQQPGQWLQPGQ | | glutenin | | | | | | |
| QQPGQLQQPAQGQQGYYPTSPQQPGQEQQG | 119 | HMW | 308 | 364 | L0G7U5\|308-364 | 97.93 | 5 | no |
| YYPTSPQQPGQGQQPGQWQQPGQGQQ | | glutenin | | | | | | |
| ASQQQPGQGQQGHYPASQQQPGQRQQGHY | 117 | HMW | 308 | 364 | A9QUS3\|308-364 | 19.7 | 5 | no |
| PASQQQPGQGQQGHYPASQQEPGQGQQ | | glutenin | | | | | | |
| LPGQLQQPAQGQQGYYPTSPQQPGQGQQKY | 116 | HMW | 588 | 644 | Q9SDM3\|588-644 | 20.8175 | 8 | no |
| YPTSPQQPGQWQQPGQGQQGYYITSP | | glutenin | | | | | | |
| GQGQLEYYPTSPQQPGQGQPGYYPTFPQLPG | 116 | HMW | 560 | 616 | Q9SDM3\|560-616 | 38.33 | 11 | no |
| QLQQPAQGQQGYYPTSPQQPGQGQQ | | glutenin | | | | | | |
| YPTSLQQPGQGQQIGKGQQGYYPTSLQQPG | 116 | HMW | 168 | 224 | A9YSK3\|168-224 | 21.67 | 10 | no |
| QGQQGYYPTSLQHTGQRQQPVQGQQP | | glutenin | | | | | | |
| QGQQPGQGQRPGQGQQGYYPTSPQQPGQG | 116 | HMW | 252 | 308 | A5HMG1\|252-308 | 40.52 | 11 | no |
| QQSGQGQPGYYPTSLRQPGQWQQPGQG | | glutenin | | | | | | |
| QGYYPTSPQQPGQGQQPGQGQQPGEGQPGY | 116 | HMW | 168 | 224 | L0G7U5\|168-224 | 13.285 | 14 | no |
| YPTSPQQPGQGQQPGQGQPGYYPTSS | | glutenin | | | | | | |
| GQLGYYPTSPQQPGQGQPGYYPTSPQLPGQL | 115 | HMW | 616 | 672 | Q94IJ7\|616-672 | 111.83 | 5 | no |
| QQPAQGQQGYYPTTPQQPGQGQQPG | | glutenin | | | | | | |
| QQPGQGQQGYYPTSPQQSGQAQQPGQWQQ | 115 | HMW | 392 | 448 | Q94IJ9\|392-448 | 41.14 | 7 | no |
| PGQGQSGYYPTSQQQPGQGQQPGQGQQ | | glutenin | | | | | | |
| GYYPTSPQQPEQGQQPGQGQQPGQGQPGYY | 114 | HMW | 532 | 588 | Q94IJ7\|532-588 | 28.88 | 8 | no |
| PTSPQQPGQGQQPGQEQQPGQGQQPG | | glutenin | | | | | | |
| PGQGQQPGQGQQGYYPTSPQQPGQGQQGH | 114 | HMW | 280 | 336 | Q94IJ8\|280-336 | 81.67 | 4 | no |
| YPGSLQQPGQGQPGQRQQPGHGQQTGQ | | glutenin | | | | | | |
| QGQQPGQGQRPGQGQQGYYPISPQQPGQGQ | 114 | HMW | 252 | 308 | M4M8L5\|252-308 | 28.46 | 10 | no |
| QSGQGQPGYYPTSFAQPRTMAATQDK | | glutenin | | | | | | |
| QQPGQGKQPGQGQQRYYPTSSQQSGQGQQP | 114 | HMW | 364 | 420 | Q6UKZ5\|364-420 | 25.815 | 10 | no |
| GQGQPGYYPTSPQQSGQGQQSGQAQQ | | glutenin | | | | | | |
| QPGQGQPGQRQQPGQGQHPEQGQQPGQGQ | 114 | HMW | 448 | 504 | D2CPI7\|448-504 | 33.665 | 7 | no |
| QGYYPTSPQQPGQGQQLGQGQQGYYPT | | glutenin | | | | | | |
| QGRQPRQGQQGYYPISPQQSGQGQQPGQGQ | 114 | HMW | 644 | 700 | A5HMG1\|644-700 | 15.48 | 13 | no |
| QGYYPTSPQQSGQGQQPGHEQQPGQW | | glutenin | | | | | | |
| GQGQQTRQGQQLEQGQQPGQGQQGYYPTS | 114 | HMW | 476 | 532 | A5HMG2\|476-532 | 14.03 | 13 | no |
| PQQSGQGQQPGQSQQPGQGQQGYYSTS | | glutenin | | | | | | |
| PGQGQQPGQGQQGQQPGQGQQPGQGQQGY | 112 | HMW | 364 | 420 | Q94IJ7\|364-420 | 40.465 | 6 | no |
| YPTSPQHLGQGQQPGQGKPGSYPTSPQ | | glutenin | | | | | | |
| GQQPEQEQQPGQGQQGYYPTSPQQPGRGQQ | 112 | HMW | 336 | 392 | Q94IJ8\|336-392 | 81.2975 | 6 | no |
| PGQGQQGYYPTSLQQPGQGQQPHYPA | | glutenin | | | | | | |
| QQPGQGQQGQQPGQRQQSGQGQQGYYPTS | 112 | HMW | 308 | 364 | G3FLC5\|308-364 | 13.56 | 15 | no |
| LQQPGQGQQLGQGQPGYYPTSQQSEQG | | glutenin | | | | | | |
| QQPGQGQQGQQPGQGQQPGQGQQGYYPTS | 112 | HMW | 308 | 364 | A5HMG1\|308-364 | 12.14 | 11 | no |
| LQQPGQGQQPGQGQPGYYPTSQQSEQG | | glutenin | | | | | | |
| QPGQGQQQGQGQQGYYPTSLQQPGQGQQG | 111 | HMW | 448 | 504 | A9QUS3\|448-504 | 8.825 | 10 | no |
| HYPASLQQPGQGQPGQRQQPGQGQHPE | | glutenin | | | | | | |
| GQQSGQGQPGYYPTSFAQPRTMAATQDKGS | 111 | HMW | 280 | 336 | M4M8L5\|280-336 | 65.08 | 4 | no |
| NQDKGNKVSSQDKDNNQDKDNKDTTQ | | glutenin | | | | | | |
| GQQPGQWQQSGQGQQGYYPTSQQQPGQGQ | 111 | HMW | 280 | 336 | A5HMG2\|280-336 | 15.5 | 9 | no |
| QGQYPASQQQPGQGQQGQYPASQQQPG | | glutenin | | | | | | |
| YPSVTCPQQVSYYPGQASPQRPGQGQQPGQ | 110 | HMW | 112 | 168 | Q38LF5\|112-168 | 16.3375 | 8 | no |
| GQQGYYPTSPQQPGQWQQPEQGQQGY | | glutenin | | | | | | |
| GYYPTSPQQPGQGQQPGQGQPGYYPTSSQQ | 110 | HMW | 196 | 252 | L0G7U5\|196-252 | 25.665 | 8 | no |
| PGQGQQPGQGQQPGQGQQGQQPGQGQ | | glutenin | | | | | | |
| YYPTSPQQLGQGQQPGQGQQPGQGQPGYYP | 109 | HMW | 476 | 532 | Q9SDM3\|476-532 | 25.57 | 7 | no |
| TSPQQPGQGQQTGQGQQPGQGQQGQQ | | glutenin | | | | | | |
| SLQQPGQGQQLGQGQPGYYPTSQQSEQGQQ | 109 | HMW | 336 | 392 | A9YSK5\|336-392 | 29.89 | 11 | no |
| PGQGQQGYYPTSPQQSGQGQQLGQGQ | | glutenin | | | | | | |
| LQQPGQGQQGHYPASLQQPGQGHPGQRQQP | 109 | HMW | 532 | 588 | A5HMG2\|532-588 | 43.23 | 7 | no |
| GQGQQPKQGQQPGQGQQGYYPTSSQQ | | glutenin | | | | | | |
| PGQGQQIGQGQQGYYPTSPQHTGQRQQPVQ | 108 | HMW | 196 | 252 | A4URY8\|196-252 | 23.87 | 7 | no |
| GQQIGQGQQPEQGQQPGQWQQGYYPT | | glutenin | | | | | | |
| YPSVTSPQQVSYYPGQASPQRPGQGQQPGQ | 108 | HMW | 112 | 168 | A9YSK4\|112-168 | 112.09 | 5 | no |
| GQQGYYPTSPQQPGQWQQPEQGQPRY | | glutenin | | | | | | |
| YPSVTSPQQVSYYPGQASPQRPGQGQQPGQ | 107 | HMW | 112 | 168 | B1B520\|112-168 | 91.53 | 5 | no |
| GQQSGQGQQGYYPTSPQQPGQWQQPE | | glutenin | | | | | | |
| GQQGHCPMSPQQTGQAQQQGQGQQIGQVQ | 106 | HMW | 532 | 588 | A4URY8\|532-588 | 78.18 | 5 | no |
| QPGQGQQGYYPTSLQQPGQGQQSGQGQ | | glutenin | | | | | | |
| SPQQSGQGQPGYYPTSSQQPTQSQQPGQGQ | 106 | HMW | 364 | 420 | A9YSK4\|364-420 | 31.2025 | 6 | no |
| QGQQVGQGQQAQQPGQGQQPGQGQPG | | glutenin | | | | | | |
| QQPGQGQQPGQGKPGYYPTSPQLPGQGQQP | 106 | HMW | 308 | 364 | Q94IJ7\|308-364 | 53.21 | 6 | no |
| GQGQSGYYPTSPQQLGQGQQPGQGWQ | | glutenin | | | | | | |
| EQGQQPGQWQQGYYPTSPQQLGQGQQPRQ | 103 | HMW | 224 | 280 | A9YSK3\|224-280 | 47.085 | 4 | no |
| WQQSGQGQQGHYPTSLQQPGQGQQGHY | | glutenin | | | | | | |
| AGQGQQVQQPGQGQQSGQGQQGYYPTSPQ | 103 | HMW | 448 | 504 | Q94IJ7\|448-504 | 33.31 | 10 | no |
| LSGQAQQPGQWQQPGQGQPGYYPTSQQ | | glutenin | | | | | | |
| GQQPGEGQQGYYPTFPQQPGQVQQPGQGQQ | 103 | HMW | 280 | 336 | Q94IJ9\|280-336 | 36.43 | 8 | no |
| PGQGQPGYYPTSPQQPGQGQQPGQEQ | | glutenin | | | | | | |
| PGQRQQPGQGQQTEQGQQLEQGQQPGQGQ | 102 | HMW | 448 | 504 | B8PSA6\|448-504 | 42.725 | 6 | no |
| QGYYPTSPQQSGQGQQPGQSQQPGQGQ | | glutenin | | | | | | |
| QIGQGQQIRQGQQPGQGQQGYYQTHPQQPG | 102 | HMW | 252 | 308 | Q94IJ8\|252-308 | 83.6 | 7 | no |
| QGQQPGQGQQGYYPTSPQQPGQGQQG | | glutenin | | | | | | |
| IGQPGQKQQPGQGQQTGQGQQPEQEQQPGQ | 100 | HMW | 392 | 448 | A4URY8\|392-448 | 14.3 | 10 | no |
| GQQGYYPTSLQQPGQGQQQGQGQQGY | | glutenin | | | | | | |
| QWQQSGQGQQGHYPTSLQQPGQGQQGHYL | 100 | HMW | 252 | 308 | A9YSK3\|252-308 | 18.63 | 5 | no |
| ASQQQPGQGQQGHYPASQQQPGQGQQG | | glutenin | | | | | | |
| HCPTSPQQSGQAQQPGQGQQIGQVQQPGQG | 100 | HMW | 532 | 588 | A9YSK3\|532-588 | 63.32 | 4 | no |
| QQGYYPTSVQQPGQGQQSGQGQQSGQ | | glutenin | | | | | | |
| AQQPGQGQQPGQGQPGYYPTSPRQPGQGQQ | 100 | HMW | 196 | 252 | Q94IJ7\|196-252 | 31.275 | 8 | no |
| SGQGQQGQLPGQWQQPGQEQPGYNPT | | glutenin | | | | | | |
| GYYPTSPQQSGQGQQPGQGQSGYFPTSRQQS | 100 | HMW | 420 | 476 | A5HMG1\|420-476 | 19.615 | 10 | no |
| GQGQQPGQGQQSGQGQQGQQPGQGQ | | glutenin | | | | | | |
| IGQPGQRQQPGQGQQTGQGQQPEHEQQPGQ | 99 | HMW | 392 | 448 | Q52JL3\|392-448 | 14.19 | 9 | no |
| GQQGYYPTSLQQPGQGQRQGQGQQGY | | glutenin | | | | | | |
| TSPQQSGQGQQGYYPTSPQQSGQGQQPGQG | 99 | HMW | 616 | 672 | A5HMG1\|616-672 | 40.5 | 7 | no |
| RQPRQGQQGYYPISPQQSGQGQQPGQ | | glutenin | | | | | | |
| QQPGQGQQPGQGQQSQQPGQGQHPGQGQQ | 99 | HMW | 140 | 196 | L0G7U5\|140-196 | 69.0025 | 4 | no |
| GYYPTSPQQPGQGQQPGQGQQPGEGQP | | glutenin | | | | | | |
| QGQQLGQGQQGYYPTSPQQPGQGQQPGQG | 98 | HMW | 504 | 560 | A4URY8\|504-560 | 49.285 | 6 | no |
| QQGHCPMSPQQTGQAQQQGQGQQIGQV | | glutenin | | | | | | |
| QQPGQGQQGYYPTSLQQPGQGQQSGQGQQS | 98 | HMW | 560 | 616 | A4URY8\|560-616 | 55.17 | 5 | no |
| GQGHQPGQGQQSGQEKQGYDSPYHVS | | glutenin | | | | | | |
| MSPQQTGQAQQLGQGQQIGQVQQPGQGQQ | 98 | HMW | 560 | 616 | A9QUS3\|560-616 | 42.26 | 6 | no |
| GYYPTSLQQPGQGQQSGQGQQSGQGHQ | | glutenin | | | | | | |
| QPGQGQQPVQGQSGYYPTSPQQPGQGQQAG | 98 | HMW | 420 | 476 | Q94IJ7\|420-476 | 30.345 | 8 | no |
| QGQQVQQPGQGQQSGQGQQGYYPTSP | | glutenin | | | | | | |
| KYYPTSPQQPGQWQQPGQGQQGYYITSPQQ | 97 | HMW | 616 | 672 | Q9SDM3\|616-672 | 39.58 | 9 | no |
| SGQGQQPGQGQQPGQGQEGYYPTLGQ | | glutenin | | | | | | |
| GQQGQQPGQGQQPGQGQPWYYPTSPQESG | 97 | HMW | 812 | 868 | B1B520\|812-868 | 70.46 | 6 | no |
| QGQQPGQWQQPGQWQQPGQGQPGYYLT | | glutenin | | | | | | |
| VQGQQIGQGQQPEQGQQPGQWQQGYYPTSP | 96 | HMW | 224 | 280 | A4URY8\|224-280 | 36.52 | 6 | no |
| QQLGQGQQPGQWQQSGQGQQGHYPTS | | glutenin | | | | | | |
| QPGQLQQSAQGQKGQQPGQGQQPGQGQQG | 96 | HMW | 448 | 504 | A9YSK4\|448-504 | 8.85 | 14 | no |
| QQPGQGQQGQQPGQGQPGYYPTSPQQS | | glutenin | | | | | | |
| GQQPGQWQQSGQGQQRYYPTSQQQPGQGQ | 96 | HMW | 280 | 336 | B8PSA6\|280-336 | 43.32 | 4 | no |
| QGQYPASQQQPAQGQQGQYPASQQQPA | | glutenin | | | | | | |
| LQQPGQGQQGHYLASQQQPAQGQQGHYPA | 95 | HMW | 280 | 336 | A4URY8\|280-336 | 19.875 | 6 | no |
| SQQQPGQGQQGHYPASQQQPGQGQQGH | | glutenin | | | | | | |
| YPTSPQQPGQGQQGHHPASLQQPGQGQPGQ | 95 | HMW | 448 | 504 | Q52JL3\|448-504 | 44.8 | 5 | no |
| RQQPGQRQHPEQGQQPGQGQQGYYPT | | glutenin | | | | | | |
| YPTSLQQPGQGQQGHYPASLQQPGQGQPGQ | 93 | HMW | 448 | 504 | E4W506\|448-504 | 61.22 | 5 | no |
| RQQPGQGQHPEQGQQPGQGQQGYYPT | | glutenin | | | | | | |
| QLGQGQQIGQPGQKQQPGQGQQTGQGQQPE | 93 | HMW | 364 | 420 | D2CPI7\|364-420 | 95.41 | 4 | no |
| QEQQPGQGQQGYYPTSLQQPGQGQQQ | | glutenin | | | | | | |
| QGQQPGQGQQGQQPGQGQQPGQGQPWYYP | 92 | HMW | 532 | 588 | C6L669\|532-588 | 57.44 | 5 | no |
| TSPQESGQGQQPGQWQQPGQWQQPGQG | | glutenin | | | | | | |
| AQQLGQGQQIGQVQQPGQGQQGYYPTSLQQ | 92 | HMW | 532 | 588 | D2CPI7\|532-588 | 32.695 | 6 | no |
| PGQGQQSGQGQQSGQGHQPGQGQQSG | | glutenin | | | | | | |
| QPGEGQQGQQPGQGQQPGQGQPGYYPTSLQ | 91 | HMW | 588 | 644 | A0MZ38\|588-644 | 26.495 | 10 | no |
| QSGQGQQPGQWQQPGQGQPGYYPTSS | | glutenin | | | | | | |
| QGSYYPGQASPQQLGQGQQPGQGQQPRQEQ | 91 | HMW | 112 | 168 | A5HMG1\|112-168 | 45.33 | 4 | no |
| QDQQPGQRQQGYYPTSPQQPGQGQQL | | glutenin | | | | | | |
| PGQGQQPGQGQQGQQPGQGQQAGQGQQGY | 90 | HMW | 448 | 504 | Q9SDM3\|448-504 | 16.76 | 10 | no |
| YPTSPQQLGQGQQPGQGQQPGQGQPGY | | glutenin | | | | | | |
| QPGQGQQPGQEQQDQQPGQGQQQGQGQQG | 90 | HMW | 504 | 560 | Q94IJ7\|504-560 | 13.735 | 7 | no |
| YYPTSPQQPEQGQQPGQGQQPGQGQPG | | glutenin | | | | | | |
| QQPGQGQQGHYTASLQQPGQGQQGHYPAS | 88 | HMW | 392 | 448 | A5HMG2\|392-448 | 42.065 | 4 | no |
| LQQVGQGQQIGQLGQRQQPGQGQQTRQ | | glutenin | | | | | | |
| QQSGQAQQPGQWQQPGQGQSGYYPTSQQQ | 86 | HMW | CTERM | NA | Q94IJ9\|CTERM | 31.665 | 8 | no |
| PGQGQQPGQGQQPGQGQQPGQGQQGQ | | glutenin | | | | | | |
| QGQQGQYPASQQQPGQGQQGHYLASQQQP | 86 | HMW | 336 | 392 | Q03871\|336-392 | 30.87 | 4 | no |
| GQGQQRHYPASLQQPGQGQQGHYTASL | | glutenin | | | | | | |
| QGQQPGQGQQGQQPGQGQQGQQPGQGQQP | 84 | HMW | 616 | 672 | A9YSK4\|616-672 | 62.85 | 5 | no |
| GQGQPWYYPTSPQESGQGQQPGQWQQP | | glutenin | | | | | | |
| PGYYPISPQQSEQWQQPGQGQQPGQGQQSG | 84 | HMW | 224 | 280 | Q6UKZ5\|224-280 | 37.07 | 9 | no |
| QGQQGQQPGQGQRPGQGQQGYYPTSL | | glutenin | | | | | | |
| QQPGQGQQPGQGQQPGQGQQGQQPGQGQQ | 84 | HMW | 224 | 280 | L0G7U5\|224-280 | 80.725 | 4 | no |
| PGQGQQGYYPTSPQQLGQGQQPGQWQQ | | glutenin | | | | | | |
| QGSYYPGQASPQQSGQGQQPGQEQQPGQGQ | 81 | HMW | 112 | 168 | A9YSK5\|112-168 | 20.2 | 5 | no |
| QDQQPGQRQQGYYPTSPQQPGQGQQL | | glutenin | | | | | | |
| QQSGQGQQPGQGQQSGQGQQGQQPGQGQQ | 81 | HMW | 448 | 504 | A5HMG1\|448-504 | 14.54 | 7 | no |
| AYYPTSSQQSGQRQQAGQWQRPGQGQS | | glutenin | | | | | | |
| QGQQGQQPGQGQQGQQPGQGQQGQQPGQG | 78 | HMW | 588 | 644 | G3K725\|588-644 | 68.37 | 5 | no |
| QQPGQGQPGYYPTSLQQSGQGQQPGQW | | glutenin | | | | | | |
| SLQQPGQGQQGHYPASLQQVGQGQQIGQPG | 78 | HMW | 420 | 476 | B8PSA6\|420-476 | 17.55 | 5 | no |
| QRQQPGQGQQTEQGQQLEQGQQPGQG | | glutenin | | | | | | |
| YPTTPQQPGQGQQPEQGQPGYYLTSSQQPG | 78 | HMW | 168 | 224 | Q94IJ9\|168-224 | 29.1475 | 8 | no |
| QGQQPGRGQPGYYPTSSQQLGQGQQL | | glutenin | | | | | | |
| QQGQYPASQQQPGQGQQGQYPASQQQPGQ | 78 | HMW | 308 | 364 | A5HMG2\|308-364 | 23.0825 | 6 | no |
| GQQGQYPASQQQPAQGQQGQYPASQQQ | | glutenin | | | | | | |
| GQGQQIGQVQQPGQGQQGYYPISLQQSGQG | 77 | HMW | 616 | 672 | B8PSA6\|616-672 | 25.6 | 9 | no |
| QQSGQGQQSGQGHQLGQGQQSGQEQQ | | glutenin | | | | | | |
| QGQQGQYPASQQQPAQGQQGQYPASQQQP | 77 | HMW | 336 | 392 | A5HMG2\|336-392 | 24.01 | 5 | no |
| GQGQQGHYLASQQQPGQGQQRHYPASL | | glutenin | | | | | | |
| PGQGQQPGQGQQGHCPTSPQQTGQAQQPGQ | 75 | HMW | 588 | 644 | B8PSA6\|588-644 | 42.8 | 5 | no |
| GQQIGQVQQPGQGQQGYYPISLQQSG | | glutenin | | | | | | |
| GQQSGQGQPGYYPTSLRQPGQWQQPGQGQ | 75 | HMW | 280 | 336 | A5HMG1\|280-336 | 17.915 | 12 | no |
| QPGQGQQGQQPGQGQQPGQGQQGYYPT | | glutenin | | | | | | |
| QLGQGQQGQQPGQGQQPAQGQQGQQPGQG | 73 | HMW | 784 | 840 | B1B520\|784-840 | 31.645 | 6 | no |
| QQGQQPGQGQQPGQGQPWYYPTSPQES | | glutenin | | | | | | |
| TSPQQSGQLQQPAQGQQPGQEQQGQQPGQG | 72 | HMW | 476 | 532 | G3K725\|476-532 | 143.152 | 4 | no |
| QQGQQPGQGQQPGQGQPGYYPTSPQQ | | glutenin | | | | 5 | | |
| QQGQYPASQQQPAQGQQGQYPASQQQPAQ | 71 | HMW | 308 | 364 | B8PSA6\|308-364 | 25.7875 | 4 | no |
| GQQGQYPASQQQPGQGQQGQYPASQQQ | | glutenin | | | | | | |
| GHYPTSLQQLGQGQQIGQPGQKQQPGQGQQ | 70 | HMW | 392 | 448 | A9QUS3\|392-448 | 81.42 | 5 | no |
| TGQGQQPEQEQQPGQGQQGYYPTSLQ | | glutenin | | | | | | |
| QGQQGQQPGQGQQGQQPGQGQQPGQGQP | 67 | HMW | 616 | 672 | D7REK2\|616-672 | 83.73 | 4 | no |
| WYYPTSPQQPGQWQQPGQWQQPGQGQPG | | glutenin | | | | | | |
| YPTSPQQSGQLQQPAQGQQPGQGQQGQQPG | 65 | HMW | 168 | 224 | A9YSK4\|168-224 | 24.615 | 5 | no |
| QGQPGYYPTSSQLQPGQLQQPAQGQQ | | glutenin | | | | | | |
| QPGQGQQPGQGQQGQQPGQGQQGQQPGQG | 59 | HMW | 252 | 308 | Q94IJ9\|252-308 | 73.86 | 4 | no |
| QQPGEGQQGYYPTFPQQPGQVQQPGQG | | glutenin | | | | | | |
| GQGQQPGQLQQPTQGQQGQQPGQGQQGQQ | 57 | HMW | 560 | 616 | A0MZ38\|560-616 | 35.6325 | 8 | no |
| PGEGQQGQQPGQGQQPGQGQPGYYPTS | | glutenin | | | | | | |
| QGQPGYDPTSPQQPGQGQQPGQLQQPAQGQ | 53 | HMW | 532 | 588 | A9YSK4\|532-588 | 22.475 | 4 | no |
| QGQQLAQGQQGQQPAQVQQGQRPAQG | | glutenin | | | | | | |
| GQQLEQGQQPGQGQQTRQGQQLEQGQQPG | 51 | HMW | 448 | 504 | A5HMG2\|448-504 | 45.745 | 6 | no |
| QGQQTRQGQQLEQGQQPGQGQQGYYPT | | glutenin | | | | | | |
| GQQPGQGQQGQQPGQGQQPGQGQQGQQPG | 47 | HMW | 224 | 280 | A9YSK4\|224-280 | 8.32 | 9 | no |
| QGQQPGQGQQGQQLGQGQQGYYPTSLQ | | glutenin | | | | | | |
| PQQPGQGQQPGQLQQPAQGQQPGQGQQGQ | 45 | HMW | 308 | 364 | A9YSK4\|308-364 | 84.935 | 4 | no |
| QPGQGQQGQQPGQGQQPGQGQPGYYPT | | glutenin | | | | | | |
| VTSPHQGSYYPGQTSPQQPGQAQQPGQEQQ | 43 | HMW | 112 | 168 | Q94IJ7\|112-168 | 48.795 | 6 | no |
| PGQGQQDQQPEQGQQPGKGQQGYYPT | | glutenin | | | | | | |
| QQLGQGQQGQQPGQGQQGQQPAQGQQGQ | 30 | HMW | 588 | 644 | E2CT66\|588-644 | 69.515 | 4 | no |
| QPGQGQQGQQPGQGQQPGQGQPWYYPTS | | glutenin | | | | | | |
| QGQQPGQGQQGYDSPYHVSAEYQAARLKV | 29 | HMW | CTERM | NA | A5HMG1\|CTERM | 16.315 | 4 | no |
| AKAQQLAASLPAMCRLEGSDALSTRQ | | glutenin | | | | | | |
| QQGQQPGQGQQGQQPGQGQQGQQPAQGQQ | 7 | HMW | 588 | 644 | A9YSK4\|588-644 | 45.22 | 4 | no |
| GQQPGQGQQGQQPGQGQQGQQPGQGQQ | | glutenin | | | | | | |
| SLQQVGQGQQIGQLGQRQQPGQGQQTRQG | 2 | HMW | 420 | 476 | A5HMG2\|420-476 | 16.995 | 6 | no |
| QQLEQGQQPGQGQQTRQGQQLEQGQQP | | glutenin | | | | | | |
| QQQQQPILLQQPPFSQHQQPVLPQQQIPSVQP | 71 | LMW | 140 | 196 | B2BZC4\|140-196 | 37.15 | 9 | no |
| SILQQLNPCKVFLQQQCSPVAMPQ | | glutenin | | | | | | |
| QQQQPVLPQQPPFSQQQQQQPILPQQPPFSQ | 63 | LMW | 140 | 196 | B2BZD1\|140-196 | 76.95 | 13 | no |
| HQQPVLPQQQIPYVQPSILQQLNPC | | glutenin | | | | | | |
| QPPFSQQQQPVLPQQPSFSQQQLPPFSQQLPP | 59 | LMW | 112 | 168 | Q8W3W9\|112-168 | 60.925 | 8 | no |
| FSQQQQPVLLQQQIPFVHPSILQQ | | glutenin | | | | | | |
| QQPLLPQQPPFSQQRPPFSQQQQQPVLPQQPP | 47 | LMW | 112 | 168 | Q0GNG1\|112-168 | 54.2 | 13 | no |
| FSQHQQPVLPQQQIPYVQPSILQQ | | glutenin | | | | | | |
| QQPLLLQQPPLSQQQPPFSRQQQQPPFSQQQ | 43 | LMW | 112 | 168 | C3VN79\|112-168 | 68.23 | 8 | no |
| QQPILLQQPPFSQHQQPVLPQQQIP | | glutenin | | | | | | |
| QQPIQQQPQPFPQQPPCSQQQQPPLSQQQQPP | 43 | LMW | 28 | 84 | D3U326\|28-84 | 29.5325 | 16 | no |
| FSQQQPPFSQQELPILPQQPPFSQ | | glutenin | | | | | | |
| QQRPPFSQQQQQPVLPQQPPFSQQQQQQPIL | 43 | LMW | 112 | 168 | D3U326\|112-168 | 69.04 | 12 | no |
| PQQPPFSQHQQPVLPQQQIPYVQPS | | glutenin | | | | | | |
| QQPLLPQQPPFSQQQPPFSQQQQQPILPQQPP | 43 | LMW | 112 | 168 | F8SGL5\|112-168 | 36.82 | 17 | no |
| FSQHQQPVLPQQQIPSVQPSILQQ | | glutenin | | | | | | |
| FSQQQQPVLPQQPPFSQQQQQPVLPQQQIPF | 42 | LMW | 140 | 196 | Q6QGV8\|140-196 | 48.17 | 10 | no |
| VHPSILQQLNPCKVFLQQQCSPVAM | | glutenin | | | | | | |
| QQPLLPQQPPFSQQQPPFSQQQQQPPFSQHQ | 41 | LMW | 112 | 168 | F8SGL7\|112-168 | 32.31 | 15 | no |
| QPVLPQQQIPSVQPSILQQLNPCKV | | glutenin | | | | | | |
| QQQLAQGTFLQPHQIAQLEVMTSIALRTLPM | 40 | LMW | 308 | 364 | B2Y2Q2\|308-364 | 94.225 | 10 | no |
| MCRVNVPLYRTTTSVPFGVGTGVGA | | glutenin | | | | | | |
| PPFLQQQQPSLPQQPPFSQQQQQLVLPQQQIP | 40 | LMW | 168 | 224 | B2Y2Q6\|168-224 | 41.1175 | 12 | no |
| FVHPSILQQLNPCKVFLQQQCSPV | | glutenin | | | | | | |
| QQLPPFSQQQQVLPQQPPFSQQQQPVLLQQQ | 40 | LMW | 168 | 224 | Q8W3X1\|168-224 | 82.7 | 11 | no |
| IPFVHPSILQQLNPCKVFLQQQCSP | | glutenin | | | | | | |
| QQPLLPQQPPFSQQQPPFSQQQQQPPFSQQQ | 39 | LMW | 112 | 168 | B2BZC4\|112-168 | 61.6075 | 12 | no |
| QQPILLQQPPFSQHQQPVLPQQQIP | | glutenin | | | | | | |
| QQQPILPQQPPFSQQQQPVLLQQQIPFVHPSIL | 39 | LMW | 196 | 252 | B2Y2R3\|196-252 | 83.125 | 10 | no |
| QQLNPCKVFLQQQCSPVAMPQSL | | glutenin | | | | | | |
| GQQPQQQQLAQGTFLQPHQIAQLEVMTSIAL | 39 | LMW | CTERM | NA | D3UAL8\|CTERM | 45.16 | 14 | no |
| RTLPTMCNVNVSLYRTTTRVPFGV | | glutenin | | | | | | |
| GQCSFQQPQQQLGQQPQQQQVQKGTFLQPH | 38 | LMW | 0 | NA | Q8W3V4_9 | 48.3775 | 14 | no |
| QIARLEVMTSIALRTLPTMCSVNVPL | | glutenin | | | | | | |
| QLAQGTFLQPHQIAQLEVMTSIALRILPTMCR | 38 | LMW | CTERM | NA | B2BZD0\|CTERM | 31.4075 | 8 | no |
| VNVPLYRTTTSVPFDVGTGVGAY | | glutenin | | | | | | |
| QLAHGTFLQPHQIAQLEVMTSIALHNLPMM | 38 | LMW | CTERM | NA | B3EY91\|CTERM | 26.29 | 15 | no |
| CSVNVPLYETTTSVPLGIGIGVGVY | | glutenin | | | | | | |
| QLAHGTFLQPHQIAQLEVMTSIALRTLPKMC | 38 | LMW | CTERM | NA | Q5MFH0\|CTERM | 51.5875 | 14 | no |
| SVNVPLYETPPSVPLGVGIGVGVY | | glutenin | | | | | | |
| QLTHGAFLQPHQIAQLEVMTSIALRNLPRMC | 38 | LMW | CTERM | NA | C3VN74\|CTERM | 33.79 | 15 | no |
| SVNVPLYETTTSVPLGVGIGVGVY | | glutenin | | | | | | |
| QLAHGTFLQPHQIAQLEVMTSIALRTLPTMC | 38 | LMW | CTERM | NA | C3VN76\|CTERM | 51.4875 | 14 | no |
| SVNVPLYETTTSVPLGVGIGVGVY | | glutenin | | | | | | |
| QLAQGTFLQPHQIAQLEVMTSIALRTLPMMC | 38 | LMW | CTERM | NA | B2Y2Q1\|CTERM | 45.95 | 15 | no |
| RVNVPLYRTTTSVPFGVGTGVGAY | | glutenin | | | | | | |
| QQLAQGTFLQPHQIAQLEVMTSIALRTLPTM | 38 | LMW | 336 | 392 | B2Y2R3\|336-392 | 36.75 | 9 | no |
| CNVNVPLYRTTTRVPFGVGTGVGGY | | glutenin | | | | | | |
| QLAQGTFLQPHQIAQLELMTSIALRTLPTMC | 38 | LMW | CTERM | NA | Q9ZNY0\|CTERM | 37.92 | 17 | no |
| NVNVPLYRTTTRVPFGVGTGVGAY | | glutenin | | | | | | |
| QLAQGTFLQPHQIAQLEVMTSIALRTLPTMC | 38 | LMW | CTERM | NA | F8SGN5\|CTERM | 37.6375 | 14 | no |
| RVNVPLYRTTTSVPFGVSAGVGAY | | glutenin | | | | | | |
| QQLAQGTFLQPHQIAQLEVMTSIALRTLPTM | 38 | LMW | CTERM | NA | Q8W3W8\|CTERM | 53.84 | 16 | no |
| CNVNVPLYRTTTRVPFGVGTGVGG | | glutenin | | | | | | |
| QPQQLGQCVSQPQQQLQQQLGQQPQQQQL | 37 | LMW | 308 | 364 | B3EY91\|308-364 | 48.13 | 17 | no |
| AHGTFLQPHQIAQLEVMTSIALHNLPM | | glutenin | | | | | | |
| QLGQCVSQPQQQLQQQLGQQPQQQQLAHG | 37 | LMW | 280 | 336 | P93794\|280-336 | 45.53 | 21 | no |
| TFLQPHQIAQLEVMTSIAPRTLPTMCS | | glutenin | | | | | | |
| QIPQGIFLQPHQISQLEVMTSIALRTLPTMCG | 37 | LMW | CTERM | NA | Q571Q5\|CTERM | 61.5475 | 10 | no |
| VNVPLYSSTTIMPFSIGTGVGGY | | glutenin | | | | | | |
| QVPQGTLLQPHQIAQLELMTSIALRTLPMMC | 36 | LMW | CTERM | NA | B2BZD1\|CTERM | 53.565 | 5 | no |
| SVNVPVYGTTTSVPFGVGTQVGAY | | glutenin | | | | | | |
| QVPQGTLLQPHQIAQLEVMTSIALRTLPTMC | 36 | LMW | CTERM | NA | B2BZC4\|CTERM | 30.4125 | 12 | no |
| SVNVPVYGTTTIVPFGVGTRVGAY | | glutenin | | | | | | |
| LGQWPQQQQVPQGTLLQPHQIAQLEVMTSI | 36 | LMW | 280 | 336 | B2Y2S2\|280-336 | 42.0325 | 8 | no |
| ALRTLPTMCSVNVPVYGTTTIVPFGV | | glutenin | | | | | | |
| CSFQQPQQLQQLGQQPQQQQIPQGIFLQPHQI | 36 | LMW | 252 | 308 | Q571Q5\|252-308 | 30.83 | 17 | no |
| SQLEVMTSIALRTLPTMCGVNVPL | | glutenin | | | | | | |
| QQQQPVLPQQPPFSQQQQQPILPQQPPFSQQ | 35 | LMW | 140 | 196 | D3UAL8\|140-196 | 20.35 | 18 | no |
| QQPVLLQQQIPFVHPSILQQLNPCK | | glutenin | | | | | | |
| QQQPFPQQQQPLRPQQPPFSQQQPPFSQQQQ | 35 | LMW | 112 | 168 | C8KIL6\|112-168 | 25.615 | 21 | no |
| QPVLPQQPPFSQHQQPVLPQQQIPS | | glutenin | | | | | | |
| PPFSQQQQTPFSQQQQIPVIHPSVLQQLNPCK | 34 | LMW | 196 | 252 | B3EY91\|196-252 | 23.81 | 5 | no |
| VFLQQQCIPVAMQRCLARSQMLQQ | | glutenin | | | | | | |
| LPPFSQQLPPFSQQQQPVLPQQPPFSQQQQQP | 34 | LMW | 168 | 224 | B2Y2R3\|168-224 | 17.6225 | 20 | no |
| ILPQQPPFSQQQQPVLLQQQIPFV | | glutenin | | | | | | |
| QQPPFSQQQQPSFSQQQQPPFSQQQPPFSQQ | 34 | LMW | 84 | 140 | Q8W3W3\|84-140 | 24.425 | 16 | no |
| QQQPVLPQQQIPFVHPSILQQLNPC | | glutenin | | | | | | |
| FSQQPPISQQQQPPFSQQQQPPFSQQQQIPVIH | 33 | LMW | 168 | 224 | C3VN76\|168-224 | 47.345 | 11 | no |
| PSVLQQLNPCKVFLQQQCIPVAM | | glutenin | | | | | | |
| SQPQQQQKQLGQCSFQRPQQQQLGQWPQQ | 32 | LMW | 280 | 336 | B2BZD1\|280-336 | 51.4825 | 10 | no |
| QQVPQGTLLQPHQIAQLELMTSIALRT | | glutenin | | | | | | |
| FSQQQQPVLPQQPPFSQQQQPILPQQPPFSQQ | 31 | LMW | 140 | 196 | B2BZD0\|140-196 | 54.5475 | 14 | no |
| QQQPVLPQQQIPFVHPSILQQLNP | | glutenin | | | | | | |
| PFTQQQQPPFSQQPPISQQQQPPFSQQQQPPF | 31 | LMW | 168 | 224 | C3VN74\|168-224 | 23.825 | 8 | no |
| SQQQQIPVIHPSVLQQLNPCKVFL | | glutenin | | | | | | |
| SQQQQPVLPQQPSFSQQQLPPFSQQQPPFSQQ | 31 | LMW | 112 | 168 | F8SGM4\|112-168 | 55.56 | 13 | no |
| QQQPVLPQQQILFVHPSILQQLNP | | glutenin | | | | | | |
| QQPVLPQQPSFSQQQLPPFSQQLPPFSQQQQP | 30 | LMW | 84 | 140 | D3UAL8\|84-140 | 38.395 | 14 | no |
| VLPQQPPFLQQQLPPFSQQLPPFS | | glutenin | | | | | | |
| FSQQQQQPVLPQQTPLSQQQQPVLQQQPPFS | 30 | LMW | 168 | 224 | D3U328\|168-224 | 41.6825 | 10 | no |
| QQQQQPVLPQQQIPFVHPSILQQLN | | glutenin | | | | | | |
| GSIQTPQQQPQQLGQCVSQPQQQSQQQLGQ | 29 | LMW | 280 | 336 | B2Y2Q6\|280-336 | 109.53 | 14 | no |
| QPQQQQLAQGTFLQPHQIAQLEVMTS | | glutenin | | | | | | |
| VQGVSQPQQQQKQLGQCPFQQPQQQQLGQ | 29 | LMW | 252 | 308 | F8SGL5\|252-308 | 95.82 | 4 | no |
| WPQQQQVPQGTLLQPHQIAQLEVMTSI | | glutenin | | | | | | |
| QPPFSQQQPPFSQQQQQPPISQQQQQQIIPQQ | 28 | LMW | 112 | 168 | B2Y2S2\|112-168 | 80.88 | 11 | no |
| PPFSQHQQPVLPQQQIPSVQPSIL | | glutenin | | | | | | |
| QQQGQSVIQYQQQQPQQLGQCVSQPQQQLQ | 27 | LMW | 280 | 336 | C3VN76\|280-336 | 20.52 | 11 | no |
| QQLGQQPQQQQLAHGTFLQPHQIAQL | | glutenin | | | | | | |
| QQQLPPFSQQQQPPFSQQQQPVLPQQPSFSQ | 27 | LMW | 84 | 140 | Q571Q5\|84-140 | 82.61 | 10 | no |
| QQLPPFSQQLPPFSQQQQPVLLQQQ | | glutenin | | | | | | |
| PVLPQQPSFSQQQLPPFSQQQQPPFSQQQQPV | 26 | LMW | 56 | 112 | D3UAL8\|56-112 | 62.5 | 11 | no |
| LPQQPSFSQQQLPPFSQQLPPFSQ | | glutenin | | | | | | |
| QQQQQPFTQQQQPPFSQQPPISQQQQPPFLQ | 22 | LMW | 140 | 196 | P10385\|140-196 | 63.275 | 6 | no |
| QQRPPFSRQQQIPVIHPSVLQQLNP | | glutenin | | | | | | |
| NQPLLPQQPPFSQQQPPFSQQQQQPPFSQQQ | 20 | LMW | 112 | 168 | Q8W3X5\|112-168 | 25.105 | 16 | no |
| QPPFSQHQQPVLPQQQIPSVQPSIL | | glutenin | | | | | | |
| QQLPPFSQQQQQVLPQQPPFSQQQQQQPIPP | 19 | LMW | 168 | 224 | Q8W3X4\|168-224 | 103.297 | 8 | no |
| QQPPFSQQQQPVLLQQQIPFVHPSI | | glutenin | | | | 5 | | |
| PQQFPQQQPCSQQQQQQQQQQQQQQQPLSQ | 18 | LMW | 56 | 112 | Q18NR2\|56-112 | 78.5675 | 12 | no |
| QQQPPFSQQQPPFSQQQQPVLPQQPS | | glutenin | | | | | | |
| FSQQQLPPFSQQQPPFSQQQQPVLPQQPPFSQ | 18 | LMW | 84 | 140 | B5ANT6\|84-140 | 72.405 | 11 | no |
| QQPVLPPQQSPFPQQQQHQQLVQQ | | glutenin | | | | | | |
| FSQQQLPPFSQQQPPFSQQQQPVLPQQPPFSQ | 17 | LMW | 112 | 168 | Q18NR2\|112-168 | 52.6875 | 12 | no |
| QQQQQPILPQQPSFSQQQQQLVLP | | glutenin | | | | | | |
| LERPWQQQPLPPQQTFPQQPPFSQQQQQPFP | 17 | LMW | 28 | 84 | B5ANT6\|28-84 | 45.29 | 11 | no |
| QQPPFSQQQPPFSQQQQPVLPQQPS | | glutenin | | | | | | |
| PFPQQPPFSQQQPPFSQQQQPVLPQQPSFSQQ | 17 | LMW | 56 | 112 | B5ANT6\|56-112 | 49.53 | 15 | no |
| QLPPFSQQQPPFSQQQQPVLPQQP | | glutenin | | | | | | |
| QQPPFSQQQQPSFSQQQQPPFSQQQPPFSQQ | 17 | LMW | 84 | 140 | B2BZD0\|84-140 | 25.72 | 16 | no |
| QQPVIPQQPSFSQQQLPPFSQQQPP | | glutenin | | | | | | |
| FSQQQQPPFSQQQPPFSQQQQPVLPQQPSFSQ | 17 | LMW | 112 | 168 | B2Y2Q6\|112-168 | 60.66 | 13 | no |
| QQLPPFSQQQSPFSQQQQIVLQQQ | | glutenin | | | | | | |
| PPFSQQLPPFLQQQQPVLPQQPPFSQQQLPPF | 17 | LMW | 140 | 196 | B2Y2R3\|140-196 | 46.19 | 13 | no |
| SQQLPPFSQQQQPVLPQQPPFSQQ | | glutenin | | | | | | |
| QPFPQQPPCSQQQQPPLSQQQQPPFSQQQPPF | 15 | LMW | 56 | 112 | B2BZC4\|56-112 | 41.22 | 14 | no |
| SQQQQPVLPQQPPFSQQQQQFPQQ | | glutenin | | | | | | |
| SQQQQPVIPQQPSFSQQQLPPFSQQQPPFSQQ | 13 | LMW | 112 | 168 | B2BZD0\|112-168 | 57.87 | 13 | no |
| QQPVLPQQPPFSQQQQPILPQQPP | | glutenin | | | | | | |
| QQPPFSQQQQPVLPQQPPFSQQQQQQPILPQ | 13 | LMW | 140 | 196 | B2Y2Q1\|140-196 | 55.77 | 11 | no |
| QPSFSQQQQQLVLPQQQIPFVHPSI | | glutenin | | | | | | |
| QPFPQQPPCSQQQQPPLLQQQQPPFSQQQPPF | 13 | LMW | 56 | 112 | B2Y2S2\|56-112 | 22.01 | 19 | no |
| SQQQQPVLPQQPPFSQQQQPLLPQ | | glutenin | | | | | | |
| QQQPVLPQQPPFLQQQLPPFSQQLPPFSQQQ | 13 | LMW | 112 | 168 | D3UAL8\|112-168 | 37.88 | 17 | no |
| QPVLPQQPPFSQQQQQPILPQQPPF | | glutenin | | | | | | |
| QPFPQQPACSQQQQPPLLQQQQPPFSQQQPP | 13 | LMW | 56 | 112 | C8KIL6\|56-112 | 26.11 | 19 | no |
| FSQQQQPVLPQQPPFSQQQQPQFAQ | | glutenin | | | | | | |
| QQQQQQQPLSQQQQPPFSQQQQPPFSQQQPP | 12 | LMW | 84 | 140 | B2Y2Q1\|84-140 | 20.03 | 17 | no |
| FSQQQQPVLPQQPSFSQQQLPPFSQ | | glutenin | | | | | | |
| QQQPPFSQQQQQPLSQQQQPPFSQQQPPFSQ | 11 | LMW | 84 | 140 | B2Y2Q6\|84-140 | 34.715 | 17 | no |
| QQQPPFSQQQPPFSQQQQPVLPQQP | | glutenin | | | | | | |
| QPPFSQQELPVLPQQPPFSQQQQPFPQQQQPL | 10 | LMW | 84 | 140 | Q0GNG1\|84-140 | 35.85 | 13 | no |
| LPQQPPFSQQRPPFSQQQQQPVLP | | glutenin | | | | | | |
| FSQQQQPVLPQQPPFSQQQQPILPQQPPFSQQ | 10 | LMW | 140 | 196 | D3U328\|140-196 | 50.3475 | 14 | no |
| QQQPVLPQQTPLSQQQQPVLQQQP | | glutenin | | | | | | |
| QPFPQQPPCSQQQQPPLSQQQQPPFSQQQPPF | 9 | LMW | 56 | 112 | B2BZD1\|56-112 | 30.675 | 16 | no |
| SQQELPILPQQPPFSQQQQPQFSQ | | glutenin | | | | | | |
| PQPFPQQQPCSQQQQPPLSQQQQPPFSQQQP | 9 | LMW | 56 | 112 | B2BZD0\|56-112 | 67.33 | 11 | no |
| PFSQQQQPSFSQQQQPPFSQQQPPF | | glutenin | | | | | | |
| PQQPPFSQQQQLVLPQQPPFSQQQQPVLPPQ | 9 | LMW | 84 | 140 | A9Z176\|84-140 | 78.695 | 10 | no |
| QSPFPQQQRHQQLVQQQIPVVQPSI | | glutenin | | | | | | |
| PQPFSQQQPCSQQQQQQPLSQQQQPPFSQQQ | 9 | LMW | 56 | 112 | B2Y2Q6\|56-112 | 78.8725 | 12 | no |
| PPFSQQQQQPLSQQQQPPFSQQQPP | | glutenin | | | | | | |
| SFSQQQLPPFSQQQSPFSQQQQIVLQQQPPFL | 8 | LMW | 140 | 196 | B2Y2Q6\|140-196 | 42.35 | 13 | no |
| QQQQPSLPQQPPFSQQQQQLVLPQ | | glutenin | | | | | | |
| QQPPFSQQQPPFSQQELPILPQQPPFSQQQQP | 8 | LMW | 56 | 112 | D3U326\|56-112 | 32.93 | 12 | no |
| QFSQQQQPFPQQQQPLLLQQPPFS | | glutenin | | | | | | |
| LERPWQQQPLPPQQTLAQQPLFSQQQQLFAK | 7 | LMW | 28 | 84 | Q5MFH4\|28-84 | 11.6375 | 8 | no |
| QPSFSQQQPPFWQQQPPFSHQQPIL | | glutenin | | | | | | |
| QPPFSQQQQPILPQQPPFSQQQQQFPQQQQPL | 7 | LMW | 84 | 140 | C3VN79\|84-140 | 15.9 | 17 | no |
| LLQQPPLSQQQPPFSRQQQQPPFS | | glutenin | | | | | | |
| QQQQPPLSQQQQPPFSQQQQPPFSQQQQPVL | 7 | LMW | 28 | 84 | D3UAL8\|28-84 | 43.2075 | 12 | no |
| PQQPSFSQQQLPPFSQQQQPPFSQQ | | glutenin | | | | | | |
| PFSQQQQPVLPQQPPFSQRQLPPFSQQQQPPF | 7 | LMW | 84 | 140 | Q8W3W8\|84-140 | 70.98 | 13 | no |
| SQQQQPVLPQQPPFSQQQQPVLLQ | | glutenin | | | | | | |
| QPPFSQQELPILPQQPPFSQQQQPQFSQQQQP | 6 | LMW | 84 | 140 | B2BZD1\|84-140 | 34.5875 | 16 | no |
| FPQQQQPLLLQQPPFSQQRPPFSQ | | glutenin | | | | | | |
| LERPWQQQPLPPQQTFPQQPLFSQQQQLFPQ | 6 | LMW | 28 | 84 | A9Z176\|28-84 | 40.555 | 9 | no |
| QPSFSQQQPPFWQQQPPFSQQQPIL | | glutenin | | | | | | |
| QPPFSQQQQPVLPQQPPFSQQQQPLLPQQPPF | 6 | LMW | 84 | 140 | B2Y2S2\|84-140 | 35.665 | 16 | no |
| SQQQPPFSQQQQQPPISQQQQQQI | | glutenin | | | | | | |
| QPPFSQQQQPVLPQQPPFSQQQQPQFAQQQQ | 6 | LMW | 84 | 140 | C8KIL6\|84-140 | 33.3 | 17 | no |
| PFPQQQQPLRPQQPPFSQQQPPFSQ | | glutenin | | | | | | |
| LERPWQQQPLQQKETFPQQPPSSQQQQPFPQ | 5 | LMW | 0 | NA | Q8W3V4_2 | 48.1275 | 6 | no |
| QPPFLQQQPSFSQQPLFSQKQQPVL | | glutenin | | | | | | |
| LERPWQQQPLQQKETFPQQPPSSQQQQPLPQ | 5 | LMW | 28 | 84 | D0EVN9\|28-84 | 27.6 | 4 | no |
| QPPFLQQQPSFSQQPLFSQKQQPVL | | glutenin | | | | | | |
| QPPFSQQQQPVLPQQPPFSQQQQQFPQQQQP | 5 | LMW | 84 | 140 | B2BZC4\|84-140 | 44.36 | 15 | no |
| LLPQQPPFSQQQPPFSQQQQQPPFS | | glutenin | | | | | | |
| PFSQQQQPPFSQQQQPPFSQQQQSPFSQQQE | 5 | LMW | 28 | 84 | B3EY91\|28-84 | 50.67 | 13 | no |
| QQQQPPFLQQQQPPFSQQPPISQQQ | | glutenin | | | | | | |
| FAKQPSFSQQQPPFWQQQPPFSHQQPILPQQP | 5 | LMW | 56 | 112 | Q5MFH4\|56-112 | 16.2 | 17 | no |
| PFSQQQQLVLPQQPPFSQQQQPVL | | glutenin | | | | | | |
| QQQPQFSQQQQPFPQQQQPLLLQQPPFSQQR | 5 | LMW | 84 | 140 | D3U326\|84-140 | 37.33 | 12 | no |
| PPFSQQQQQPVLPQQPPFSQQQQQQ | | glutenin | | | | | | |
| QQQPFPQQQQPLLLQQPPFSQQRPPFSQQQQ | 4 | LMW | 112 | 168 | B2BZD1\|112-168 | 37.06 | 12 | no |
| QPVLPQQPPFSQQQQQQPILPQQPP | | glutenin | | | | | | |
| SQQQQPPFSQQQQPPFSQQQQPPFSQQQQQQ | 4 | LMW | 112 | 168 | B3EY91\|112-168 | 34.0425 | 16 | no |
| QQQQQQPFTQQQPPFSQQPPISQQQ | | glutenin | | | | | | |
| LERPWHHHPLPPQHTFPQQPLFSQQQQLFPQ | 4 | LMW | 28 | 84 | Q5MFK3\|28-84 | 30.87 | 7 | no |
| QPSFSQQQPPFWQQQPPFSQQQPIL | | glutenin | | | | | | |
| QQPIIILQQSPFSQQQQIVLQQQPPFLQQQQPS | 4 | LMW | 56 | 112 | Q5MFQ0\|56-112 | 26.25 | 12 | no |
| LPQQPPFSQQQQQLVLPQQQIPF | | glutenin | | | | | | |
| LFSQQQQPPFSQQQQPPFSQQQQSPFSQQQQ | 4 | LMW | 28 | 84 | C3VN74\|28-84 | 45.61 | 9 | no |
| QPPFLQQQQPPFSQQPPISQQQQPP | | glutenin | | | | | | |
| PFSQQQQPPFSQQQQSPFSQQQQPPFLQQQQ | 4 | LMW | 28 | 84 | C3VN76\|28-84 | 30.01 | 9 | no |
| PPFSQQPPISQQQQPPFSQQQQPQF | | glutenin | | | | | | |
| QPPFSQQQQPPFSQQQQPPFSQQQQQQQPPF | 4 | LMW | 112 | 168 | D2DII1\|112-168 | 26.61 | 13 | no |
| TQQQQPPFSQQPPISQQQQPPFSQQ | | glutenin | | | | | | |
| PFSQQQQPPFSQQQQPPFSQQQQSPFSQQQQ | 4 | LMW | 28 | 84 | D2DII2\|28-84 | 35.6225 | 14 | no |
| QPPFSQQQQPPFSQQPLISQQQQLP | | glutenin | | | | | | |
| QQPPFSQQQQPPFSQQQQQPPFTQQQQQQQQ | 3 | LMW | 112 | 168 | P10385\|112-168 | 28.51 | 15 | no |
| QQPFTQQQQPPFSQQPPISQQQQPP | | glutenin | | | | | | |
| QPPFSQEQQPPFSQQQQPPFSQQQQPPYSQQ | 3 | LMW | 84 | 140 | B3EY91\|84-140 | 21.01 | 17 | no |
| QQPPFSQQQQPPFSQQQQPPFSQQQ | | glutenin | | | | | | |
| FSQQQQPQFSQQQQPPYSQQQQPPYSQQQQP | 3 | LMW | 84 | 140 | C3VN74\|84-140 | 29.04 | 12 | no |
| PFSQQQQPPFSQQQQPPFSQQQQQP | | glutenin | | | | | | |
| QQPPFSQQQQPPFSQQQQPPFSQQQQQPPFT | 3 | LMW | 112 | 168 | C3VN74\|112-168 | 37.68 | 9 | no |
| QQQQPSFSQQPPISQQQQQQQQQQQ | | glutenin | | | | | | |
| QQQPPFSQQPPISQQQQPPFSQQQQPQFSQQQ | 3 | LMW | 56 | 112 | C3VN76\|56-112 | 37.24 | 10 | no |
| QPPYSQQQQPPYSQQQQPPFSQQQ | | glutenin | | | | | | |
| SQQQQPPYSQQQQPPYSQQQQPPFSQQQQPP | 3 | LMW | 84 | 140 | C3VN76\|84-140 | 19.18 | 15 | no |
| FSQQQQPPFLQQQQQPPFTQQQQPS | | glutenin | | | | | | |
| QPPFSQQQQPPFLQQQQQPPFTQQQQPSFSQ | 3 | LMW | 112 | 168 | C3VN76\|112-168 | 39.92 | 8 | no |
| RPPISQQQQQQQQQQQPFTQQQQPP | | glutenin | | | | | | |
| FSQRPPISQQQQQQQQQQQPFTQQQQPPFSQ | 3 | LMW | 140 | 196 | C3VN76\|140-196 | 58.14 | 11 | no |
| QPPISQQQQPPFSQQQQPPFSQQQQ | | glutenin | | | | | | |
| PQQFPQQQPCSQQQQQQQQQQQQQQQQQQ | 3 | LMW | 56 | 112 | B2Y2Q1\|56-112 | 48.855 | 9 | no |
| QQQQQQPLSQQQQPPFSQQQQPPFSQQ | | glutenin | | | | | | |
| QQTLSHHHQQQPIQQQPQQFPQQQPCSQQQ | 3 | LMW | 0 | 56 | D3UAL8\|0-56 | 33.45 | 10 | no |
| QQPPLSQQQQPPFSQQQQPPFSQQQQ | | glutenin | | | | | | |
| PFSQQQQPQFSQQPPFSQQQQPPFSQQQQQP | 3 | LMW | 28 | 84 | P93794\|28-84 | 30.3475 | 12 | no |
| PFAQQQQPPFSQQPPISQQQQPPFS | | glutenin | | | | | | |
| QQQPPFSQQQQPPFSQQPPISQQQQPQFLQQ | 3 | LMW | 56 | 112 | D2DII1\|56-112 | 40.22 | 13 | no |
| QQPPFSQQQQPPFSQQQQPPYSQQQ | | glutenin | | | | | | |
| QQQQPPFSQQQQPPFSQQPLISQQQQLPFSQQ | 3 | LMW | 56 | 112 | D2DII2\|56-112 | 50.34 | 8 | no |
| QQPQFSQQQQPPYSQQQQPPYSQQ | | glutenin | | | | | | |
| QQPPFSQQQQPPFSQQQQPSFSQQQQQPPFT | 3 | LMW | 112 | 168 | D2DII2\|112-168 | 48.89 | 7 | no |
| QQQQPPFSQQSPISQQQQQQQQQQQ | | glutenin | | | | | | |
| SQQQQPPFSQQQQPPFSQQQQPPFSQQQQQT | 3 | LMW | 112 | 168 | Q8W3V0\|112-168 | 44.09 | 11 | no |
| NKQQQQQPFTQQQPPFSQQPPISQQ | | glutenin | | | | | | |
| QQTNKQQQQQPFTQQQPPFSQQPPISQQQQQ | 3 | LMW | 140 | 196 | Q8W3V0\|140-196 | 63.33 | 9 | no |
| QQQQQQPFTQQQPPFSQQPPISQQQ | | glutenin | | | | | | |
| LEKPSQQQPLPLQQTLSHHQQQQPVQQQPQP | 2 | LMW | 28 | 84 | B2BZD0\|28-84 | 45.475 | 8 | no |
| FPQQQPCSQQQQPPLSQQQQPPFSQ | | glutenin | | | | | | |
| QQQQPPFLQQQQPPFSQQPPISQQQQPPFSQQ | 2 | LMW | 56 | 112 | C3VN74\|56-112 | 19.955 | 13 | no |
| QQPQFSQQQQPPYSQQQQPPYSQQ | | glutenin | | | | | | |
| PFTQQQQPSFSQQPPISQQQQQQQQQQQPFT | 2 | LMW | 140 | 196 | C3VN74\|140-196 | 27.34 | 11 | no |
| QQQQPPFSQQPPISQQQQPPFSQQQ | | glutenin | | | | | | |
| LEKPLQQQPLPLQQILWYQQQQPIQQQPQPF | 1 | LMW | 28 | 84 | B2BZC4\|28-84 | 47.86 | 7 | no |
| PQQPPCSQQQQPPLSQQQQPPFSQQ | | glutenin | | | | | | |
| QQQQQQQQQPFTQQQPPFSQQPPISQQQQQQ | 1 | LMW | 140 | 196 | B3EY91\|140-196 | 51.975 | 6 | no |
| QQQPQPFTQQQPPFSQQPPISQQQQ | | glutenin | | | | | | |
| LERPSQQQPLPPQQTLSHHHHQQPIQQQPQQ | 1 | LMW | 28 | 84 | B2Y2R5\|28-84 | 13.385 | 4 | no |
| FPQQQPCSQQQQQPPLSQQQQPPFS | | glutenin | | | | | | |
| PQQFPQQQPCSQQQQQQQQQQQQQQQQQQ | 1 | LMW | 56 | 112 | B2Y2Q2\|56-112 | 48.055 | 11 | no |
| QQQQQQQQQQPLSQQQQPPFSQQQQPP | | glutenin | | | | | | |
| LERPSQQQPLPPQQTLSHHQQQQPIQQQPQPF | 1 | LMW | 28 | 84 | B2Y2Q6\|28-84 | 19.49 | 9 | no |
| SQQQPCSQQQQQQPLSQQQQPPFS | | glutenin | | | | | | |
| LEKPSQQQPLPLQQILWYHQQQPIQQQPQPF | 1 | LMW | 28 | 84 | C8KIL6\|28-84 | 14.545 | 5 | no |
| PQQPACSQQQQPPLLQQQQPPFSQQ | | glutenin | | | | | | |
| LEKPWQQQPLPPQQQPPCSQQQQPFPQQQQP | 0 | LMW | 28 | 84 | Q5MFQ0\|28-84 | 26.7 | 10 | no |
| IIILQQSPFSQQQQIVLQQQPPFLQ | | glutenin | | | | | | |
| MGRLLSPRGKELHTPQEQFPQQQQFPQPQQF | 2 | LMW | 0 | 56 | B6ETS0\|0-56 | 20.97 | 9 | no |
| PQQQILQQHQIPQQPQQFPQQQQFL | | glutenin | | | | | | |
| QQQFPQQQFPQQQFPQQEFPQQQQFPQQQIA | 2 | LMW | 196 | 252 | B6ETS0\|196-252 | 54.5 | 8 | no |
| QQPQQLPQQQFPIPYPPQQSQEPSP | | glutenin | | | | | | |
| QQQQIPQQQIPQQHQIPQQPQQFPQQQFPQQ | 1 | LMW | 56 | 112 | B6ETS0\|56-112 | 63.7375 | 8 | no |
| QQFPQQHQSPQQQFPQQQFPQQQLP | | glutenin | | | | | | |
| PQQQQFPQQHQSPQQQFPQQQFPQQQLPQQ | 1 | LMW | 84 | 140 | B6ETS0\|84-140 | 59.515 | 5 | no |
| EFSQQQISQQPQQLPQQQQIPQQPQQ | | glutenin | | | | | | |
| IPQQQQIPQQPQQIPQQQQIPQQPKQFPQQQF | 0 | LMW | 168 | 224 | B6ETS0\|168-224 | 83.11 | 5 | no |
| PQQQFPQQQFPQQEFPQQQQFPQQ | | glutenin | | | | | | |
| AACQASQLAVCASAILSGAKPSGECCGNLRA | 0 | LTP2G | 28 | 84 | P82900\|28-84 | 27.485 | 4 | no |
| QQGCFCQYAKDPTYGQYIRSPHARD | | | | | | | | |
| QFPQQQFPQQKLPQQEFPQQQISQQPQQLPQ | 5 | Omega | 112 | 168 | Q40215\|112-168 | 51.535 | 6 | no |
| QQQIPQQPQQFLQQQQFPQQQPPQQ | | gliadin | | | | | | |
| LQSQQPFHQQPEQIISQQPQQPFSLQPQQPFS | 5 | Omega | 252 | 308 | Q571R2\|252-308 | 32.45 | 13 | no |
| QPQQPFPQQPGQIIPQQPQQPFPL | | gliadin | | | | | | |
| PQQPFPQPQLPFPQQSEQIIPQQLQQPFPLQPQ | 4 | Omega | 140 | 196 | Q9FUW7\|140-196 | 38.84 | 14 | no |
| QPFPQQPQQPFPQPQQPIPVQPQ | | gliadin | | | | | | |
| QQQQIPQQPQQFPQQQFPQQQFPQQQFPQQE | 4 | Omega | 196 | 252 | Q40215\|196-252 | 64.595 | 8 | no |
| FPQQQQFPQQQIARQPQQLPQQQQI | | gliadin | | | | | | |
| PQQPFPQQPQRPQQSFPQQPEQIIPQQPQQPFP | 4 | Omega | 140 | 196 | Q571R2\|140-196 | 72.39 | 7 | no |
| LQPQQQFPEQSEQIISQQRQQPF | | gliadin | | | | | | |
| QPSPLQPQQPFPQQPEQIIPQQPQQPFLLQSQQ | 4 | Omega | 224 | 280 | Q571R2\|224-280 | 44.75 | 9 | no |
| PFHQQPEQIISQQPQQPFSLQPQ | | gliadin | | | | | | |
| PAQQPFPQQPGQIIPQQPQQPLPLQPQQPFPW | 3 | Omega | CTERM | NA | Q6PNA3\|CTERM | 37.82 | 14 | no |
| QPEQRSSQQPQQPFSLQPQQPFS | | gliadin | | | | | | |
| PFPWQPQQPFPQTQQSFPLQPQQPFPQQPQQ | 3 | Omega | 112 | 168 | Q9FUW7\|112-168 | 42.6025 | 12 | no |
| PFPQPQLPFPQQSEQIIPQQLQQPF | | gliadin | | | | | | |
| QQPQQPFPLQPQQPFPQQPQQPFPQQPQQSFP | 3 | Omega | CTERM | NA | Q9FUW7\|CTERM | 63.33 | 13 | no |
| QQPQQPYPQQQPYGSSLTSIGGQ | | gliadin | | | | | | |
| PQQQQLTQQQFPRPQQSPEQQQFPQQQFPQQ | 3 | Omega | 364 | 420 | Q40215\|364-420 | 56.16 | 8 | no |
| PPQQFPQQQFPIPYPPQQSEEPSPY | | gliadin | | | | | | |
| QPQQPFPQQPQQPFPLQPQQSFPQQPQQPFPQ | 3 | Omega | 308 | 364 | Q571R2\|308-364 | 54.19 | 15 | no |
| PQQAFPEQGEQIIPQQPQQPFPLQ | | gliadin | | | | | | |
| PFPLQPQQSFPQQPQQPFPQPQQAFPEQGEQII | 3 | Omega | CTERM | NA | Q571R2\|CTERM | 53.01 | 13 | no |
| PQQPQQPFPLQPHQPQQPYPQQ | | gliadin | | | | | | |
| ELQSPQQSFSYQQQPFPQQPYPQQPYPSQQP | 2 | Omega | 28 | 84 | Q9FUW7\|28-84 | 23.785 | 13 | no |
| YPSQQPFPTPQQQFPEQSQQPFTQP | | gliadin | | | | | | |
| QQPTPIQPQQPFPQQPQQPQQPFPQPQQPFPW | 2 | Omega | 84 | 140 | Q9FUW7\|84-140 | 41.0125 | 18 | no |
| QPQQPFPQTQQSFPLQPQQPFPQQ | | gliadin | | | | | | |
| PLQPQQPFPQQPQQPFPQPQQPIPVQPQQSFP | 2 | Omega | 168 | 224 | Q9FUW7\|168-224 | 49.29 | 10 | no |
| QQSQQSQQPFAQPQQLFPELQQPI | | gliadin | | | | | | |
| PQQPQQPFPLQPQQPFPQQPQQPFPQQPQQSF | 2 | Omega | 224 | 280 | Q9FUW7\|224-280 | 42.37 | 16 | no |
| PQQPQQPYPQQQPYGSSLTSIGGQ | | gliadin | | | | | | |
| QQFPQQQFPQQPPQQFPQQQFPIPYPPQQSEE | 2 | Omega | CTERM | NA | Q40215\|CTERM | 28.24 | 9 | no |
| PSPYQQYPQQQPSGSDVISISGL | | gliadin | | | | | | |
| PYPQQAFPIPQQYSPHQPQQPFPQPQRPTPLQ | 2 | Omega | 28 | 84 | Q571R2\|28-84 | 34.3225 | 14 | no |
| SQQPFPQQPKQPQQSFSQPQQQFP | | gliadin | | | | | | |
| TPLQSQQPFPQQPKQPQQSFSQPQQQFPLQP | 2 | Omega | 56 | 112 | Q571R2\|56-112 | 35.52 | 14 | no |
| QQPFPQPQQPIPQQPRQPFPEQPQR | | gliadin | | | | | | |
| PQQPQQSFPQPQQQFPLQPQQPFPQPQQPQQ | 2 | Omega | 112 | 168 | Q571R2\|112-168 | 105.285 | 8 | no |
| PFPQQPQRPQQSFPQQPEQIIPQQP | | gliadin | | | | | | |
| QPFSQPQQPFPQQPGQIIPQQPQQPFPLQPQQ | 2 | Omega | 280 | 336 | Q571R2\|280-336 | 40.875 | 14 | no |
| PFPQQPQQPFPLQPQQSFPQQPQQ | | gliadin | | | | | | |
| APTIISPATRTNNSLATPTTIPPATATTIPPATR | 1 | Omega | 224 | 280 | Q6PNA3\|224-280 | 18.4475 | 4 | no |
| TNNSPATATTIPPAPQQRFPHT | | gliadin | | | | | | |
| RQKFPRNPNNHSLCSTHHFPAQQPFPQQPGQ | 1 | Omega | 280 | 336 | Q6PNA3\|280-336 | 15.09 | 15 | no |
| IIPQQPQQPLPLQPQQPFPWQPEQR | | gliadin | | | | | | |
| QSFPQQSQQSQQPFAQPQQLFPELQQPIPQQP | 1 | Omega | 196 | 252 | Q9FUW7\|196-252 | 36.83 | 13 | no |
| QQPFPLQPQQPFPQQPQQPFPQQP | | gliadin | | | | | | |
| QQEFPQQQQFPQQQIARQPQQLPQQQQIPQQ | 1 | Omega | 224 | 280 | Q40215\|224-280 | 24.58 | 11 | no |
| PQQFPQQQQFPQQQSPQQQQFPQQQ | | gliadin | | | | | | |
| PQQPQQFPQQQQFPQQQSPQQQQFPQQQFPQ | 1 | Omega | 252 | 308 | Q40215\|252-308 | 32.85 | 8 | no |
| QQQLPQKQFPQPQQIPQQQQIPQQP | | gliadin | | | | | | |
| FPQQQQLPQKQFPQPQQIPQQQQIPQQPQQFP | 1 | Omega | 280 | 336 | Q40215\|280-336 | 84.58 | 7 | no |
| QQQFPQQQQFPQQQEFPQQQFPQQ | | gliadin | | | | | | |
| QQFPQQQFPQQQQFPQQQEFPQQQFPQQQFH | 1 | Omega | 308 | 364 | Q40215\|308-364 | 37.785 | 10 | no |
| QQQLPQQQFPQQQFPQQQFPQQQQF | | gliadin | | | | | | |
| LQPQQPFPQPQQPIPQQPRQPFPEQPQRPQQP | 1 | Omega | 84 | 140 | Q571R2\|84-140 | 64.9725 | 12 | no |
| QQSFPQPQQQFPLQPQQPFPQPQQ | | gliadin | | | | | | |
| QQPYPSQQPFPTPQQQFPEQSQQPFTQPQQPT | 0 | Omega | 56 | 112 | Q9FUW7\|56-112 | 32.76 | 14 | no |
| PIQPQQPFPQQPQQPQQPFPQPQQ | | gliadin | | | | | | |
| QFHQQQLPQQQFPQQQFPQQQFPQQQQFPQ | 0 | Omega | 336 | 392 | Q40215\|336-392 | 40.72 | 11 | no |
| QQQLTQQQFPRPQQSPEQQQFPQQQF | | gliadin | | | | | | |
| HQFPQQQLPQQQQIPQQQQIPQQPQQIPQQQ | 0 | Omega | 168 | 224 | Q40215\|168-224 | 43.75 | 7 | no |
| QIPQQPQQFPQQQFPQQQFPQQQFP | | gliadin | | | | | | |
| SLQPQQPFSQPQQPLSQQPGQIIPQQPQQPSPL | 0 | Omega | 196 | 252 | Q571R2\|196-252 | 36.35 | 10 | no |
| QPQQPFPQQPEQIIPQQPQQPFL | | gliadin | | | | | | |
| SGPWMCYPGQAFQVPALPGCRPLLKLQCNG | 11 | Uncharac- | 28 | 84 | A0A077RSX3\|28- | 25.165 | 2 | no |
| SQVPEAVLRDCCQQLADISEWCRCGA | | terized | | | 84 | | | |
| | | protein | | | | | | |

TABLE 2

| | | | | | IgE | IgG |
|---|---|---|---|---|---|---|
| | | pos_ | pos_ | | Reactivity- | Reactivity- |
| Amino Acid Sequence | product | start | end | pep_id | Allerscan | Allerscan |
| ISPECHPVVVSPVAGQYEQQIVVPPKGGSFY | Gamma gliadin | 56 | 112 | A9YSK4\|56-112 | 0 | 4 |
| PGETTPPQQLQQRIFWGIPALLKRY | | | | | | |
| MGSKGFKGVIVCLLILGLVLEQLQVEGKSCC | Uncharacterized | | | Q9T0P1_1 | 0 | 0 |
| RSTLGRNCYNLCRARGAQKLCAGVC | protein | | | | | |
| RCKISSGLSCPKGFPKLALESNSDEPDTIEY | Uncharacterized | | | Q9T0P1_3 | 0 | 0 |
| CNLGCRSSVCDYMVNAAADDEEMKL | protein | | | | | |
| MGSKGLKGVMVCLLILGLVLEQVQVEGKSCC | Alpha-gliadin | 0 | 56 | P32032\|0-56 | 0 | 0 |
| RTTLGRNCYNLCRSRGAQKLCSTVC | | | | | | |
| PDTIEYCNLGCRSSVCDYMVNAAADDEEMKL | Alpha-gliadin | CTERM | | P32032\|CTERM | 0 | 0 |
| YVENCGDACVNFCNGDAGLTSLDA* | | | | | | |
| AQKLCAGVCRCKIASGLSCPKGFPKLALESN | Dy-type | 0 | 56 | Q9S6Y2\|0-56 | 0 | 0 |
| SDEPDTIEYCNLGCRSSVCDYMVNA | HMW protein | | | | | |
| MKPHHDGYKYTCSIIVTFHYPNFKHQDQKHQ | Peroxidase | 0 | 56 | Q6PNA3\|0-56 | 0 | 0 |
| FQESIKHKSKMKTFIIFVLLSMPMS | | | | | | |
| KHQFQESIKHKSKMKTFIIFVLLSMPMSIVI | Peroxidase | 28 | 84 | Q6PNA3\|28-84 | 0 | 0 |
| AARHLNPSDQELQSPQQQFLEKTII | | | | | | |
| IVIAARHLNPSDQELQSPQQQFLEKTIISAA | Peroxidase | 56 | 112 | Q6PNA3\|56-112 | 0 | 0 |
| TISTSTIFTTTTISHTPTIFPPSTT | | | | | | |
| SAATISTSTIFTTTTISHTPTIFPPSTTTTI | Peroxidase | 84 | 140 | Q6PNA3\|84-140 | 0 | 0 |
| SPTPTTNPPTTTMTIPLATPTTTTT | | | | | | |
| TTISPTPTTNPPTTTMTIPLATPTTTTTFSP | Peroxidase | 112 | 168 | Q6PNA3\|112-168 | 0 | 0 |
| APTTISLATTTTISLAPTTNSPITT | | | | | | |
| QQQLIPCRDVVLQQPNIAHASSQVSQQSYQL | LMW Glutenin | 112 | 168 | D2T2K3\|112-168 | 0 | 0 |
| LQQLCCQQLWQTPEQSRCQAIHNVI | | | | | | |
| YQLLQQLCCQQLWQTPEQSRCQAIHNVIHAI | LMW Glutenin | 140 | 196 | D2T2K3\|140-196 | 0 | 1 |
| ILHHQQQQQQQQQQQQQQQQQQQQQ | | | | | | |
| SQQNPQAQGFVQPQQLPQFEEIRNLALQTLP | LMW Glutenin | CTERM | | D2T2K3\|CTERM | 0 | 0 |
| AMCNVYIPPYCSTTIAPFGIFSTN* | | | | | | |
| MKKFLVLALIVVAATTTAAEPFTAFRSAWEP | LMW Glutenin | | | D2KFG9_1 | 0 | 0 |
| QHPSSPEHQPTPQPQEHPVPHQKLN | | | | | | |
| CLVMWEQCCQQLKAIPKQSRCEAIHNVVHAI | LMW Glutenin | | | D2KFG9_4 | 0 | 0 |
| ILQQQQQLVQATSTQPQQQQQQGQQ | | | | | | |
| HAIILQQQQQLVQATSTOPQQQQQQGQQQQQ | LMW Glutenin | | | D2KFG9_5 | 0 | 1 |
| GLGSSQPQQQTOLDQGWIAVIGTWV | | | | | | |
| QQQGLGSSQPQQQTOLDQGWIAVIGTWVIQT | LMW Glutenin | | | D2KFG9_6 | 0 | 0 |
| IPAMCDVHVPPYCYTTISPSSDVTT | | | | | | |
| IQTIPAMCDVHVPPYCYTTISPSSDVTTDMG | LMW Glutenin | | | D2KFG9_7 | 0 | 0 |
| GY | | | | | | |
| MKTFLIFALLAVAATSAIAQMENSHIPGLER | Gamma gliadin | 0 | 56 | A2IBV5\|0-56 | 0 | 0 |
| PSQQQPLPPQQTLTHHQQQQPIQQQ | | | | | | |
| SSCHVMQQQCCRQLPQIPQQSRYEAIRAIVY | Gamma gliadin | 224 | 280 | A2IBV5\|224-280 | 0 | 0 |
| SIILQEQQQVQGSIQTQQQQPQQLG | | | | | | |
| IVYSIILQEQQQVQGSIQTQQQQPQQLGQCV | Gamma gliadin | 252 | 308 | A2IBV5\|252-308 | 0 | 2 |
| SQPQQQSQQQLGQQPQQQQLAQGTF | | | | | | |
| MKTLLILTILVMAVTIGTANMQVDPSGQVQW | HMW Glutenin | 0 | 56 | B6UKN8\|0-56 | 0 | 0 |
| PQQQPVLLPQQPFSQQPQQTFPRPQ | | | | | | |
| QRSFIQPSLQQQLNPCKNILLQQCKPASLVS | HMW Glutenin | 168 | 224 | B6UKN8\|168-224 | 0 | 0 |
| SLWSIIWPQSDCQVMRQQCCQQLAQ | | | | | | |
| LVSSLWSIIWPQSDCQVMRQQCCQQLAQIPQ | HMW Glutenin | 196 | 252 | B6UKN8\|196-252 | 0 | 0 |
| QLQCAAIHSVVHSIIMQQQQQQQQQ | | | | | | |
| QPQQSFPQQQQPLIQLSLQQQMNPCKNFLLQ | HMW Glutenin | 112 | 168 | B6DQB1\|112-168 | 0 | 0 |
| QCNPVSLVSSLISMILPRSDCQVMQ | | | | | | |
| LLQQCNPVSLVSSLISMILPRSDCQVMQQQC | HMW Glutenin | 140 | 196 | B6DQB1\|140-196 | 0 | 0 |
| CQQLAQIPQQLQCAAIHSVVHSIIM | | | | | | |
| VQGQGIIQPQQPAQLEVIRSLALRTLPTMCN | HMW Glutenin | CTERM | | B6DQB1\|CTERM | 0 | 0 |
| VYVSPDCSTINAPFASIVVGIGGQ* | | | | | | |
| IQTQQQQPQQLGQCVSQPQQQSQQQLGQCSF | Peroxidase | 224 | 280 | Q571Q5\|224-280 | 0 | 2 |
| QQPQQLQQLGQQPQQQQIPQGIFLQ | | | | | | |
| MAKRLVLFVAVVVALVALTVAEGEASEQLQC | Gamma gliadin | 0 | 56 | A9YSK4\|0-56 | 0 | 0 |
| ERELQELQERELKACQQVMDQQLRD | | | | | | |
| LQCERELQELQERELKACQQVMDQQLRDISP | Gamma gliadin | 28 | 84 | A9YSK4\|28-84 | 0 | 1 |
| ECHPVVVSPVAGQYEQQIVVPPKGG | | | | | | |

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for detecting the presence of an antibody against an allergen in a subject, the method comprising:
(a) contacting a reaction sample comprising a display library with a biological sample comprising antibodies, wherein the display library comprises a plurality of peptides derived from a plurality of allergens; and
(b) detecting at least one antibody bound to at least one peptide expressed by the display library, thereby detecting an antibody against the at least one peptide in the biological sample.

2. The method of claim 1, wherein the display library is a phage display library.

3. The method of claim 1, wherein the antibodies are immobilized to a solid support adapted for binding immunoglobulin E (IgE) subclass.

4. The method of claim 1, wherein the antibodies are immobilized by contacting the display library and antibodies from the biological sample with anti-IgE antibodies

5. The method of claim 4, wherein the anti-IgE antibodies are immobilized to a solid support.

6-7. (canceled)

8. The method of claim 1, wherein the antibodies are immobilized by contacting the display library and antibodies from the biological sample with anti-IgG antibodies.

9-11. (canceled)

12. The method of claim 1, wherein the detection of the antibody comprises a step of lysing the phage and amplifying the DNA.

13. The method of claim 1, further comprising removing unbound antibody and peptides of the display library.

14. (canceled)

15. The method of claim 1, wherein the plurality of peptides are each less than 75 amino acids long.

16. The method of claim 1, wherein deoxyribonucleic acid (DNA) within a vector of the display library encoding each peptide of the plurality of peptides comprises common adapter regions flanking the ends of the nucleic acid sequences encoding the peptides.

17. The method of claim 1, wherein at least two antibodies are detected.

18. The method of claim 17, wherein the at least two antibodies are detected simultaneously.

19. The method of claim 1, wherein the display library comprises at least 10 allergenic peptides.

20. (canceled)

21. The method of claim 1, wherein the detection step comprises amplifying DNA within the display library vector that encodes the displayed peptide.

22. The method of claim 21, further comprising the step of sequencing the amplified DNA.

23. (canceled)

24. The method of claim 21, further comprising the step of performing microarray hybridization to detect the amplified sequences.

25. (canceled)

26. The method of claim 1, wherein the detection step comprises amplifying a DNA proxy within the library display vector that encodes the displayed peptide.

27. (canceled)

28. The method of claim 26, further comprising the step of sequencing the amplified DNA proxy.

29-34. (canceled)

35. A phage library displaying a plurality of allergen peptides, wherein the plurality of allergen peptides represents a set of peptides from allergens known to affect humans.

36-46. (canceled)

47. A method of producing a library of proteins, comprising:
synthesizing synthetic oligonucleotides encoding for one or a plurality of peptides, wherein the oligonucleotides comprise from about one hundred to about three hundred nucleotides;
amplifying the oligonucleotides;
cloning the amplified oligonucleotides into a display library; thereby, producing a library of proteins.

48-58. (canceled)
